(19) United States
(12) Patent Application Publication
(10) Pub. No.: US 2025/0319243 A1
(43) Pub. Date: Oct. 16, 2025

(54) CONTRAST COMPATIBLE GUIDEWIRE AND CATHETER STACK

(71) Applicant: Imperative Care, Inc., Campbell, CA (US)

(72) Inventors: Craig Mar, Fremont, CA (US); Lilip Lau, Los Altos, CA (US); Ian Peter Van Sciver, Tollhouse, CA (US)

(21) Appl. No.: 19/249,206

(22) Filed: Jun. 25, 2025

Related U.S. Application Data
(63) Continuation-in-part of application No. 18/395,127, filed on Dec. 22, 2023.
(60) Provisional application No. 63/477,370, filed on Dec. 27, 2022.
(60) Provisional application No. 63/664,613, filed on Jun. 26, 2024.

(51) Int. Cl. A61M 5/00; A61M 5/142

(52) U.S. Cl. CPC A61M 5/007; A61M 5/142; A61M 2202/0007; A61M 2202/04; A61M 2205/3334; A61M 2205/3344; A61M 2205/50

(57) ABSTRACT

A system for delivering a fluid to a target site of a patient's vasculature having a catheter having a proximal end, a distal end, and a lumen defined by an interior surface of the catheter extending longitudinally through the catheter between the proximal end and the distal end, a guiding element having a distal end, a proximal end, and an exterior surface, the guiding element positioned in the lumen creating an area, between the exterior surface of the guiding element and the interior surface of the catheter, having an effective cross-sectional area sized fluid communication while the guiding element is in the lumen. Also, a method of performing a neurovascular procedure includes providing a multi-catheter assembly including an access catheter, a guide catheter, and a procedure catheter, coupling the assembly to a robotic drive system, and injecting contrast media into the assembly without removing a guiding element.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. The present application is a continuation-in-part of U.S. application Ser. No. 18/395,127, filed Dec. 22, 2023, which claims the benefit of U.S. Provisional Application No. 63/477,370, filed Dec. 27, 2022. The present application also claims the benefit of U.S. Provisional Application No. 63/664,613, filed Jun. 26, 2024. The entire contents of the above-listed applications are incorporated by reference herein for all purposes and forms a part of this specification.

### BACKGROUND

### Field

The present application relates to neurovascular procedures, and more particularly, to catheter assemblies and robotic control systems for neurovascular site access.

### Description of the Related Art

A variety of neurovascular procedures can be accomplished via a transvascular access, including thrombectomy, diagnostic angiography, embolic coil deployment and stent placement. However, the delivery of neurovascular care is limited or delayed by a variety of challenges. For example, there are not enough trained interventionalists and centers to meet the current demand for neurointerventions. Neurointerventions are difficult, with complex set up requirements and demands on the surgeon's dexterity. With two hands, the surgeon must exert precise control over 3-4 coaxial catheters plus manage the fluoroscopy system and patient position. Long, tortuous anatomy, requires delicate, precise maneuvers. Inadvertent catheter motion can occur due to energy storage and release caused by frictional interplay between coaxial shafts and the patient's vasculature. Supra-aortic access necessary to reach the neurovascular is challenging to achieve, especially Type III arches. For example, during current neurothrombectomy procedures physicians must remove the guidewire from the catheter to perform dye injections. Many dye injections are needed to navigate through the complex vasculature up to the brain, so guidewires must be removed over 10 times a procedure. The extra step of removing the guidewire increases the duration of the procedure increasing costs time and induces risk of introducing an air bubble to the patient. Even once supra-aortic access is achieved, adapting the system for neurovascular treatments remains time consuming, and requires guidewire and access catheter removal and addition of a procedure catheter (and possibly one or more additional catheters) to the stack.

Thus, there remains a need for a supra-aortic access and neurovascular site access system that addresses some or all these challenges and increases the availability of neurovascular procedures. Preferably, the system is additionally capable of driving devices further distally through the supra-aortic access to accomplish procedures in the intracranial vessels.

### SUMMARY

There is provided in accordance with one aspect of the present disclosure a supra-aortic access robotic control system. The supra-aortic robotic control system may deliver a fluid to a target site of a patient's vasculature. The system may include a catheter comprising a tubular shaft, a lumen extending longitudinally through the shaft, proximal and distal ends, and a guiding element, which is often a guidewire, but it can be another elongated medical device including but not limited to a catheter. The guiding element and the catheter are sized such that the guiding element can be positioned in the lumen of the catheter. For ease of reference in describing aspects of providing fluid through the lumen of the catheter to a target site while a guiding element is positioned inside the lumen, the “guiding element” is often referred to herein simply as a “guidewire” and in many uses the guiding element is a guidewire. Accordingly, unless context of the description indicates otherwise, a reference to a “guidewire” or a “guiding element” can be referring to a guidewire, a catheter, or another elongated medical device (EMD). The guidewire may be configured to remain inside the lumen of the catheter creating a space with an annular cross-section, and the guidewire may be navigated to the target site within a vasculature of a patient by the user, such that the distal end of the catheter is also advanced to the target site. A guiding element can include surfaces having different properties, for example, a hydrophilic coating on its distal end and a hydrophobic coating on its proximal end. In an example, the hydrophobic coating is polytetrafluoroethylene (PTFE) or comprises a PTFE-based composition. The term guidewire and guiding element may be used interchangeably unless the context and/or the specific use of the term indicates otherwise. In some embodiments, the distal end of the catheter may be heat shaped. In some embodiments, the distal end of the catheter may comprise a hypotube. In some embodiments, the hypotube may be laser cut. In some embodiments, the catheter wall may include a braided reinforcement layer of stainless steel wire surrounding part or all of the lumen. In some embodiments, the guidewire may have a hydrophilic coating.

The system may also include a pump, syringe or injector configured to deliver the fluid through the proximal end of the catheter at a pressure to achieve a desired or predetermined flow rate (or “target flow rate”) such that the fluid is propelled through the lumen and out of the distal end at the target site. In some embodiments, the lumen of the catheter communicating fluid may have an inside diameter (ID) of at least about 0.045″. For example, the ID may be between about 0.045″ and about 0.048″. The catheter and the guidewire are configured to allow sufficient fluid to flow through the lumen even when the guidewire is positioned all or partially in the lumen, which reduces an effective cross-sectional area of the lumen. “Effective cross-sectional area” as used herein is a broad term that refers to a cross-sectional area of a catheter lumen that is available to communicate fluid. In an example, if the lumen of a catheter does not include an object (e.g., an elongated medical device or “EMD”), the effective cross-sectional area is the cross-sectional area of the lumen. In another example, if the lumen of a catheter includes an object (e.g., an EMD such as a guiding element), the effective cross-sectional area of a catheter lumen can be determined by subtracting a cross-sectional area of the guiding element in the lumen from the cross-sectional area of the lumen of the catheter. In certain implementations of a robotic catheter system, it is desired that a fluidics system provide a contrast flow rate of, for example, at least about 1 milliliter/second (mL/second), 2 mL/second, or 3 mL/second. Also, it can be operationally advantageous to to provide contrast from a catheter without removing another device (e.g., catheter, guidewire) from the lumen of the catheter. To achieve a desired flow rate out of the distal end of a catheter while another device is in the lumen of the catheter providing the contrast, given a certain viscosity of contrast media, catheter length, and feasible pressure provided by a contrast pump, it has been discovered that such operations can achieve a desired flow rate if there is a certain effective cross-sectional area of the lumen available to communicate contrast. In some embodiments, a catheter and an EMD that can be positioned in the lumen of the catheter are structured such that the lumen of the catheter has an effective cross-sectional area greater than about 0.001 square inches (e.g., greater than about 0.001257 square inches, or greater than about 0.001407 square inches) when the EMD is positioned in the lumen. In some embodiments, the fluid may be a contrast media. In some embodiments, the guidewire in the lumen of the catheter providing the contrast may have a diameter between about 0.014 inches and about 0.024 inches.

It is to be understood that although some of the embodiments disclosed herein are described as comprising a guidewire, any suitable guiding element (for example, a guidewire, a catheter, etc.) that is able to aid in navigating a catheter through human anatomy may also be used, where the cross-sectional shape (e.g., circular) and size (e.g., diameter) of the guiding element relative to the cross-sectional shape and size of the lumen of the catheter, when the guiding element is positioned in the lumen, provides a space in the lumen sufficient for the flow of a liquid at a desired flow rate. The desired flow rate can be a predetermined flow rate. The desired flow rate of the fluid can be provided at a certain pressure for a determined amount of time to provide a desired amount of fluid (e.g., contrast media). In an example, the flow rate can be 1 mL/second. In another example, the flow rate can be 2 mL/second. In another example, the flow rate can be 3 mL/second. In some embodiments, the desired flow rate can be a predetermined rate. A predetermined rate can be a flow rate desired by a user. In some embodiments for contrast media, a predetermined flow rate of contrast media out of the distal end of a catheter is provided by a fluidic system by controlling the pressure to be applied in a contrast pump based on one or more of the viscosity of the contrast media, the effective cross-sectional area of the lumen of the catheter receiving the contrast (which is affected by an EMD in the lumen), and the length of the catheter.

There is further provided in accordance with another aspect of the present disclosure a method for delivering a fluid to a target site of a patient's vasculature, comprising navigating a guidewire to the target site, navigating an lumen of a catheter over the guidewire until a distal end of the catheter is positioned at the target site, coupling a proximal end of the catheter to a fluid pump or injector, and pumping the fluid through proximal end of the catheter, along the annular lumen, and out of the distal end to the target site. In some embodiments, the lumen may have an inside diameter of at least 0.046 inches. In some embodiments, the lumen may have an effective cross-sectional area greater than 0.001 square inches. In some embodiments, the fluid may be a contrast media. In some embodiments, the guidewire may have a diameter of 0.018 inches, 0.020 inches, or between 0.018 inches and 0.024 inches. In some embodiments, the guidewire may have a diameter of 0.024 inches, or between 0.020 inches and 0.024 inches. In some embodiments, the guidewire may have a diameter of 0.014 inches, 0.018 inches, or between 0.014 inches and 0.018 inches. In some embodiments, the distal end may be heat shaped. In some embodiments, the distal end may comprise a hypotube. In some embodiments, the hypotube may be laser cut. In some embodiments, the lumen may further comprise a braided reinforcement layer of stainless steel wire. In some embodiments, the guidewire may have a hydrophilic coating.

Another innovation includes a system for delivering fluid to a target site of a patient's vasculature at a target flow rate, the system comprising a concentric stack of elongated medical devices including a first catheter, a second catheter, a third catheter, and a guiding element. The system further includes a fluidic system including a contrast pump and a plurality of fluidic channels connecting the contrast pump to each of the first, second, and third catheter. The fluidics system can be configured to selectively provide, at an operating pressure, contrast media from the contrast pump to a lumen of the second catheter, while the third catheter or the guiding element is positioned at least partially in the lumen of the second catheter such that the provided contrast media flows out of a distal end of the second catheter at the target flow rate, or to a lumen of the third catheter, while the guiding element is positioned at least partially in the lumen of the third catheter such that the provided contrast media flows out of a distal end of the third catheter at the target flow rate. In some embodiments, the system is further configured to selectively provide contrast media from the contrast pump to the first catheter while the second catheter, the third catheter, or the guiding element is at least partially in a lumen of the first catheter such that provided contrast media flows out of a distal end of the first catheter at the target flow rate. In some embodiments, the second catheter and the third catheter are configured with dimensions such that when the third catheter is positioned in the lumen of the second catheter an effective cross-sectional area for fluid communication in the lumen of the second catheter is about 0.001 square inches or higher, wherein the second catheter and the guiding element are configured with dimensions such that when the guiding element is positioned in the lumen of the second catheter an effective cross-sectional area for fluid communication, is about 0.001 square inches or higher, and wherein the third catheter and the guiding element are configured with dimensions such that when the guiding element is positioned in the lumen of the third catheter an effective cross-sectional area for fluid communication, is about 0.001 square inches or higher. In some embodiments, the target flow rate is at least about 3 mL/second per second and the operating pressure is greater than or equal to about 300 PSI. In some embodiments, the third catheter is an insert catheter with an inner diameter of between about 0.035″ and about 0.055″ and an outer diameter of about 0.068″.

Another innovation includes a system for delivering a fluid to a target site of a patient's vasculature, the system comprising a catheter including a tubular catheter shaft having a proximal end, a distal end, and a lumen defined by an interior surface of the catheter shaft extending longitudinally through the catheter shaft between the proximal end and the distal end; a guiding element having a distal end, a proximal end, and an exterior surface, the guiding element configured to be positioned in the lumen creating an area, between the exterior surface of the guiding element and the interior surface of the catheter, having an effective cross-sectional area greater than or equal to about 0.001 square inches (e.g., 0.001257 square inches) for fluid communication, the system configured to move the catheter and guiding element such that the distal ends of the guiding element and the catheter advance towards the target site while the guiding element is at least partially in the lumen of the catheter; and a contrast pump coupled to the catheter in fluid communication with the lumen, the system configured to actuate the contrast pump to provide contrast media into the proximal end of the catheter at a pump pressure of less than or equal to about 400 PSI while the guiding element is positioned at least partially in the lumen of the catheter such that the provided contrast media propagates through the lumen of the catheter along the exterior surface of the guiding element and flows out of the distal end of the catheter, wherein the effective cross-sectional area allows a predetermined flow rate of the contrast media out of the distal end of the catheter. In some examples, the fluid is a fluid other than contrast media. In some embodiments, the predetermined flow rate is 3 cc's per second, or about 3 cc's per second. In some embodiments, the predetermined flow rate is at least 3 cc's per second. In some embodiments, the effective cross-sectional area (in the lumen when the guiding element is positioned at least partially in the catheter) can be annular shaped or eccentrically annular shaped. In some embodiments, the effective cross-sectional area can be greater than or equal to about 0.001 square inches which provides a channel in the lumen to communicate the fluid (e.g., contrast media) through the catheter and out of the distal end of the catheter at the desired rate of flow. In some specific examples of configurations, the effective cross-sectional area can be greater than or equal to about 0.001257 square inches, or greater than 0.001407 square inches which provides a channel in the lumen to communicate fluid (e.g., contrast media) through the catheter and out of the distal end of the catheter at the desired rate of flow. In an example, the desired flow rate is at least about 2 cc's per second. In another example, the desired flow rate is at least about 3 cc's per second. The guiding element can be, for example, a guidewire, a catheter, or another elongated medical device. In some examples, the guiding element has a diameter of about 0.014 inches or about 0.024 inches. In some examples, the diameter of the lumen of the catheter (i.e., the inside diameter of the catheter) is about 0.045 inches or about 0.049 inches. In some examples, the distal end of the catheter is heat shaped. In some examples, the distal end of the catheter comprises a hypotube, and the hypotube can be a laser cut hypotube. In some examples, the catheter comprises a braided reinforcement layer of stainless steel wire around at least part of the lumen. In some embodiments of a system with multiple catheters, the catheter is a first catheter and the system further comprises a second catheter and a third catheter positioned such that the guiding element, the first catheter, the second catheter and the third catheter are arranged concentrically such that at least a portion of the guiding element, first catheter, and the second catheter are inside the third catheter when providing the contrast media through the lumen of the first catheter. In some embodiments, the guiding element comprises a hydrophilic coating.

Another innovation includes a method for delivering a fluid to a target site of a patient's vasculature using a robotic catheter system, the method comprising moving a distal end of a guiding element towards the target site; moving a distal end of a catheter to the target site while at least a portion of the guiding element is positioned in a lumen of the catheter; and providing contrast media into a proximal end of the catheter while at least a portion of the guiding element is positioned in the lumen of the catheter such that the provided contrast media propagates through the lumen along an exterior surface of the guiding element and out of the distal end of the catheter, wherein the lumen and the guiding element are dimensioned to create an area, between an exterior surface of the guiding element and an interior surface of the catheter, that provides a predetermined flow rate of the contrast media out of the distal end of the catheter at a pump pressure of less than or equal to 400 PSI. The flow rate of the fluid is affected by the pump pressure and the effective cross-sectional area. Accordingly, with a pump pressure of less than or equal to 400 PSI, the effective cross-sectional area is sized such that the flow rate is, or about, 3 cc's per second. In some embodiments, the effective cross-sectional area is sized such that the predetermined flow rate is at least about 3 cc's per second. In an example, the desired flow rate is at least about 2 cc's per second. In some embodiments, the is annular shaped, or the effective cross-sectional area is eccentrically annular shaped. The method can further comprise providing contrast media while moving at least one of the guiding element or the catheter towards the target site. In some embodiments, the catheter is a first catheter and a second catheter and a third catheter positioned such that the guiding element, the first catheter, the second catheter and the third catheter are arranged concentrically such that at least a portion of the guiding element, first catheter, and the second catheter are inside the third catheter when providing the contrast media through the lumen of the first catheter. In some embodiments, the guiding element is coupled to a first hub and the catheter is coupled to a second hub, the first hub magnetically coupled to a first carriage of drive assembly through a sterile barrier, the second hub is magnetically coupled to a second carriage of the drive assembly through the sterile barrier, and wherein moving the distal end of the guiding element and the distal end of the catheter comprises moving the first carriage and moving the second carriage.

Another innovation includes a system for delivering a fluid to a target site of a patient's vasculature, the system comprising a fluidics system having a contrast pump and a plurality of contrast fluid channels to provide contrast to a plurality of catheters; a concentric stack having two or more catheters; a controller configured to selectively cause any one or more of the two or more catheters in the concentric stack to be in fluid communication with the contrast fluid channels, and actuate the contrast pump to provide contrast to any one or more of the two or more catheters. The contrast pump can be configured to provide contrast a high pressure above about 300 PSI. In some embodiments, any two adjacent catheters in the concentric stack of two or more catheters are sized to have an effective cross-sectional area between them of about 0.001257 square inches or higher. In some embodiments, the concentric stack includes three catheters and a guide wire. The three catheters can include a guide catheter, a procedure catheter, and an insert catheter. In some embodiments, the controller is configured to cause at least a portion of the procedure catheter to be positioned in the guide catheter while contrast is being injected through the lumen of the guide catheter in the space between the guide catheter and the procedure catheter. In some embodiments, the controller is configured cause at least a portion of the insert catheter to be positioned in the procedure catheter while contrast is being injected through the lumen of the procedure catheter in the space between the procedure catheter and the insert catheter. In some embodiments, the controller is configured cause at least a portion of the guide wire to be positioned in the lumen of the insert catheter while contrast is being injected through the lumen of the insert catheter in the space between the insert catheter and the guide wire.

Another innovation includes a method for delivering a fluid to a target site of a patient's vasculature using a robotic catheter system. In some embodiments, the method comprises causing movement, by a controller, of a concentric stack of elongated devices towards the target site, the concentric stack of elongated devices including two or more catheters; causing, by the controller, a selected catheter in the concentric stack of elongated devices to be in fluid communication with contrast fluid channels of a fluidics system; and actuating, by the controller, a high pressure contrast pump of the fluidics system to provide contrast at a pressure above about 300 PSI to a lumen of the selected catheter for injecting contrast into a patient while the selected catheter has positioned at least partially in its lumen another elongated device of the concentric stack of elongated devices.

Another innovation includes a method for delivering a fluid to a target site of a patient's vasculature using a robotic catheter system, the method comprising moving a distal end of a concentric stack of elongated devices towards the target site, the concentric stack of elongated devices including two or more catheters; selectively causing, by a controller, any one or more of the two or more catheters in the concentric stack to be in fluid communication with contrast fluid channels of a fluidics system; and actuating a high pressure contrast pump of the fluidics system to selectively provide contrast to a lumen of any one or more of the two or more catheters while the lumen contains a catheter or a guide wire of the concentric stack of elongated devices. The high pressure contrast pump can provide contrast at above about 300 PSI.

Another innovation includes a method for delivering a fluid to a target site of a patient's vasculature using a robotic catheter system, the method comprising moving a distal end of a concentric stack of elongated devices towards the target site, the concentric stack of elongated devices including two or more catheters; selectively causing, by a controller, any one or more of the two or more catheters in the concentric stack to be in fluid communication with contrast fluid channels of a fluidics system; and actuating a high pressure contrast pump of the fluidics system to selectively provide contrast to a lumen of any one or more of the two or more catheters while the lumen contains a catheter or a guide wire of the concentric stack of elongated devices such that contrast flows out of a distal end of the lumen at 1 mL/second or greater. In some embodiments, the flow rate is at least about 2 mL/second. In some embodiments, the flow rate is at least about 3 mL/second. In some embodiments, the lumen that contains a catheter or a guidewire of the concentric stack of elongated devices has an effective cross-sectional area for fluid communication of greater than or equal to about 0.001 square inches. In some embodiments of such methods, actuating a high pressure contrast pump includes operating the contrast pump to provide contrast at a pressure of greater than 250 PSI. In some embodiments of such methods, actuating a high pressure contrast pump includes operating the contrast pump to provide contrast at a pressure of less than or equal to about 400 PSI.

### BRIEF DESCRIPTION OF THE DRAWINGS

FIG. 1 is a schematic perspective view of an interventional setup having an imaging system, a patient support table, and a robotic drive system in accordance with the present disclosure.

FIG. 2 is a longitudinal cross section showing the concentric relationship between a guidewire having two degrees of freedom, an access catheter having 3 degrees of freedom and a guide catheter having one degree of freedom.

FIG. 3A is an exploded schematic view of interventional device hubs separated from a support table by a sterile barrier.

FIGS. 3B-3F show an alternate sterile barrier in the form of a shipping tray having one or more storage channels for carrying interventional devices.

FIGS. 3G-3K show embodiments of an alternate sterile barrier having a convex drive surface.

FIGS. 3L and 3M depict an example of a hub that may be used with the sterile barriers of FIGS. 3G-3K.

FIG. 4 is a schematic elevational cross section through a hub adapter having a drive magnet separated from an interventional device hub and driven magnet by a sterile barrier.

FIGS. 5A and 5B schematically illustrate a three interventional device and a four interventional device assembly.

FIG. 6 is a perspective view of a support table.

FIG. 7 is a close-up view of the motor drive end of a support table.

FIG. 8 is an elevational cross section through a motor and belt drive assembly.

FIG. 9 is a close-up view of a pulley end of the support table.

FIG. 10 is an elevational cross section through a belt pully.

FIG. 11 is a side elevational cross-section through a distal portion of a catheter such as any of those shown in FIGS. 5A and 5B.

FIGS. 12A and 12B schematically illustrate a force sensor integrated into the sidewall of the catheter.

FIGS. 13A and 13B schematically illustrate a sensor for measuring elastic forces at the magnetic coupling between the hub and corresponding carriage.

FIG. 14 schematically illustrates a dual encoder torque sensor for use with a catheter of the present disclosure.

FIG. 15 illustrates a clot capture and visualization device that can be integrated into a hub and/or connected to an aspiration line.

FIGS. 16A-16C illustrate an example control mechanism for manipulating interventional devices driven by respective hubs.

FIG. 17 illustrates a side elevational schematic view of an interventional device assembly for supra-aortic access and neuro-interventional procedures.

FIG. 18 illustrates a side cross-sectional view of an example of two catheters where at least a portion of catheters (e.g., the tip) is a laser cut hypotube.

FIG. 19 shows a magnified view of an example of a laser cut hypotube.

FIGS. 20A-20E depict an example sequence of steps of introducing a catheter assembly configured to achieve supra-aortic access and neurovascular site access.

FIG. 21 schematically illustrates an embodiment of a mechanical coupling between a drive mechanism and a driven mechanism.

FIGS. 22A-22C depict an example sequence of steps of priming a catheter assembly in a stacked configuration.

FIGS. 23A-23B depict an example sequence of steps of priming a catheter assembly in a stacked configuration.

FIG. 24 depicts an example test system for the priming process depicted in FIGS. 23A-23B.

FIG. 25A depicts an example of a catheter assembly.

FIG. 25B depicts an example of a catheter assembly after a priming procedure.

FIG. 25C depicts an example of a catheter assembly after a priming procedure including relative movement between adjacent catheters.

FIG. 25D-F illustrate the example catheter assembly of FIGS. 25A-25C.

FIG. 26A illustrates one embodiment of a one channel fluidics management system.

FIG. 26B illustrates one embodiment of a three channel fluidics management system.

FIG. 27 shows a schematic of an example of a three channel fluidics system.

FIG. 28 shows another schematic of an example of a fluidics system.

FIG. 29A shows another schematic of an example of a fluidics system.

FIG. 29B is a schematic illustrating an example of a robotic catheter system that includes another embodiment of a fluidics system.

FIG. 29C is a schematic illustrating an example of a fluidics assembly that includes a cassette that couples to a pump station and couples to a saline source, a contrast source, and a vacuum source, the fluidics assembly also including a plurality of fluid communication channels to provide saline, contrast, and vacuum to mounts and catheters coupled to the mounts.

FIG. 29D is a schematic illustrating an example of a fluidics assembly that includes a cassette, a splitter, and one or more mounts, a first tubing set coupled to the cassette and splitter, and a second tubing set coupled between the splitter and the one or more mounts, the first tubing set having channels for providing saline, contrast and vacuum to the splitter, and the second tubing set having groups of channels (or subchannels) providing saline, contrast, and vacuum to each of the one or more mounts, the first and second tubing set and the splitter also providing electrical connections between the cassette and one or more connected mounts.

FIG. 29E is a schematic illustrating an example of a cassette of the fluidics assembly, the cassette including a housing that can support, all or part of a saline subsystem, a contrast subsystem, and a vacuum subsystem.

FIG. 29F is a schematic illustrating an example of a fluidic system components in one or more mounts, which can be coupled to the second tubing set, this particular example including three mounts each having a fluidic connection for saline, contrast, and vacuum, and an electrical connection that includes one or more electrical leads, the electrical connection coupled to one or more sensors in the mounts to communicate signals from the one or more sensors to the cassette, and ultimately a station (e.g., a pump station) when the cassette is coupled to the station.

FIG. 30 illustrates an example of an example of a fluidics system.

FIG. 31 illustrates an example of an embodiment of a saline subsystem.

FIG. 32 illustrates an example of an embodiment of a contrast subsystem.

FIG. 33 illustrates an example of an embodiment of a vacuum/aspiration (“V/A”) subsystem (“vacuum subsystem”).

FIG. 34 illustrates an example of a catheter coupled to an embodiment of a hub, and a tubing set connected at a distal end to the hub and at a proximal end to a cassette, where the tubing set includes a saline tube, a contrast tube, and a vacuum tube, and can include electrical connections, where the tubing set forms a portion of a fluid communication system that connects the hub and to a saline subsystem, a contrast subsystem, and a vacuum subsystem.

FIG. 35 illustrates another example of a catheter coupled to an embodiment of a hub.

FIG. 36 illustrates an example of a tubing set that is a portion of a fluid communication system that provides channels for communicating substances (e.g., air, fluids, and/or materials) between a plurality of catheters and a saline subsystem, a contrast subsystem, and a vacuum subsystem, the tubing set coupled, on a proximal end of the tubing set, to a cassette and coupled, on a distal end of the tubing set, to a plurality of hubs, and in this example the tubing set also includes electrical connections between the cassette and the plurality of hubs.

FIG. 37 illustrates a schematic of an example of a robotic catheter system that includes a remotely located system (“remote system”) and a locally located system (“local system”), and illustrates an example of certain components of the local system that includes certain components of a fluidic management system (“fluidics system”) including actuatable components that are actuated by a controller and sensors that provide information to a controller for the controller to control the fluidics systems and other aspects of the robotic catheter system, according to some embodiments.

FIG. 38 illustrates an example of an embodiment of a cassette and a pump station illustrating certain components of the cassette (e.g., valves, electrical connections) and corresponding components (e.g., motors, electrical connections) of the pump station.

FIG. 39 illustrates an example of a contrast injection process that can be performed when the fluidic system is in state contrast injection.

FIG. 40 illustrates a process for determining if it's safe for the system to inject contrast.

FIG. 41 illustrates an example of the contrast subsystem configuration where an insert catheter coupled to hub has been selected for injecting contrast.

FIG. 42 illustrates an example of a configuration of the hub corresponding to the selected catheter when contrast is injected.

FIG. 43 illustrates an example of a configuration of the hub of a selected catheter configured to inject a saline bolus.

FIG. 44 illustrates an overview of an example trial procedure for a contrast injection study.

FIG. 45 is a graphical representation of the relationship between flow rate and catheter length.

FIG. 46 is a graphical representation of the relationship between flow rate and guidewire diameter.

FIG. 47 is a graphical representation of the relationship between output flow rate and input flow rate.

FIG. 48 is a graphical representation of the relationship between output flow rate and injection volume.

FIG. 49 is a graphical representation of the relationship between pressure, flow rate and viscosity.

FIG. 50 depicts a schematic of an example of a control system.

FIG. 51 depicts a setup of an example test system for glycerin viscosity testing.

FIG. 52 is a representation of an example of a distal end view of a concentric stack of one or more elongated medical devices (EMDs) that can be used in the systems and methods disclosed herein, this example, including a guide catheter, a procedure catheter, an insert catheter, and a guiding element (e.g., a guidewire).

FIG. 53 is a representation of an example of a distal end view of a concentric stack of one or more elongated medical devices (EMDs) that can be used in the systems and methods disclosed herein for a medical procedure including injecting contrast, this example, including a guide catheter, a procedure catheter, an insert catheter, and a guiding element (e.g., a guidewire).

FIG. 54 is a representation of an example of a distal end view of a concentric stack (“stack”) of one or more elongated medical devices that can be in a patient during contrast injection through a guide catheter.

FIG. 55 is a representation of an example of a distal end view of a concentric stack of one or more elongated medical devices that can be in a patient during contrast injection through a guide catheter.

FIG. 56 is a representation of an example of a distal end view of a concentric stack of one or more elongated medical devices that can be in a patient during contrast injection through a guide catheter.

FIG. 57 is a representation of an example of a distal end view of a concentric stack of one or more elongated medical devices that can be used in a medical procedure during contrast injection through a guide catheter.

FIG. 58 is a representation of an example of a distal end view of a concentric stack of one or more elongated medical devices that can be used in a medical procedure during contrast injection through a procedure catheter.

FIG. 59 is a representation of an example of a distal end view of a concentric stack of one or more elongated medical devices that can be used in a medical procedure during contrast injection through a procedure catheter.

FIG. 60 is a representation of an example of a distal end view of a concentric stack of one or more elongated medical devices that can be used in a medical procedure during contrast injection through a procedure catheter.

FIG. 61 is a representation of an example of a distal end view of a concentric stack of one or more elongated medical devices that can be used in a medical procedure during contrast injection through a procedure catheter.

FIG. 62 is a representation of an example of a distal end view of a concentric stack of one or more elongated medical devices that can be used in a medical procedure during contrast injection through an insert catheter.

FIG. 63 is a representation of an example of a distal end view of a concentric stack of one or more elongated medical devices that can be used in a medical procedure during contrast injection through an insert catheter but not including the guiding element (depicted in a dashed line), and optionally including a guide catheter and a procedure catheter, the contrast being injected into the patient through the lumen of the insert catheter.

FIG. 64 is a representation of an example of a distal end view of a concentric stack of one or more elongated medical devices that can be used in a medical procedure during contrast injection through an insert catheter but not including the guiding element (depicted in a dashed line), and optionally including a guide catheter and a procedure catheter, the contrast being injected into the patient through the lumen of the insert catheter.

### DETAILED DESCRIPTION

In certain embodiments, a system is provided for advancing a guide catheter from a femoral artery or radial artery access into the ostium of one of the great vessels at the top of the aortic arch, thereby achieving supra-aortic access. A surgeon can then take over and advance interventional devices into the cerebral vasculature via the robotically placed guide catheter.

In some implementations, the system may additionally be configured to robotically gain intra-cranial vascular access and to perform an aspiration thrombectomy or other neuro vascular procedure.

A drive table can be positioned over or alongside the patient, and configured to axially advance, retract, and in some cases rotate and/or laterally deflect two or three or more different (e.g., concentrically or side by side oriented) intravascular devices. The hub is moveable along a path along the surface of the drive table to advance or retract the interventional device as desired. Each hub may also contain mechanisms to rotate or deflect the device as desired and is connected to fluid delivery tubes (not shown) of the type conventionally attached to a catheter hub. Each hub can be in electrical communication with an electronic control system, either via hard wired connection, RF wireless connection or a combination of both.

Each hub is independently movable across the surface of a sterile field barrier membrane carried by the drive table. In some embodiments, each hub is releasably magnetically coupled to a unique drive carriage on the table side of the sterile field barrier. The drive system independently moves each hub in a proximal or distal direction across the surface of the barrier, to move the corresponding interventional device proximally or distally within the patient's vasculature.

The carriages on the drive table, which magnetically couple with the hubs to provide linear motion actuation, are universal. Functionality of the catheters/guidewire are provided based on what is contained in the hub and the shaft designs. This allows flexibility to configure the system to do a wide range of procedures using a wide variety of interventional devices on the same drive table. Additionally, the interventional devices and methods disclosed herein can be readily adapted for use with any of a wide variety of other drive systems (e.g., any of a wide variety of robotic surgery drive systems).

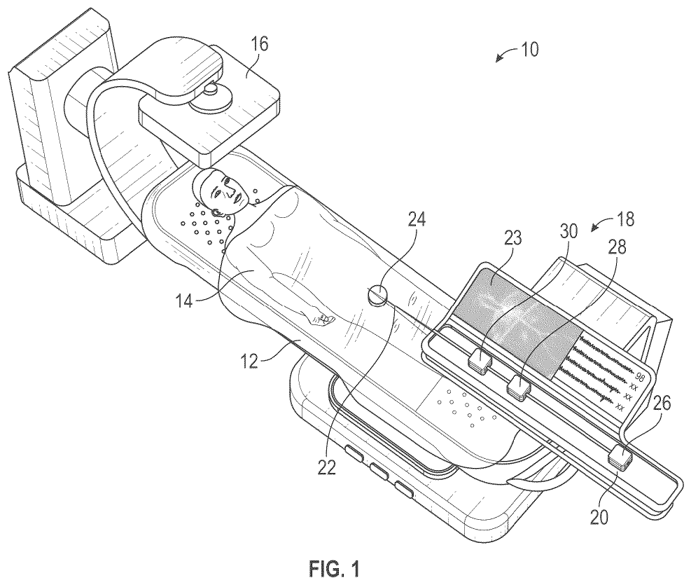

FIG. 1 is a schematic perspective view of an interventional setup 10 having a patient support table 12 for supporting a patient 14. An imaging system 16 may be provided, along with a robotic interventional device drive system 18 in accordance with the present disclosure.

The drive system 18 may include a support table 20 for supporting, for example, a guiding element (‘guidewire’) hub 26, an access catheter hub 28 and a guide catheter hub 30. In the present context, the term ‘access’ catheter can be any catheter having a lumen with at least one distally facing or laterally facing distal opening, which may be utilized to aspirate thrombus, provide access for an additional device to be advanced therethrough or therealong, or to inject saline or contrast media or therapeutic agents. In the present context, term ‘guiding element’ is a broad term that refers to an elongated guiding element, for example, a guidewire, a catheter, or another elongated medical device. For ease of reference, as used herein ‘guiding element’ is used synonymously with ‘guidewire’ unless otherwise indicated explicitly or by context.

More or fewer interventional device hubs may be provided depending upon the desired clinical procedure. For example, in certain embodiments, a diagnostic angiogram procedure may be performed using only a guidewire hub 26 and an access catheter hub 28 for driving a guidewire and an access catheter (in the form of a diagnostic angiographic catheter), respectively. Multiple interventional devices 22 extend between the support table 20 and (in the illustrated example) a femoral access point 24 on the patient 14. Depending upon the desired procedure, access may be achieved by percutaneous or cut down access to any of a variety of arteries or veins, such as the femoral artery or radial artery. Although disclosed herein primarily in the context of neuro vascular access and procedures, the robotic drive system and associated interventional devices can readily be configured for use in a wide variety of additional medical interventions, in the peripheral and coronary arterial and venous vasculature, gastrointestinal system, lymphatic system, cerebral spinal fluid lumens or spaces (such as the spinal canal, ventricles, and subarachnoid space), pulmonary airways, treatment sites reached via trans ureteral or urethral or fallopian tube navigation, or other hollow organs or structures in the body (for example, in intra-cardiac or structural heart applications, such as valve repair or replacement, or in any endoluminal procedures).

A display 23 such as for viewing fluoroscopic images, catheter data (e.g., fiber Bragg grating fiber optics sensor data or other force or shape sensing data) or other patient data may be carried by the support table 20 and or patient support 12. Alternatively, the physician input/output interface including display 23 may be remote from the patient, such as behind radiation shielding, in a different room from the patient, or in a different facility than the patient.

In the illustrated example, a guidewire hub 26 is carried by the support table 20 and is moveable along the table to advance a guidewire into and out of the patient 14. An access catheter hub 28 is also carried by the support table 20 and is movable along the table to advance the access catheter into and out of the patient 14. The access catheter hub may also be configured to rotate the access catheter in response to manipulation of a rotation control and may also be configured to laterally deflect a deflectable portion of the access catheter, in response to manipulation of a deflection control.

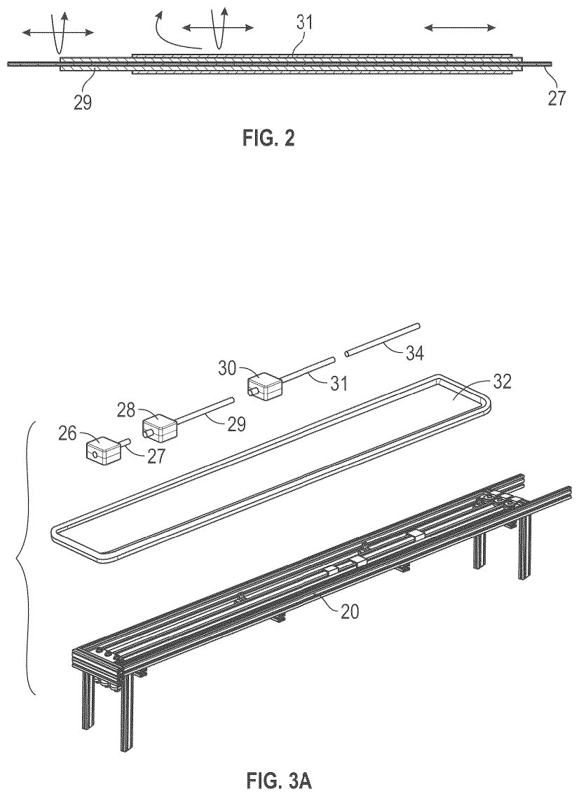

FIG. 2 is a longitudinal cross section schematically showing the motion relationship between a guidewire 27 having two degrees of freedom (axial and rotation), an access catheter 29 having three degrees of freedom (axial, rotational and lateral deflection) and a guide catheter 31, having one degree of freedom (axial).

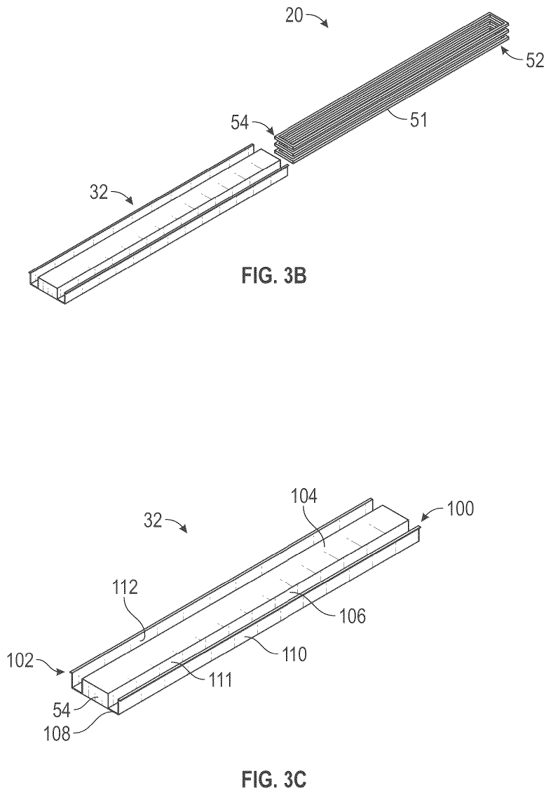

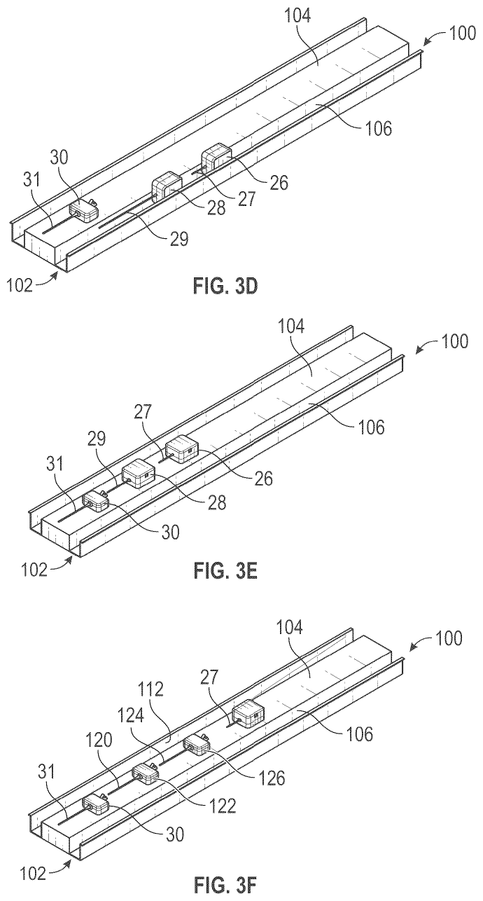

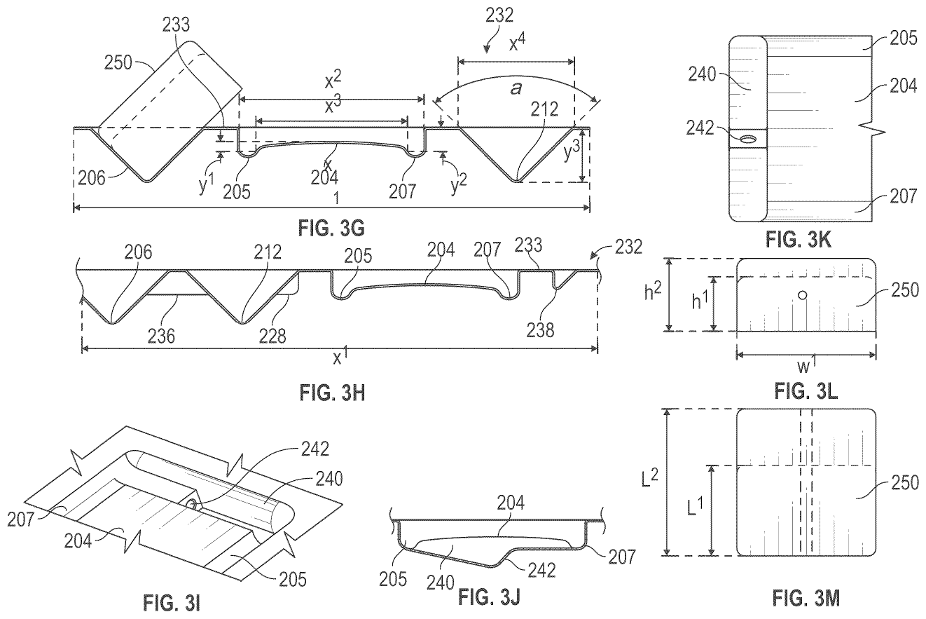

Referring to FIG. 3A, the support table 20 includes a drive mechanism described in greater detail below, to independently drive the guidewire hub 26, access catheter hub 28, and guide catheter hub 30. An anti-buckling feature 34 may be provided in a proximal anti-buckling zone for resisting buckling of the portion of the interventional devices spanning the distance between the support table 20 and the femoral artery access point 24. The anti-buckling feature 34 may comprise a plurality of concentric telescopically axially extendable and collapsible tubes through which the interventional devices extend.

Alternatively, a proximal segment of one or more of the device shafts may be configured with enhanced stiffness to reduce buckling under compression. For example, a proximal reinforced segment may extend distally from the hub through a distance of at least about 5 centimeters or 10 centimeters but typically no more than about 120 centimeters or 100 centimeters to support the device between the hub and the access point 24 on the patient. Reinforcement may be accomplished by using metal or polymer tubing or embedding at least one or two or more axially extending elements into the wall of the device shafts, such as elongate wires or ribbons. In some implementations, the extending element may be hollow and protect from abrasion, buckling, or damage at the inputs and outputs of the hubs. In some embodiments, the hollow extending element may be a hollow and flexible coating attached to a hub. The hollow, extending element (e.g., a hollow and flexible coating) may cover a portion of the device shaft when threaded through the hubs. In some embodiments, the hollow extending element is a set of telescoping portions that nest inside each other and enclose a shaft between the hubs. In some embodiments, the hollow extending element has a proximal (closest to insertion point) and distal end (farthest from insertion point) and each end is coupled to a hub. In some embodiments, the extending element is releasably coupled to a hub on at least one end. In some embodiments in which the hollow extending element is a coating, the coating may be attached to a portion of a hub such that threading the catheter device through the hub 26, 28, or 30 threads the catheter device through the coating as well. In some implementations, an anti-buckling device may be installed on or about or surrounding a device shaft to avoid misalignment or insertion angle errors between hubs or between a hub and an insertion point. The anti-buckling device may be a laser cut hypotube, a spring, telescoping tubes, tensioned split tubing, or the like.

In some implementations, a number of deflection sensors may be placed along a catheter length to identify buckling. Identifying buckling may be performed by sensing that a hub is advancing distally, while the distal tip of the catheter or interventional device has not moved. In some implementations, the buckling may be detected by sensing that an energy load (e.g., due to friction) has occurred between catheter shafts.

Alternatively, thin tubular stiffening structures can be embedded within or carried over the outside of the device wall, such as a tubular polymeric extrusion or length of hypo-tube. Alternatively, a removable stiffening mandrel may be placed within a lumen in the proximal segment of the device, and proximally removed following distal advance of the hub towards the patient access site, to prevent buckling of the proximal shafts during distal advance of the hub. Alternatively, a proximal segment of one or more of the device shafts may be constructed as a tubular hypo tube, which may be machined (e.g., with a laser) so that its mechanical properties vary along its length. This proximal segment may be formed of stainless steel, nitinol, and/or cobalt chrome alloys, optionally in combination with polymer components which may provide for lubricity and hydraulic sealing. In some embodiments, this proximal segment may be formed of a polymer, such as polyether ether ketone (PEEK). Alternatively, the wall thickness or diameter of the interventional device can be increased in the anti-buckling zone.

In certain embodiments, a device shaft having advanced stiffness (e.g., axially and torsionally) may provide improved transmission of motion from the proximal end of the device shaft to the distal end of the device shaft. For example, the device shafts may be more responsive to motion applied at the proximal end. Such embodiments may be advantageous for robotic driving in the absence of haptic feedback to a user.

In some embodiments, a flexible coating can be applied to a device shaft and/or hub to reduce frictional forces between the device shaft and/or hub and a second device shaft when the second device shaft passes therethrough.

The interventional device hubs may be separated from the support table 20 by sterile barrier 32. Sterile barrier 32 may comprise a thin plastic membrane such as polyethylene terephthalate (PET), polyethylene terephthalate glycol (PETG), polyethylene terephthalate (PETE), high-density polyethylene (HDPE), polyvinyl chloride (PVC), low-density polyethylene (LDPE), polypropylene (PP), polystyrene (PS), or styrene. This allows the support table 20 and associated drive system to reside on a non-sterile (lower) side of sterile barrier 32. The guidewire hub 26, access catheter hub 28, guide catheter hub 30 and the associated interventional devices are all on a sterile (top) side of the sterile barrier 32. The sterile barrier is preferably waterproof and can also serve as a tray used in the packaging of the interventional devices, discussed further below. The interventional devices can be provided individually or as a coaxially preassembled kit that is shipped and stored in the tray and enclosed within a sterile packaging.

FIGS. 3B-3F schematically illustrate an alternate sterile barrier in the form of a dual function sterile barrier for placement on the support table during the interventional procedure, and shipping tray, having one or more storage channels for carrying sterile interventional devices. The sterile barrier may also act as a sterile work surface for preparation of catheters or other devices during a procedure.

Referring to FIGS. 3B and 3C, there is illustrated a sterile barrier 32 in the form of a pre-shaped tray, for fitting over an elongate support table 20. In use, the elongate support table 20 would be positioned below the sterile barrier 32. The sterile barrier 32 extends between a proximal end 100 and a distal end 102 and includes an upper support surface 104 for supporting the interventional device hubs. In one implementation, the support surface 104 has an axial length greater than the length of the intended interventional devices, in a linear drive configuration.

The length of support surface 104 will typically be at least about 100 centimeters and within the range of from about 100 centimeters to about 2.7 meters. Shorter lengths may be utilized in a system configured to advance the drive couplers along an arcuate path. In some embodiments, two or more support surfaces may be used instead of a single support surface 104. The two or more support surfaces may have a combined length between 100 centimeters to about 2.7 meters. The width of the linear drive table is preferably no more than about 30 to about 80 centimeters.

At least a first channel 106 may be provided, extending axially at least a portion of the length of the support table 20. In the illustrated implementation, first channel 106 extends the entire length of the support table 20. Preferably, the first channel 106 has a sufficient length to hold the interventional devices, and sufficient width and depth to hold the corresponding hubs (for example, by providing lateral support to prevent dislodgment of the hubs when forces are applied to the hubs). First channel 106 is defined within a floor 108, outer side wall 110 and inner side wall 111, forming an upwardly facing concavity. Optionally, a second channel 112 may be provided. Second channel 112 may be located on the same side or the opposite side of the upper support surface 104 from the first channel 106. Two or three or more additional recesses such as additional channels or wells may be provided, to hold additional medical devices or supplies that may be useful during the interventional procedure as well as to collect fluids and function as wash basins for catheters and related devices.

Referring to FIG. 3D, the guide catheter hub 30 is shown positioned on the upper support surface 104, and magnetically coupled to the corresponding coupler holding the drive magnets, positioned beneath the sterile barrier 32. The access catheter hub 28 and access catheter 29, and guidewire hub 26 and guidewire 27 are illustrated residing within the first channel 106 such as before introduction through the guide catheter 31 or following removal from the guide catheter 31.

The interventional devices may be positioned within the channel 106 and enclosed in a sterile barrier for shipping. At the clinical site, an upper panel of the sterile barrier may be removed, or a tubular sterile barrier packaging may be opened and axially removed from the support table 20 and sterile barrier 32 assembly, exposing the sterile top side of the sterile barrier tray and any included interventional devices. The interventional devices may be separately carried in the channel, or preassembled into an access assembly or procedure assembly, discussed in additional detail below.

FIGS. 3D-3F illustrate the support table with sterile barrier in place, and in FIG. 3E, the interventional devices configured in an access assembly for aortic access, following coupling of the access assembly to the corresponding carriages beneath the sterile barrier. The access assembly may be preassembled with the guidewire fully advanced through the access catheter which is in turn fully advanced through the guide catheter. In embodiments in which the access catheter or other catheters are pre-shaped (i.e., pre-curved or not straight), the guidewire and/or outer catheters may be positioned so that relatively stiff sections are not superimposed with curved stiffer sections of the pre-shaped catheter, for example, to avoid creep or straightening of the pre-shaped catheter and/or introduction of a curve into an otherwise straight catheter. This access assembly may be lifted out of the channel 106 and positioned on the support surface 104 for coupling to the respective drive magnets and introduction into the patient. The guide catheter hub 30 is the distal most hub. Access catheter hub 28 is positioned proximally of the guide catheter hub, so that the access catheter 29 can extend distally through the guide catheter. The guidewire hub 26 is positioned most proximally, in order to allow the guidewire 27 to advance through the access catheter 29 and guide catheter 31.

A procedure assembly is illustrated in FIG. 3F following introduction of the procedure assembly through the guide catheter 31 that was used to achieve supra-aortic access. In this implementation, guide catheter 31 remains the distal most of the interventional devices. A first procedure catheter 120 and corresponding hub 122 is illustrated extending through the guide catheter 31. An optional second procedure catheter 124 and corresponding hub 126 is illustrated extending through the first procedure catheter 120. The guidewire 27 extends through at least a portion of the second procedure catheter 124 in a rapid exchange version of second procedure catheter 124, or the entire length of second procedure catheter 124 in an over the wire implementation.

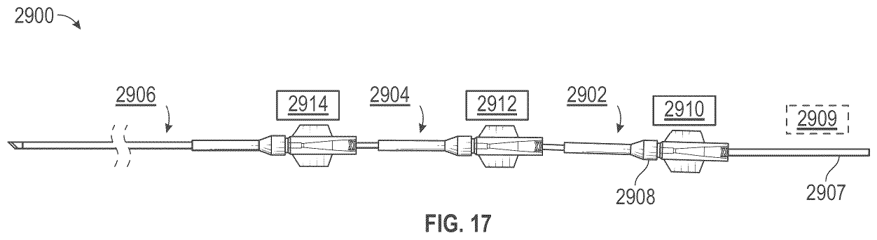

As is discussed in greater detail in connection with FIG. 17, the multi catheter stack may be utilized to achieve both access and the intravascular procedure without the need for catheter exchange. This may be accomplished in either a manual or a robotically driven procedure. In some embodiments, determination of a suitable catheter stack each having certain dimensions, along with a fluidics system configured to perform contrast injections utilizing the configuration of the catheter stack facilitates access, intravascular procedure, and contrast injection without the need for catheter exchange. In one example, the guide catheter 31 may comprise a catheter having an inner diameter of at least about 0.08 inches and in one implementation about 0.088 inches. The first procedure catheter 120 may comprise a catheter having an inner diameter within the range of from about 0.065 inches to about 0.075 inches and in one implementation catheter 120 has an inner diameter of about 0.071 inches. The second procedure catheter 124 may be an access catheter having an OD sized to permit advance through the first procedure catheter 120. The second procedure catheter may be steerable, having a deflection control 2908 configured to laterally deflect a distal end of the catheter. The second procedure (access) catheter may also have an inner lumen sized to allow an appropriately sized guidewire to remain inside the second procedure catheter while performing contrast injections through the second procedure catheter.

In certain embodiments, the catheter 31 may be a ‘large bore’ access catheter or guide catheter having an inner diameter of at least about 0.075 inches or at least an inner diameter of about 0.080 inches. The catheter 120 may be an aspiration catheter having an inner diameter within the range of from about 0.060 to about 0.075 inches. The catheter 124 may be a steerable catheter with a deflectable distal tip, having an inner diameter within the range of from about 0.025 to about 0.050 inches. The guidewire (or guiding element) 27 may have an outer diameter within the range of from about 0.014 to about 0.020 inches. In one example, the catheter 31 may have an inner diameter of about 0.088 inches, the catheter 120 an inner diameter of about 0.071 inches, the catheter 124 an inner diameter of about 0.035 inches, and the guidewire 27 may have an outer diameter of about 0.018 inches. In another example, the catheter 31 may have an inner diameter of about 0.088 inches, the catheter 120 an inner diameter of about 0.071 inches, the catheter 124 an inner diameter of about 0.045 inches, and the guidewire 27 may have an outer diameter of about 0.018 inches.

In one commercial execution, a preassembled access assembly (guide catheter, access catheter and guidewire) may be carried within a first channel on the sterile barrier tray and a preassembled procedure assembly (one or two procedure catheters and a guidewire) may be carried within the same or a different, second channel on the sterile barrier tray. One or two or more additional catheters or interventional tools may also be provided, depending upon potential needs during the interventional procedure.

FIGS. 3G-3K illustrate embodiments of an alternate sterile barrier having a convex drive surface (e.g., a convex, crowned road like drive surface). FIG. 3G is a cross-sectional view of a sterile barrier 232. The sterile barrier 232 includes a convex upper support surface 204. Fluid channels 205 and 207 are positioned laterally of and below the support surface 204 for self-clearing or draining of fluids from the support surface 204 (for example, during an interventional procedure). The fluid channels 205 and 207 may extend axially at least a portion of the length of the sterile barrier.

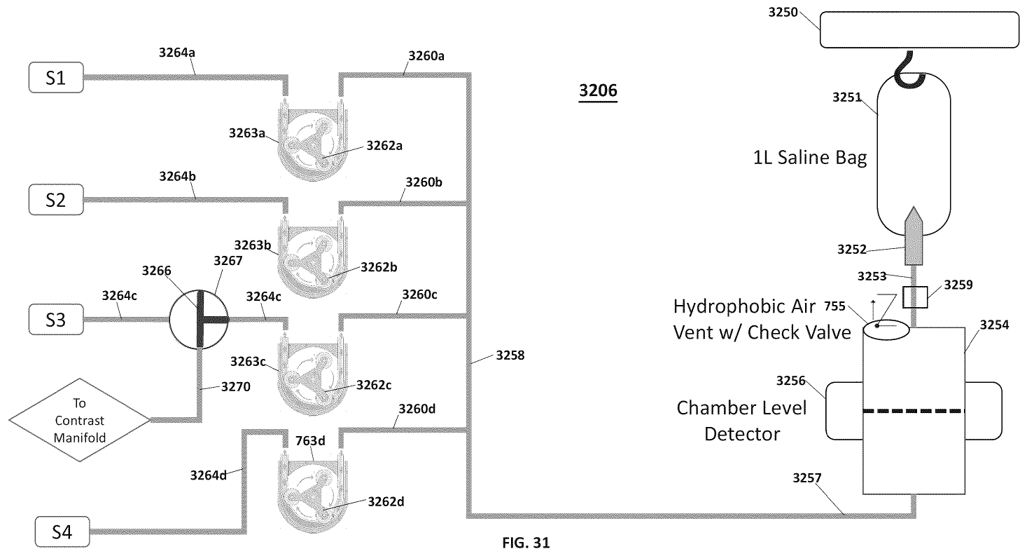

FIGS. 3I, 3J, and 3K illustrate a sectional perspective view, a cross-sectional view, and a top sectional view, respectively, of a proximal end of the sterile barrier 232. As shown, in FIGS. 31-3K, the sterile barrier 232 can include a trough 240 in communication with the fluid channels 205 and 207. The trough 240 can receive fluids from the channels 205 and 207 (for example, during an interventional procedure). The trough 240 may be positioned at least partially below the fluid channels 205 and 207 so that fluid within the channels 205 and 207 flows into the trough 240. In certain embodiments, the fluid channels 205 and 207 may be angled relative to a horizontal plane (for example, may decline from an end of the channel furthest from the trough 240 to the trough 240) so that fluid within the channels 205 and 207 is directed to the trough 240. For example, the channels 205 and 207 may increase in depth from an end of the channels furthest from the trough 240 to the trough 240. Alternatively, the sterile barrier 232 and/or support table may be positioned at an angle relative to a horizontal plane, during part of or an entirety of an interventional procedure, such that the end of the channels 205 and 207 furthest from the trough 240 is positioned higher than the trough 240. For example, the sterile barrier 232 and/or support table may be constructed or arranged in an angled arrangement so that an end of the sterile barrier 232 and/or support table opposite the trough 240 is positioned higher than the trough 240. Alternatively or additionally, a drive mechanism may temporarily tilt the sterile barrier 232 and/or support table so that an end of the sterile barrier 232 and/or support table opposite the trough 240 is positioned higher than the trough 240 (for example, by lifting an end of the sterile barrier and/or support table opposite the trough 240 or lowering an end of the sterile barrier 232 and/or support table at which the trough 240 is positioned) so that fluids within the channels 205 and 207 flow into the trough 240.

The trough 240 can include a drain hole 242. The trough 240 can be shaped, dimensioned, and/or otherwise configured so that fluid within the trough 240 empties to the drain hole 242. The drain hole 242 can include tubing, a barb fitting, and/or an on-off valve for removal of fluids from the trough 240. As shown in FIGS. 31-3K, the trough 240 can be positioned at the proximal end of the sterile barrier 232. In alternate embodiments, the trough 240 may be positioned at a distal end of the sterile barrier 232. In some embodiments, the sterile barrier 232 can include a first trough 240 at the proximal end and a second trough 240 at the distal end. In some embodiments, the trough 240 can also be used as a wash basin.

A first channel 206 may extend axially at least a portion of the length of the sterile barrier 232. The channel 206 can have a sufficient length to hold the interventional devices, and sufficient width and depth to hold the corresponding hubs (for example, by providing support to prevent dislodgement of the hubs when forces are applied to the hubs). Optionally, a second channel 212 may be provided. The second channel 212 may be located on the same side or the opposite side of the upper support surface 204 from the first channel 206. FIG. 3G illustrates the channel 212 located on the opposite side of the support surface 204 from the channel 206. FIG. 3H is a cross-sectional view illustrating an alternate embodiment of the sterile barrier 232 in which the channel 212 is on the same side of the support surface 204 as the channel 206.

As shown in FIGS. 3G and 3H, the channels 206 and 212 can have generally triangular, wedge-shaped, or otherwise angled cross-sections, so as to hold the hubs at an angle relative to a horizontal plane. Holding the hubs at an angle relative to the horizontal plane can allow for smaller width of the sterile barrier 232.

Two or three or more additional recesses such as additional channels or wells may be provided, to hold additional medical devices or supplies that may be useful during the interventional procedure as well as to collect fluids and function as wash basins for catheters and related devices.

In some embodiments, the sterile barrier 232 can include one or more structural ribs 236. The sterile barrier 232 can further include one or more frame support bosses 228 and 238.

In the embodiment of the sterile barrier 232 shown in FIG. 3G, a width x₁ can be 14 in, about 14 in, between 12 in and 16 in, between 10 in and 18 in, or any other suitable width. In the embodiment of the sterile barrier 232 shown in FIG. 3H, the width x₁ can be 15 in, about 15 in, between 13 in and 17 in, between 11 in and 19 in, or any other suitable width. A height y₁ of the support surface 204 can be 0.125 in, about 0.125 in, between 0.1 and 0.15 in, or any other suitable height. In some embodiments, the support surface 204 can be recessed from a top surface 233 of the sterile barrier 232. A height y₂ between a bottom of the support surface 204 and the top surface 233 can be 0.5 in, about 0.5 in, between 0.25 in and 0.75 in, or any other suitable height. A width x₂ from a lateral edge of the channel 205 to a lateral edge of the channel 207 can be 5 in, about 5 in, between 4 in and 6 in, or any other suitable width. A width x₃ of the support surface 204 can be 4 in, about 4 in, between 3 in and 5 in, or any other suitable width. A height y₃ of the channel 206 and/or channel 212 can be 1.5 in, about 1.5 in, between 1 in and 2 in, or any other suitable height. A width x₄ of the channel 206 and/or channel 212 can be 3 in, about 3 in, between 2 in and 4 in, or any other suitable width. The channel 206 and/or channel 212 can be defined by an arc angle α of 90°, about 90°, between 80° and 100°, or any other suitable angle, and a radius of curvature of 0.125 in, about 0.125 in, between 0.1 and 0.15 in, or any other suitable radius of curvature. In certain embodiments, an arc angle α of 90° or about 90° may be used to hold a hub having a rectangular or generally rectangular cross-section. The support surface 204 can be defined by a radius of curvature of 13 in, about 13 in, between 11 in and 15 in, or any other suitable radius of curvature. The channel 205 and/or channel 207 can be defined by a radius of curvature of 0.25 in, about 0.25 in, between 0.15 in and 0.35 in, or any other suitable radius of curvature.

FIGS. 3L and 3M depict example dimensions of a hub 250 that may be used with the sterile barrier 232 as shown in FIGS. 3G-3K. The hub 250 may be any of the hubs described herein. In certain embodiments, the hub 250 can have a width w1 of 3.75 in, about 3.75 in, between 3.25 in and 4.25 in, or any other suitable width. The hub 250 can have a height h1 of 1.5 in, about 1.5 in, between 1.25 in and 1.75 in, or any other suitable height. Alternatively, the hub 250 can have a height h2 of 2 in, about 2 in, between 1.75 in and 2.25 in, or any other suitable height. In some embodiments, the hub 250 can have a length L1 of 2.5 in, about 2.5 in, between 2 in and 3 in or any other suitable length. Alternatively, the hub 250 can have a length L2 of 4 in, about 4 in, between 3.25 in and 4.75 in, or any other suitable length.

In some embodiments, a top surface of the support table can include surface features that generally correspond to those of the sterile barrier 232. For example, the support table can include a convex surface configured to correspond to the shape, size, and location of the support surface 204 and/or one or more recesses configured to correspond to the shape, size, and location of the channels 205 and 207.

In alternate embodiments, a planar support surface (for example, support surface 104 of sterile barrier 32) can be positioned at an angle to a horizontal plane to facilitate the draining of fluids. In some embodiments, the sterile barrier and/or support table may be positioned, during part of or the entirety of an interventional procedure, at an angle to a horizontal plane to facilitate the draining of fluids. For example, the sterile barrier and/or support table may be constructed or arranged in an angled arrangement (for example, so that one lateral side of the planar support surface is positioned higher than the other lateral side of the planar support surface, the proximal end is higher than the distal end, or the distal end is higher than the proximal end) to facilitate the drainage of fluids. Alternatively, or additionally, a drive mechanism may temporarily tilt the sterile barrier and/or support table (for example, so that one lateral side of the planar support surface is positioned higher than the other lateral side of the planar support surface, the proximal end is higher than the distal end, or the distal end is higher than the proximal end) to facilitate the drainage of fluids. For example, the drive mechanism may raise or lower one lateral side of the sterile barrier and/or support table, the proximal end of the sterile barrier and/or support table, and/or the distal end of the sterile barrier and/or support table.

In certain embodiments, a support surface (for example, support surface 104 of sterile barrier 32) can be positioned in a vertical configuration instead of in the horizontal configuration shown, for example, in FIGS. 3A-3F. For example, the support surface 104 can be positioned at about 90 degrees (or any other suitable angle) from a horizontal plane (e.g., rotated 90 degrees about a long axis of the support surface 104 relative to the embodiment shown in of FIGS. 3A-3F). A vertical configuration may provide for easier interaction with the drive system 18 by a physician. A vertical configuration may also provide for a lower axis of catheter travel closer to a patient without adding standoff height to the drive system 18.

In some embodiments, the drive system 18 may be positioned, during part of, or the entirety of, an interventional procedure, at an angle to a horizontal plane to facilitate the draining of fluids. For example, the drive system 18 may be constructed or arranged in an angled arrangement (for example, so that one lateral side of the planar support surface is positioned higher than the other lateral side of the planar support surface, the proximal end is higher than the distal end, or the distal end is higher than the proximal end) to facilitate the drainage of fluids. Alternatively, or additionally, a drive mechanism may temporarily tilt the drive system 18 (for example, so that one lateral side of the drive system 18 is positioned higher than the other lateral side of the drive system 18, the proximal end is higher than the distal end, or the distal end is higher than the proximal end) to facilitate the drainage of fluids. For example, the drive mechanism may raise or lower one lateral side of the system 18, the proximal end of the drive system 18, and/or the distal end of the drive system 18. In some embodiments, the drive system 18 may be angled so that it extends at an angle away from axis point 24 (for example, so that the proximal end is higher than the distal end), for example, to allow for clearance of a patient's feet.

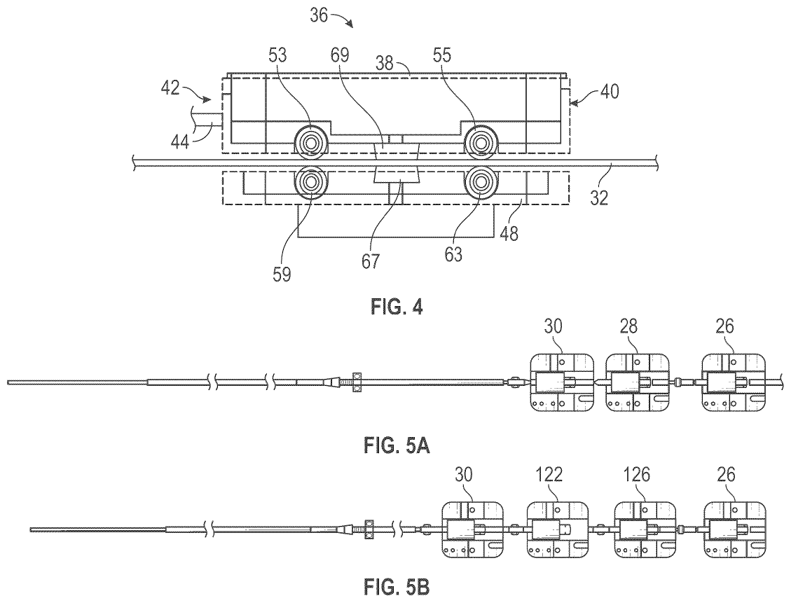

Referring to FIG. 4, hub 36 may represent any of the hubs previously described. Hub 36 includes a housing 38 which extends between a proximal end 40 and a distal end 42. An interventional device 44, which could be any of the interventional devices disclosed herein, extends distally from the hub 36 and into the patient 14 (not illustrated). A hub adapter 48 or carriage acts as a shuttle by advancing proximally or distally along a track in response to operator instructions or controller manipulations. The hub adapter 48 includes at least one drive magnet 67 configured to couple with a driven magnet 69 carried by the hub 36. This provides a magnetic coupling between the drive magnet 67 and driven magnet 69 through the sterile barrier such that the hub 36 is moved across the top of the sterile barrier 32 in response to movement of the hub adapter 48 outside of the sterile field. Movement of the hub adapter is driven by a drive system carried by the support table and described in additional detail below. The hub adapter may act as a robotic drive for an interventional device coupled thereto.

To reduce friction in the system, the hub 36 may be provided with at least a first roller 53 and a second roller 55 which may be in the form of wheels or rotatable balls or drums. The rollers space the sterile barrier apart from the surface of the driven magnet 69 by at least about 0.02 centimeters (about 0.008 inches) and generally no more than about 0.08 centimeters (about 0.03 inches). In some implementations, the space is within the range of from about 0.03 centimeters (about 0.010 inches) and about 0.041 centimeters (about 0.016 inches). The space between the drive magnet 67 and driven magnet 69 is generally no more than about 0.38 centimeters (about 0.15 inches) and in some implementations is no more than about 0.254 centimeters (about 0.10 inches) such as within the range of from about 0.216 centimeters (about 0.085 inches) to about 0.229 centimeters (about 0.090 inches). The hub adapter 48 may similarly be provided with at least a first hub adapter roller 59 and the second hub adapter roller 63, which may be positioned opposite the respective first roller 53 and second roller 55 as illustrated in FIG. 4.

Referring to FIG. 6, there is schematically illustrated one example of a low-profile linear drive support table 20. Support table 20 comprises an elongated frame 51 extending between a proximal end 52 and a distal end 54. At least one support table support 56 is provided to stabilize the support table 20 with respect to the patient (not illustrated). Support 56 may comprise one or more legs or preferably an articulating arm configured to allow movement and positioning of the frame 51 over or adjacent to the patient.

One example of a linear drive table 20 illustrated in FIG. 7 includes three distinct drives. However, two drives or four or more drives (e.g., up to eight drives) may be included depending upon the desired clinical performance. A first drive pulley 58 engages a first drive belt 60. A first carriage bracket 61 is secured to the first drive belt 60 such that rotation of the first drive pulley 58 causes rotation of the first drive belt 60 through an elongate closed loop path. The first carriage bracket 61 may be advanced in a proximal or distal direction along the longitudinal axis of the support table 20 depending upon the direction of rotation of the drive pully 58. In the illustrated implementation, the drive pulley 58 is provided with surface structures such as a plurality of drive pulley teeth 62 for engaging complementary teeth on the first drive belt 60.

A second drive pulley 64 may engage a second drive belt 66 configured to axially move a second carriage bracket 68 along an axial path on the support table 20. A third drive pulley 70 may be configured to drive a third drive belt 72, to advance a third carriage bracket 73 axially along the support table 20. Each of the carriage brackets may be provided with a drive magnet assembly discussed previously but not illustrated in FIG. 7, to form couplers for magnetically coupling to a corresponding driven magnet within the hub of an interventional device as has been discussed.

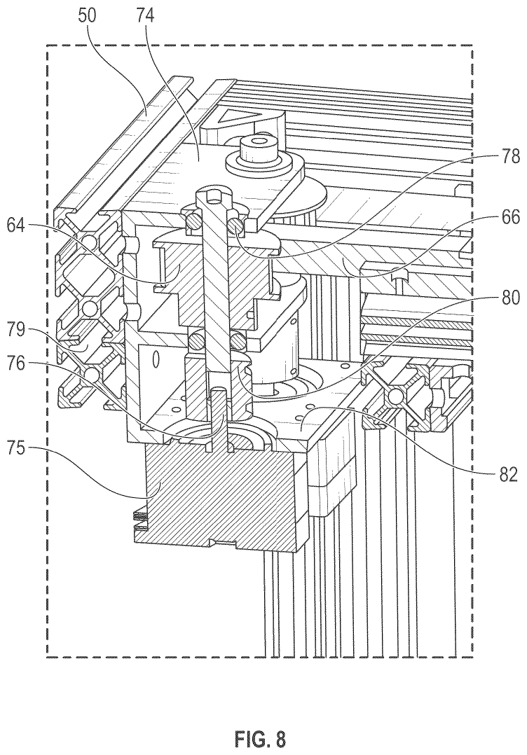

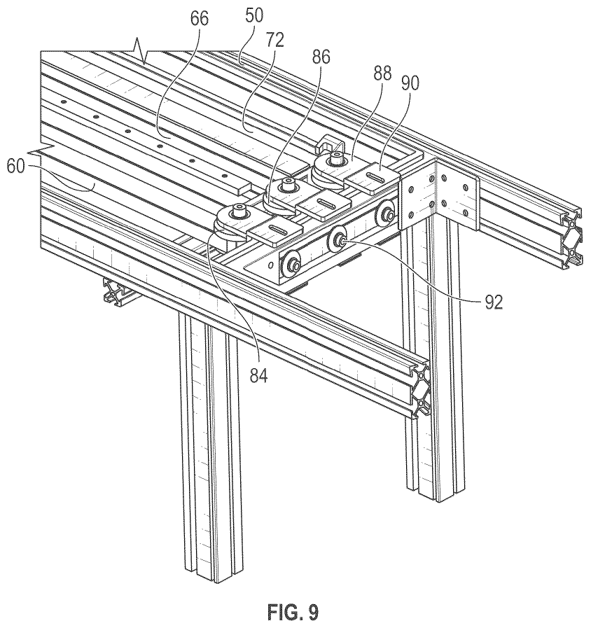

A detailed view of a drive system is shown schematically in FIG. 8. A drive support 74 may be carried by the frame 51 for supporting the drive assembly. The second drive pulley 64 is shown in elevational cross section as rotationally driven by a motor 75 via a rotatable shaft 76. The rotatable shaft 76 may be rotatably carried by the support 74 via a first bearing 78, a shaft coupling 80 and second bearing 79. Motor 75 may be stabilized by a motor bracket 82 connected to the drive support 74 and or the frame 51. The belt drive assemblies for the first drive belt 60 and third drive belt 72 may be similarly constructed and are not further detailed herein. In some embodiments, the drive systems described herein may be a rack and pinion drive table system that is foldable. In such embodiments, motors 75 may be attached to and move with the carriages.

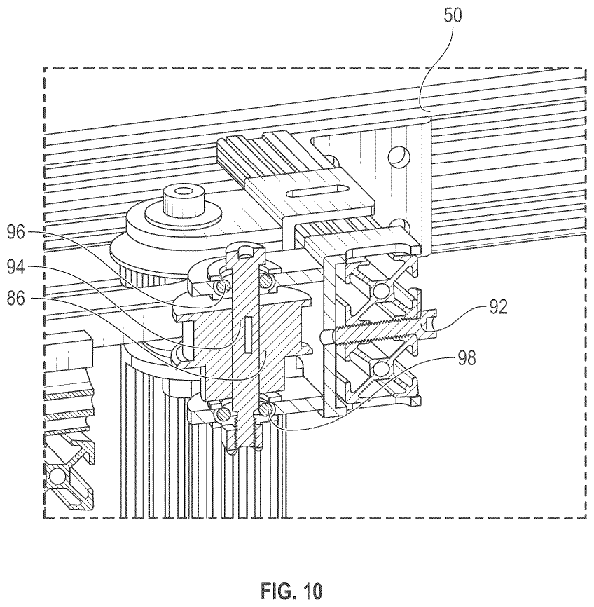

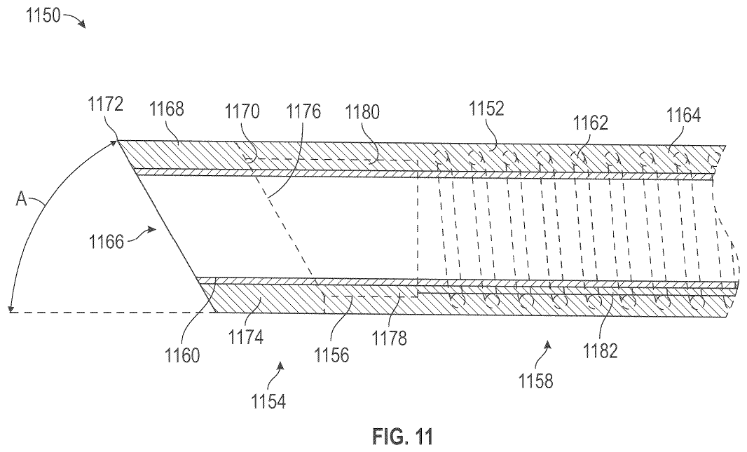

Referring to FIGS. 9 and 10, each of the first second and third drive belts extends around a corresponding first idler pulley 84 second idler pulley 86 and third idler pulley 88. Each idler pulley may be provided with a corresponding tensioning bracket 90, configured to adjust the idler pulleys in a proximal or distal direction in order to adjust the tension of the respective belt. Each tensioning bracket 90 is therefore provided with a tensioning adjustment 92 such as a rotatable screw.

As seen in FIG. 10, the second idler pulley 86, for example, may be carried by a rotatable shaft 94, rotatably secured with respect to the mounting bracket by a first bearing 96 and second bearing 98.

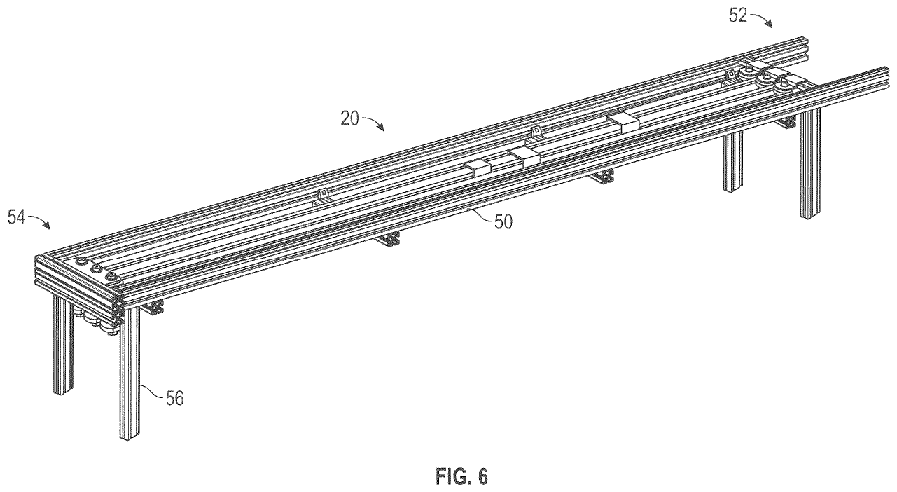

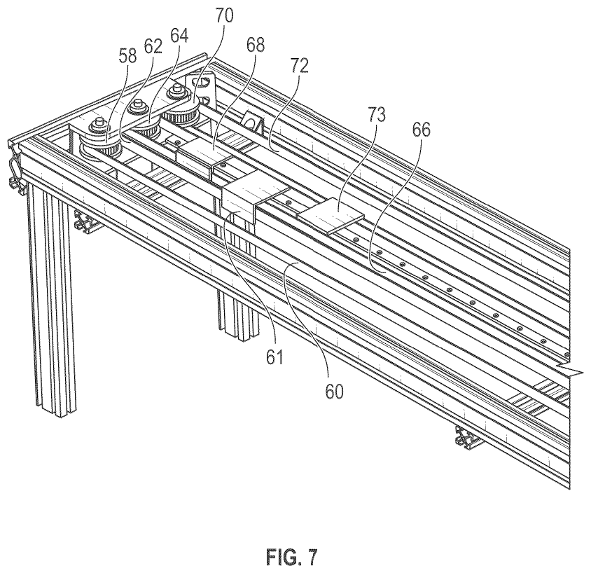

Any of the catheters illustrated, for example, in FIG. 5A, 5B or 11 generally comprise an elongate tubular body extending between a proximal end and a distal functional end. The length and diameter of the tubular body depends upon the desired application. For example, lengths in the area of from about 90 centimeters to about 195 centimeters or more are typical for use in femoral access percutaneous transluminal coronary applications. Intracranial or other applications may call for a different catheter shaft length depending upon the vascular access site.

Any of the catheters disclosed herein may be provided with an inclined distal tip. Referring to FIG. 11, distal catheter tip 1150 comprises a tubular body 1152 which includes an advance segment 1154, a marker band 1156 and a proximal segment 1158. An inner tubular liner 1160 may extend throughout the length of the distal catheter tip 1150, and may comprise dip coated or extruded PTFE or other lubricious material.

A reinforcing element 1162 such as a braid and/or spring coil is embedded in an outer jacket 1164 which may extend the entire length of the catheter.

The advance segment 1154 terminates distally in an angled face 1166, to provide a leading side wall portion 1168 having a length measured between the distal end 130 of the marker band 1156 and a distal tip 1172. In some embodiments, the entire distal tip may be shaped to avoid snagging the tip in areas of arterial bifurcation. A trailing side wall portion 1174 of the advance segment 1154, has an axial length in the illustrated embodiment of approximately equal to the axial length of the leading side wall portion 1168 as measured at approximately 180 degrees around the catheter from the leading side wall portion 1168. The leading side wall portion 1168 may have an axial length within the range of from about 0.1 millimeters to about 5 millimeters and generally within the range of from about 1 to 3 millimeters. The trailing side wall portion 1174 may be equal to or at least about 0.1 or 0.5 or 1 millimeter or 2 millimeters or more shorter than the axial length of the leading side wall portion 1168, depending upon the desired performance.

The angled face 1166 inclines at an angle A within the range of from about 45 degrees to about 80 degrees from the longitudinal axis of the catheter. For certain implementations, the angle is within the range of from about 55 degrees to about 65 degrees from the longitudinal axis of the catheter. In one implementation, the angle A is about 60 degrees. One consequence of an angle A of less than 90 degrees is an elongation of a major axis of the area of the distal port which increases the surface area of the port and may enhance clot aspiration or retention. Compared to the surface area of the circular port (angle A is 90 degrees), the area of the angled port is generally at least about 105 percent, and no more than about 130 percent, in some implementations within the range of from about 110 percent and about 125 percent, and in one example is about 115 percent of the area of the corresponding circular port (angle A is 90 degrees).

In the illustrated embodiment, the axial length of the advance segment is substantially constant around the circumference of the catheter, so that the angled face 1166 is approximately parallel to the distal surface 1176 of the marker band 1156. The marker band 1156 has a proximal surface approximately transverse to the longitudinal axis of the catheter, producing a marker band 1156 having a right trapezoid configuration inside elevational view. A short sidewall 1178 is rotationally aligned with the trailing side wall portion 1174 and has an axial length within the range of from about 0.2 millimeters to about 4 millimeters, and typically from about 0.5 millimeters to about 2 millimeters. An opposing long sidewall 1180 is rotationally aligned with the leading side wall portion 1168. Long sidewall 1180 of the marker band 1156 is generally at least about 10 percent or 20 percent longer than short sidewall 1178 and may be at least about 50 percent or 70 percent or 90 percent or more longer than short sidewall 1178, depending upon desired performance. Generally, the long sidewall 1180 will have a length of at least about 0.5 millimeters or 1 millimeter and less than about 5 millimeters or 4 millimeters.

The marker band may be a continuous annular structure, or may have at least one and optionally two or three or more axially extending slits throughout its length. The slit may be located on the short sidewall 1178 or the long sidewall 1180 or in between, depending upon desired bending characteristics. The marker band may comprise any of a variety of radiopaque materials, such as a platinum/iridium alloy, with a wall thickness preferably no more than about 0.003 inches and in one implementation is about 0.001 inches.

The fluoroscopic appearance of the marker bands may be unique or distinct for each catheter size or type when a plurality of catheters is utilized so that the marker bands can be distinguishable from one another by a software algorithm. Distinguishing the marker bands of a plurality of catheters may be advantageous when the multiple catheters are used together, for example, in a multi catheter assembly or stack as described herein. In some embodiments, the marker band of a catheter may be configured so that a software algorithm can detect motion of the catheter tip.

The marker band zone of the assembled catheter may have a relatively high bending stiffness and high crush strength, such as at least about 50 percent or at least about 100 percent less than proximal segment 18 but generally no more than about 200 percent less than proximal segment 1158. The high crush strength may provide radial support to the adjacent advance segment 1154 and particularly to the leading side wall portion 1168, to facilitate the functioning of distal tip 1172 as an atraumatic bumper during transluminal advance and to resist collapse under vacuum. The proximal segment 1158 preferably has a lower bending stiffness than the marker band zone, and the advance segment 1154 preferably has even a lower bending stiffness and crush strength than the proximal segment 1158.

The advance segment 1154 may comprise a distal extension of the outer tubular jacket 1164 and optionally the inner liner 1160, without other internal supporting structures distally of the marker band 1156. Outer jacket 1164 may comprise extruded polyurethane, such as Tecothane® or NEUsoft™. The advance segment 1154 may have a bending stiffness and radial crush stiffness that is no more than about 50 percent, and in some implementations no more than about 25 percent or 15 percent or 5 percent or less than the corresponding value for the proximal segment 1158.

The catheter may further comprise an axial tension element or support such as a ribbon or one or more filaments or fibers for increasing the tension resistance and/or influencing the bending characteristics in the distal zone. The tension support may comprise one or more axially extending mono strand or multi strand filaments. The one or more tension element 1182 may be axially placed inside the catheter wall near the distal end of the catheter. The one or more tension element 1182 may serve as a tension support and resist tip detachment or elongation of the catheter wall under tension (e.g., when the catheter is being proximally retracted through a kinked outer catheter or tortuous or narrowed vasculature).

At least one of the one or more tension element 1182 may proximally extend along the length of the catheter wall from within about 1.0 centimeters from the distal end of the catheter to less than about 10 centimeters from the distal end of the catheter, less than about 20 centimeters from the distal end of the catheter, less than about 30 centimeters from the distal end of the catheter, less than about 40 centimeters from the distal end of the catheter, or less than about 50 centimeters from the distal end of the catheter.

The one or more tension element 1182 may have a length greater than or equal to about 40 centimeters, greater than or equal to about 30 centimeters, greater than or equal to about 20 centimeters, greater than or equal to about 10 centimeters, or greater than or equal to about 5 centimeters.

At least one of the one or more tension element 1182 may extend at least about the most distal 50 centimeters of the length of the catheter, at least about the most distal 40 centimeters of the length of the catheter, at least about the most distal 30 centimeters or 20 centimeters or 10 centimeters of the length of the catheter.

In some implementations, the tension element extends proximally from the distal end of the catheter along the length of the coil 24 and ends proximally within about 5 centimeters or 2 centimeters or less either side of a transition between a distal coil and a proximal braid. The tension element may end at the transition without overlapping with the braid.

The one or more tension element 1182 may be placed near or radially outside the inner liner 1160. The one or more tension element 1182 may be placed near or radially inside the braid and/or the coil. The one or more tension element 1182 may be carried between the inner liner 1160 and the helical coil and may be secured to the inner liner or other underlying surface by an adhesive prior to addition of the next outer adjacent layer such as the coil. Preferably, the tension element 1182 is secured to the marker band 1156 such as by adhesives or by mechanical interference. In one implementation, the tension element 1182 extends distally beyond the marker band on a first (e.g., inside) surface of the marker band, then wraps around the distal end of the marker band and extends along a second (e.g., outside) surface in either, or both, a proximal inclined or circumferential direction to wrap completely around the marker band.

When more than one tension element 1182 or filament bundles are spaced circumferentially apart in the catheter wall, the tension elements 1182 may be placed in a radially symmetrical manner. For example, the angle between two tension elements 1182 with respect to the radial center of the catheter may be about 180 degrees. Alternatively, depending on desired clinical performances (e.g., flexibility, trackability), the tension elements 1182 may be placed in a radially asymmetrical manner. The angle between any two tension elements 1182 with respect to the radial center of the catheter may be less than about 180 degrees, less than or equal to about 165 degrees, less than or equal to about 135 degrees, less than or equal to about 120 degrees, less than or equal to about 90 degrees, less than or equal to about 45 degrees or, less than or equal to about 15 degrees.

The one or more tension element 1182 may comprise materials such as Vectran®, Kevlar®, Polyester®, Spectra®, Dyneema®, Meta-Para-Aramide®, or any combinations thereof. At least one of the one or more tension element 1182 may comprise a single fiber or a multi-fiber bundle, and the fiber or bundle may have a round or rectangular (e.g., ribbon) cross section. The terms fiber or filament do not convey composition, and they may comprise any of a variety of high tensile strength polymers, metals or alloys depending upon design considerations such as the desired tensile failure limit and wall thickness. The cross-sectional dimension of the one or more tension element 1182, as measured in the radial direction, may be no more than about 2 percent, 5 percent, 8 percent, 15 percent, or 20 percent of that of the catheter 10.

The cross-sectional dimension of the one or more tension element 1182, as measured in the radial direction, may be no more than about 0.03 millimeters (about 0.001 inches), no more than about 0.0508 millimeters (about 0.002 inches), no more than about 0.1 millimeters (about 0.004 inches), no more than about 0.15 millimeters (about 0.006 inches), no more than about 0.2 millimeters (about 0.008 inches), or about 0.38 millimeters (about 0.015 inches).

The one or more tension element 1182 may increase the tensile strength of the distal zone of the catheter before failure under tension (e.g., marker band detachment) to at least about 1 pound, at least about 2 pounds, at least about 3 pounds, at least about 4 pounds, at least about 5 pounds, at least about 6 pounds, at least about 7 pounds, at least about 8 pounds, or at least about 10 pounds or more.

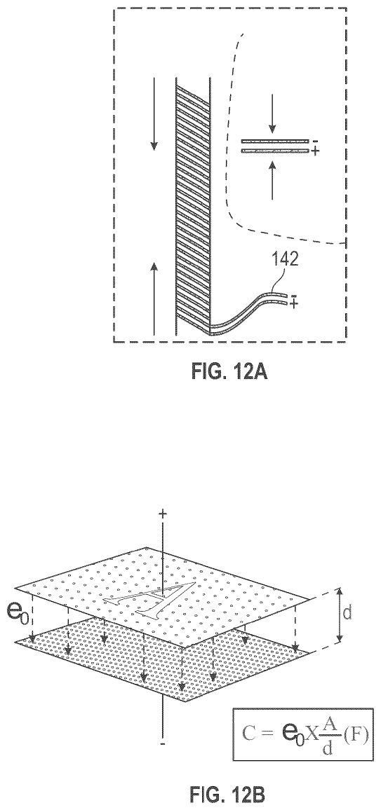

Any of a variety of sensors may be provided on any of the catheters, hubs, carriages, or table, depending upon the desired data. For example, in some implementations, it may be desirable to measure axial tension or compression force applied to the catheter such as along a force sensing zone. The distal end of the catheter would be built with a similar construction as illustrated in FIG. 11, with a helical coil distal section. But instead of using a single helical coil of nitinol wire, a first conductor 140 and second conductor 142 are wrapped into intertwined helical coils and electrically isolated from each other such as by the plastic/resin of the tubular body. See FIG. 12A. Each coil is in electrical communication with the proximal hub by a unique electrical conductor such as a conductive trace or proximal extension of the wire.

This construction of double, electrically isolated helical coils creates a capacitor. This is roughly equivalent to two plates of nitinol with a plastic layer between them, illustrated in FIG. 12B. The capacitance is inversely proportional to the distance between wires. The only variable that would be changing would be d, the distance between the plates. If an axial compressive force is applied to the catheter, the wires (e.g., conductor 140 and conductor 142) will move closer together, thus increasing the capacitance. If an axial tensile force is applied, the wires will get further apart, decreasing the capacitance. This capacitance can be measured at the proximal end of the catheter, giving a measurement of the force at the helical capacitor. Although referred to as a capacitor, in various embodiments, this sensor can be any sensor measuring the electrical interaction between the two coils of wire. There may be a measurable change in inductance or other resulting change due to applied axial forces.

At least a first helical capacitor may have at least one or five or ten or more complete revolutions of each wire. A capacitor may be located within the distal most 5 or 10 or 20 centimeters of the catheter body to sense forces experienced at the distal end. At least a second capacitor may be provided within the proximal most 5 or 10 or 20 centimeters of the catheter body, to sense forces experienced at the proximal end of the catheter.

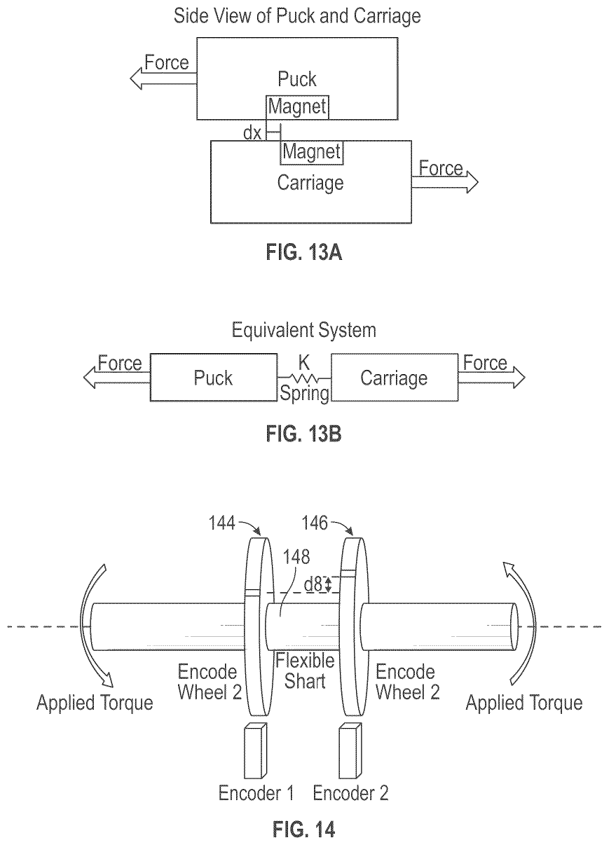

It may also be desirable to measure elastic forces across the magnetic coupling between the hub and corresponding carriage, using the natural springiness (compliance) of the magnetic coupling to measure the force applied to the hub. The magnetic coupling between the hubs and carriages creates a spring. When a force is applied to the hub, the hub will move a small amount relative to the carriage. See FIG. 13A. In robotics, this is called a series elastic actuator. This property can be used to measure the force applied from the carriage to the hub. To measure the force, the relative distance between the hub and the carriage (dx shown in FIG. 13A) is determined and characterize some effective spring constant k between the two components. See FIG. 13B.

The relative distance could be measured in multiple different ways. One method for measuring the relative distance between the hub and carriage is a magnetic sensor (e.g., a Hall effect Sensor between hub and carriage). A magnet is mounted to either the hub or carriage, and a corresponding magnetic sensor is mounted on the other device (carriage or hub). The magnetic sensor might be a hall effect sensor, a magneto-resistive sensor, or another type of magnetic field sensor. Generally, multiple sensors may be used to increase the reliability of the measurement. This reduces noise and reduces interference from external magnetic fields.

Other non-contact distance sensors can also be used. These include optical sensors, inductance sensors, and capacitance sensors. Optical sensors would preferably be configured in a manner that avoids accumulation of blood or other fluid in the interface between the hub assemblies and carriages. In some implementations, wireless (i.e., inductive) power may be used to translate movement and/or transfer information across the sterile barrier between a drive carriage and a hub, for example.

The magnetic coupling between the hub and the carriage has a shear or axial break away threshold which may be about 300 grams or 1000 grams or more. The processor can be configured to compare the axial force applied to the catheter to a preset axial trigger force which if applied to the catheter is perceived to create a risk to the patient. If the trigger force is reached, the processor may be configured to generate a response such as a visual, auditory, or tactile feedback to the physician, and/or intervene and shut down further advance of the catheter until a reset is accomplished. An override feature may be provided so the physician can elect to continue to advance the catheter at forces higher than the trigger force, in a situation where the physician believes the incremental force is warranted.

Force and or torque sensing fiber optics (e.g., Fiber Bragg Grating (FBG) sensors) may be built into the catheter side wall to measure the force and/or torque at various locations along the shaft of a catheter or alternatively may be integrated into a guidewire. The fiber measures axial strain, which can be converted into axial force or torque (when wound helically). At least a first FBG sensor can be integrated into a distal sensing zone, proximal sensing zone and/or intermediate sensing zone on the catheter or guidewire, to measure force and or torque in the vicinity of the sensor.

It may also be desirable to understand the three-dimensional configuration of the catheter or guidewire during and/or following transvascular placement. Shape sensing fiber optics such as an array of FBG fibers to sense the shape of catheters and guidewires. By using multiple force sensing fibers that are a known distance from each other, the shape along the length of the catheter/guidewire can be determined.

A strain gauge may be integrated into the body of the catheter or guidewire to measure force or torque. In an example, the string gauge is a resistive strain gauge. In some embodiments, the strain gauge is incorporated in the distal tip of the catheter, or incorporated in the proximal end of the catheter, and/or incorporated in the proximal end of the catheter and the proximal end of the catheter. In some embodiments, a strain gauge could be deposited on a wall of the catheter via thin film deposition technologies.

Measurements of force and/or torque applied to the catheter or guidewire shafts can be used to determine applied force and/or torque above a safety threshold. When an applied force and/or torque exceeds a safety threshold, a warning may be provided to a user. Applied force and/or torque measurements may also be used to provide feedback related to better catheter manipulation and control. Applied force and/or torque measurements may also be used with processed fluoroscopic imaging information to determine or characterize distal tip motion.

Absolute position of the hubs (and corresponding catheters) along the length of the table may be determined in a variety of ways. For example, a non-contact magnetic sensor may be configured to directly measure the position of the hubs through the sterile barrier. The same type of sensor can also be configured to measure the position of the carriages. Each hub may have at least one magnet attached to it. The robotic table would have a linear array of corresponding magnetic sensors going the entire length of the table. A processor can be configured to determine the location of the magnet along the length of the linear sensor array and display axial position information to the physician.

The foregoing may alternatively be accomplished using a non-contact inductive sensor to directly measure the position of the hubs through the sterile barrier. Each hub or carriage may be provided with an inductive “target” in it. The robotic table may be provided with an inductive sensing array over the entire working length of the table. As a further alternative, an absolute linear encoder may be used to directly measure the linear position of the hubs or carriages. The encoder could use any of a variety of different technologies, including optical, magnetic, inductive, and capacitive methods.

In one implementation, a passive (no electrical connections) target coil may be carried by each hub. A linear printed circuit board (PCB) may run the entire working length of the table (e.g., at least about 1.5 meters to about 1.9 meters) configured to ping an interrogator signal which stimulates a return signal from the passive coil. The PCB is configured to identify the return signal and its location.

Axial position of the carriages may be determined using a multi-turn rotary encoder to measure the rotational position of the pulley, which directly correlates to the linear position of the carriage. Direct measurement of the location of the carriage may alternatively be accomplished by recording the number of steps commanded to the stepper motor to measure the rotational position of the pulley, which directly correlates to the linear position of the carriage.

The location of the catheters and guidewires within the anatomy may also be determined by processing the fluoroscopic image with machine vision, such as to determine the distal tip position, distal tip orientation, and/or guidewire shape. Comparing distal tip position or movement or lack thereof to commanded or actual proximal catheter or guidewire movement at the hub, may be used to detect a loss of relative motion, which may be indicative of a device shaft buckling, prolapse, kinking, or a similar outcome (for example, along the device shaft length inside the body (e.g., in the aorta) or outside the body between hubs. The processing may be done in real time to provide position/orientation data at up to 30 Hertz, although this technique would only provide data while the fluoroscopic imaging is turned on. In some embodiments, machine vision algorithms can be used to generate and suggest optimal catheter manipulations to access or reach anatomical landmarks, similar to driver assist. The machine vision algorithms may utilize data to automatically drive the catheters depending on the anatomy presented by fluoroscopy. Machine vision could also be used to analyze catheter straightness relative to build up tension. As tension builds up in the catheter due to navigating anatomical tortuosity, the catheter will begin to bow and buckle in a sinusoidal manner thus becoming less straight. This tension build can lead to more severe prolapse if built up enough. Measuring the loss of straightness could be a signal to stop before prolapse can happen. This can be particularly helpful when the area of prolapse is outside of the current x-ray view.

Proximal torque applied to the catheter or guidewire shaft may be determined using a dual encoder torque sensor. Referring to FIG. 14, a first encoder 144 and a second encoder 146 may be spaced axially apart along the shaft 148, for measuring the difference in angle over a length of flexible catheter/tube. The difference in angle is interpolated as a torque since the catheter/tube has a known torsional stiffness. As torque is applied to the shaft, the slightly flexible portion of the shaft will twist. The difference between the angles measured by the encoders (dθ) tells us the torque. T=k*dθ, where k is the torsional stiffness.

Confirming the absence of bubbles in fluid lines may also be accomplished using bubble sensors, particularly where the physician is remote from the patient. This may be accomplished using a non-contact ultrasonic sensor that measures the intensity and doppler shift of the reflected ultrasound through the sidewall of fluid tubing to detect bubbles and measure fluid flow rate or fluid level. An ultrasonic or optical sensor may be positioned adjacent an incoming fluid flow path within the hub, or in a supply line leading to the hub. To detect the presence of air bubbles in the infusion line (that is formed of ultrasonically or optically transmissive material) the sensor may include a signal source on a first side of the flow path and a receiver on a second side of the flow path to measure transmission through the liquid passing through the tube to detect bubbles. Alternatively, a reflected ultrasound signal may be detected from the same side of the flow path as the source due to the relatively high echogenicity of bubbles.

Preferably, a bubble removal system is automatically activated upon detection of in line bubbles. A processor may be configured to activate a valve positioned in the flow path downstream of the bubble detector, upon the detection of bubbles. The valve diverts a column of fluid out of the flow path to the patient and into a reservoir. Once bubbles are no longer detected in the flow path and after the volume of fluid in the flow path between the detector and the valve has passed through the valve, the valve may be activated to reconnect the source of fluid with the patient through the flow path. In some embodiments, bubbles could be dislodged from the catheter wall via a beam ultrasonic energy coming from an ultrasound transducer. In other embodiments, the bubble removal system can include a pump and control system upstream of the bubble detector for removal of in line bubbles. A processor may be configured to activate the pump upon detection of bubbles to reverse the fluid flow and clear the bubbles into a waste reservoir before reestablishing bubble free forward flow.

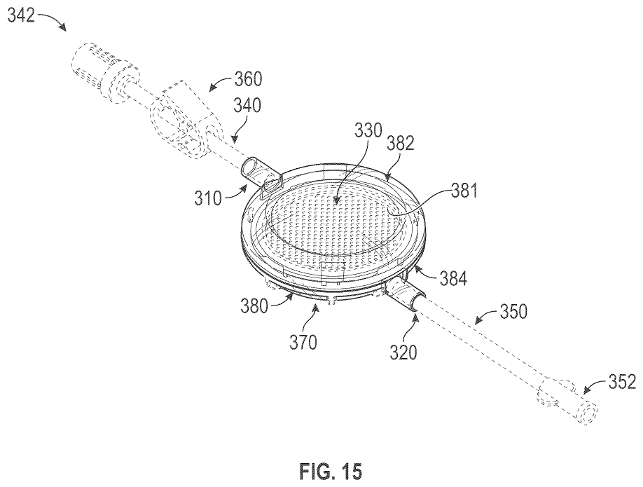

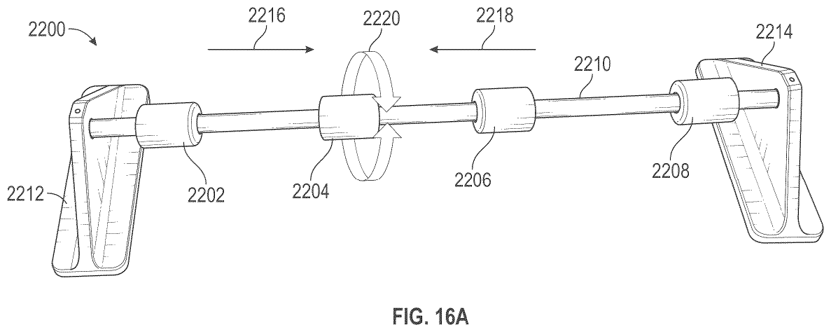

It may additionally be desirable for the physician to be able to view aspirated clot at a location within the sterile field and preferably as close to the patient as practical for fluid management purposes. This may be accomplished by providing a clot retrieval device mounted on the hub, or in an aspiration line leading away from the hub in the direction of the pump. Referring to FIG. 15, one example of a clot retrieval device 370 can include a body 380 enclosing a chamber 381 which communicates with a first port 310 and a second port 320.

In some embodiments, the body 380 includes a housing having a top portion 382 and a bottom portion 384. The body 380 may include a filter 330 positioned in the chamber 381 between the top portion 382, and the bottom portion 384. In some examples, the first port 310 is configured to connect to a first end of a first tube 340 that is fluidly connected to a proximal end of an aspiration catheter.

In an embodiment that is configured to be connected downstream from the hub, the first tube 340 includes a connector 342 positioned at a second end of the first tube 340 that is configured to engage or mate with a corresponding connector on or in communication with the hub. The first port 310 directly communicates with the chamber on the upstream (e.g., top side) of the filter, and the second port 320 directly communicates with the chamber on the downstream (e.g., bottom side) of the filter to facilitate direct visualization of material caught on the upstream side of the filter.

In an implementation configured for remote operation, any of a variety of sensors may be provided to detect clot passing through the aspiration line and/or trapped in the filter, such as an optical sensor, pressure sensor, flow rate sensor, ultrasound sensor or others known in the art.

In some embodiments, the second port 320 is configured to connect to a first end of a second tube 350 that is fluidly connected to an aspiration source (e.g., a pump). In some embodiments, the second tube 350 includes a connector 352 positioned at a second end of the second tube 350 that is configured to engage or mate with a corresponding connector on the pump.

In some examples, the system 300 can include an on-off valve 360 such as a clamp 360. The clamp 360 can be positioned in between the filter 330 and the patient, such as over the first tube 340 to allow the user to engage the clamp and provide flow control by isolating the patient from the clot retrieval device 370. Closing the valve 360 and operating the remote vacuum pump (not illustrated) causes the canister associated with the vacuum pump and the chamber 381 to reach the same low pressure. Due to the short distance and small line volume of the lumen between the chamber 381 end the distal end of the catheter, a sharp negative pressure spike is experienced at the distal end of the catheter rapidly following opening of the valve 360. Additional details are disclosed in U.S. Pat. No. 11,259,821 issued Mar. 1, 2022, to Buck et al., entitled Aspiration System with Accelerated Response, the entirety of which is hereby expressly incorporated by reference herein. In some embodiments, a vacuum may be cycled against a clot to retrieve the clot. The vacuum may be automatically and robotically controlled to remove the clot.

The body 380 can have a top surface spaced apart from a bottom surface by a tubular side wall. In the illustrated implementation, the top and bottom surfaces are substantially circular, and spaced apart by a cylindrical side wall. The top surface may have a diameter that is at least about three times, or five times or more than the axial length (transverse to the top and bottom surfaces) of the side wall, to produce a generally disc shaped housing. Preferably at least a portion of the top wall is optically transparent to improve clot visualization once it is trapped in the clot retrieval device 370. Additional details may be found in PCT/US2022/078113, filed on Oct. 14, 2022, the entirety of which is hereby incorporated by reference herein.

In some examples, the body 380 can include a flush port (not illustrated) that is configured to allow the injection of an optically transparent media such as air, saline or other fluid into the chamber 381 to clear an optical path between the window and the filter to improve clot visualization once it is trapped in the filter 330.

The foregoing represents certain specific implementations of a drive table and associated components and catheters. A wide variety of different drive table constructions can be made, for supporting and axially advancing and retracting two or three or four or more drive magnet assemblies to robotically drive interventional devices, fluid elements, and electrical umbilical elements for communicating electrical signals and fluids to the catheter hubs, as will be appreciated by those of skill in the art in view of the disclosure herein. Additional details may be found in U.S. patent application Ser. No. 17/527,393, filed on Nov. 16, 2021, the entirety of which is hereby incorporated by reference herein.

While the foregoing describes robotically driven interventional devices and manually driven interventional devices, the devices may be manually driven, robotically driven, or a combination of both manually and robotically driven interventional devices, as will be appreciated by those of skill in the art in view of the disclosure herein.

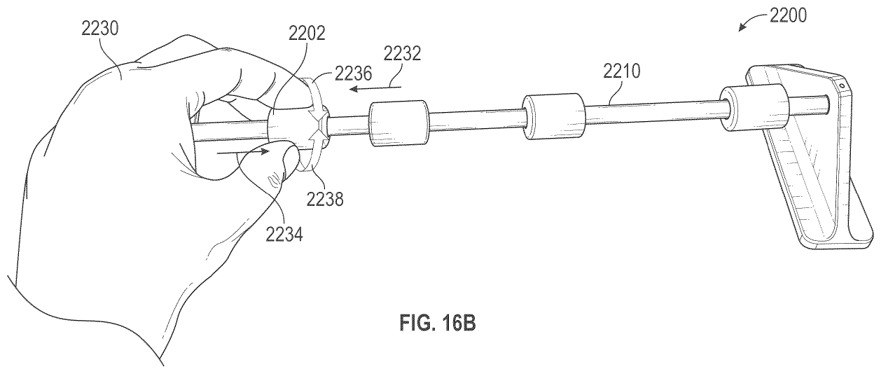

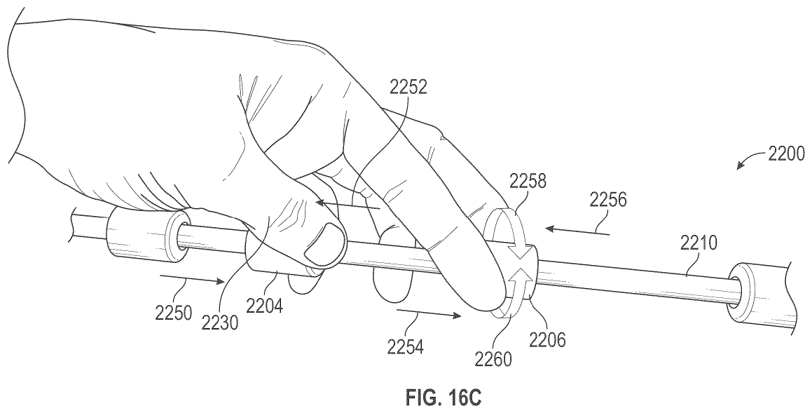

FIGS. 16A-16C illustrate an example control mechanism 2200 for manipulating interventional devices driven by (or otherwise associated with) respective hubs. For example, each hub may be manipulated and/or otherwise moved using at least one control installed in control mechanism 2200. Each control may be adapted to move a unique hub and associated interventional device during an interventional procedure.

As shown in FIG. 16A, the control mechanism 2200 include a first control 2202, a second control 2204, a third control 2206, and a fourth control 2208. More or fewer controls may be provided, depending upon the intended interventional devices configuration. Each control 2202-2208 is movably carried on a shaft 2210 that is coupled to a distal bracket 2212 and to a proximal bracket 2214. The controls 2202-2208 may advance distally or retract proximally on the shaft 2210, as indicated by arrow 2218 and arrow 2216. In addition, each control 2202-2208 may also be rotated about the shaft 2210, as indicated by arrow 2220. Each control movement may trigger a responsive movement in a corresponding carriage on the support table, which may in turn drive movement of a corresponding hub as has been discussed.

The control mechanism 2200 may be positioned on or near to a patient support table having a set of hubs and catheters/interventional devices. In some implementations, the control mechanism 2200 may be positioned remote from the support table such as behind a radiation shield or in a different room or different geographical location in a telemedicine implementation.

Each control 2202-2208 may correspond to and drive movement of a hub and/or a hub and interventional device combination. For example, the control 2202 may be configured to drive hub 30 (FIG. 3F) to move an interventional device such as an 0.088-inch guide catheter corresponding to the hub 30. Similarly, the control 2204 may be configured to drive hub 28 (122) to move an interventional device such as an 0.071-inch procedure catheter. The control 2206 may be configured to drive hub 126 to move an interventional device such as a steerable access catheter. The control 2208 may be configured to drive hub 26 to axially and rotationally move an interventional device such as a guidewire.

FIG. 16B illustrates an example of manually manipulating the control 2202 on control mechanism 2200. In operation, if the user 2230 moves the control 2202 axially along shaft 2210 and distally, as shown by arrow 2232, a corresponding coupled hub and/or interventional device may move responsively in the same direction by a same or scaled amount. If the user 2230 rotates the control 2202 about the shaft 2210 and advances the control proximally, as shown by arrow 2234, a corresponding coupled interventional device will responsively move rotationally and proximally by a same or scaled amount. If the user 2230 moves the control 2202 rotationally about the shaft 2210, as shown by arrow 2236 or arrow 2238, a corresponding coupled hub will drive the corresponding interventional device rotationally in the same direction and/or by a same or scaled amount.

Other axes and degrees of freedom may be defined to enable control 2202 to perform movements that may be translated to movement of hubs and/or interventional devices. For example, the control mechanism may be provided with one or more deflection controls configured to initiate a lateral deflection in a deflection zone on the corresponding interventional device.

Axial movement of a control may be configured to move the coupled hub on a 1:1 basis, or on a non 1:1 scaled basis. For example, if the user 2230 advances the control 2022 about 5 millimeters distally along the shaft 2210, then the corresponding hub may responsively move 5 millimeters in the distal direction.

If the user 2230 rotates the control 2022 about its rotational axis by 5 degrees, the coupled hub will cause the corresponding interventional device to rotate on a 1:1 basis or on a non 1:1 scaled basis. The scaled amount may be selected to reduce or increase the amount of distance and rotation that a hub and/or interventional device moves in accordance with the control movement.

In some implementations, the scaled amount described herein may be determined using a scale factor. The scale factor may apply to one or both translational and rotational movement. In some implementations, a first scale factor is selected for translational movement and a second scale factor, different than the first scale factor, is selected for rotational movement. The axial scaling factor may drive proximal catheter movement at a faster speed than distal catheter movement for a given proximal or distal manipulation of the control.

The rotational scale factor may be 1:1 while the axial scale factor may move the hub by a greater distance than movement of the control such that hub travel to control travel is at least about 2:1 or 5:1 or 10:1 or more depending upon the desired axial length of the control assembly.

The control mechanism 2200 may be configured to enable the clinician to adjust the scale factor for different parts of the procedure. For example, distal advance of the procedure catheter and access catheter through the guide catheter and up to the selected ostium may desirably be accomplished in a ‘fast’ mode. But more distal travel into the neuro vasculature may desirably be accomplished in a relatively slow mode by actuation of a speed control.

In another implementation, one or more controls may be configured to progressively drive advance or retraction speeds of the corresponding hub and associated catheter. For example, distal control 2202 may drive the guide catheter. A slight distal movement of the control 2202 may advance the guide catheter distally at a slow speed, while advancing the control 2202 by a greater distance distally increases the rate of distal travel of the guide catheter.

Controlling the speed of the corresponding hubs either axially or both axially and rotationally may enhance the overall speed of the procedure. For example, advance of the various devices from the femoral access point up to the aortic arch may desirably be accomplished at a faster rate than more distal navigation closer to the treatment site. Also, proximal retraction of the various devices, particularly the guidewire, access catheter and procedure catheter may be desirably accomplished at a relatively higher speeds than distal advance.

FIG. 16C illustrates another example of manually manipulating a control on the control mechanism 2200 to move hubs and/or other interventional devices. In some implementations, two or more controls 2202-2208 may be moved in combination to trigger movement of one or more hubs and/or related interventional devices. In the depicted example, the user 2230 moves control 2204 and control 2206 in combination (e.g., sequentially, simultaneously) such as to simultaneously move the 0.088 guide catheter and the 0.071 aspiration catheter as a unit. Example movement of control 2204 may include axial proximal movement in the directions shown by arrows 2250. Sequentially or simultaneously, the user 2230 may move control 2206 axially in either of the directions shown by arrows 2254 and 2256 while also moving control 2206 rotationally in either of the directions shown by arrows 2258 and 2260.

In some implementations, each control mechanism and/or additional controls (not shown) may be color coded, shaped coded, tactile coded, or other coding to indicate to the user 2230 which color is configured to move which hub or interventional device. In some implementations, the control color coding may also be applied to the hubs and/or interventional devices such that a user may visually match a particular hub/device with a particular control.

In some implementations, other control operations beyond translational movement and rotational movement may be carried out using controls 2202-2208. For example, controls 2202-2208 may be configured to drive a shape change and/or stiffness change of a corresponding interventional device. Controls 2202-2208 may be toggled between different operating modes. For example, controls 2202-2208 may be toggled between movement driven by acceleration and velocity to movement that reflects actual linear displacement or rotation.

In some implementations, the control mechanism 2200 may be provided with a visual display or other indicator of the relative positions of the controls which may correspond the relative positions of the interventional devices. Such displays may depict any or all movement directions, instructions, percentage of movements performed, and/or hub and/or catheter indicators to indicate which device is controlled by a particular control. In some implementations, the display may depict applied force or resistance encountered by the catheter or other measurement being detected or observed by a particular hub or interventional component.

In some implementations, the control mechanism 2200 may include haptic components to provide haptic feedback to a user operating the controls. For example, if the control 2202 is triggering movement of a catheter and the catheter detects a large force at the tip, the control 2202 may generate haptic feedback to indicate to the user to stop or reverse a performed movement. In some implementations, haptic feedback may be generated at the control to indicate to the user to slow or speed a movement using the control. In some implementations, haptics may provide feedback on a large torsional strain buildup that might precede an abrupt rotation, or a large axial force buildup that may be a prelude to buckling of the catheter.

The systems described herein may compare an actual fluoroscopic image position to an input displacement from the controller. A static fluoroscopic image of the patient may be captured in which the patient's vasculature is indexed relative to bony landmarks or one or more implanted soft tissue fiducial markers. Then a real time fluoroscopic image may be displayed as an overlay, aligned with the static image by registration of the fiducial markers.

Visual observation of conformance of the real time movement with the static image, assisted by detected force data can help confirm proper navigation of the associated catheter or guidewire. The systems described herein can also display a comparison of an input proximal mechanical translation of a catheter or guidewire and a resulting distal tip output motion or lack thereof. A loss of relative motion at the distal tip may indicate shaft buckling, prolapse, kinking, or a similar outcome, either inside or outside the body. Such a comparison may be beneficial when the shaft buckling, prolapse, kinking, or similar outcome occurs outside of a current fluoroscopic view.

FIG. 17 illustrates a side elevational schematic view of a multi catheter interventional device assembly 2900 for combined supra-aortic access and/or neurovascular site access and procedure (e.g., aspiration), as described herein. The multi catheter assembly 2900 may be configured for either a manual or a robotic procedure.

The interventional device assembly 2900 includes an insert or access catheter 2902, a procedure catheter 2904, and a guide catheter 2906. Other components are possible including, but not limited to, one or more guidewires (e.g., optional guidewire 2907), one or more guide catheters, an access sheath and/or one or more other procedure catheters and/or associated catheter (control) hubs. In some embodiments, the assembly 2900 may also be configured with an optional deflection control 2908 for controlling deflection of one or more catheters of assembly 2900.

In operation, the multi-catheter assembly 2900 may be used without having to exchange hub components. For example, in the two-stage procedure disclosed previously, a first stage for achieving supra-aortic access, includes mounting an access catheter, guide catheter and guidewire to the support table. Upon gaining supra-aortic access, the access catheter and guidewire were typically removed from the guide catheter. Then, a second catheter assembly is introduced through the guide catheter after attaching a new guidewire hub and a procedure catheter hub to the corresponding drive carriage on the support table.

The single multi catheter assembly 2900 of FIG. 17 is configured to be operated without having to remove hubs and catheters, and without the addition of additional assemblies and/or hubs. Thus, the multicomponent access and procedure configuration of assembly 2900 may utilize a guidewire (or guiding element) 2907 manufactured (configured) to function as both an access guidewire and a navigation guidewire to allow for sufficient access and support, and navigation to the particular distal treatment site. In a non-limiting example configured for robotic implementation, a catheter assembly 2900 may include a guidewire hub (e.g., guidewire hub 2909 or guidewire hub 26 (e.g., FIG. 3A) positioned on a drive table and to the right of catheter 2902 relative to the orientation of FIG. 17), an insert or access catheter hub 2910, a procedure catheter hub 2912, a guide catheter hub 2914 and corresponding catheters. In certain embodiments, one or more of the hubs may include, or be coupled to, a hemostasis valve (e.g., a rotating hemostasis valve) to accommodate introduction of interventional devices therethrough, and/or introduction of fluids (e.g., saline, contrast). In certain embodiments, one or more of the hubs may include, or be coupled to, a fluidic system (which may include a hemostasis valve) for the introduction of a fluid (e.g., saline, contrast) and the application of a vacuum for aspiration functions. Additional examples regarding hemostasis valves, fluidic systems, and aspiration systems are included in U.S. patent application Ser. No. 17/879,614, entitled Multi Catheter System With Integrated Fluidics Management, filed Aug. 2, 2022, and hereby expressly incorporated by reference in its entirety herein.

Once access above the aortic arch has been achieved, the insert or access catheter 2902 (associated with insert catheter hub 2910) may be “parked,” for example, in the vicinity of a carotid artery ostia, and the remainder or a subset of the catheter assembly may be guided more distally toward a particular site (e.g., a clot site, a surgical site, a procedure site, etc.).

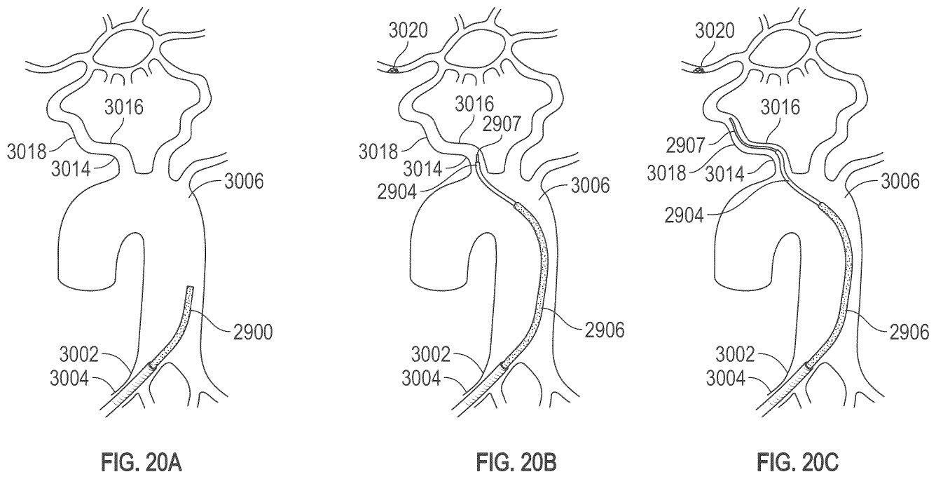

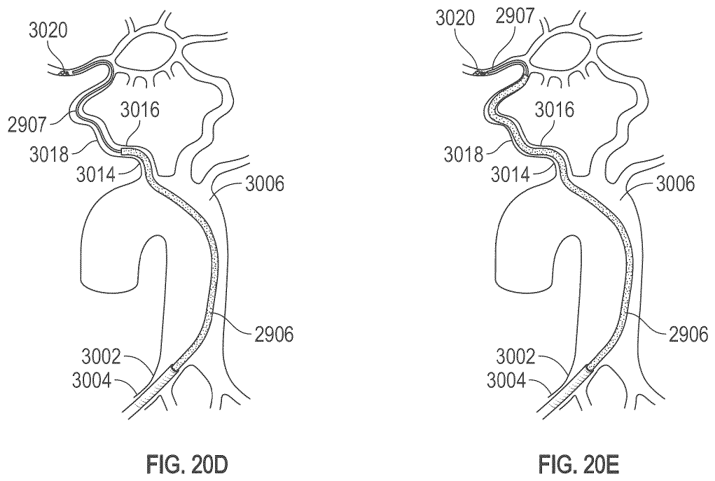

In some embodiments, other smaller procedure catheters may also be added and used at the site. In some implementations, for catheter assembly 2900, in a robotic configuration of assembly 2900, the catheter 2906 may function as a guide catheter. The catheter 2904 may function as a procedure (e.g., aspiration) catheter. In some embodiments, the catheter 2906 may function to perform aspiration in addition to functioning as a guide catheter, either instead of, or in addition to, the catheter 2904. The access catheter 2902 may have a distal deflection zone and can function to access a desired ostium. One of skill in the art will appreciate from FIGS. 20A-20E that either manual manipulation or robotic manipulation of the multi-catheter stack are contemplated herein.

In some embodiments, the catheter assembly 2900 (or other combined catheter assemblies described herein) may be driven as a unit to a location. However, each catheter (or guidewire) component may instead be operated and driven independent of one another to the same or different locations. Since each catheter will have its own stiffness profile relative to length, the position and superposition of the catheters can be adjusted to find the corresponding optimal stiffness profile to help navigate the catheter stack past various anatomical obstacles.

In a non-limiting example, the catheter assembly 2900 may be used for a diagnostic angiogram procedure. In some embodiments, the assembly 2900 may include only the guidewire 2907 and access catheter 2902 (in the form of a diagnostic angiographic catheter) for performing the diagnostic angiogram procedure or only the guidewire 2907 and the access catheter 2902 may be utilized during the procedure. Alternatively, the guide catheter 2906 and procedure catheter 2904 may be retracted proximally to expose the distal end of the access catheter 2902 (e.g., a few centimeters of the distal end of the access catheter) to perform the diagnostic angiography.

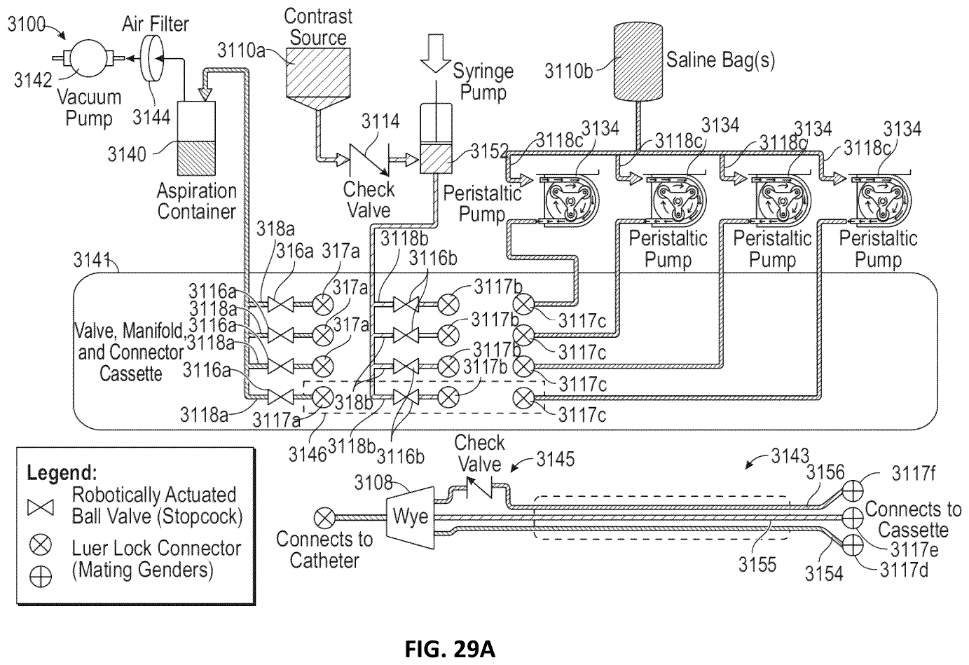

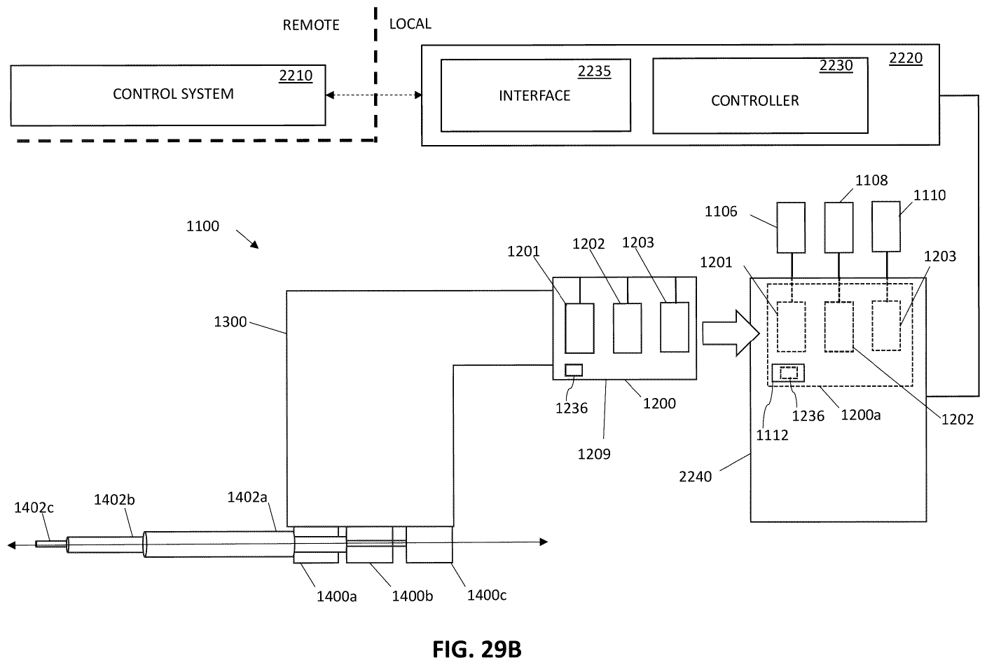

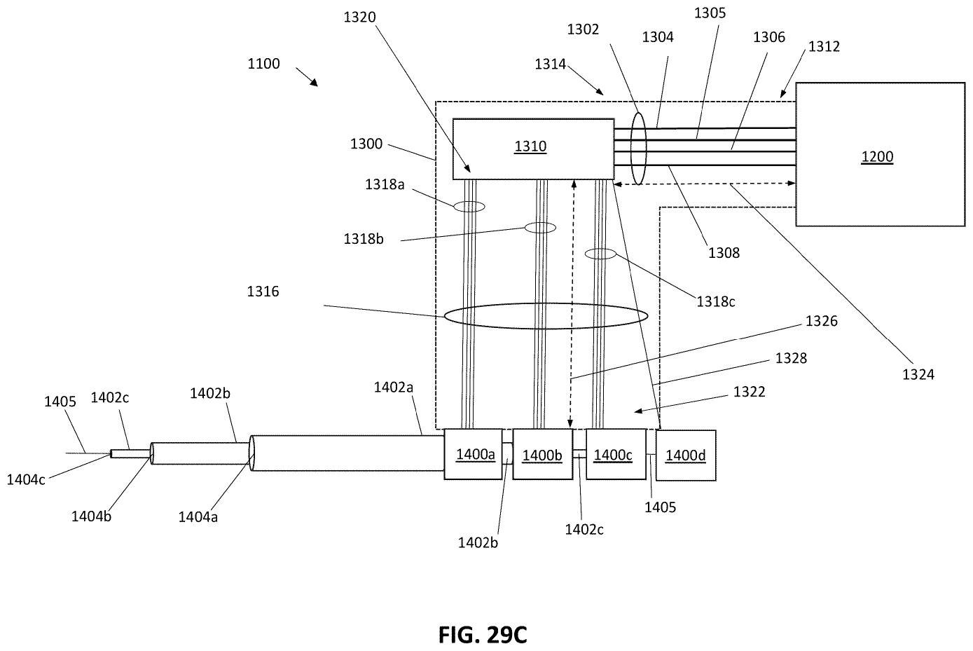

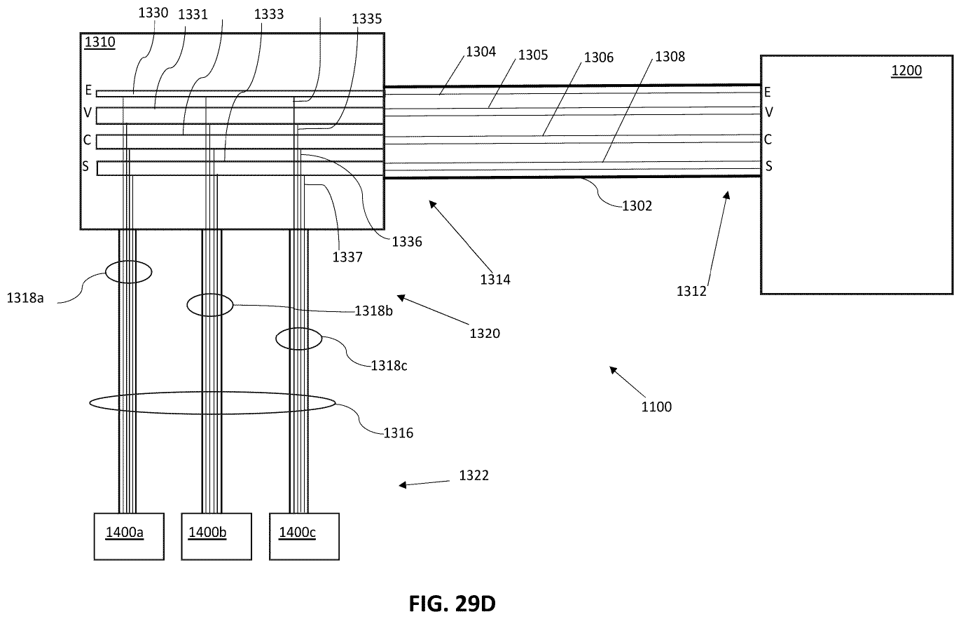

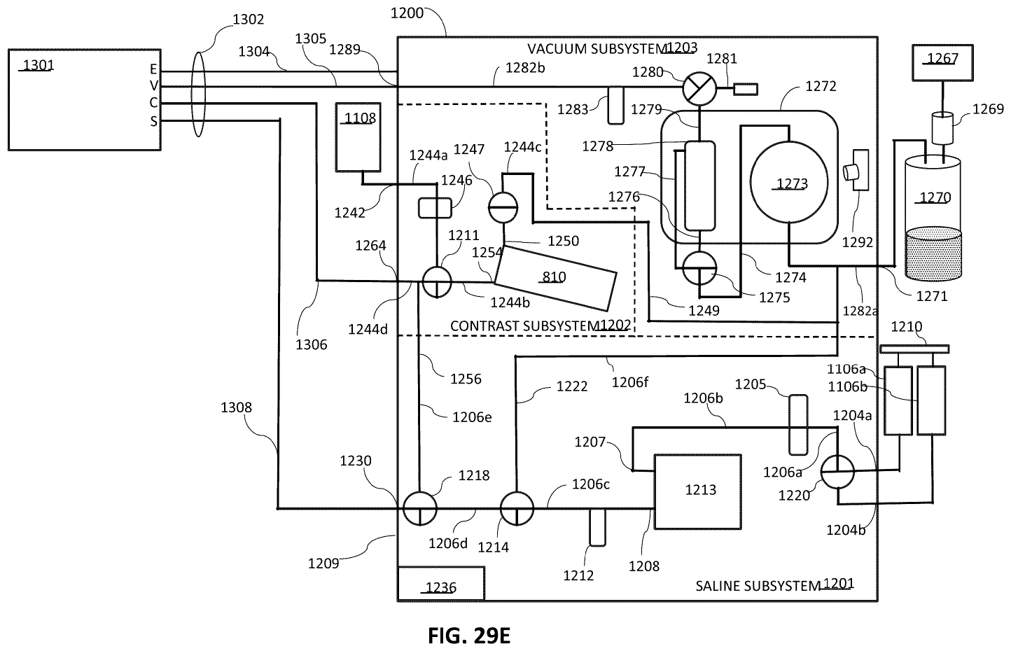

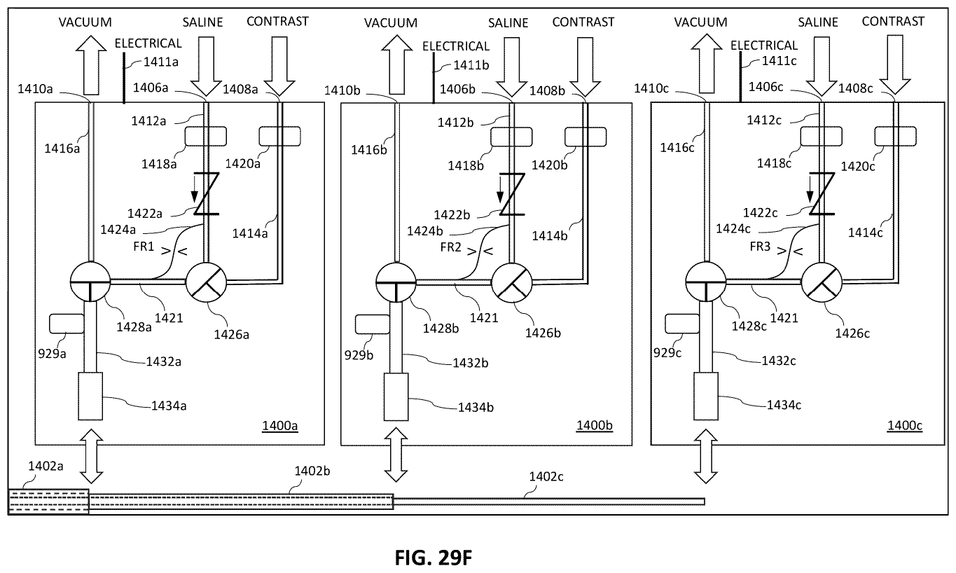

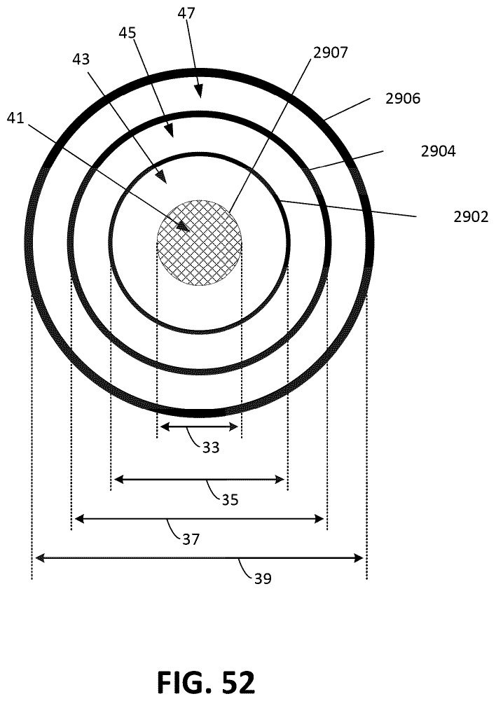

A set of elongated devices can be configured such that they can be arranged concentrically, that is, in a concentric stack. For example, the set of elongated devices can include at least two elongated devices, for example, a catheter and a guidewire, two or more catheters, or two or more catheters and a guidewire. As shown in the example in FIG. 17, a concentric stack can include a guide catheter 2906, procedure catheter 2904, access catheter 2902, and guidewire (or guiding element) 2907. A controller of the robotic interventional device drive system can cause one or more elongated devices in the concentric stack to be positioned in various locations with respect to other elongated devices in the concentric stack certain processes of a procedure, including injecting contrast. For example, a controller of the robotic interventional device drive system can cause one or more elongated devices in the concentric stack to be positioned partially or fully in the lumen of another (e.g., larger inner diameter) elongated device, or positioned not in the lumen of another elongated device, during a contrast injection. A controller can also cause the fluidics system (for example, fluidic systems illustrated in FIGS. 29A-29F) to align fluid channels to provide contrast injection through any one or more of the catheters in the concentric stack (although typically contrast is provided through one catheter). The catheters in the concentric stack are designed/selected to have inner diameters that are sized to allow them to provide a contrast injection when another catheter or guidewire is positioned in their lumen. FIGS. 52-63 illustrate representations of examples of a distal end view of a concentric stack of one or more elongated medical devices where contrast can be provided through a selected catheter while one or more other elongated devices are positioned in the lumen of the selected catheter and a desired flow rate out of contrast of the distal end of the catheter can be achieved. For example, a desired flow rate of at least about 1 mL/second, at least about 2 mL/second, or at least about 3 mL/second. The flow rate can be affected by various items, for example, the viscosity of the contrast media, the length of the catheter being used to inject the contrast, the pressure provided by the contrast pump to provide contrast, coatings used on a lumen or an exterior surface of a catheter, and/or the effective cross-sectional area of the catheter providing the contrast (which changes when another EMD is in the lumen of the catheter). In some embodiments, information relating to contrast viscosity, the length of EMDs in the stack, whether surfaces of catheters and guiding elements have coatings to lower friction of contrast flowing along the surfaces, and/or the effective cross-sectional areas of various arrangements of EMD stacks, and other information can be predetermined and provided to the system, and the system can use such information (and other information) to determine how much pressure is needed to achieve a desired flow rate. However, because a contrast pump typically can provide only a certain amount of pressure and a fluidics system can only handle a certain amount of pressure without breaking, selecting certain configurations of the EMD stack can ensure a sufficient effective cross-sectional area is available for a selected catheter to provide a desired contrast flow rate at an operating pressure. In these examples, one or more catheters in the concentric stacks of EMDs can be used to inject contrast into a patient during a medical procedure.

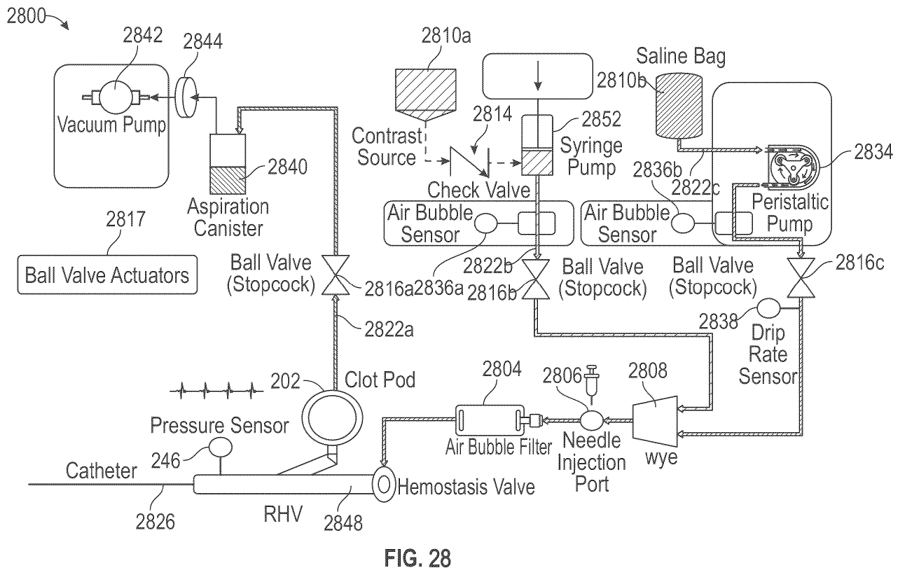

The catheters can each be in fluid communication with the fluidic system that is controlled to be configured to selectively provide (inject) contrast through one or more of the catheters. For example, the fluidics system includes valves and fluid communication channels in the robotic system that can be aligned to inject contrast through any one or more of the catheters in the concentric stack of one or more catheters. For example, as described in reference to FIGS. 28-29F. Typically, the fluidics system can be configured to inject contrast through only one catheter at a time. However, in some instances, the fluidics system can be configured (e.g., by aligning valves, by positioning catheters in the concentric stack) to provide contrast through two or more catheters of the concentric stack simultaneously for injection into a patient. In an example, a user of a robotic system (for example, the interventional setup 10 illustrated in FIG. 1) can select one or more of the catheters in the concentric stack of EMDs to inject contrast into a patient, and the fluidics system aligns valves to provide contrast through the selected one or more catheters. For example, the fluidics system can be configured to inject contrast through the lumen of one of the catheters in the concentric stack of catheters, or the fluidics system can be configured to inject contrast though two or more of the catheters in the concentric stack. A contrast pump (e.g., a syringe pump) can be actuated based on an input from a user to provide the contrast injection. The user can be remotely located from the interventional system 10, for example, in the same room but not right beside the interventional system e.g., behind a barrier), in a different room than the interventional system but in the same building as the interventional system, at a different site or building than the interventional system where the site or building is hundreds of yards, miles, hundreds of miles, or thousands of miles from the interventional system.

In some embodiments, a concentric stack of one or more EMDs can include a guide catheter 2906, a procedure catheter 2904, an access catheter 2902, and a guiding element (e.g., guidewire) 2907. In some examples, the EMDs can be referred to as a first catheter (or EMD), a second catheter (or EMD), a third catheter (or EMD), and a guiding element (or guidewire or fourth EMD). As illustrated in the example of FIG. 52, the outside diameter 33 of the guiding element 2907 is smaller than the inside diameter 35 of the access catheter 2902, and the system is configured such that the guidewire 2907 can be at least partially positioned in the lumen of the access catheter 2902. The outside diameter of the access catheter 2902 is smaller than the inside diameter 37 of the procedure catheter 2904, and the system is configured such that the access catheter 2902 can be at least partially positioned in the lumen of the procedure catheter 2904. The outside diameter of the procedure catheter 2904 is smaller than the inside diameter 39 of the guide catheter 2906, and the system is configured such that the procedure catheter 2904 can be at least partially positioned in the lumen of the guide catheter 2906. In certain embodiments, the guide catheter 2906 may be a ‘large bore’ guide catheter or access catheter having a bore (inner) diameter 39 of, for example, at least about 0.075, or at least about 0.080 inches in diameter. In an example, the inner diameter 39 of the guide catheter 2906 is 0.088″. The procedure catheter 2904 may be an aspiration catheter having an inner diameter 37 within the range of from about 0.060 to about 0.075 inches. The access catheter 2902 may be a steerable catheter with a deflectable distal tip, having an inner diameter 35 within the range of, for example, from about 0.035 to about 0.050 inches. In some examples, the access catheter 2902 has an inner diameter 35 of between about 0.045 inches and about 0.049 inches. This configuration allows for an effective cross-sectional area of the lumen of the access catheter for fluid communication when a suitably sized guiding element 2907 is positioned in the access catheter, as described further herein. In some examples, a suitably-sized guidewire 2907 has an outer diameter 35 within the range of from about 0.014 to about 0.024 inches. In some examples, the guiding element 2907 may have an outer diameter 35 of about 0.014″, 0.015″, 0.016″, 0.017″, 0.018″, 0.019″, 0.020″, 0.021″, 0.022″, 0.023″, or 0.024″ plus or minus 0.0005″. In one example of a system configuration where the system can inject contrast media (or another fluid) into a patient through an access catheter 2902 while a guiding element 2907 is positioned partially or fully in the lumen of the access catheter 2902, the guide catheter 2906 may have an inner diameter 39 of about 0.088 inches, the procedure catheter 2904 an inner diameter 37 of about 0.071 inches, the access catheter 2902 an inner diameter 35 of between about 0.047 inches, and the guiding element 2907 may have an outer diameter 33 of about 0.024 inches. In one example, the guide catheter 2906 can have an outside diameter of about 0.110 inches, the procedure catheter 2904 can have an outside diameter of about 0.083 inches, and the access catheter 2902 can have an outside diameter of about 0.061 inches.

Still referring to FIG. 52, in some embodiments the access catheter 2902 may have an inner diameter 35 of about between about 0.045 to about 0.049 inches, and the guidewire 2907 may have an outer diameter 33 of less than or equal to 0.024 inches, for example, an outer diameter 33 in the range between about 0.014 and about 0.024 inches. These configuration provides sufficient annular space (effective cross-sectional area) within the lumen of the access catheter 2902 for contrast media to propagate through the lumen of the access catheter 2902 so the guiding element 2907 may remain in place while contrast media is injected into the catheter assembly 2900. This advantageously shortens the time of the overall procedure by eliminating the need to remove the guiding element 2907 each time contrast media is to be injected to create enough annular space within the lumen of the access catheter 2902 for the contrast media to flow through the lumen of the access catheter 2902. For example, these configurations provide allow for contrast to flow inside a catheter at a flow rate of about 3 cc's per second, at a pressure not to exceed 400 PSI. In some examples, such a catheter is between about 100 cm and 160 cm in length. Additionally, this configuration lowers the risk of air embolisms associated with the repeated removal and re-insertion of the guidewire 2907 from, and into, the catheter assembly 2900.

As described further in reference to the examples, to provide a desired fluid flow rate through the access catheter 2902 while the guiding element 2907 is positioned within the access catheter 2902, a certain effective cross-sectional area 43 of the access catheter 2902 is available to communicate fluid from the proximal end of the catheter to the distal end and out of the access catheter. In reference to FIG. 52, the effective cross-sectional area 43 of the access catheter 2902 (i.e., the space in the lumen of the access catheter 2902 where fluid can be communicated when the guiding element 2970 is positioned in the lumen) is determined by subtracting the cross-sectional area 41 of the guiding element 2907 from the cross-sectional area of the access catheter 2902. Through testing related to designed configurations of providing fluid communication through the access catheter with the guide element positioned in its lumen, working pressures can be determined that are feasible to generate with a contrast pump and the fluidics system. Typically, the working pressure is a relatively high pressure. In some examples, the high pressure can be less than or equal to about 400 PSI. For example, above about 200 PSI, above about 250 PSI, above about 300 PSI, between about 350 PSI and about 400 PSI, between about 300 PSI and about 400 PSI, between about 250 PSI and about 400 PSI, between about 200 PSI and about 400 PSI, or between about 150 PSI and about 400 PSI. In some embodiments, where the portion of a fluidics system has been designed to use higher pressures, the working pressure can be greater than about 400 PSI, or even greater than about 500 PIS. Typically, the pressure is on the higher side of the range of about 250-about 400 PSI for proper operation (for example, a quick injection of contrast to facilitate a medical practitioner to see the patient's anatomy).

In some embodiments, it was determined that these working pressures, an effective cross-sectional area 43 in a lumen of at least about 0.001 square inches is desired. In an example, it was determined that at these pressure ranges an effective cross-sectional area 43 of at least about 0.001257 square inches (e.g., about 0.001 square inches) is desired. In another example, an effective cross-sectional area 43 of at least 0.001407 square inches (e.g., about 0.001 square inches) is desired to produce a desired contrast fluid flow at less than or equal to about 400 PSI. For the example where it was determined that an effective cross-sectional area 43 of at least about 0.001257 square inches was desired, various dimensions of the outside diameter 33 of the guiding element 2907 and the inside diameter 35 of the access catheter 2902 (or any two adjacent EMDs where the smaller EMD is in the lumen of the larger EMD) can be utilized to achieve an effective cross-sectional area of at least about 0.001257 square inches as shown in Table IA below, which shows examples of determined effective cross-sectional areas of the access catheter for various access catheter inner diameter dimensions and various guiding element (GE) outer diameter dimensions, where the shaded cells indicate effective cross-sectional areas that fall below the threshold of 0.001257 square inches. In an example, as illustrated in Table IA an access catheter ID of 0.045″ and a guiding element OD of 0.020″ provides an effective cross-sectional area of the access catheter of 0.001276 square inches which is acceptable (un-shaded) because it is above the threshold value of 0.001257 square inches. In another example, as illustrated in Table IA an access catheter ID of 0.045″ and a guiding element OD of 0.021″ provides an effective cross-sectional area of the access catheter of 0.001244 square inches which is unacceptable (shaded) because it is below a (predetermined) threshold value of 0.001257 square inches. For configurations where it is determined that an effective cross-sectional area of at least about 0.001 square inches in needed to produce a desired flow rate (e.g., of about 1 mL/sec., 2 mL/sec., or 3 mL/sec.) all of the configurations shown in Table 1A with access catheter inner diameters (ID) of 0.045″ to 0.055″ and guiding element outer diameters (OD) of 0.014″ to 0.025″ result in an effective cross-sectional area of the access catheter of at least 0.001 square inches. Although Table IA illustrates some examples, others example configurations are also possible that meet this threshold. A controller can cause the fluidics system to inject contrast through the procedure catheter 2904 when the insert catheter 2902 and/or the guidewire 2907 is positioned in the lumen of the procedure catheter 2904. A controller can cause the fluidics system to inject contrast though the guide catheter 2906 when the procedure catheter 2904 is positioned in the lumen of the guide catheter 2906 and/or the insert catheter 2902 and/or the guidewire is positioned in the lumen of the guide catheter 2906 because cross-sectional areas 45 and 47 are also greater than at least about 0.001257.

TABLE IA
EFFECTIVE CROSS-SECTIONAL AREA OF ACCESS CATHETER

| | Catheter Inner Diameter (inches) | | | | | |
|---|---|---|---|---|---|---|
| GE OD | 0.045″ | 0.046″ | 0.047″ | 0.048″ | 0.049″ | 0.055″ |
| 0.014″ | 0.001436 | 0.001508 | 0.001581 | 0.001656 | 0.001732 | 0.002222 |
| 0.015″ | 0.001414 | 0.001485 | 0.001558 | 0.001633 | 0.001709 | 0.002199 |
| 0.016″ | 0.001389 | 0.001461 | 0.001534 | 0.001608 | 0.001685 | 0.002175 |
| 0.017″ | 0.001363 | 0.001435 | 0.001508 | 0.001583 | 0.001659 | 0.002149 |
| 0.018″ | 0.001336 | 0.001407 | 0.001480 | 0.001555 | 0.001631 | 0.002121 |
| 0.019″ | 0.001307 | 0.001378 | 0.001451 | 0.001526 | 0.001602 | 0.002092 |
| 0.020″ | 0.001276 | 0.001348 | 0.001421 | 0.001495 | 0.001572 | 0.002062 |
| 0.021″ | 0.001244 | 0.001316 | 0.001389 | 0.001463 | 0.001539 | 0.002029 |
| 0.022″ | 0.001210 | 0.001282 | 0.001355 | 0.001429 | 0.001506 | 0.001996 |
| 0.023″ | 0.001175 | 0.001246 | 0.001319 | 0.001394 | 0.001470 | 0.001960 |
| 0.024″ | 0.001138 | 0.001210 | 0.001283 | 0.001357 | 0.001433 | 0.001923 |
| 0.025″ | 0.001100 | 0.001171 | 0.001244 | 0.001319 | 0.001395 | 0.001885 |

Table B (below) illustrates an example of effective cross-sectional areas of a procedure catheter (“P Cath”) and a guide catheter (“G Cath”) that are in a concentric stack when an EMD (e.g., a guiding element or a catheter) is in the lumen of the procedure catheter or the guide catheter, for certain embodiments. The EMD outer diameters (“OD”) are shown in two columns to the left of the P Cath and G Cath columns, the EMD outer diameters in the range of 0.014 inches to and the 0.083 inches. As illustrated in Table IB, for a procedure catheter having an ID of 0.071 inches, an EMD having an OD of 0.014″ to 0.061 inches results in an effective diameter (in the lumen of the procedure catheter) of at least about 0.001 square inches. These are examples of configurations of a concentric stack of EMDs that can be used to provide a desired flow rate of at least about 1 mL/second. Table IB shows that an EMD having an OD of 0.068 inches falls below a 0.001 square inches “threshold”-in examples with this configuration a higher contrast pump working pressure may be needed to produce the desired contrast flow rate (which as indicated above, such a higher working pressure may not be possible or desirable due to the design of the contrast pump and the rest of a fluidics system). Also as illustrated in Table IB, for a guide catheter having an ID of 0.088 inches, an EMD having an OD of 0.014 inches to 0.08 inches results in an effective diameter (in the lumen of the guide catheter) of at least about 0.001 square inches. Table IB also shows that an EMD having an OD of 0.083 inches falls below the 0.001 square inches “threshold”-in examples with this configuration a higher contrast pump working pressure may be needed to produce the desired contrast flow rate (which as indicated above, such a higher working pressure may not be possible or desirable due to the design of the contrast pump and the rest of a fluidics system).

TABLE IB
EFFECTIVE CROSS-SECTIONAL AREA OF PROCEDURE
CATHETER AND A GUIDE CATHETER

| | Catheter Inner Diameter (inches) | | | | |
|---|---|---|---|---|---|
| | P Cath | G Cath | | P Cath | G Cath |
| EMD OD | 0.071″ | 0.088″ | EMD OD | 0.071″ | 0.088″ |
| 0.014″ | 0.003805 | 0.005928 | 0.022″ | 0.003579 | 0.005702 |
| 0.015″ | 0.003782 | 0.005905 | 0.023″ | 0.003544 | 0.005667 |
| 0.016″ | 0.003758 | 0.005881 | 0.024″ | 0.003507 | 0.005630 |
| 0.017″ | 0.003732 | 0.005855 | 0.025″ | 0.003468 | 0.005591 |
| 0.018″ | 0.003705 | 0.005828 | 0.061″ | 0.001037 | 0.003160 |
| 0.019″ | 0.003676 | 0.005799 | 0.068″ | 0.000328 | 0.002450 |
| 0.020″ | 0.003645 | 0.005768 | 0.08″ | — | 0.001056 |
| 0.021″ | 0.003613 | 0.005736 | 0.083″ | — | 0.000672 |

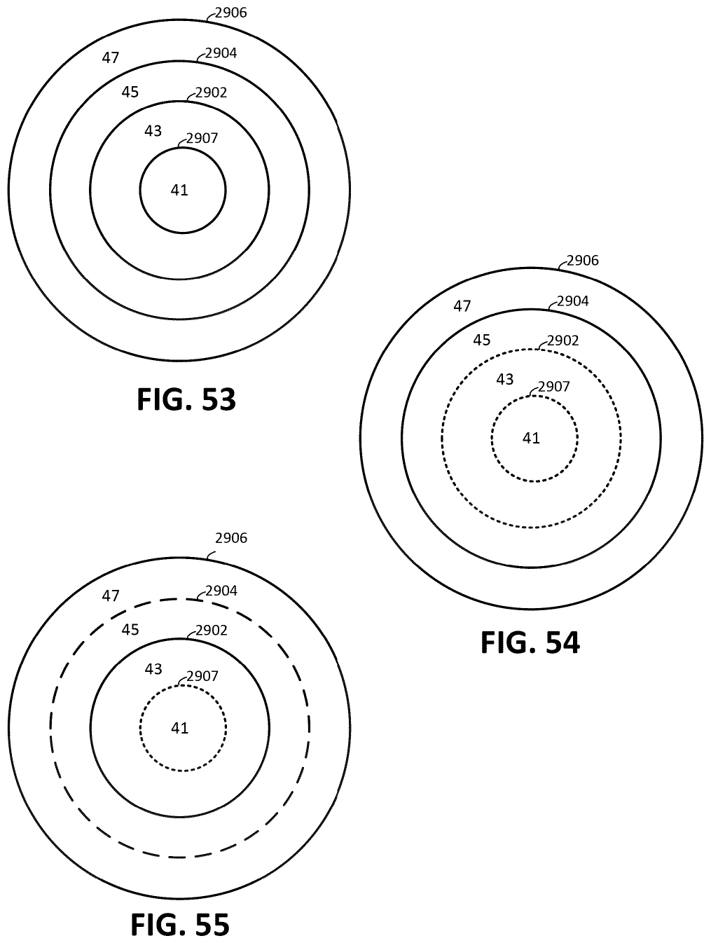

FIG. 53 is a representation of an example of a distal end view of a concentric stack of one or more EMDs that can be used in the systems and methods disclosed herein for a medical procedure including injecting contrast. In this example, the concentric stack of one or more EMDs includes a guide catheter 2906, a procedure catheter 2904, an insert catheter 2902, and a guiding element (e.g., a guidewire) 2907. In this example, the guiding element is a guidewire having a cross-sectional area 41 defined by its outer diameter (OD). In other examples, the guiding element can be a catheter, which has a cross-sectional area 41 defined by its OD. The guide catheter 2906, procedure catheter 2904, and insert catheter 2902 can each be coupled to a hub (for example hub assemblies 1400a, 1400b, 1400c, respectively, FIG. 29). An effective cross-sectional area 47 between the guide catheter 2906 and the procedure catheter 2904 can provide a fluid channel to inject contrast into a patient, for example, in a high pressure contrast system (e.g., contrast provided at between about 250 PSI and about 400 PSI, or higher). An effective cross-sectional area 45 between the procedure catheter 2904 and the insert catheter 2902 can also provide a fluid channel to inject contrast into a patient, for example, in a high pressure contrast system (e.g., contrast provided at between about 250 PSI and about 400 PSI, or higher). The effective cross-sectional areas 47, 45, and 41 can be at least or greater than about 0.001 square inches to provide an effective fluid channel for contrast injection. The guide catheter 2906, procedure catheter 2904, and insert catheter 2902 can receive contrast via the hubs when fluidic channels are connected (e.g., via valves) to provide contrast from the contrast pump in the contrast subsystem to the lumen of one or more of the guide catheter 2906, procedure catheter 2904, and the insert catheter 2902, as described herein (for example, in reference to FIGS. 29B-29F). In an example of a fluidics system with hub assemblies similar to those hub assemblies 1400a-c illustrated in FIG. 29FA a controller can cause the fluidics system to provide, to a patient during a procedure, a contrast injection through any one or more of the guide catheter 2906, procedure catheter 2904, and insert catheter 2902. For example, to provide a contrast injection from the guide catheter 2906, a controller can align control valve 1426a and valve 1428a in hub assembly 1400a to align the contrast fluid channel 1414a to be in fluid communication with the lumen of the guide catheter, and then cause the contrast pump to actuate and provide contrast at a high pressure (e.g., about 250-400 PSI) through the lumen of the guide catheter 2906 and into the patient. Similarly, to provide a contrast injection from the procedure catheter 2904, a controller can align valve 1426b and valve 1428b in hub assembly 1400b to align the contrast fluid channel 1414b to be in fluid communication with the lumen of the procedure catheter 2904, and then cause the contrast pump to actuate and provide contrast at a high pressure (e.g., 300-400 PSI) through the lumen of the procedure catheter 2904 and into the patient. To provide a contrast injection from the insert catheter 2902, a controller can align valve 1426c and valve 1428c in hub assembly 1400c to align the contrast fluid channel 1414c to be in fluid communication with the lumen of the insert catheter 2902, and then cause the contrast pump to actuate and provide contrast at a high pressure (e.g., about 250-400 PSI) through the lumen of the insert catheter 2902 and into the patient. The catheters have outside diameters there are sized such that the effective cross-sectional areas 47 and 45 (between the guide catheter 2906 and the procedure catheter 2904 when the procedure catheter is positioned in the lumen of the guide catheter 2906, and between the procedure catheter 2904 and the insert catheter 2902 when the insert catheter 2902 is positioned in the lumen of the procedure catheter 2904, respectively) are at least about 0.001 square inches (e.g., 0.001257 square inches), which is large enough to allow a high pressure contrast injection to effectively be performed through the guide catheter 2906 and/or the procedure catheter 2904 without removing any elongated device from the lumen of the guide catheter and procedure catheter, respectively, according to some embodiments.

FIGS. 54-63 illustrate examples of a configuration of a concentric stack of elongated devices during contrast injection. In an example, a concentric stack of catheters can be the concentric stack illustrated in FIG. 53, which shows the concentric stack with the guide catheter 2906, that is, having a guide catheter 2906, a procedure catheter 2904, an insert catheter 2902, and a guide wire 2907. In some examples, instead of a guide wire the 2907 the concentric stack includes another EMD (e.g., a small diameter catheter or other device). A fluidics system can be configured to provide, individually or simultaneously, contrast to any one or more of the guide catheter 2906, a procedure catheter 2904, and the insert catheter 2902. For example, one or more of the control valves 1426a-c in the hub assemblies (or mounts) 1400a-c can be aligned to provide contrast to a catheter coupled to the hub assemblies 1400a-c such that the fluidic system can provide contrast to a patient via one or more of the guide catheter 2906, the procedure catheter 2904, and/or the insert catheter 2902. In operation, the elongated devices of the concentric stack can be moved along a longitudinal axis in a distal (e.g., towards patient) or proximal (e.g., away from patient) direction either together or individually. In some examples, the fluidic system provides contrast through the innermost catheter that has a distal end near a target area. In various examples of configurations, all of the four EMDs in FIG. 53 do not have to be part of the concentric stack. For example, if a controller causes contrast to be injected through the guide catheter 2906 through cross-sectional area 47 and the procedure catheter 2094 is in the concentric stack and positioned in the lumen of the guide catheter 2906, having the insert catheter 2902 or the guide wire 2907 in the concentric stack does not change the lumen size of the guide catheter 2906. The controller can cause the contrast fluidic channel to be aligned to provide contrast through any one or more of the catheters in the concentric stack. In FIGS. 54-63, the catheters and guide wire may be depicted with a dashed line which indicates it is not part of the stack (the dashed line included merely for spatial context), or the catheters and guide wire may be depicted by a dotted line which indicates it is optionally in the concentric stack but its presence does not affect the size of the lumen through which contrast is provided. For example, FIG. 54 is a representation of an example of a distal end view of a concentric stack (“stack”) of one or more elongated medical devices that can be in a patient during contrast injection through a guide catheter, the stack including the guide catheter 2906 and a procedure catheter, and optionally an insert catheter 2902 and a guiding element 2907 (both depicted with dotted lines), and where contrast is injected into the patient through the lumen of the guide catheter 2906 in the effective cross-sectional area 47 between an inner surface of the guide catheter 2906 and an outer surface of the procedure catheter 2904 which is positioned partially or fully in the lumen of the guide catheter 2906. In some embodiments, the catheters or the guide wire depicted in dashed lines can indicate that although it is part of the stack, it may be retracted such that its distal end is not at the most distal end of a catheter in the stack (and it does not affect the size of the lumen through which contrast is provided.

FIG. 55 is a representation of another example of a distal end view of a concentric stack of one or more elongated medical devices that can be in a patient during contrast injection through a guide catheter 2906, the stack including the guide catheter 2906 and an insert catheter 2902, but not a procedure catheter 2904 (depicted with a dashed line), and optionally a guiding element 2907 (depicted with a dotted line), the contrast being injected into the patient through the space between an inner surface of the guide catheter 2906 and an outer surface of the insert catheter 2902. In this example, the effective cross-sectional area is the sum of the effective cross-sectional areas 47 and 45.

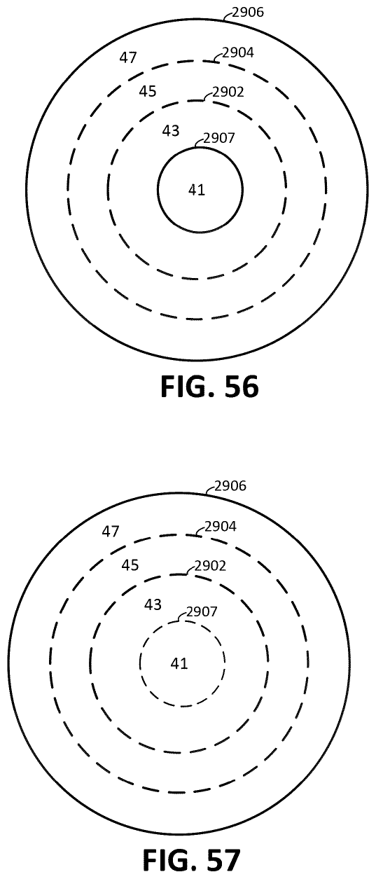

FIG. 56 is a representation of an example of a distal end view of a concentric stack of one or more elongated medical devices that can be in a patient during contrast injection through a guide catheter 2906, the stack including the guide catheter 2906 and a guiding element 2907 but not a procedure catheter 2904 or an insert catheter 2902 (both depicted with dashed lines), the contrast injected into the patient through the space between an inner surface of the guide catheter 2906 and the outer surface of the guiding element 2907. In this example, the effective cross-sectional area is the sum of the effective cross-sectional areas 47 and 45 and 43.

FIG. 57 is a representation of an example of a distal end view of a concentric stack of one or more elongated medical devices that can be used in a medical procedure during contrast injection through a guide catheter 2906, the stack including the guide catheter 2906, but not a procedure catheter 2904, an insert catheter 2902, or a guiding element 2907 (all depicted with dashed lines), the contrast being injected into the patient through the lumen of the guide catheter 2906. In this example, the effective cross-sectional area is the sum of the effective cross-sectional areas 47 and 45 and 43 and 41.

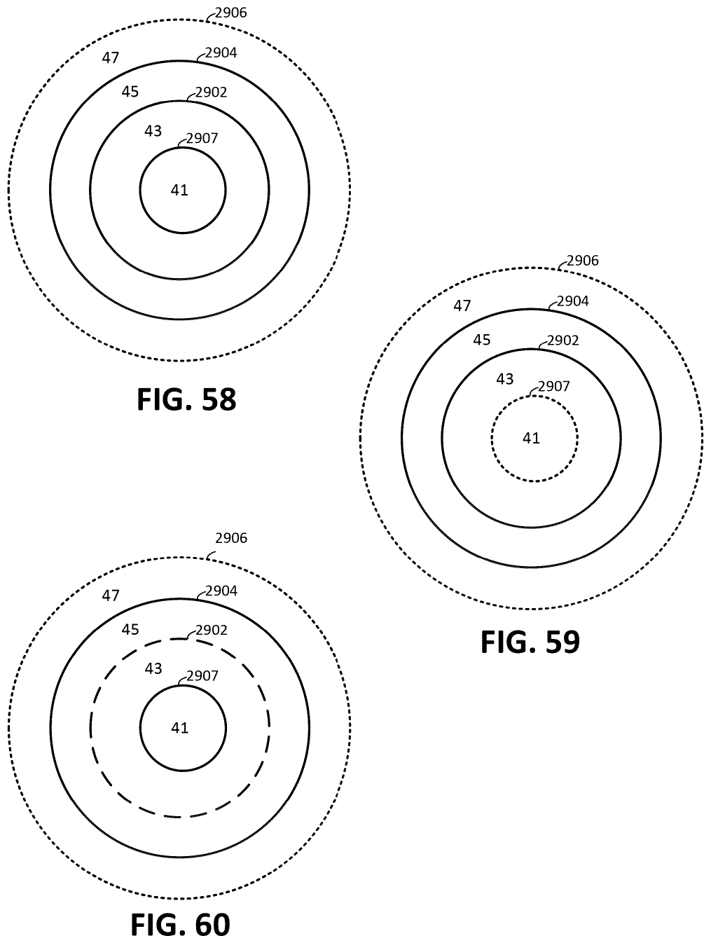

FIG. 58 is a representation of an example of a distal end view of a concentric stack of one or more elongated medical devices that can be used in a medical procedure during contrast injection through a procedure catheter 2904. The stack in this example included the procedure catheter 2904, an insert catheter 2902, and a guiding element and optionally a guide catheter 2907, the contrast being injected into the patient through the lumen of the procedure catheter 2904 in the space between an inner surface of the procedure catheter 2904 and an outer surface of the insert catheter 2902 (i.e., effective cross-sectional area 45.

FIG. 59 is a representation of an example of a distal end view of a concentric stack of one or more elongated medical devices that can be used in a medical procedure during contrast injection through a procedure catheter 2904. The stack in this example includes the procedure catheter 2904, the insert catheter 2902, and optionally the guiding element 2907 (depicted with a dotted line) and the guide catheter 2906 (depicted with a dotted line), the contrast being injected into the patient through the lumen of the procedure catheter 2904 in the space between an inner surface of the procedure catheter 2904 and an outer surface of the insert catheter 2902 (i.e., through effective cross-sectional area 45).

FIG. 60 is a representation of an example of a distal end view of a concentric stack of one or more elongated medical devices that can be used in a medical procedure during contrast injection through a procedure catheter 2904 with a guiding element 2907 in the lumen of the procedure catheter, but not an insert catheter 2902 (depicted with a dashed line) and optionally the guide catheter 2906 (depicted with a dotted line). In this example, contrast can be injected into the patient through the lumen of the procedure catheter 2904 in the space between an inner surface of the procedure catheter 2904 and an outer surface of the guiding element 2907 (i.e., through the sum of effective cross-sectional areas 45 and 43).

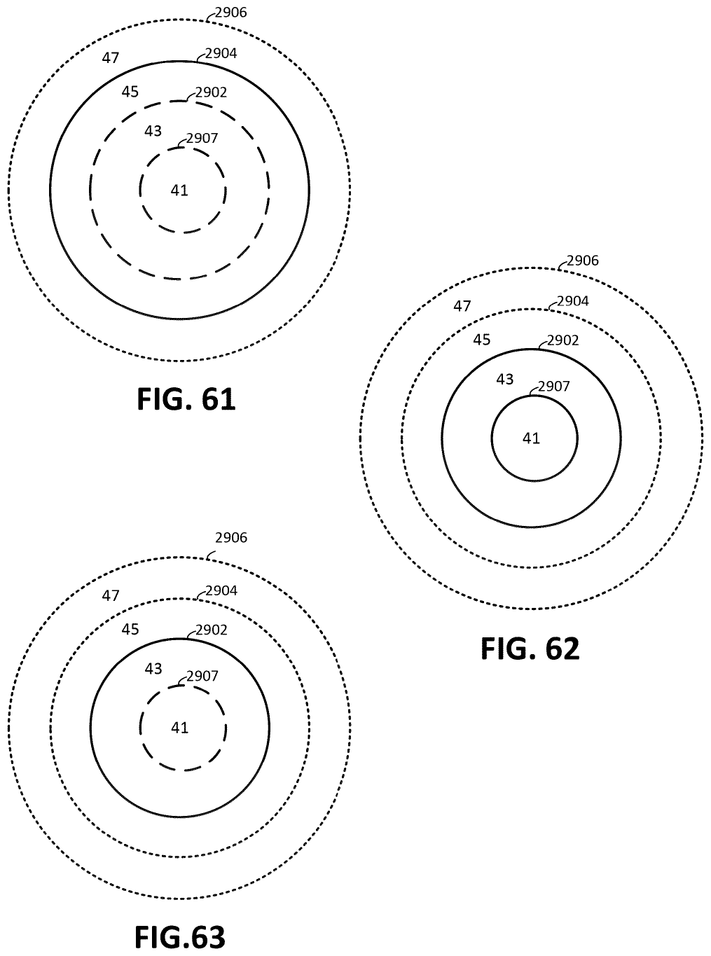

FIG. 61 is a representation of an example of a distal end view of a concentric stack of one or more elongated medical devices that can be used in a medical procedure during contrast injection through a procedure catheter 2904, the stack including the procedure catheter 2904, optionally including a guide catheter 2906 (depicted with a dotted line) but not including an insert catheter 2902 (depicted with a dashed line) or a guiding element 2907 (depicted with a dashed line), the contrast being injected into the patient through the lumen of the procedure catheter 2904 (i.e., through the sum of the effective cross-sectional areas 45 and 43 and 41.

FIG. 62 is a representation of an example of a distal end view of a concentric stack of one or more elongated medical devices that can be used in a medical procedure during contrast injection through an insert catheter 2902, the stack including the insert catheter 2902 and the guiding element 2907, and optionally including the guide catheter 2906 and the procedure catheter 2904 (both depicted with dotted lines), the contrast being injected into the patient through the lumen of the insert catheter 2902 in the space between an inner surface of the insert catheter 2902 and an outer surface of the guiding element 2907 (i.e., through the effective cross-sectional area 43.

FIG. 63 is a representation of an example of a distal end view of a concentric stack of one or more elongated medical devices that can be used in a medical procedure during contrast injection through an insert catheter 2902 but not including the guiding element 2907 (depicted in a dashed line), and optionally including a guide catheter 2906 and a procedure catheter 2904, the contrast being injected into the patient through the lumen of the insert catheter 2902 (i.e., through the sum of the effective cross-sectional areas 43 and 41).

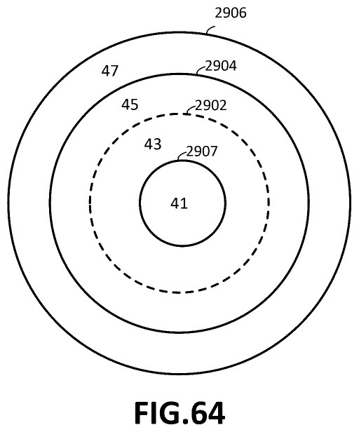

FIG. 64 is a representation of an example of a distal end view of a concentric stack of one or more elongated medical devices that can be used in a medical procedure during contrast injection through a procedure catheter 2904. In this configuration of the concentric stack, a guidewire 2907 is in the lumen of the procedure catheter 2904 but the concentric stack does not include the insert catheter 2902 (depicted in a dashed line). Contrast can be injected into the patient through the lumen of the procedure catheter 2904 (i.e., through the sum of the effective cross-sectional areas 45 and 43). In an example, such a configuration may be used when the procedure catheter 2904 is corked (i.e., obstructed). In such circumstances, the procedure catheter 2904 may be completely removed to clear it, then it may be advanced into the guide catheter 2906 to gain the appropriate distal access. Then, the procedure catheter 2904 could provide contrast (e.g., a puff of contrast) to determine if there was still in obstruction or if it was clear.

In some embodiments, the length of the access catheter 2902 may be between about 100 and 193 centimeters. In certain embodiments, a wall of the access catheter 2902 surrounding the lumen may include a braided reinforcement layer and an interior liner (e.g., PEBAX). The braided reinforcement layer may include stainless metal ribbon wire in a tight braid pattern. In an example, the metal ribbon wire can have a cross-sectional dimension of about 0.002″×about 0.005″. In other embodiments, the braided reinforcement layer may include a stainless metal round wire having a 0.002 inch diameter in a tight braid pattern (e.g., a 1:1 braid pattern, or a 2:2 braid pattern). In various examples, the ribbon material could be made from, or include, stainless steel or other materials, including titanium, CoCr alloys, Elgiloy, Hasteloy (Hastelloy alloy), or the like.

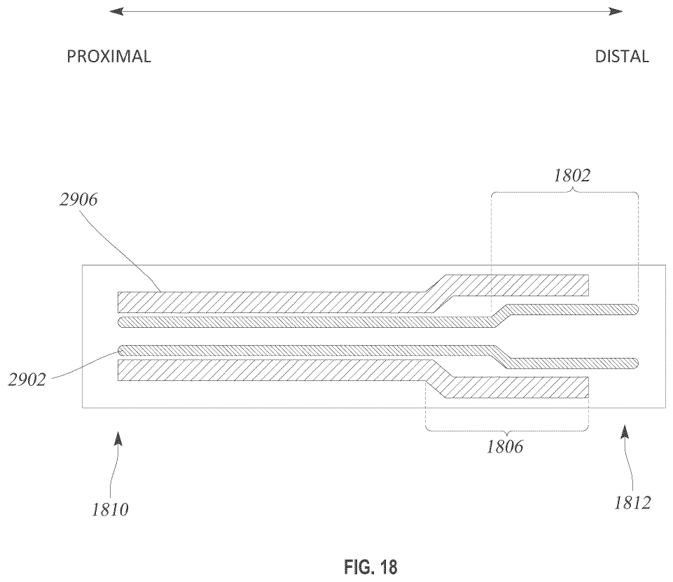

The guide catheter 2906 and the access catheter 2902 may each have a proximal end 1810 and a distal end 1812. In some embodiments, the distal ends 1812 of guide catheter 2906 and access catheter 2902 may each have a hypotube tip, 1806 and 1802 respectively, as shown in FIG. 18. In some example, the tip may include Nitinol. In an example, a catheter tip can include a laser-cut hypotube comprised of Nitinol, which is bonded to another proximal laser-cut hypotube comprised of stainless steel. This advantageously allows for better pushability and torqueability due to the enhanced stiffness material characteristics of stainless steel. Flexibility of a metal hypotube may be achieved through a pattern of cuts in its walls. In an example, an interrupted spiral pattern can achieve this. In another example, a plurality of cuts (for example, a pattern of cuts or apertures) can be used to achieve a desired stiffness/flexibility of the catheter tip. The length of the cuts and the pitch between the cuts can help define the stiffness of the catheter. One or more lasers can be used to make the cuts. In some embodiments, the hypotube tip is the same diameter (for example, outside diameter of the hypotube tip is the same diameter as the portion of the catheter adjacent to the hypotube tip). In some embodiments, the hypotube tip has a smaller diameter than the portion of the catheter proximal to the tip (for example, the outside diameter of the hypotube tip is smaller than the outside diameter the portion of the catheter proximal to the hypotube tip). This advantageously allows for the distal tip to be small enough to engage with distal anatomy while having a stiffer proximal end that enables better pushability and torque control, as well as reduced susceptibility to buckling.

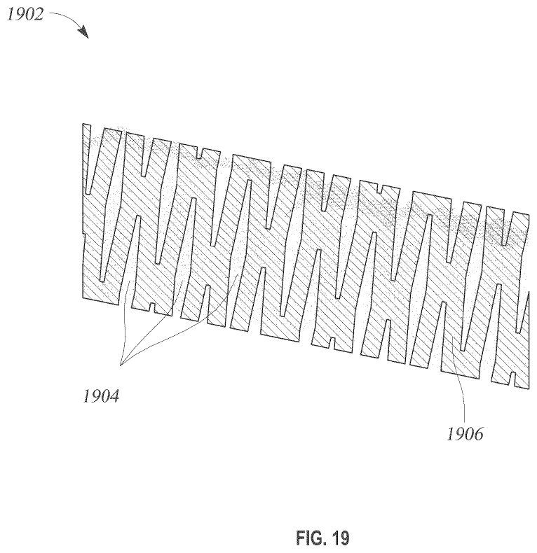

FIG. 19 shows a cross-sectional view of a laser cut hypotube 1902. In some embodiments, a laser cut hypotube may be comprised of a metal (e.g., Nitinol, stainless steel, etc.) scaffold 1906 defining a plurality of apertures 1904. In some embodiments, the distal end of access catheter 2902 may comprise a laser cut hypotube 1902, for enhanced heat-setting properties to allow the access catheter to be shaped appropriately to navigate a patient's vasculature. In some embodiments, using a Nitinol hypotube allows the catheter to be folded back, or bent, while packaged. This is advantageous as the catheters could be too long to be packaged straight. The super-elastic nature of Nitinol, further allows for the catheter to torque without the catheter whipping.

While the embodiments disclosed herein are described with respect to the injection of contrast media, those of skill in the art would appreciate that these concepts may be applicable to any fluid injected into the catheter assembly 2900 (e.g., saline, medicament, etc.). The viscosity of the liquid being injected into the catheter assembly 2900 may affect the annular space required in the lumen of the access catheter 2902 to allow for sufficient flow rate. Accordingly, access catheters 2902 of different diameters and/or lengths than those described above may be desirable depending on the viscosity of the liquid being injected into the catheter assembly 2900. Similarly, guidewires of different diameters or having different properties (e.g, having a hydrophilic coating) may be desirable depending on the properties of the liquid being injected into the catheter system to facilitate desired flow characteristics. In some embodiments, a guiding element (e.g., guidewire) can include different surface properties on its proximal end and distal end. In an example, a guiding element can include a hydrophilic coating on its distal end and a hydrophobic coating on its proximal end. In an example, the hydrophobic coating is polytetrafluoroethylene (PTFE). In an example, the hydrophobic coating comprises a PTFE-based composition. In an example, the PTFE-based composition is Teflon™. The examples detailed at the end of this disclosure illustrate different configurations of some embodiments of the catheter assembly 2900 that may be used.

FIGS. 20A-20E depict an example sequence of steps of introducing a multi-catheter assembly configured to achieve access all the way to the clot, either manually or robotically. FIGS. 20A-20E may be described using the interventional device assembly of FIG. 17. Other combinations of catheters may be substituted for the interventional device assembly, as will be appreciated by those of skill in the art in view of the disclosure herein.

Referring to FIG. 20A, the three-catheter interventional device assembly 2900 is shown driven through an introducer sheath 3002, up through the iliac artery 3004 and into the descending aorta. Next, the access catheter 2902, the procedure catheter 2904 (e.g., 0.071 inch) and the guide catheter 2906 (e.g., 0.088 inch) are tracked up to the aortic arch 3006, as shown in FIG. 20B. Here, the distal end of the guide catheter 2906 may be parked below the aortic arch 3006 and the procedure catheter 2904, access catheter 2902 (positioned within the procedure catheter 2904 and not visible in FIG. 20B) and a guidewire 2907 can be driven into the ostium (e.g., simultaneously or separately). In some embodiments, the access catheter 2902 is advanced out of the procedure catheter 2904 and the guide catheter 2906 to engage the ostium first. After the distal end of the access catheter 2902 is positioned within the desired ostium, the guidewire 2907 can be advanced distally into the ostium to secure (or guide) access, and/or to confirm proper vessel selection and positioning. After the access catheter 2902 and guidewire 2907 are positioned within the desired ostium, the procedure catheter 2904 and/or guide catheter 2906 can be advanced into the ostium (and, in some embodiments, beyond), while using the support of the access catheter 2902 and/or guidewire 2907 to maneuver through the aorta and into the ostium. In the embodiment shown in FIG. 20B, the procedure catheter 2904 has been advanced into the ostium while the guide catheter 2906 has remained parked below the aortic arch 3006.

Referring to FIG. 20C, the guidewire 2907 may be distally advanced and the radiopacity of the guidewire 2907 may be used to confirm under fluoroscopic imaging that access through the desired ostia has been attained. The guidewire 2907 engages the origin of the brachiocephalic artery 3014. The guidewire 2907 is then advanced up to the petrous segment 3018 of the internal carotid artery 3016.

Referring to FIG. 20D, the guide catheter 2906 and the procedure catheter 2904 (positioned within the guide catheter 2906 and not visible in FIG. 20D) are both advanced (e.g., simultaneously or sequentially) over the guidewire 2907 and over the insert or access catheter 2902 (positioned within the procedure catheter 2904 and not visible in FIG. 20D) while the access catheter 2902 remains at the ostium for support. The guidewire 2907 may be further advanced past the petrous segment 3018 to the site of the clot 3020, such as the Ml segment.

Referring to FIG. 20E, the guide catheter 2906 and the procedure catheter 2904 (positioned within the guide catheter 2906 and not visible in FIG. 20D) are advanced (e.g., simultaneously or sequentially) to position the distal tip of the procedure catheter 2904 at the procedure site, for example on the face of the clot 3020. The guidewire 2907 and access catheter 2902 (positioned within the procedure catheter 2904 and not visible in FIG. 20E) are removed, and aspiration of the clot 3020 commences through the procedure catheter 2904. That is, the guidewire 2907 and the access catheter 2902 are proximally retracted to allow aspiration through the procedure catheter 2904. After aspiration of the clot, the procedure catheter 2904 and guide catheter 2906 can be removed (e.g., simultaneously or sequentially). For example, in some embodiments, the procedure catheter 2904 may be removed before removing the guide catheter 2906.

The catheter assembly 2900 may be used to perform a neurovascular procedure, as described in FIGS. 20A-20E. For example, the neurovascular procedure may be a neurovascular thrombectomy. The steps of the procedure may include providing an assembly that includes at least a guidewire, an access catheter, a guide catheter, and a procedure catheter. For example, the catheter assembly 2900 includes a guidewire 2907, an access catheter 2902, a guide catheter 2906, and at least one procedure catheter 2904. The procedure catheter 2904 may include an aspiration catheter, an embolic deployment catheter, a stent deployment catheter, a flow diverter deployment catheter, a diagnostic angiographic catheter, a stent retriever catheter, a clot retriever catheter a balloon catheter, a catheter to facilitate percutaneous valve repair or replacement, an ablation catheter, and/or an RF ablation catheter or guidewire.

The neurovascular procedure may further include steps of coupling the assembly to a non-robotic or a robotic drive system and driving the assembly to achieve supra-aortic access. The steps may further include driving a subset of the assembly to a neurovascular site and performing the neurovascular procedure using a subset of the assembly. The subset of the assembly may include the guidewire, the guide catheter, and the procedure catheter.

Each of the guidewire 2907, the access catheter 2902, the procedure catheter 2904, and the guide catheter 2906 is configured to be adjusted by a respective hub. For example, the guidewire 2907 may include (or be coupled to) a hub installed on one of the tray assemblies described herein (for example, hub 26, FIG. 3A). Similarly, the access catheter 2902 may include, or be coupled to catheter hub 2910. The procedure catheter 2904 may include, or be coupled to, the procedure catheter hub 2912. The guide catheter 2906 may include, or be coupled to, the guide catheter hub 2914.

In general, coupling of the assembly 2900 may include magnetically coupling a first hub 2909, which is coupled to the guidewire 2907, to a first drive magnet; magnetically coupling a second hub 2910, which is coupled to the access catheter 2902, to a second drive magnet; magnetically coupling a third hub 2912, which is coupled to the procedure catheter 2904, to a third drive magnet; and magnetically coupling a fourth hub 2914, which is coupled to the guide catheter 2906, to a fourth drive magnet. In various embodiments, there can be one layer of material, or multiple layers of material (that is, one or more layers of material), between each of the first, second, third, and fourth hubs, and their corresponding first, second, third, and fourth drive magnets, such that the magnetic couplings are through the layer(s) of material. In various embodiments, each layer may be flexible, semi-rigid, or rigid. A layer can be a sterile barrier. When the system is configured for use, the first drive magnet, the second drive magnet, the third drive magnet, and the fourth drive magnet are each independently movable, and movably carried by (or on) a drive table, for example, as described with respect to tray assemblies and controls described herein. In some embodiments, the first drive magnet, the second drive magnet, the third drive magnet, and the fourth drive magnet are coupled to their respective catheter hubs through a sterile barrier (e.g., a sterile and fluid barrier). Each of the first, second, third, and fourth drive magnets may be controlled to be independently movable relative to the other drive magnets. Each of the first, second, third, and fourth drive magnets may be incorporated on a drive table having a plurality of drive magnets that are controlled to move along the drive table (for example, along a longitudinal axis of the drive table. In some embodiments, two or more drive magnets can be tethered or otherwise coupled together to move as a unit in response to commands from a single controller. In some examples, the drive magnets can be coupled together to move together (e.g., physically coupled together or configured to move together by a controller). In an example, the first and second drive magnets may be configured to move together along the drive table. In another example, the first, second, third, and fourth drive magnets may be configured to move together. In another example, any two or more of the first, second, third, and fourth drive magnets (and correspondingly, the hubs the first, second, third, and fourth drive magnets are coupled to) may be configured to be moved together along a drive table.

In some implementations, the steps of performing the neurovascular procedure may include driving the assembly in response to movement of hub adapters along a support table until the assembly is positioned to achieve supra-aortic vessel access. The hub adapters may include, for example, a coupler/carriage that acts as a shuttle by advancing proximally or distally along a track in response to operator instructions. The hub adapters described herein may each include at least one drive magnet configured to couple with a magnet (sometimes referred to herein as a “driven magnet”) carried by the respective hub. This provides a magnetic coupling between the drive magnet and driven magnet through the sterile barrier such that the respective hub is moved across the top of the sterile barrier (within the sterile field) in response to movement of the hub adapter which is positioned outside of the sterile field (as described in detail in FIG. 4). Movement of the hub adapter is driven by a drive system carried by the support table in which the guidewire hub 2909, the access catheter hub 2910, the procedure catheter hub 2912, and the guide catheter hub 2914 are installed upon.

Movement of the catheter assembly 2900 during a procedure may include moving including a portion on the catheter assembly 2900. For example, moving the catheter assembly 2900 during a procedure may include driving a subset of the assembly in response to movement of one or more of the hub adapters along the support table until the subset of the assembly is positioned to perform a neurovascular procedure at a neurovascular treatment site. The subset of the assembly may include the guidewire 2907, the guide catheter 2906, and/or the procedure catheter 2904.

In some embodiments, the guidewire 2907, the guide catheter 2906 and the procedure catheter 2904 are advanced as a unit through (with respect to the guidewire 2907) and over (with respect to the guide catheter 2906 and the procedure catheter 2904) at least a portion of a length of the access (e.g., insert) catheter 2902 after supra-aortic access is achieved.

In some embodiments, the catheter assembly 2900 may be part of a robotic control system for achieving supra-aortic access and neurovascular treatment site access, as described in FIGS. 20A-20E. In some embodiments, the catheter assembly 2900 may be part of a manual control system for achieving supra-aortic access and neurovascular treatment site access. In some embodiments, the catheter assembly 2900 may be part of a hybrid control system (with manual and robotic components) for achieving supra-aortic access and neurovascular treatment site access. For example, in such hybrid systems, supra-aortic access may be robotically driven while neurovascular site access and embolectomy or other procedures may be manual. Alternatively, in such hybrid systems, supra-aortic access may be manual while neurovascular site access may be robotically achieved. Still further, in such hybrid systems, any one or more of: the guidewire, access catheter, guide catheter, or procedure catheter may be robotically driven or manually manipulated.

An example robotic control system may include at least a guidewire hub (e.g., guidewire hub 2909) configured to adjust each of an axial position and a rotational position of a guidewire 2907. The robotic control system may also include an access catheter hub 2910 configured to adjust axial and rotational movement of an access catheter 2902. The robotic control system may also include a guide catheter hub 2914 configured to control axial movement of a guide catheter 2906. The robotic control system may also include a procedure catheter hub 2912 configured to adjust an axial position and a rotational position of a procedure catheter 2904.

In some embodiments, the procedure catheter hub 2912 is further configured to laterally deflect the procedure catheter 2904 through a distal deflection zone.

In some embodiments, the guidewire hub 2909 is configured to couple to a guidewire hub adapter by magnetically coupling the guidewire hub to a first drive magnet. The access catheter hub 2910 is configured to couple to an access catheter hub adapter by magnetically coupling the access catheter hub 2910 to a second drive magnet. The procedure catheter hub 2912 is configured to couple to a procedure catheter hub adapter by magnetically coupling the procedure catheter hub 2912 to a third drive magnet. The guide catheter hub 2914 is configured to couple to a guide catheter hub adapter by magnetically coupling the guide catheter hub 2914 to a fourth drive magnet. In some embodiments, the first drive magnet, the second drive magnet, the third drive magnet, and the fourth drive magnet are independently movably carried by a drive table.

In some embodiments, the robotic control system includes a first driven magnet on the guidewire hub 2909. The first driven magnet may be configured to cooperate with the first drive magnet such that the first driven magnet is configured to move in response to movement of the first drive magnet. In some embodiments, the first drive magnet is configured to move outside of a sterile field separated from the first driven magnet by a barrier while the first driven magnet is within the sterile field. In some embodiments, a position of the first driven magnet is movable in response to manipulation of a procedure drive control on a control console associated with the drive table. Drive magnets and driven magnet interactions are described in detail in FIG. 4 above.

In some embodiments, the robotic control system includes a second driven magnet on the access catheter hub 2910. The second driven magnet may be configured to cooperate with the second drive magnet such that the second driven magnet is configured to move in response to movement of the second drive magnet. In some embodiments, the second drive magnet is configured to move outside of a sterile field separated from the second driven magnet by a barrier while the second driven magnet is within the sterile field.

In some embodiments, the robotic control system includes a third driven magnet on the procedure catheter hub 2912. The third driven magnet may be configured to cooperate with the third drive magnet such that the third driven magnet is configured to move in response to movement of the third drive magnet. In some embodiments, the third drive magnet is configured to move outside of a sterile field separated from the third driven magnet by a barrier while the third driven magnet is within the sterile field.

In some embodiments, the robotic control system includes a fourth driven magnet on the guide catheter hub 2914. The fourth driven magnet may be configured to cooperate with the fourth drive magnet such that the fourth driven magnet is configured to move in response to movement of the fourth drive magnet. In some embodiments, the fourth drive magnet is configured to move outside of a sterile field separated from the fourth driven magnet by a barrier while the fourth driven magnet is within the sterile field. In some embodiments, there may be more than four driven magnets and corresponding catheter hubs for control of additional catheters.

In some embodiments, devices (e.g., hubs, hub adapters, interventional devices, and/or trays) described herein may be used during a robotically driven procedure. For example, in a robotically driven procedure, one or more of the interventional devices may be driven through vasculature and to a procedure site. Robotically driving such devices may include engaging electromechanical components that are controlled by user input. In some implementations, users may provide the input at a control system that interfaces with one or more hubs and hub adapters.

In some embodiments, the hubs, hub adapters, interventional devices, and trays described herein may be used during a non-robotic (e.g., manually driven) procedure. Manually driving such devices may include engaging manually with the hubs to affect movement of the interventional devices.

In some embodiments, the devices described herein may be used to carry out a method of performing an intracranial procedure at an intracranial site. The method of performing the intracranial procedure may include any of the same steps as described herein for performing a neurovascular procedure. The procedure may be robotically performed, manually performed, or a hybridized combination of both.

While the foregoing describes magnetic coupling of hubs to drive magnets, in other embodiments, any of the interventional devices and/or hubs may be mechanically coupled to a drive system. Any of the methods described herein may include steps of mechanically coupling one or more interventional devices (e.g., the guidewire 2907, the access catheter 2902, the procedure catheter 2904, and/or the guide catheter 2906) and/or one or more hubs (e.g., the guidewire hub 2909, the access catheter hub 2910, the procedure catheter hub 2912, and/or the guide catheter hub 2914) with one or more drive mechanisms.

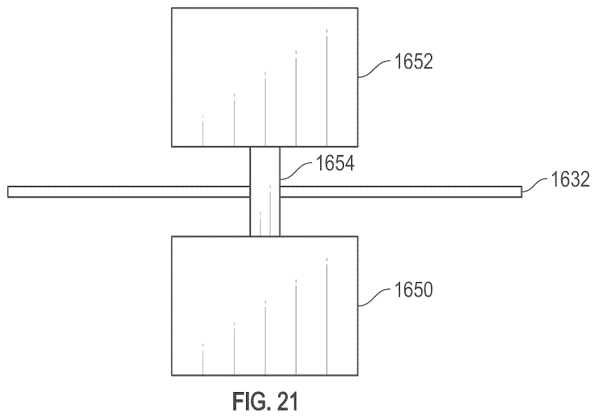

FIG. 21 illustrates an embodiment of a mechanical coupling mechanism 1654 between a drive mechanism 1650 and a driven mechanism 1652. Drive mechanism 1650 and driven mechanism 1652 may have any of the same or similar features or functions as the drive magnet 67 and driven magnet 69, respectively, except as otherwise described herein. The drive mechanism 1650 may be part of or coupled to a hub adapter (e.g., the hub adapter 48). The driven mechanism 1652 may be part of or coupled to a hub (e.g., the hub 36, the guidewire hub 2909, the access catheter hub 2910, the procedure catheter hub 2912, or the guide catheter hub 2914). In some instances, the mechanical coupling mechanism 1654 may comprise a structural support (e.g., a support rod or support strut) extending transversely through a seal in a sterile barrier 1632. The seal may permit the structural support to be advanced along a length of the sterile barrier 1632, while still maintaining a seal with the structural support to maintain the sterile field, as the drive mechanism 1650 and driven mechanism 1652 are advanced and/or retracted as described herein. For example, the seal may comprise a tongue and groove closure mechanism along the sterile barrier 1632 that is configured to close on either side of the structural support while permitting passage of the structural support through the sterile barrier 1632 and maintaining a seal against the structural support as the structural support is advanced along the length of the sterile barrier 1632.

In some embodiments, the structural support can extend through an elongate self-closing seal between two adjacent coaptive edges of flexible material (e.g., similar in shape to a duckbill valve) that extends along an axis. As the structural support advances along the axis between the coaptive edges, the coaptive edges may permit the structural support to advance, and then may be biased back into a sealing engagement with each other as the structural support passes any given point along the axis.

In some embodiments, the drive mechanism may be a splined drive shaft (e.g., a non-sterile splined drive shaft). The mechanical coupling 1654 can include a pulley within a plate that serves as the sterile barrier 1632 and a sterile splined shaft configured to couple to the driven mechanism 1652. The driven mechanism 1652 can be a sterile pulley that receives the sterile splined shaft from the sterile barrier. In some embodiments, one or more splined drive shafts can engage and turn corresponding pulleys in the plate that serves as the sterile barrier. Each hub can have a sterile pulley that is configured to receive a sterile splined shaft from the sterile barrier plate. Rotation of the splined drive shaft can turn the pulley in the sterile barrier plate which can, in turn, turn the sterile pulley in the hub via the sterile splined shaft.

It will be understood by one having skill in the art that any embodiment as described herein may be modified to incorporate a mechanical coupling mechanism, for example, as shown in FIG. 21.

The interventional devices described herein may be provided individually, or at least some of the interventional devices can be provided in a preassembled (e.g., nested or stacked) configuration, for example, as part of a sterile kit. In an example, the interventional devices may be provided in the form of an interventional device assembly, such as interventional device assembly 2900, in a concentric nested or stacked configuration. If provided individually, each catheter (and in some embodiments, each corresponding catheter hub) can be unpackaged and primed to remove air from its inner lumen, for example, by flushing the catheter (and in some embodiments, each corresponding catheter hub) to remove air by displacing it with a fluid, such as saline, contrast media, or a mixture of saline and contrast media. After priming, the interventional devices can be manually assembled into a stacked configuration so that they are ready for introduction into the body for a surgical procedure, for example, via an introducer sheath.

Assembling the devices into a stacked configuration can include individually inserting interventional devices into one another by order of size. For example, an interventional device having a second largest diameter can be inserted into the lumen of an interventional device having a largest diameter. An interventional device having a third largest diameter can then be inserted into the interventional device having the second largest diameter and so on.

For example, with respect to FIG. 17, assembly can be performed by first inserting a distal end of the catheter 2904 through the hub 2914 and into the catheter 2906. The catheter 2904 can be advanced through the catheter 2906 until the distal tip of the catheter 2904 is flush with or extends beyond the distal tip of the catheter 2906, and/or until the catheter 2904 cannot be inserted any further. Then, the distal end of the catheter 2902 can be inserted through the hub 2912 and into the catheter 2904. The catheter 2902 can be advanced through the catheter 2904 until the distal tip of the catheter 2902 is flush with or extends beyond the distal tip of the catheter 2904, and/or until the catheter 2902 cannot be inserted any further. Then, the distal end of the guidewire 2907 can be inserted through the hub 2910 and into the catheter 2902. The guidewire 2907 can be advanced through the catheter 2902 until the distal tip of the guidewire 2907 is flush with or extends beyond the distal tip of the catheter 2902, and/or until the guidewire 2907 cannot be inserted any further.

Embodiments in which two or more of the interventional devices are packaged together as a single unit in an assembled (e.g., nested or stacked) configuration may provide efficient unpackaging and preparation prior to use and efficient assembly within a robotic control system. The interventional devices may be pre-mounted to their respective hubs prior to packaging. In certain embodiments, two or three or more interventional devices may be packaged in a fully nested (i.e., fully axially inserted) configuration or nearly fully nested configuration. In a fully nested configuration, each interventional device is inserted as far as possible into an adjacent distal hub and interventional device. Such a fully nested configuration may minimize a total length of the interventional device assembly and minimize the size of the packaging required to house the interventional device assembly.

In some embodiments, the interventional devices may also be sterilized prior to packaging while in the assembled configuration, for example, using ethylene oxide gas. In some embodiments, the interventional devices may be packaged while in the assembled configuration before sterilization with ethylene oxide gas. For interventional devices in a nested or stacked configuration, ethylene oxide gas can be provided in a space between adjacent interventional devices (for example, an annular lumen between an outer diameter of a first interventional device nested within a second interventional device and the inner diameter of the second interventional device) for sterilization. In some embodiments, the interventional device assembly can be packaged in a thermoformed tray and sealed with an HDPE (e.g., Tyvek®) lid. The interventional device assembly can be unpackaged by removal (e.g., opening or peeling off) of the lid by a user in a sterile field. A user in the sterile field can then remove the interventional device assembly and place it on the sterile work surface, for example, of a robotic drive table, as described herein.

Packaging the interventional devices in an assembled configuration and sterilized state can reduce the time associated with unpackaging and assembly of individual interventional devices and facilitate efficient connection to a robotic drive system. Each interventional device and hub combination may further be packaged with a fluidics connection for coupling to a fluid source, or one or more fluid sources, and/or a vacuum source. In some embodiments, each hub, or a hemostasis valve coupled to the hub, may include the fluidics connection.

After the interventional device assembly is unpackaged (e.g., after the interventional device assembly is positioned on the robotic drive table), priming can be performed while the devices are concentrically nested or stacked. This is preferably accomplished in each catheter lumen, for example, the annular lumen between the guide catheter 2906 and the procedure catheter 2904, and in between each of the additional concentric interventional devices in the catheter stack. In certain embodiments, fluid can be introduced in one or more lumens of the catheter stack to prime one or more interventional devices. For example, fluid can be introduced in a lumen between a distal hub and a proximal interventional device. For example, the lumen between the hub 2914 and the catheter 2904. In certain embodiments, priming can be performed while the devices are in the sterile packaging. More typically, priming of one of more of the catheters in the catheter stack can be performed when the catheter assembly has been removed from its packaging.

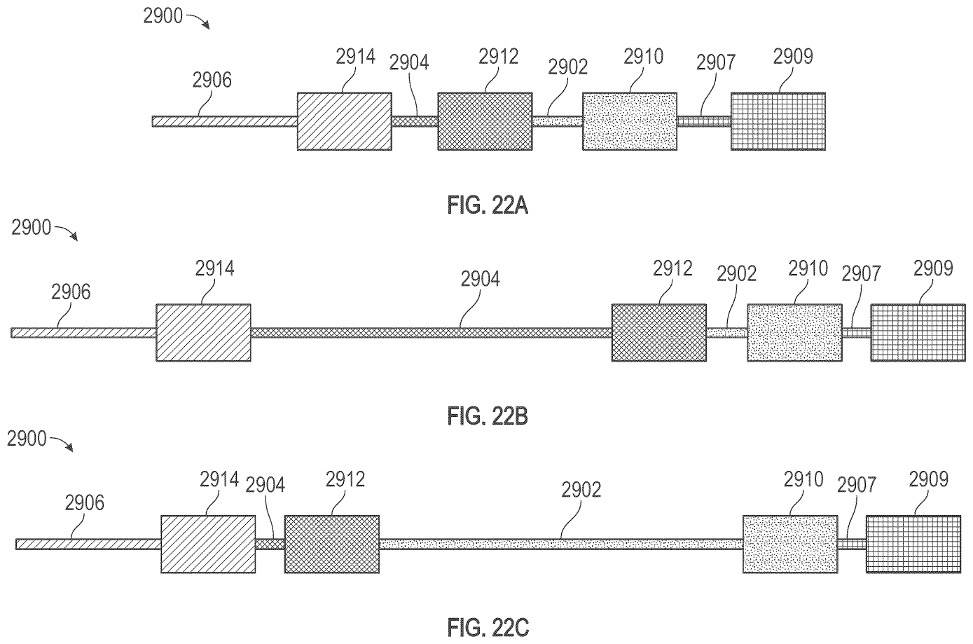

The fluidics connections of the catheter assembly (for example, the fluidics connection to one or more of the hubs) can be connected to a fluidics system for delivering saline and contrast media to the catheters, and providing aspiration. In some embodiments, one or more of the fluidics connections (e.g., to saline, contrast, or aspiration) may extend from the sterile field to outside the sterile field for connection to the fluidics system. Once connected, the fluidics system can perform a priming sequence to flush each catheter of the interventional device assembly with fluid (e.g., saline, contrast media, or a mixture of saline and contrast media). The priming sequence may also include flushing each corresponding catheter hub with fluid. The fluid may be de-aired or de-gassed by the fluidics system prior to priming. In some embodiments, a vacuum source of the fluidics system can also be used to evacuate air from each catheter while flushing with fluid. In certain embodiments, a tip of the catheter can be placed into a container of fluid, such as saline, contrast media, or a mixture of saline and contrast media, during priming so that the fluid in the container, and not air, is aspirated through the tip of the catheter when the vacuum source is applied. In other embodiments, the tip of the catheter may be blocked (for example, using a plug) so that air is not aspirated from the tip of the catheter when the vacuum source is applied. In certain embodiments, the priming process may be automated such that a user can provide a single command and each catheter (and in some embodiments, each corresponding catheter hub) can be primed, sequentially (for example, as described with respect to FIGS. 22A-22C) or simultaneously.

Additional details regarding fluidics systems are disclosed in U.S. patent application Ser. No. 17/879,614, entitled Multi Catheter System With Integrated Fluidics Management, filed Aug. 2, 2022, which is hereby expressly incorporated in its entirety herein.

Fluid resistance within a lumen may be greater when there is a reduction in cross sectional luminal area for flow, for example, when a second interventional device (e.g., a catheter or guidewire) extends within the lumen of a first interventional device. The amount of fluid resistance can be affected by the length of the cross sectional narrowing, for example, due to a depth of axial insertion of the second interventional device within the first interventional device. A second interventional device extending partially through the lumen of a first interventional device will provide a smaller length of cross-sectional narrowing, and accordingly may result in a lower fluid resistance within the lumen of the first catheter, than if the second interventional device were to extend entirely through the lumen of the first interventional device. Thus, fluid resistance can be lowered by at least partially decreasing a depth of axial insertion (i.e., axial overlap) of a second interventional device into the lumen through which fluid is to be injected (e.g., a length of the second interventional device into its concentrically adjacent lumen).

In some embodiments, over certain depths of insertion of a second interventional device within a first interventional device (for example, when the second interventional device is at or near a maximum insertion depth within the first interventional device), the size of the fluid channel between the devices (e.g., the annular lumen between the first interventional device and the second interventional device) can lead to higher than desirable amounts of fluid resistance during a priming procedure. In some embodiments, the depth of insertion of the second interventional device within the first interventional device can be decreased to reduce the pressure needed to prime the catheter and reduce internal interference.

In some embodiments, a catheter in the interventional device assembly can be separated from the other interventional devices for priming to reduce the pressure needed to prime the catheter and reduce internal interference. The catheter being primed may be separated from the interventional devices within the lumen of the catheter by proximally retracting the interventional devices within the lumen of the catheter. For example, the interventional devices within the lumen of the catheter being primed can be proximally retracted from the catheter being primed as far as possible while still maintaining a nested or stacked relationship (e.g., at least about 2 cm or 5 cm or more axial overlap) in order to minimize the pressure needed to prime the catheter and minimize internal interference. In other words, a catheter can be separated from more proximal interventional devices for priming while a distal tip of an adjacent proximal interventional device is still positioned within the lumen of the catheter. Maintaining at least some of the distal tip of an adjacent proximal interventional device within the lumen of the catheter may allow for easier reinsertion and advancement of the proximal interventional device after priming.

In some embodiments, the axial overlap may be between about 2 cm and about 20 cm, between about 2 cm and 10 cm, between about 2 cm and 5 cm, between about 5 cm and 20 cm, between about 5 cm and 10 cm, or any other suitable range. In some embodiments, the axial overlap may be at least about 2 cm, at least about 5 cm, at least about 10 cm, at least about 20 cm, no more than 2 cm, no more than 5 cm, no more than 10 cm, no more than 20 cm, about 2 cm, about 5 cm, about 10 cm, about 20 cm, or any other suitable amount. For example, in some embodiments the axial overlap may be about 2 cm, 3 cm, 4 cm, 5 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm, plus or minus ½ cm.

In some embodiments, the robotic drive table can be programed to proximally retract the inner interventional device(s) from the catheter being primed as much as possible while still maintaining a nested or stacked relationship. In other embodiments, the robotic drive table can be programmed to separate inner devices from the catheter being primed to a distance sufficient to optimize the length of the unobstructed lumen and result in an amount of fluid resistance lower than a threshold value. After the catheter being primed is separated from the other interventional devices, the catheter can be primed by flushing the catheter with fluid, such as saline, contrast media, or a mixture of saline and contrast media.

After the catheter is primed, it may be returned to an initial position and a next catheter of the interventional device assembly can be separated from the other interventional devices within its lumen for priming. This sequence can be repeated for each catheter of the interventional device assembly. In other embodiments, after a catheter is primed, it may be advanced to a ready or drive position to begin insertion into the patient. While the foregoing describes separating catheters to be primed by retraction of inner interventional devices, an outer catheter may also be separated from inner interventional devices by distally axially advancing the outer catheter relative to the inner interventional devices. An example of a priming process is described with respect to FIGS. 22A-22C.

FIG. 22A depicts the interventional device assembly 2900 assembled in a concentric stack and axially compressed configuration. As shown in FIG. 22A, the interventional devices can be fully nested within each other. This may be the configuration following unpackaging of the device assembly 2900 and placement onto the robotic drive table. A priming sequence may begin by distally axially advancing the catheter 2906 and hub 2914 relative to the catheter 2904, hub 2912, catheter 2902, hub 2910, guidewire 2907, and hub 2909, for example, as far as possible while maintaining a distal tip of the catheter 2904 within the lumen of the catheter 2906, as shown in FIG. 22B, or to a distance that will result in a desirable amount of fluid resistance for priming. In some embodiments, the catheter 2906 is advanced in response to a control signal from a control system. The catheter 2906 can then be primed by introducing priming fluid using the fluidics system. In some embodiments, priming fluid is introduced in response to a control signal from a control system. Priming the catheter 2906 can include priming the hub 2914. For example, in certain embodiments, the hub 2914 or a hemostasis valve coupled thereto can include fluidics connections to receive priming fluid from the fluidics system. After priming, the catheter 2906 can be returned to its initial position (e.g., the fully axially compressed configuration) as shown in FIG. 22A. In some embodiments, the catheter 2906 is returned to its initial position in response to a control signal from a control system.

After the catheter 2906 is primed and returned to its initial position, the catheter 2904 and hub 2912 can be distally axially advanced relative to the catheter 2902, hub 2910, guidewire 2907 and hub 2909 (also distally axially advancing the catheter 2906 and hub 2914 without changing or minimally changing their relative position with respect to catheter 2904), for example, as far as possible while maintaining a distal tip of the catheter 2902 within the lumen of the catheter 2904, as shown in FIG. 22C, or to a distance that will result in a desirable amount of fluid resistance for priming. In some embodiments, the catheter 2904 and the catheter 2906 are advanced in response to a control signal from a control system. The catheter 2904 can then be primed by introducing priming fluid using the fluidics system. In some embodiments, priming fluid is introduced in response to a control signal from a control system. Priming the catheter 2904 can include priming the hub 2912. For example, in certain embodiments, the hub 2912 or a hemostasis valve coupled thereto can include fluidics connections to receive priming fluid from the fluidics system. After priming, the catheter 2904 and catheter 2906 can be returned to their initial positions (e.g., the fully axially compressed configuration) as shown in FIG. 22A. In some embodiments, the catheter 2904 and the catheter 2906 are returned to their initial position in response to a control signal from a control system.

After the catheter 2904 is primed and returned to its initial position, the catheter 2902 and hub 2910 can be distally axially advanced relative to the guidewire 2907 and hub 2909 (also distally axially advancing the catheter 2906, hub 2914, catheter 2904, and hub 2912 without changing or minimally changing their relative positions with respect to the catheter 2902), for example, as far as possible while maintaining a distal tip of the guidewire 2907 within the lumen of the catheter 2902, or to a distance that will result in a desirable amount of fluid resistance for priming. In some embodiments, the catheter 2902, the catheter 2904, and the catheter 2906 are advanced in response to a control signal from a control system. The catheter 2902 can then be primed by introducing priming fluid using the fluidics system. In some embodiments, priming fluid is introduced in response to a control signal from a control system. Priming the catheter 2902 can include priming the hub 2910. For example, in certain embodiments, the hub 2910 or a hemostasis valve coupled thereto can include fluidics connections to receive priming fluid from the fluidics system. After priming, the catheter 2902 and catheters 2904 and 2906 can be returned to their initial positions (e.g., the fully axially compressed configuration) shown in FIG. 22A. In some embodiments, the catheter 2902, the catheter 2904, and the catheter 2906 are returned to their initial position in response to a control signal from a control system.

In some embodiments, the priming procedure described with respect to FIGS. 20A-20C may be performed in response to a single control signal from a control system. In other embodiments, various steps of the priming procedure may be performed in response to unique control signals. In some embodiments, priming of each unique interventional device can be performed in response to a unique control signal.

In alternative embodiments, each of the catheters can be distally separated from one another simultaneously for priming. For example, the catheter 2902 can be distally separated from the guidewire 2907 while maintaining the distal tip of the guidewire 2907 in the lumen of the catheter 2902, the catheter 2904 can be distally separated from the catheter 2902 while maintaining the distal tip of the catheter 2902 in the lumen of the catheter 2904, and the catheter 2906 can be distally separated from the catheter 2904 while maintaining the distal tip of the catheter 2904 in the lumen of the catheter 2906 simultaneously. However, an embodiment in which only one set of adjacent hubs is separated at a time, as described with respect to FIGS. 22A-22C, can provide a smaller overall length of the assembly at any particular time, which can allow for use with a smaller robotic drive system. While separation of outer catheters from their inner interventional devices is described as distally axially advancing the catheters relative to their inner interventional devices, separation can include proximally retracting the inner interventional devices from the outer catheters.

In alternative embodiments, one or more of the catheter 2902, the catheter 2904, and the catheter 2906 can be advanced to a ready or drive position to begin insertion into the patient after priming (e.g., prior to priming a subsequent catheter). In such embodiments, the catheters may advance to the ready or drive position without returning to their initial position after priming.

As described above, in some embodiments, the catheters 2902, 2904, and 2906 may be assembled into the concentric stack orientation illustrated in FIG. 17 prior to flushing the catheters to remove air by displacing it with a fluid such as saline, contrast media, or a mixture of saline and contrast media. This is preferably accomplished in each fluid lumen, such as, for example, the annular lumen between the catheter 2906 and the catheter 2904 and in between each of the additional concentric interventional devices in the concentric stack. Infusing fluid (e.g., saline, contrast media, or a mixture of saline and contrast media) under pressure may displace substantially all of the air but some small bubbles may remain, adhering to the inside wall of an outer catheter (e.g., the guide catheter 2906), the outside wall of an inner catheter (e.g., the procedure catheter 2904), or both.

While fluid is being introduced under pressure into the proximal end of the annular lumen (e.g., into a hub of the outer catheter or a hemostasis valve coupled thereto), the inner catheter may be moved with respect to the outer catheter, to disrupt the holding forces between the microbubbles and adjacent wall and allow the bubbles to be carried downstream and out through the distal opening of the lumen or removed via aspiration. The catheters may be moved axially, rotationally or both with respect to each other. In certain embodiments, the catheters may be reciprocated axially, rotationally, or both with respect to each other. In some embodiments, the catheters may be moved intermittently axially, rotationally, or both. In other embodiments, the catheters may be rotated continuously or in a constant direction. In some embodiments, the catheters are moved using a driving mechanism that moves the catheter hubs, for example, a magnetically coupled drive system

In some implementations, a first catheter is moved reciprocally with respect to an adjacent catheter or guidewire such as axially over a stroke length in a range of from about 1 mm to about 250 mm, from about 10 mm to about 250 mm, from about 5 mm to about 125 mm, from about 25 mm to about 125 mm, from about 10 mm to about 50 mm, from about 15 mm to about 30 mm, from about 5 mm to about 30 mm, from about 15 mm to about 25 mm, from about 20 mm to about 40 mm, or any other suitable range. In some implementations, a first catheter is moved reciprocally with respect to an adjacent catheter or guidewire such as axially over a stroke length of at least 5 mm, at least 10 mm, at least 15 mm, at least 20 mm, at least 25 mm, at least 30 mm, at least 50 mm, no more than 10 mm, no more than 20 mm, no more than 25 mm, no more than 30 mm, no more than 50 mm, no more than 125 mm, no more than 150 mm, about 5 mm, about 10 mm, about 15 mm, about 20 mm, about 25 mm, about 30 mm, about 50 mm, or any other suitable stroke length.

In some implementations, a first catheter is moved reciprocally with respect to an adjacent catheter or guidewire such as axially at a reciprocation frequency in a range of from about 0.5 Hz to about 1 Hz, from about 1 Hz to about 5 Hz, from about 1 Hz to about 10 Hz, from about 1 Hz to about 25 Hz, from about 5 Hz to about 10 Hz, from about 10 Hz to about 25 Hz, or any other suitable range of frequencies. In some implementations, the first catheter is moved reciprocally with respect to an adjacent catheter or guidewire such as axially at a reciprocation frequency of at least 0.5 Hz, at least 1 Hz, at least 2 Hz, at least 5 Hz, at least 10 Hz, at least 25 Hz, no more than 0.5 Hz, no more than 1 Hz, no more than 2 Hz, no more than 5 Hz, no more than 10 Hz, no more than 25 Hz, about 0.5 Hz, about 1 Hz, about 2 Hz, about 5 Hz, about 10 Hz, about 25 Hz or any other suitable frequency.

In one implementation, a first catheter is moved reciprocally with respect to the adjacent catheter or guidewire such as axially over a stroke length in a range of from about 0.5 inches to about 10 inches, or from about one inch to about 5 inches at a reciprocation frequency of no more than about 5 cycles per second or two cycles per second or less.

In some implementations, a first catheter is moved reciprocally with respect to an adjacent catheter or guidewire such as rotationally over an angle of rotation per stroke in a range of from about 5 degrees to about 180 degrees, from about 5 degrees to about 360 degrees, from about 15 degrees to about 180 degrees, from about 15 degrees to about 150 degrees, from about 15 degrees to about 120 degrees, from about 15 degrees to about 90 degrees, form about 15 degrees to about 60 degrees, from about 15 degrees to about 30 degrees, from about 30 degrees to about 180 degrees, from about 30 degrees to about 150 degrees, from about 30 degrees to about 120 degrees, from about 30 degrees to about 90 degrees, form about 30 degrees to about 60 degrees, from about 60 degrees to about 180 degrees, from about 60 degrees to about 150 degrees, from about 60 degrees to about 120 degrees, from about 60 degrees to about 90 degrees, from about 90 degrees to about 180 degrees, from about 90 degrees to about 150 degrees, from about 90 degrees to about 120 degrees, from about 120 degrees to about 180 degrees, from about 120 degrees to about 150 degrees, from about 150 degrees to about 180 degrees or any other suitable range. In some implementations, a first catheter is moved reciprocally with respect to an adjacent catheter or guidewire such as rotationally over an angle of rotation per stroke of at least 5 degrees, at least 15 degrees, at least 30 degrees, at least 60 degrees, at least 90 degrees, at least 120 degrees, at least 150 degrees, at least 180 degrees, at least 360 degrees, no more than 5 degrees, no more than 15 degrees, no more than 30 degrees, no more than 60 degrees, no more than 90 degrees, no more than 120 degrees, no more than 150 degrees, no more than 180 degrees, no more than 360 degrees, about 5 degrees, about 15 degrees, about 30 degrees, about 60 degrees, about 90 degrees, about 120 degrees, about 150 degrees, about 180 degrees, about 360 degrees, or any other suitable angle.

In some implementations, a first catheter is moved reciprocally with respect to an adjacent catheter or guidewire such as rotationally at a reciprocation frequency in a range of from about 0.5 Hz to about 1 Hz, from about 1 Hz to about 5 Hz, from about 1 Hz to about 10 Hz, from about 1 Hz to about 25 Hz, from about 5 Hz to about 10 Hz, from about 10 Hz to about 25 Hz, or any other suitable range of frequencies. In some implementations, the first catheter is moved reciprocally with respect to an adjacent catheter or guidewire such as rotationally at a reciprocation frequency of at least 0.5 Hz, at least 1 Hz, at least 2 Hz, at least 5 Hz, at least 10 Hz, at least 25 Hz, no more than 0.5 Hz, no more than 1 Hz, no more than 2 Hz, no more than 5 Hz, no more than 10 Hz, no more than 25 Hz, about 0.5 Hz, about 1 Hz, about 2 Hz, about 5 Hz, about 10 Hz, about 25 Hz or any other suitable frequency.

In some implementations, a first catheter is moved reciprocally with respect to an adjacent catheter or guidewire for a number of reciprocations between 1 and 200, between 1 and 100, between 1 and 50, between 1 and 25, between 1 and 15, between 1 and 10, between 1 and 5, between 5 and 25, between 5 and 15, between 5 and 10, or any other suitable range. In some implementations, a first catheter is moved reciprocally with respect to an adjacent catheter or guidewire for at least 1 reciprocation, at least 2 reciprocations, at least 5 reciprocations, at least 10 reciprocations, at least 15 reciprocations, at least 25 reciprocations, at least 50 reciprocations, no more than 5 reciprocations, no more than 10 reciprocations, no more than 15 reciprocations, no more than 25 reciprocations, no more 50 than reciprocations, no more than 100 reciprocations, no more than 200 reciprocations, about 1 reciprocation, about 2 reciprocations, about 5 reciprocations, about 10 reciprocations, about 25 reciprocations, about 50 reciprocations, about 100 reciprocations, about 200 reciprocations, or any other suitable number. One reciprocation can include a movement (axially or rotationally) from a first position to a second position followed by a return from the second position to the first position.

In some implementations, a first catheter is moved reciprocally or rotationally with respect to an adjacent catheter or guidewire over a length of time in a range of from 1 about second to about 60 seconds, from about 1 second to about 45 seconds, from about 1 second to about 30 seconds, from about 1 second to about 20 seconds, from about 1 second to about 15 seconds, from about 1 second to about 10 seconds, from about 5 seconds to about 45 seconds, from about 5 seconds to about 30 seconds, from about 5 seconds to about 20 seconds, from about 5 seconds to about 15 seconds, from about 5 seconds to about 10 seconds, from about 10 seconds to about 30 seconds, form about 10 seconds to about 20 seconds, or any other suitable range. In some implementations, a first catheter is moved reciprocally with respect to an adjacent catheter or guidewire over a length of time of at least 1 second, at least 5 seconds, at least 10 seconds, at least 15 seconds, at least 20 seconds, at least 30 seconds, at least 45 seconds, at least 60 seconds, no more than 5 seconds, no more than 10 seconds, no more than 15 seconds, no more than 20 seconds, no more than 30 seconds, no more than 45 seconds, no more than 60 seconds, about 5 seconds, about 10 seconds, about 15 seconds, about 20 seconds, about 30 seconds, about 45 seconds, about 60 seconds, or any other suitable length of time.

Reciprocation of adjacent catheters to disrupt microbubbles may be accomplished manually by grasping the corresponding catheter hubs and manually moving the catheters axially or rotationally with respect to each other while delivering pressurized fluid (e.g., saline, contrast media, or a mixture of saline and contrast media). Alternatively, such as in a robotically driven system, a processor may be configured to robotically drive at least one of two adjacent catheter hubs (for example, at least one of hub 2914 and hub 2912) to achieve relative movement between the adjacent catheters thereby disrupting and expelling microbubbles, such as in response to user activation of a flush control. For example, in certain embodiments, two adjacent interventional devices may be moved relative to one another in response to a control signal from a control system. In certain embodiments, delivery of pressurized fluid may be performed in response to a control signal from a control system.

The reciprocation of adjacent catheters may generate shear forces that dislodge the air bubbles. For example, relative movement of the inner and outer surfaces of adjacent catheters may increase the fluid shear rate between the adjacent catheters during priming in comparison to static surfaces. In some embodiments, the shear force can be increased by increasing the flow rate of the solution (e.g., saline, contrast media, or a mixture of saline and contrast media) being provided by the fluidics system. In certain embodiments, both flow rate and relative movement between adjacent catheters are controlled to dislodge air bubbles.

In some embodiments, after each catheter is primed by the fluidics system, an ultrasound bubble detector may be used to confirm that the catheters are substantially free of air bubbles. For example, an ultrasound chip (such as mounted within a hub adjacent a catheter receiving lumen) may be run along the length of the catheters to confirm that no air bubbles remain in the system.

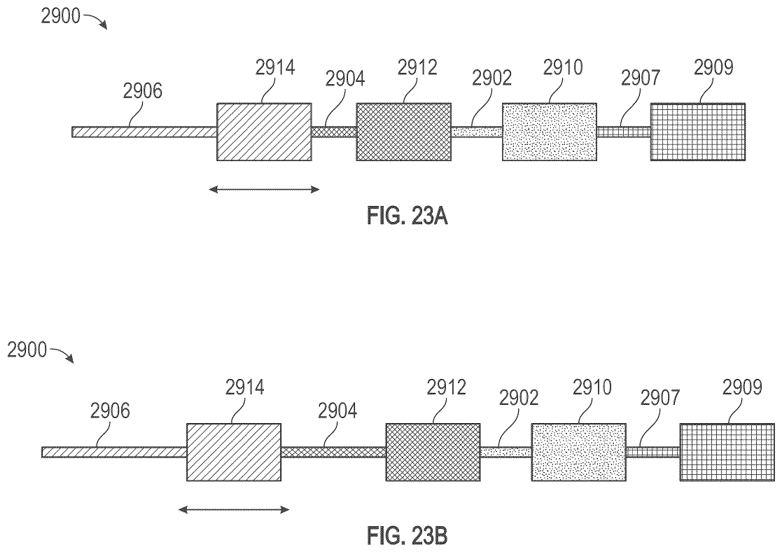

An example of a priming process including reciprocal movement of adjacent catheters is described with respect to FIGS. 23A-23B.

FIG. 23A depicts the interventional device assembly 2900 assembled in a concentric stack configuration. As shown in FIG. 23A, the interventional devices can be fully nested within each other. This may be the configuration following unpackaging of the device assembly 2900 and placement onto the robotic drive table. Alternatively, individual interventional devices of the device assembly 2900 can be assembled into the device assembly 2900 on the drive table.

A priming sequence may begin by priming the catheter 2906. In some embodiments, the catheter 2906 can be primed by introducing fluid (e.g., saline, contrast media, or a mixture of saline and contrast media) under pressure into the lumen of the catheter 2906 while generating reciprocal movement of catheter 2906 and/or hub 2914, axially, rotationally or both, relative to the catheter 2904. Priming the catheter 2906 can include priming the hub 2914. For example, in certain embodiments, the hub 2914 or a hemostasis valve coupled thereto can include fluidics connections to receive priming fluid from the fluidics system. In certain embodiments, the catheter 2906 and/or hub 2914 can be axially agitated back and forth along a longitudinal axis of the catheter 2906 (e.g., between the position of FIG. 23A and the position of FIG. 23B). Axial and/or rotational reciprocal motion of the catheter 2906 and/or hub 2914 can be performed manually or by a robotic drive table. Reciprocal movement may be generated in response to a control signal from a control system. Introducing fluid under pressure may be performed in response to a control signal from a control system.

In some embodiments, priming of the catheter 2906 may be performed by introducing fluid (e.g., saline, contrast media, or a mixture of saline and contrast media) under pressure into the lumen of the catheter 2906 while generating reciprocal movement of the catheter 2904 and/or hub 2912, axially, rotationally, or both, relative to the catheter 2906. Axial and/or rotational reciprocal motion of the catheter 2904 and/or hub 2912 can be performed manually or by a robotic drive table. Reciprocal movement may be generated in response to a control signal from a control system. Introducing fluid under pressure may be performed in response to a control signal from a control system.

In some embodiments, priming of the catheter 2906 may be performed by introducing fluid (e.g., saline, contrast media, or a mixture of saline and contrast media) under pressure into the lumen of the catheter 2906 while generating reciprocal movement of both the catheter 2906 (and/or hub 2914) and the catheter 2904 (and/or hub 2912), axially, rotationally, or both, relative to one another. Reciprocal movement may be generated in response to a control signal from a control system. Introducing fluid under pressure may be performed in response to a control signal from a control system.

In some embodiments, after priming the catheter 2906, the catheter 2906 can be returned to an initial position as shown in FIG. 23A. In other embodiments, after priming the catheter 2906, the catheter 2906 can be advanced to a ready or drive position to begin insertion into the patient.

In some embodiments, after the catheter 2906 is primed, the catheter 2904 can be primed. Priming the catheter 2904 can include priming the hub 2912. For example, in certain embodiments, the hub 2912 or a hemostasis valve coupled thereto can include fluidics connections to receive priming fluid from the fluidics system. In some embodiments, the catheter 2904 can be primed by introducing fluid (e.g., saline, contrast media, or a mixture of saline and contrast media) under pressure into the lumen of the catheter 2904 while generating reciprocal movement of the catheter 2904 and/or hub 2912, axially, rotationally or both, relative to the catheter 2902. Reciprocal movement may be generated in response to a control signal from a control system. Introducing fluid under pressure may be performed in response to a control signal from a control system.

In some embodiments, priming of the catheter 2904 may be performed by introducing fluid (e.g., saline, contrast media, or a mixture of saline and contrast media) under pressure into the lumen of the catheter 2904 while generating reciprocal movement of the catheter 2902 and/or hub 2910, axially, rotationally, or both, relative to the catheter 2904. Axial and/or rotational reciprocal motion of the catheter 2902 and/or hub 2910 can be performed manually or by a robotic drive table. Reciprocal movement may be generated in response to a control signal from a control system. Introducing fluid under pressure may be performed in response to a control signal from a control system.

In some embodiments, priming of the catheter 2904 may be performed by introducing fluid (e.g., saline, contrast media, or a mixture of saline and contrast media) under pressure into the lumen of the catheter 2904 while generating reciprocal movement of both the catheter 2904 (and/or hub 2912) and the catheter 2902 (and/or hub 2910), axially, rotationally, or both, relative to one another. Reciprocal movement may be generated in response to a control signal from a control system. Introducing fluid under pressure may be performed in response to a control signal from a control system.

In some embodiments, after priming the catheter 2904, the catheter 2904 can be returned to an initial position as shown in FIG. 23A. In some embodiments, after priming the catheter 2904, the catheter 2904 can be advanced to a ready or drive position to begin insertion into the patient.

In some embodiments, after the catheter 2904 is primed, the catheter 2902 can be primed. Priming the catheter 2902 can include priming the hub 2910. For example, in certain embodiments, the hub 2910 or a hemostasis valve, a three-way valve, or other fluid control valve coupled thereto can include fluidics connections to receive priming fluid from the fluidics system. In some embodiments, the catheter 2902 can be primed by introducing fluid (e.g., saline, contrast media, or a mixture of saline and contrast media) under pressure into the lumen of the catheter 2902 while generating reciprocal movement of the catheter 2902 and/or hub 2910, axially, rotationally or both, relative to the guidewire 2907. Reciprocal movement may be generated in response to a control signal from a control system. Introducing fluid under pressure may be performed in response to a control signal from a control system.

In some embodiments, priming of the catheter 2902 may be performed by introducing fluid (e.g., saline, contrast media, or a mixture of saline and contrast media) under pressure into the lumen of the catheter 2902 while generating reciprocal movement of the guidewire 2907 and/or hub 2909, axially, rotationally or both, relative to the catheter 2902. Axial and/or rotational reciprocal motion of the guidewire 2907 and/or hub 2909 can be performed manually or by a robotic drive table. Reciprocal movement may be generated in response to a control signal from a control system. Introducing fluid under pressure may be performed in response to a control signal from a control system.

In some embodiments, priming of the catheter 2902 may be performed by introducing fluid (e.g., saline, contrast media, or a mixture of saline and contrast media) under pressure into the lumen of the catheter 2902 while generating reciprocal movement of both the catheter 2902 (and/or hub 2910) and the guidewire 2907 (and/or hub 2909), axially, rotationally or both, relative to one another. Reciprocal movement may be generated in response to a control signal from a control system. Introducing fluid under pressure may be performed in response to a control signal from a control system.

In some embodiments, after priming the catheter 2902, the catheter 2902 can be returned to an initial position as shown in FIG. 23A. In other embodiments, after priming the catheter 2902, the catheter 2902 can be advanced to a ready or drive position to begin insertion into the patient.

In some embodiments, the priming procedure described with respect to FIGS. 23A and 23B may be performed in response to a single control signal from a control system. In other embodiments, various steps of the priming procedure may be performed in response to unique control signals. In some embodiments, priming of each unique interventional device can be performed in response to a unique control signal.

In the priming sequence described herein with respect to FIGS. 23A and 23B, the catheters are primed in order starting with the catheter 2906, followed by the catheter 2904, and then followed by the catheter 2902. However, it is contemplated that the catheters may be primed in any order. The catheters may be primed in series as described above with respect to FIGS. 23A and 23B. Alternatively, two or more of the catheters or each of the catheters may be primed in parallel.

In certain embodiments, priming the catheters can include decreasing a depth of axial insertion (i.e., axial overlap) of a second interventional device into the lumen of a first interventional device through which fluid is to be injected (e.g., a length of the second interventional device into its concentrically adjacent lumen), as described with respect.to FIGS. 22A-22C, and also generating relative reciprocal movement, axially, rotationally or both, between first interventional device and the second interventional device during priming, as discussed with respect to FIGS. 23A and 23B.

In some embodiments, a concentric catheter stack or concentric interventional device stack may be used to perform a vascular procedure, for example, a neurovascular procedure. For example, in some embodiments, a concentric catheter stack or concentric interventional device stack may be used to perform a thrombectomy procedure.

In an example of a neurovascular thrombectomy procedure, an interventional device assembly including a guide catheter 2906, a procedure catheter 2904, an access or insert catheter 2907, and a guidewire 2907 can be driven (e.g., through an introducer sheath) into the descending aorta. Next, the guide catheter 2906, the procedure catheter 2904, and the access or insert catheter 2907 can be tracked up to the aortic arch. The procedure catheter 2904, access catheter 2907, and guidewire 2907 can be driven into an ostium at the aortic arch (e.g., simultaneously or separately). In some embodiments, the access catheter 2907 is advanced out of the procedure catheter 2904 and the guide catheter 2906 to engage the ostium first. After the distal end of the access catheter 2907 is positioned within the desired ostium, the guidewire 2907 can be advanced distally into the ostium to secure access. After the access catheter 2907 and guidewire 2907 are positioned within the desired ostium, the procedure catheter 2904 and/or guide catheter 2906 can be advanced into the ostium (and, in some embodiments, beyond), while using the support of the access catheter 2907 and/or guidewire 2907 to maneuver through the aorta and into the ostium. In some embodiments, the guidewire 2907 engages the origin of the brachiocephalic artery, and is then advanced up to the petrous segment of the internal carotid artery.

In some embodiments, the guide catheter 2906 and the procedure catheter 2904 are both advanced (e.g., simultaneously or sequentially) over the guidewire 2907 and over the insert or access catheter 2907 while the access catheter 2907 remains at the ostium for support. The guidewire 2907 may be further advanced past the petrous segment to the site of a clot, such as the Ml segment.

In some embodiments the guide catheter 2906 and the procedure catheter 2904 are advanced (e.g., simultaneously or sequentially) to position the distal tip of the procedure catheter 2904 at the procedure site, for example on the face of the clot. The guidewire 2907 and access catheter 2907 can be removed, and aspiration of the clot can commence through the procedure catheter 2904. That is, the guidewire 2907 and the access catheter 2907 can be proximally retracted to allow aspiration through the procedure catheter 2906. After aspiration of the clot, the procedure catheter 2904 and guide catheter 2906 can be removed (e.g., simultaneously or sequentially). For example, in some embodiments, the procedure catheter 2904 may be removed before removing the guide catheter 2906.

In some embodiments, aspiration may be performed through two catheters (e.g., the procedure catheter 2904 and the guide catheter 2906) simultaneously. For example, during a thrombectomy procedure, a clot may become engaged with or corked at a distal end of the procedure catheter 2904 (or another inner catheter). In such cases, it may be necessary to remove the procedure catheter 2904 (or other inner catheter) from the vasculature of the patient to remove the clot. As the procedure catheter 2904 (or other inner catheter) is retracted from the guide catheter 2906 (or other outer catheter), debris from the stuck clot can dislodge. In such embodiments, application of vacuum at both the procedure catheter 2904 (or other inner catheter) and the guide catheter 2906 (or other outer catheter) can beneficially prevent the debris from flowing distally into the vasculature of the patient by aspirating the debris and thus reducing the risk of embolization.

In some embodiments, the clot may become engaged with or corked at the distal end of the guide catheter 2906 (or other outer catheter). Vacuum through the guide catheter 2906 (or other outer catheter) may prevent dislodgement of the clot from the guide catheter 2906 (or other outer catheter). In some instances, it may not be readily apparent if portions of a clot are engaged with the guide catheter 2906 (or other outer catheter) or the procedure catheter 2904 (or other inner catheter). In such instances, vacuum through both the guide catheter 2906 (or other outer catheter) and the procedure catheter 2904 (or other inner catheter) may prevent debris from flowing distally into the vasculature of the patient by aspirating the debris and thus reducing the risk of embolization.

### Fluidics Management Systems and Methods

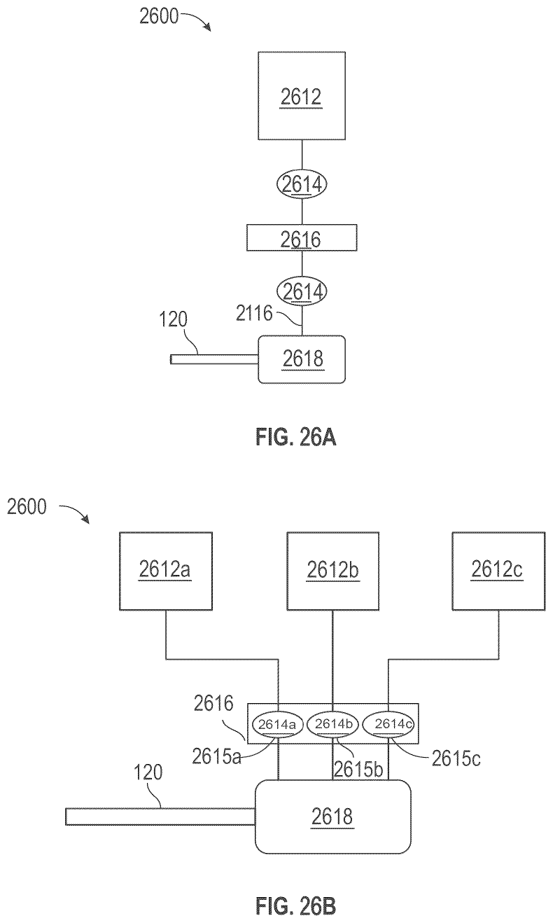

FIG. 26A illustrates one embodiment of one channel of a multi-channel fluidics management system 2600. The fluidics management system 2600 may be configured as an automated system to manage the delivery of fluids to or aspirate material from a patient via one or more interventional devices, for example catheters. As shown, the system 2600 may manage fluid delivery to a patient during a medical procedure. The fluidics management system 2600 includes at least one fluid source and/or sink 2612 coupled to a valve 2614, which is coupled to a manifold 2616. The manifold 2616 is either remote (e.g., on a support table or tower outside the sterile field) or coupled to a catheter hub 2618, which is coupled to at least one source and/or sink line 2116. The source and/or sink line 2116 is coupled through the hub 2818 to at least one catheter 120.

In some embodiments, the fluid source and/or sink 2612 includes both a reservoir of fluid volume and a means of propelling such fluid to another component of the system 2600 or a means of retrieving fluid back to the source. Example propelling means may include one or more propellers, impellers, syringes, and/or pumps to circulate and/or retrieve fluid throughout system 2600. In some embodiments, the propelling means can be used to control the volume, flow rate, and/or pressure. In certain embodiments, the propelling means can be activated to propel fluid to another component of the system or retrieve fluid from the system or deactivated to stop the movement of fluid.

In some embodiments, the fluidics management channel is substantially duplicated for each catheter configured for use in a particular medical procedure. Different channels may differ in sensors, pumps, and/or valves employed based on the interventional device that is connected to each fluidics channel. For example, a fluidics system for a procedure catheter (e.g., for aspiration) may include an inline vacuum pump and filter. Further for example, a fluidics system for a guide, access, or insert catheter may include an inline drip rate sensor, air bubble sensor, pressure sensor, and/or air bubble filter.

The source and/or sink 2612 represents either a fluid source or a fluid sink (e.g., waste cannister). For example, a fluid source may include a container adapted to house a fluid (e.g., saline, contrast, pharmaceuticals, blood, plasma, or other fluid) for use with the fluidics management system 2600. The container may be configured to release fluid into a fluid delivery line (e.g., fluid delivery tube) using active means (e.g., pumps, vacuums, etc.) or passive (e.g., gravity). The fluid sink may include a container adapted to receive fluids (e.g., aspirate, thrombus, particulate, saline, contrast, pharmaceuticals, blood, or other fluid or combination thereof) from the patient and/or from other fluidics infrastructure within the fluidics management system 2600.

The valve 2614 represents one or more valves that are coupled to the source and/or sink 2612 at a first side of the valve 2614 and coupled to the manifold 2616 at a second side of the valve 2614. The manifold 2616 is configured to connect each valve 2614 to a particular hub 2618. In some embodiments, the valve 2614 may instead couple directly to the hub 2618 to avoid the use of a separate manifold 2616. In some embodiments, the manifold 2616 may be integrated into the hub. In some embodiments, a second valve 2614 may connect the manifold 2616 to the hub 2618. For example, the second valve 2614 can be coupled to the manifold 2616 at a first side and coupled to the hub 2618 at the second side.

The hub 2618 is configured to releasably or non-releasably couple to an interventional device (catheter, guidewire, guiding element, or another medical device). For example, a catheter 120 has a proximal end attached to a unique hub 2618, sometimes referred to as a “puck.” In some embodiments, the hub 2618 is moveable along a path along the surface of a robotic drive table to advance or retract the catheter 120 (or other medical and/or interventional device). Each hub 2618 may also contain mechanisms to rotate or deflect the catheter 120 or guidewire as desired. The hub 2618 may be connected to fluid delivery tubes (e.g., source/sink line 2116) to provide fluid release or fluid capture. Each hub 2618 may be in electrical communication with an electronic control system, either via hard wired connection, RF wireless connection, or a combination of both. Additional details of the hubs, drive table and related systems are found in U.S. patent application Ser. No. 17/816,669, entitled Method of Supra-Aortic Access for a Neurovascular Procedure, filed Aug. 1, 2022, which is hereby expressly incorporated in its entirety herein.

Any of the hubs disclosed herein may further comprise one or more fluid injection ports and/or a wireless RF transceiver for communications and/or power transfer. In some embodiments, the hub 2618 may also comprise a wired electrical communications port and a power port.

In some embodiments, the hub 2618 or line 2116 leading to the hub 2618 may include a visual indicator, for indicating the presence of an aspirated clot. The visual indicator may comprise a clot chamber having a transparent window. A filter may be provided in the clot chamber. Additional details of the clot capture filter and related features may be found in U.S. Provisional Patent Application Ser. No. 63/256,743, entitled Device for Clot Retrieval, filed Oct. 18, 2021, which is hereby expressly incorporated in its entirety herein.

Any of the hubs or interventional devices disclosed herein may further comprise a sensor for detecting a parameter of interest such as a location or orientation of a distal tip or a status of the distal tip of an interventional device. The status of the distal tip may include, but not be limited to detection of an interaction between a vessel wall and the distal tip, detection of an interaction between a vessel wall and a clot, or detection of an unobstructed distal tip. The sensor, in some instances, may be positioned on a flexible body of an interventional device. The sensor may comprise a pressure sensor to capture arterial blood pressure waveform at the distal end of the catheter, or an optical sensor to determine captured clot or air bubbles. In some embodiments, the sensor may comprise one or more of: a force sensor, a positioning sensor, a temperature sensor, a torque sensor, a strain sensor, and/or an oxygen sensor. In some embodiments, the sensor may comprise a Fiber Bragg grating sensor. For example, a Fiber Bragg grating sensor (e.g., an optical fiber) may detect strain locally that can facilitate the detection and/or determination of force being applied.

FIGS. 26B-43 illustrate schematic views of multi-channel fluidics systems, parts of multi-channel fluidics system, or operations of multi-channel fluidics management systems. These fluidic systems, along with the other systems (e.g., drive systems) and components described herein can provide fluid to a target site of a patient's vasculature through one or more catheters. For example, these systems can include a catheter having a tubular catheter shaft having a proximal end, a distal end, and a lumen defined by an interior surface of the catheter shaft extending longitudinally through the catheter shaft between the proximal end and the distal end. These systems can also include a guiding element having a distal end, a proximal end, and an exterior surface, the guiding element configured to be positioned in the lumen of the catheter creating an area, between the exterior surface of the guiding element and the interior surface of the catheter, the area having an effective cross-sectional area for fluid communication of a certain amount of fluid at a certain pressure. In some embodiments, the effective cross-sectional area is equal to, or greater than 0.001257 square inches which allows a desired amount of fluid (e.g., contrast media) to flow through the lumen when the guiding element is positioned in the lumen, to provide a desired amount of fluid. For example, the systems can include a contrast pump coupled to the catheter in fluid communication with the lumen. The system is configured to receive a signal to actuate the contrast pump to provide contrast media into the proximal end of the catheter at a pump pressure of less than or equal to about 400 PSI while the guiding element is positioned at least partially in the lumen of the catheter such that the provided contrast media propagates through the lumen of the catheter along the exterior surface of the guiding element and flows out of the distal end of the catheter. The effective cross-sectional area allows a predetermined flow rate (e.g., at least about 3 cc's per second) of the contrast media out of the distal end of the catheter. For example, contrast media having a viscosity in the range of above 1 cP to about 30 cP. That is, for contrast having a viscosity of above 1 cP, or having a viscosity of about 2 cP, 2 cP, 4 cP, 5 cP, 6 cP, 7 cP, 8 cP, 9 cP, 10 cP, 11 cP, 12 cP, 13 cP, 14 cP, 15 cP, 16 cP, 17 cP, 18 cP, 19 cP, 20 cP, 21 cP, 22 cP, 23 cP, 24 cP, 25 cP, 26 cP, 27 cP, 28 cP, 29 cP, or 30 cP, plus or minus 0.5 cP. Although having the effective cross-sectional area is important for any fluids, it is generally most important for viscous fluids like contrast media which can require high pressure to communicate through a catheter lumen.

FIG. 26B illustrates a schematic view of multi-channel fluidics management system 2610 having a first source 2612a, a second source 2612b, and a sink 2612c. The first source 2612a is coupled to a valve 2614a. The second source 2612b is coupled to a valve 2614b. The sink 2612c is coupled to a valve 2614c. The valves 2614a, 2614b, and 2614c, are part of a valve manifold 2616. The valve manifold 2616 is coupled to a hub 2618. In certain embodiments, the valve manifold 2616 is part of or directly connected to the hub 2618. In other embodiments, the valve manifold 2616 is positioned remotely from the hub 2618 and is connected to the hub 2618 via one or more fluid lines. In other embodiments, the valve manifold is part of or directly connected to hemostatic valve. In other embodiments, the valve manifold 2616 is positioned remotely from the hemostatic valve and is connected to the hemostatic valve via one or more fluid lines. The valves 2614a, 2614b, and 2614c can be opened and closed to selectively place the first fluid source 2612a, the second fluid source 2612b, and the sink 2612c in communication with a lumen of the catheter 120. For example, the valve manifold 2616 can include fluid ports (e.g., a first port 2615a associated with valve 2614a, a second fluid port 2615b associated with valve 2614b, and a third port 2615c associated with valve 2614c) that can be selectively placed in communication with the lumen of the catheter 120 or blocked from communication with the lumen of the catheter 120. For example, in some embodiments, one of the first port, second port, and third port can be placed into communication with the lumen of the catheter 120 while the other two ports are blocked from communication with the catheter 120.

In certain embodiments, the first source 2612a can be a source of heparinized saline. The source 2612b can be a source of contrast solution. In certain embodiments, one or more of the source 2612a, the source 2612b, and the source 2612c can couple to a plurality of manifolds 2616, each coupled to a unique interventional device 2618. The valve manifold 2616 as shown herein may be utilized in any of the systems described herein.

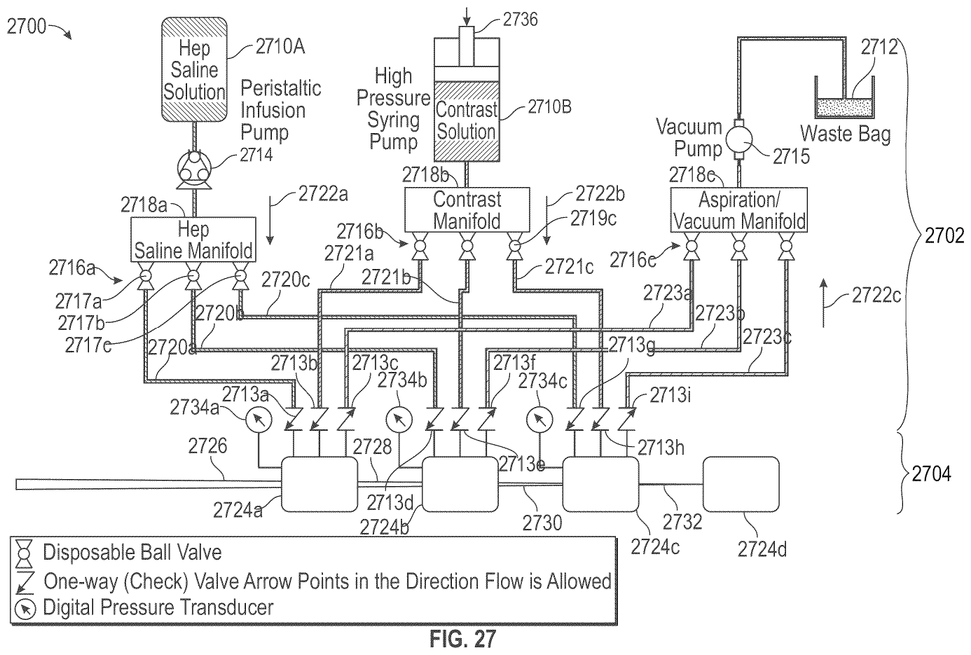

FIG. 27 illustrates a schematic view of a three channel fluidics system 2700 for use with a fluidics management system 2600 including a stack of four concentrically arranged interventional devices. The fluidics system 2700 shown here includes a fluid management portion 2702 and an interventional portion 2704. In some embodiments, the interventional portion 2704 may comprise a concentric catheter and guidewire stack configured for manual manipulation by the physician. In some embodiments, the interventional portion 2704 may comprise a concentric catheter and guidewire stack configured for manipulation by a robotic drive system. In some embodiments, the interventional portion 2704 includes a combination of both robotically driven medical devices and manually manipulated medical devices.

The components of fluid management portion 2702 may be located outside of the sterile field or within the sterile field. In some embodiments, the fluid management portion 2702 is located outside of the sterile field, but is coupled to the interventional portion 2704, which is located within the sterile field, by flexible tubing and flexible electrical conductors.

The fluid management portion 2702 may include at least two or three or more channels (e.g., parallel channels) of the type shown in FIG. 26A, each for a separate fluid source or fluid sink. In the illustrated embodiment, the fluid management portion 2702 includes three channels that each are in communication with each of three catheters via corresponding catheter hubs. Two channels provide for the delivery of two separate fluids to each of the catheters, each at a controllable pressure, volume and delivery rate. The third channel provides for aspiration from each catheter into a sink.

Each of the two or more fluid channels may be primed by completely degassing and filling with a respective fluid in order to be ready for transport into the catheter and into the bodily lumen. In some embodiments, fluid lines, catheters, and/or catheter lumens can be simultaneously flushed and primed with a fluid (e.g., saline).

In some embodiments, the systems 2700, 2800 may be configured to backfill each sink connection to each catheter with fluid (e.g., saline) at procedure initialization and/or between fluidics step. This may provide a backfilled column of saline downstream of the sink connection, for example, to ensure that contrast injections flow to a distal tip of a particular catheter rather than to the sink. In some embodiments, the systems 2600, 2700 may be configured to provide a backfilled column of saline upstream of a saline valve at the hub, for example, to ensure that contrast injections flow to the distal tip of a particular catheter or the sink rather than through the saline valve.

As shown in FIG. 27, the fluid management portion 2702 of the fluidics system 2700 includes a first source 2710a, a second source 2710b, and a sink 2712. The sources 2710a and 2710b may each be configured to hold and distribute at least one fluid (e.g., saline, contrast, pharmaceuticals, blood, or other fluid, or combinations thereof). The sink 2712 may be configured to receive waste fluid and/or waste product from a selected aspiration line leading to a corresponding catheter. Although two fluid sources and one fluid sink are shown, any number of fluid sources and/or fluid sinks is possible (e.g., one fluid source and one fluid sink, two fluid sources without a fluid sink, more than two fluid sources, etc.) corresponding to the fluid delivery and/or aspiration needs of a particular procedure.

A number of valves (and/or valve arrays) are provided to stop and start flow of each respective fluid to or through one or more fluid lines, and/or hubs within the portions 2702 and/or 2704. In the illustrated implementation, a first valve array 2716a (e.g., with three valves) is carried by a first manifold 2718a, a second valve array 2716b (e.g., with three valves) is carried by a second manifold 2718b, and a third valve array 2716c (e.g., with three valves) is carried by a third manifold 2718c. Although valve arrays with three valves are shown, any number of valves are possible and may correspond to the number of catheters and/or fluid sources being used in the procedure or a subset of the interventional devices being used in the procedure. For example, in some situations, each valve array may include at least one valve, two valves, three valves, or four or more valves.

In some embodiments, each valve in a valve array (e.g., valve array 2716a) may be configured to independently control and/or adjust fluid resistance, flow rate, and/or pressure of fluid flowing through the valve and corresponding tubing. In some embodiments, each valve in a valve array can be independently and/or simultaneously adjusted for a respective catheter and/or for more than one catheter.

In the illustrated implementation, the fluidics channel is duplicated for each catheter and will therefore be described only in connection with source 2710a below. A first outflow valve 2717a is in communication with a first catheter 2726 by a unique source line 2720a. A second outflow valve 2717b is in communication with a second catheter 2728 by a unique source line 2720b. A third outflow valve 2717c is in communication with a third catheter 2730 by a unique source line 2720c. Each valve 2717a-2717c is preferably electronically actuated in response to signals from the control system, between a fully closed, fully open, or partially open positions. Any of a variety of valve mechanisms may be utilized, such as a ball valve driven by a stepper motor, solenoid, a stopcock valve (e.g., a rotating stopcock valve), a rotary valve, or other drive mechanism known in the art. The drive mechanisms may provide for automated control and sequencing of the valves. For example, valve actuation may be achieved using stepper motors with in-built encoding to provide consistent switching and sequencing. The drive mechanisms may be controlled using motor controllers of a user control interface (for example, or a computer system). The control system may include modules that read values from sensors (e.g., flow, bubble, pressure, etc.) and display the values to control the behavior of the fluid system.

In some embodiments, a stopcock valve mechanism (e.g., a rotating stopcock valve) may be used in the manifolds described herein. For example, one or more stopcock valves may be placed adjacent to (or integrated into) a hub to avoid management of a column of fluid in particular tubing. Such tubing may be sterile disposable tubing that may offer a one-time use. Placing a manifold with stop cock valves near or integrated into the hub has the advantage of simplicity without the need to manage a column of fluid in the tubing. Having the manifold and stopcock valves away from the hub(s) may allow the manifold and the stopcock valves to both be used outside of a sterile field in conjunction with non-sterile equipment. Such a configuration may provide an advantage of preserving sterility of the components in the sterile field.

In some embodiments, the fluidics control system may further include a drive mechanism configured to adjust the sealing strength of the hemostatic valve in response to a signal from the control system, for example, from a processor of the control system. The control system (e.g., the processor) may be configured to increase the sealing strength of the hemostatic valve in response to the manipulation of the contrast control to introduce contrast into the catheter. The control system (e.g., the processor) may additionally be configured to decrease the sealing strength of the hemostatic valve in response to the manipulation of the contrast control to stop introducing contrast into the catheter. In some embodiments, the control system (e.g., the processor) may be configured to decrease the sealing strength of the hemostatic valve in response to a signal received to drive a catheter or guidewire through the hemostatic valve. Such a feature may provide the advantage of reducing friction between the hemostatic valve and a moving catheter shaft, for example.

In operation, all three valves 2717a-2717c maybe in an open configuration to flow saline through each of the three catheters. Forward flow (in the direction of arrow 2722a) of saline may be driven by a pump 2714 such as an electronically controlled peristaltic infusion pump or a rotary piston pump. Alternatively, any one of the valves may be open with the other two closed depending upon the desired performance. Alternatively or additionally, other sources of volume and/or pressure (for example, pump 2714) can be deactivated or disconnected to prevent flow.

In the concentric catheter stack illustrated in FIG. 2, the first catheter 2726 may be a ‘large bore’ guide catheter having an inner diameter of at least about 0.075 or at least about 0.080 inches in diameter. The second catheter 2728 may be an aspiration catheter having an inner diameter within the range of from about 0.060 to about 0.075 inches. The third catheter 2730 may be an access catheter. In one example, the access catheter is a steerable catheter with a deflectable distal tip, having an inner diameter within the range of from about 0.025 to about 0.050 inches; in an example, an inner diameter within the range of from about 0,045 inches to about 0.049 inches. The guidewire 2732 may have an outer diameter within the range of from about 0.014 to about 0.020 inches. In one specific example, the first catheter may have a diameter of about 0.088 inches, the second catheter about 0.071 inches, the third catheter about 0.035 inches, and the guidewire may have a diameter of about 0.018 inches.

The available lumen in the first catheter 2726 is the difference between the inner diameter (ID) of first catheter 126 and the outer diameter (OD) of second catheter 2728. That may be different than the available lumen in the second catheter 2728 (which may be the difference between the ID of second catheter 2728 and the OD of third catheter 130), which may be different than the available lumen of the third catheter 2739 (which may be the ID of the third catheter 2730 or the difference between the ID of the third catheter 130 and the OD of the guidewire 2732). In order to produce the same delivered infusion flow rate through each of the catheters, the control system may be configured to adjust the pump 2714 and/or each of the valves 2717a-2717c to compensate for differences in the effective cross sections of each respective flow path in order to achieve the same delivered flow rate through each catheter. The system can use parameter files associated with the attached devices (catheters and guidewire), the parameter files having dimension information (e.g., ID's, OD, length) of the devices. The system can adjust a pump based on the dimension information as well as information relating to the fluid being communicated inside the catheter (e.g., the density and/or the viscosity of contrast media being communicated through a catheter lumen).

In one implementation of the invention, the catheters may be assembled into the concentric stack orientation illustrated in FIG. 2 prior to flushing the catheters to remove air by displacing it with a fluid such as saline. This is preferably accomplished in each fluid lumen, such as, for example, the annular lumen between the first catheter 2726 and second catheter 2728 and in between each of the additional concentric interventional devices in the stack orientation. Infusing saline under pressure may displace substantially all of the air but some small bubbles may remain, adhering, for example, to the inside wall of the first catheter 126, the outside wall of second catheter 2728, or both.

While saline is being introduced under pressure into the proximal end of the annular lumen between two interventional devices (for example, the annular lumen between the first catheter 2726 and the second catheter 2728), the inner catheter may be moved with respect to the outer catheter (for example, the second catheter 2728 may be moved with respect to the outer catheter), to disrupt the holding forces between the microbubbles and adjacent wall and allow the bubbles to be carried downstream and out through the distal opening of the lumen. The catheters may be moved axially, rotationally or both with respect to each other. In one implementation, a first catheter is moved reciprocally with respect to the adjacent catheter or guidewire, such as axially through a range of from about 0.5 inches to about 10 inches, or from about 1 inch to about 5 inches at a reciprocation frequency of no more than about 5 cycles per second or two cycles per second or less.

Reciprocation of adjacent catheters to disrupt microbubbles may be accomplished manually by grasping the corresponding catheter hubs and manually moving the catheters axially or rotationally with respect to each other while delivering pressurized saline. Alternatively, such as in a robotically driven system, a processor may be configured to robotically drive at least one hub of two adjacent catheters (for example, at least one of hub 2724a and hub 2724b) to achieve relative movement between the adjacent catheters thereby disrupting and expelling microbubbles, such as in response to user activation of a flush control.

The source 2710b is in fluid communication with manifold 2718b, allowing fluid to flow as shown by arrow 2722b to any number of valves (e.g., three) within valve array 2716b. Forward flow (in the direction of arrow 2722b) of contrast may be driven by a pump 2736 such as a syringe pump, high pressure positive displacement pump, contrast injection pump, etc., Any one of the valves of the valve array 2716b may be open with the other two closed depending upon the desired performance. Alternatively or additionally, other sources of volume and/or pressure (for example, pump 2736) can be deactivated or disconnected to prevent flow. A proximal opening of each source line 2721a, 2721b, 2721c may be coupled to a respective output port on the corresponding valve within the valve array 2716b. A distal opening of each source line 2721a, 2721b, 2721c may be coupled to each respective hub 124a, 2724b, 2724c, and thus to the corresponding catheter 2726, catheter 2728, and/or catheter 2730. The respective catheter 2726, catheter 2728, catheter 2730, and/or guidewire 2732 may be guided into a patient (not shown). Additional hubs and/or catheters may be added to system 2700 and corresponding fluidics management system components (e.g., system 2600) may be added to system 2700. In other embodiments, the system 2700 may include less hubs and/or catheters, for example two hubs and/or catheters.

The sink 2712 is coupled to a manifold 2718c to receive fluid from aspiration lines 2723a, 2723b, 2723c in the direction shown by arrow 2722c. The aspiration lines are configured to receive fluid and embolic material from one or two or all three respective catheters 126, 128 and 130 depending upon input from the physician into the control system. Once the physician has determined which catheter(s) will be placed into aspiration mode, and actuated the corresponding aspiration control(s) the corresponding valve(s) within the valve array 2716c may be opened to allow the fluid to flow through the corresponding catheter and into the sink 2712, in response to the control system activating an aspiration pump 2715. Any one of the valves of the valve array 2716c may be open with the other two closed depending upon the desired performance. Alternatively or additionally, other sources of volume and/or pressure (for example, pump 2715) can be deactivated or disconnected to prevent flow.

In an example embodiment, the fluidics system 2700 represents an aspiration configuration in which the source 2710a contains heparinized saline and the source 2710b contains contrast solution. The sink 2712 in this example may contain waste blood/saline/embolic material that has been aspirated from a patient (not shown). Other additional sources and/or sinks may be used in combination with respective fluids.

Similarly, the contrast solution contained by source 2710b may flow in the direction of arrow 2722b and may flow into manifold 2718b. In a given procedure, the physician may determine to inject contrast through any of the three catheters, and typically through the most distal catheter at a given injection time. In response to an inject contrast command, the control system will open the valve corresponding to the selected catheter and typically maintain the other two valves closed. In some embodiments, the physician may inject contrast concurrently into two or more catheters. In some embodiments, for example, while driving catheters or guidewires, contrast or aspiration may be applied concurrently.

In some embodiments, each valve (or valve array) can be housed inside or carried by a respective hub 2724a, 2724b, 2724c. In some embodiments, each valve (or valve array) may be housed adjacent to or remote from a respective hub. In such examples, additional fluid lines (e.g., 2720, 2721, 2723) may be added between each manifold and a corresponding valve. The fluid lines 2720a-c, 2721a-c, and 2723a-c may be tubes. In some embodiments, any of the fluid lines 2720a-c, 2721a-c, and 2723a-c may be removably coupled to their respective hubs. Alternatively, any of the fluid lines 2720a-c, 2721a-c, and 2723a-c may be inseparably connected to the hubs and removably coupled to other components of the fluid management portion 2702, such as the valve arrays 2716a-c or manifolds 2718a-c.

In some embodiments, the fluidics system 2700 may also include any number of pressure sensors, volume sensors, flow rate sensors, tubing sets, connectors, bubble sensors/detectors as will be discussed. In the illustrated implementation a pressure transducer 2734a is in pressure sensing communication with the first catheter 2726 by way of hub 2724a. Additional pressure transducers 2734b, 2734c may be placed in communication with their corresponding catheters as illustrated.

The control system may be configured to automatically adjust the various manifold valves, pumps and hemostatic valves (discussed below) in response to commands input by the physician. For example, a physician might input a command to infuse contrast through the third catheter 2730. The control system may cause a series of responsive events to automatically occur. At least the saline valve 2717c would close. Valves 2717a and 2717b may be closed or may remain open to provide positive pressure through the first and second catheters, to prevent backflow of contrast.

A control signal will be sent to a hemostasis valve in each of the first catheter hub 2724a and second catheter hub 2724b, to clamp down from a low pressure sliding fit to a high pressure clamp around the second catheter 2728 and third catheter 2730 respectively. This will prevent contrast from escaping proximally through the first catheter 2726 and second catheter 2728. A control signal will additionally be sent to valve 2719c to place the third catheter 2730 in fluid communication with the second source 2710b containing contrast solution.

If the space between the OD of the guidewire 2732 and the ID of the third catheter 2730 is insufficient to allow a desired contrast infusion rate, a further signal will be sent from the control system to the drive system controlling hub 2724d, to proximally retract the guidewire 2732 from the third catheter 2730 a distance sufficient to allow the flow of contrast through catheter 2730. An additional control signal may be sent to a hemostasis valve carried by hub 2724c to clamp in a high pressure mode around a distal portion of the guidewire 2732 or to clamp into a completely closed configuration if the guidewire 2732 was fully retracted. A further control signal may be sent to an electronically activated high pressure pump 2736 such as a syringe pump, high pressure positive displacement pump, contrast injection pump, etc., to deliver contrast solution through the third catheter 2730.

If the physician initiates a command to perform aspiration through, for example, the first catheter 2726, the control system may automatically transmit another series of control signals to execute the command. Signals will be sent to each of the hemostasis valves to move them from the high pressure configuration to the low pressure configuration in which there is less friction generated against a shaft of the catheter or guidewire. Such a configuration may permit relative movement of the various devices and proximal retraction of the second catheter 2728 and third catheter 2730 from first catheter 2726 while still inhibiting proximal blood loss through the hemostasis valves. Signals will be sent to the drive system to proximally retract each of the hubs 2724b, 2724c and 2724d. Valve 2723a will be opened to place the first catheter 2726 into fluid communication with the sink 2712. A signal will be sent to actuate the vacuum pump 2715, thereby aspirating blood and thrombus into the sink 2712. In some embodiments, when performing aspiration of the first catheter 2726, for example, communication between the catheter 2726 and the first fluid source 2710a and the second fluid source 2710b may be obstructed. For example, the corresponding valves of the valve arrays 2716a and 2716b may be closed to obstruct the manifolds 2718a and 2718b. Alternatively, the sources of volume and/or pressure (for example, pumps 2714 and 2736) may be deactivated or disconnected.

All of the fluid lines between the first source 2710a and second source 2710b and each of the catheters, and all of the fluid lines between sink 2712 and each of the catheters are preferably completely flushed free of any bubbles and filled with a fluid such as saline during system preparation before the procedure. This allows seamless transition between infusion, aspiration and manipulation of the catheters and guidewire without the need to disconnect and reconnect any fluid lines between the sources, sink and catheters, eliminating the risk of introducing air emboli during such exchanges.

It may also be desirable to enable confirmation of the absence of bubbles in any of the fluid lines. This may be accomplished placing bubble sensors in bubble sensing proximity to each of the fluid lines, such as in or upstream of each of the hubs, or at the manifolds. This may be particularly desirable in a telemedicine application, where the physician is at a remote workstation, and out of direct line of sight from the patient.

This may be accomplished using a non-contact ultrasonic sensor that measures the intensity and doppler shift of the reflected ultrasound through the sidewall of fluid tubing to detect bubbles and measure fluid flow rate or fluid level. An ultrasonic or optical sensor may be positioned adjacent an incoming fluid flow path within the hub, or in a supply line leading to the hub.

For example, to detect the presence of air bubbles in the infusion line (that is formed of ultrasonically or optically transmissive material) the sensor may include a signal source on a first side of the flow path and a receiver on a second side of the flow path to measure transmission through the liquid passing through the tube to detect bubbles. Alternatively, a reflected ultrasound signal may be detected from the same side of the flow path as the source due to the relatively high echogenicity of bubbles.

Alternatively, an optical sensor may be provided to detect changes in optical transmission or reflection due to the presence of bubbles, or to transmit a visual signal to a display at the remote workstation where the physician can visually observe the presence of a bubble moving through the tubing. In a system having a bubble detector, the control system can be configured to automatically shut down all fluid flow in response to the detection of a bubble to give personnel an opportunity to plan next steps.

In one implementation, a bubble removal system is automatically activated upon detection of in line bubbles. A processor may be configured to activate a valve positioned in the flow path downstream of the bubble detector, upon the detection of bubbles. The valve diverts a column of fluid containing the detected bubble out of the flow path leading to the patient and instead into a bypass flow path or reservoir. Once bubbles are no longer detected in the flow path and after the volume of fluid in the flow path between the detector and the valve has passed through the valve, the valve may be activated to reconnect the source of fluid with the patient through the flow path. In some embodiments, the flow path may include any number of bubble filters and/or traps to remove bubbles from the flow path.

The robotic system portion 2704 may include a drive table that is configured to receive (e.g., be coupled to) any number of hubs (2724a, 2724b, 2724c, 2724d, etc.). Additional details of the hubs, drive table and related systems are found in U.S. patent application Ser. No. 17/816,669, entitled Method of Supra-Aortic Access for a Neurovascular Procedure, filed Aug. 1, 2022, which is hereby expressly incorporated in its entirety herein. Each hub is configured to be coupled to a catheter, or guidewire, one or more fluidics lines, one or more electrical lines, one or more controls, and/or one or more displays. For example, a drive table may be positioned over or alongside the patient, and configured to support axial advancement, retraction, and in some cases rotation and/or lateral deflection of two or three or more different (e.g., concentrically or side by side oriented) devices (e.g., catheters, guidewires, etc.).

The drive system independently drives movement of each hub independently in a proximal or distal direction across the surface of the table to move the corresponding interventional device (e.g., catheter 2726, catheter 2728, catheter 2730, and/or guidewire 2732) proximally or distally within the patient's vasculature.

The respective catheter 2726, catheter 2728, catheter 2730, and/or guidewire 2732 may be guided into a bodily lumen (not shown) as a single concentric catheter stack, in response to movement of the respective hubs 2724a, 2724b and 2724c as discussed elsewhere herein. The system 2700 may also include a guidewire hub 2724d for controlling the guidewire 2732, which may also be introduced into a bodily lumen along with one or more of catheter 2726, catheter 2728, and/or catheter 2730.

In some embodiments, a driven magnet is provided on each hub. Each driven magnet is configured to cooperate with a drive magnet associated with the table such that the driven magnet(s) move in response to movement of the drive magnet(s). In such examples, the drive magnet(s) may be axially movably carried by the support table.

Because multiple sources and/or sinks are configured to each be coupled (and remain coupled) to each catheter hub (e.g., hubs 2724a, 2724b, and 2724c), the fluidics system 2700 provides the advantage of enabling faster procedures than conventional fluidics systems that utilize manual removal, addition, and/or switching of fluids, catheters, hubs, and the like during the procedure. For example, the fluidics system 2700 enables each fluid line/catheter hub to be connected to each source fluid and/or sink before beginning a procedure. When the interventionalist (or other medical practitioner) performing the procedure is ready to use a particular source fluid or sink, the system 2700 is already configured and ready to allow use of the particular source fluid or sink without having to switch between different fluid lines for particular catheters. In some embodiments, the system 2700 may be used to provide a method of treatment in which fluid sources need not be connected to and/or disconnected from a medical device more than once during a procedure.

Thus, the interventionalist can inject any of the fluids contained in fluid sources 2710a, 2710b and/or collect aspirate from any of the catheters 2726, 2728, and/or 2730 at any point during the procedure because each catheter hub 2724a, 2724b, 2724c is provided access to all fluid lines at all times.

Because the multiple sources that are indicated for a particular procedure are preconfigured to be connected to each catheter/catheter hub, an interventionalist (or other medical practitioner) may be assured that there is no repetitive connecting and disconnecting of syringes or other source fluid containers, fluid lines, etc. during the procedure. This assurance removes the possibility of introducing bubbles into the catheter flow during the procedure because no connecting or disconnecting of fluid sources are needed with the use of system 2700. Instead, each fluid source and sink are connected and tested before the procedure and are not removed until after the procedure is completed. In some embodiments, the constant connection of fluid sources and sinks to catheter hubs associated with operation of system 2700 removes the variability and risk in remote procedures where the interventionalist is in a control room rather than the procedure room.

The valves within valve arrays 2716a, 2716b, and or 2716c of system 2700 are depicted at the respective manifolds 2718a, 118b, and 118c. In such a configuration the valves are near the respective source and/or sinks with about two meters to about three meters (e.g., about six to about ten feet) of fluid line between the valves of valve arrays 2716a, 2716b, and 2716c and the respective catheter hubs 2724a, 2724b, and 2724c. In some embodiments, the valves of valve arrays 2716a, 2716b, and/or 2716c may instead be located at the sources/sinks (e.g., 2710a, 2710b, and/or 2712). In some embodiments, the valve arrays 2716a, 2716b, and/or 2716c are coupled to the fluid lines at a location between the source/sink and the hubs. In some embodiments, the valve arrays 2716a, 2716b, and/or 2716c may be located at the catheter hubs 2724a, 2724b, and/or 2724c. In some embodiments, valves that are located at or near the hubs may be disposable valves. Other components of systems 2700, 2800 may also be disposable and/or re-processable for reuse.

In some embodiments, the system 2800 may additionally include valves 2713a-2713i between the valve arrays 2716a, 2716b, and 2716c and the respective hubs 2724a, 2724b, and 2724c. The valves 2713a-c can be part of a valve manifold (for example, such as the valve manifold 2616 of FIG. 26B) that is part of or coupled, directly or indirectly, to the hub 2724a. The valves 2713d-f can be part of a valve manifold (for example, such as the valve manifold 2616 of FIG. 26B) that is part of or coupled, directly or indirectly, to the hub 2724b. The valves 2713g-i can be part of a valve manifold (for example, such as the valve manifold 2616 of FIG. 26B) that is part of or coupled, directly or indirectly, to the hub 2724c. The valves 2713a-2713i may be one-way check valves. As shown in FIG. 27, the one-way check valves 2713a-2713i can allow flow in the direction in which their respective arrows are pointing.

Each hub 2724a, 2724b and 2724c may be provided with a hemostasis valve to accommodate introduction of another device therethrough, as illustrated in FIG. 28. The hemostasis valve includes a variable diameter aperture such as an aperture through a resilient gasket.

The gasket may be actuatable between a first, fully open state; a second partially open state for sealing against low pressure fluid injections from the first fluid source or the second fluid source through a first port (as described herein), for permitting fluid to flow through the first port to the sink, while allowing advancing or retracting an interventional device; and a third tightly closed state for resisting backflow of high pressure fluid (e.g. contrast media) injections from the second fluid source through the first port or for permitting fluid flow through the first port to the sink. The gasket may be manually actuatable or automatically actuatable, for example based on a user input corresponding to manipulation of one or more of the interventional devices of the system.

FIG. 28 illustrates another embodiment of a fluidics system 2800 for use with a fluidics management system. In general, the fluidics system 2800 includes two or more fluid channels that feed one or more fluid lines configured to interface with a rotating hemostatic valve. The two or more fluid channels may receive fluids from fluid sources with any number of fluid materials. The two or more fluid channels may allow different fluid materials provided by different fluid sources (of various volumes and/or under different pressures) to flow into or from a bodily lumen. Each of the two or more fluid channels may be primed with a respective fluid in order to be ready for transport into the single fluid line and into the body. Valves and/or valve arrays may be employed to switch between usage of the two or more fluid channels.

As shown in FIG. 28, the fluidics system 2800 may include a vacuum chamber and/or control 2802, within the sterile field. The vacuum chamber may have a clot filter with a window for visualizing trapped clot, and a valved vent which when momentarily opened permits entrance of air to allow direct visualization of the clot through the window. To permit inspection by a remote physician, a CCD or CMOS sensor may be mounted such that the upstream surface of the clot filter is within the sensor field of view. This permits viewing the contents of the filter on a remote monitor. Air intake to clear the optical path from the window to the filter may be controlled remotely using an electronically actuated valve.

An air bubble filter 2804 may be provided in line between a needle injection port 2806 and catheter 2826. The system 2800 further includes a line branch point 2808 (e.g., a wye) in fluid communication with a first source 2810a and a second source 2810b. The line branch point 2808 may include luer lock connectors or wye connector that interfaces with multiple fluid sources.

The system 2800 may also include a pump such as a peristaltic pump 2834 or rotary piston pump that drives fluid under pressure from the second source 2810b to the line branch point 2808 in the direction of arrow 2822c.

An air bubble sensor may be provided on an upstream or downstream side of the pump 2834. The air bubble sensors 2836a, 2836b may be non-contact ultrasonic sensors that measure an intensity and doppler shift of a reflected ultrasound through a sidewall of fluid tubing to detect bubbles and measure fluid flow rates or fluid levels as has been discussed. In some embodiments, the sensor 2836a may also be a pressure sensor or a separate pressure sensor may be provided.

A valve 2816c such as a ball valve or rotary valve can selectively open or close fluid communication between the second source 2810b and the catheter 2826. A flow rate detector such as a drip rate sensor 2838 enables determination and display of the flow rate from second source 2810b.

Fluid flow from the first source 2810a is directed through a one way check valve 2814 and on to a high pressure pump 2852, which may be a syringe pump, high pressure positive displacement pump, contrast injection pump, etc., High pressure fluid (e.g., contrast solution) is directed through an air bubble sensor 2836a and on to branch point 2808 through valve 2816b. Arrows 2822b indicate the direction of fluid flow.

Resistance to fluid flow through different catheters in a concentric catheter stack differs based upon the available lumen cross sectional area. For example, resistance measurements within an inner catheter with a fully open lumen (e.g., with guidewire removed) may be lower than resistance measurements within an outer catheter having a second catheter (or a guidewire) extending therethrough. Therefore, when performing saline flushing steps, the fluidics system 2800 may be configured to ensure a similar flow rate or a procedure appropriate flow rate through each inner and outer catheter to avoid clotting or other issues within the catheters. To do so, a valve may be adjusted for each catheter to ensure the flow rate remains constant amongst all catheters during saline flushing. The system 2800 may determine such flow rates in real time based on flow rate sensors, and the control system may be configured to automatically adjust valve settings and/or pump parameters to maintain the desired flow rate through each catheter.

In some embodiments, fluid resistance may be altered by adjusting an insertion length of each shaft into its concentrically adjacent lumen. As described herein, fluid resistance within a lumen may be greater when there is a reduction in cross sectional luminal area for flow, for example, when a second catheter (or a guidewire) extends into the lumen. The amount of fluid resistance can be affected by the length of the cross sectional narrowing, for example, due to placement of the second catheter (or guidewire) within the lumen. A second catheter (or guidewire) extending partially through the lumen of a first catheter will provide a smaller length of cross-sectional narrowing, and accordingly may result in a lower fluid resistance within the lumen of the first catheter, than if the second catheter (or guidewire) were to extend entirely through the lumen of the first catheter. Thus, fluid resistance can be lowered by partially retracting a depth of insertion of a second catheter (or guidewire) into the lumen through which fluid is to be injected.

The system 2800 further includes an aspiration canister 2840 coupled to an upstream side of filter 2844. A downstream side of the filter 2844 is coupled to a vacuum pump 2842. The aspiration canister 2840 is connected to a valve 2816a, which may be in communication with a sterile field clot capture container 2802 which has been discussed elsewhere herein. Arrow 2822a indicates the direction of fluid flow.

An optional pressure sensor 2846 is depicted on a proximal end of a catheter 2826 or hub coupled to a hemostatic valve, such as a rotating hemostatic valve (RHV) 2848.

In this example, the RHV 2848 is connected to two different fluid sources. The RHV 2848 may be carried by and at least partially disposed in a hub (e.g., hub 124a of FIG. 2). The RHV 2848 may comprise a first fluid source connection, a second fluid source connection, and a sink connection. For example, the first connection, the second connection, and the third connection may comprise respective valves (e.g., valves 2816a, 2816b, and 2816c) connected via fluid lines to RHV 2848. In some embodiments, the connection points may be formed as part of the RHV 2848 itself and fluid lines may connect directly to the connection points at proximal ends of the fluid lines and connect to sources and/or sinks at the respective distal ends of the fluid lines. In certain embodiments, the valves 2816a, 2816b, and 2816c may be arranged in a valve manifold or a valve manifold cassette. In certain embodiments, the pump 2834 may also be arranged in the valve manifold or valve manifold cassette. In certain embodiments, any of the valves 2816a, 2816b, and 2816c can be a ball valve, a stopcock valve, a rotary valve, a solenoid valve, or any other suitable valve. Any of the valves 2816a, 2816b, and 2816c can be controlled by one or more actuators 2817.

The RHV 2848 may be configured to enable a catheter or other instrument to be introduced into the body of a living being while precluding unintended back bleeding. In some embodiments, each RHV described herein may be configured with at least a fully closed configuration, a low sealing force state in which devices may be advanced therethrough without leaking, and a high sealing force state (e.g., mode) which prevents escape of fluids under high pressure and may prevent axial movement of devices therethrough.

The RHV 2848 is configured to be concurrently and fluidly connected to a first fluid source (e.g., source 2810a) via the first fluid source connection (e.g., valve 2816b). The RHV 2848 is further configured to be concurrently and fluidly connected to a second fluid source (e.g., source 2810b) via the second fluid source connection (e.g., valve 2816c). In addition, the RHV 2848 is further configured to be concurrently and fluidly connected to the sink (e.g., aspiration canister 2840) via the sink connection (e.g., valve 2816a).

In operation, the system 2800 is configured to automatically switch between introducing fluid into a lumen of the elongate body (e.g., catheter 2826) through the RHV 2848 from the first fluid source (e.g., source 2810a) or from the second fluid source (e.g., source 2810b) or to permit fluid removal from the lumen to be collected in the sink (e.g., aspiration canister/sink 2840).

In some embodiments, the optional pressure sensor 2846 is located at either the upstream side or downstream side of the RHV 2848 (as shown in FIG. 28). In some embodiments, the optional pressure sensor 2846 is located in the catheter (e.g., in a sidewall of the catheter) to measure arterial pressure at the catheter distal end. The pressure may be assessed by the interventionalist to verify that the catheter is not misaligned within the vessel and/or the thrombus.

For example, if the catheter is misaligned against a vessel wall, then the detected pressure (e.g., waveform) may be blunted. Such a detection may be provided to an algorithm performed by a processor associated with system 2800, for example, to determine the patency of the lumen of the catheter of the patency of the catheter distal tip. Such a pressure sensor and algorithm may provide an improved alternative to conventional determinations of pressure where manual operation of fluidics is occurring and an interventionalist may retract (e.g., pull back) on a syringe coupled to the catheter to verify that blood capture occurs and to assess tactile feedback of the catheter.

Such blood capture and tactile feedback assessments may indicate patency of the lumen or distal tip before an injection or aspiration is performed. However, the pressure sensor 2846 may provide for an automated and improved way to assess lumen or distal tip patency. That is, the addition of a pressure sensor 2846 (e.g., a blood pressure sensor) on the proximal end of a catheter may capture an arterial pressure waveform. The waveform can be used to determine whether the catheter distal tip is pressed against a vessel wall, the catheter tip is pressed against a thrombus, the catheter tip has full patency, or the catheter lumen is in a clogged or fully patent state, without having direct visual or tactile feedback. In some embodiments, the waveform can be used to determine a state of engagement of the catheter distal tip against the clot and/or a consistency of the clot.

In some embodiments, the fluidics systems (e.g., system 2700, system 2800) described herein include a hemostasis valve (e.g., RHV 2848) that includes a first three-way connector having a first fluid source connection (e.g., one-way valve 2716a, 2816b), a second fluid source connection (e.g., one-way valve 2716b, 2816c) and a sink connection (e.g., one-way valve 2816a).

In some embodiments, the fluidics systems described herein (e.g., system 2700, system 2800) utilize a first fluid source that comprises one of saline, heparinized saline, or a pharmaceutical. In some embodiments, the second fluid source (e.g., source 2710b, 2810b) comprises contrast.

The systems 2700, 2800 may further include a second hemostasis valve that is in communication with and may be at least partially disposed in the second hub (e.g., 2724b). The second hemostasis valve may include a third fluid source connection (e.g., valve 2716b), a fourth fluid source connection (valve 2716b), and a second sink connection (e.g., valves 2716c). In this example, the first manifold 2718a may include a second output line that is configured to connect to a third fluid source connection (not shown).

FIG. 29A illustrates an embodiment of a fluidics system 3100 for use with a fluidics management system. In general, the fluidics system 3100 includes a cassette that can couple a plurality of fluid sources and/or sinks to a plurality of interventional devices. A plurality of fluid lines may extend between a source or sink and the cassette for coupling to different interventional devices. For other sources and sinks, a single fluid line may extend between the source or sink and the cassette and may split within the cassette to connect to different interventional devices. In certain embodiments, the cassette can include connection arrays formed of connections from a plurality of fluid sources and/or sinks for coupling to a single interventional device (for example, in a row or column). Each connection array can couple to a tubing set having a tube corresponding to each connection in the connection array.

The cassette 3141 may be a self-contained unit comprising a housing having a plurality of valves, tubing and connectors as described below. A first connector array comprises a plurality of releasable connectors such as luer connectors, for placing the cassette in fluid communication with complementary connectors in fluid communication with sources of aspiration and at least one or two or more fluids. A second connector array is configured for releasable connection to a tubing set configured to extend between the cassette and at least one or two or three interventional devices.

The cassette 3141 thus forms a bridge module that when assembled resides between the various fluid and vacuum sources, and the corresponding interventional devices. The cassette 3141 may be configured for a single use, or may be re-sterilizable and reusable.

As shown in FIG. 29A, the system 3100 may include a first fluid source 3110a and a second fluid source 3110b. Fluid flow from the first source 3110a is directed through a one way check valve 3114 and on to a high pressure pump 3152, which may be a syringe pump, high pressure positive displacement pump, contrast injection pump, etc. The fluid from the first fluid source 3110a may be a contrast solution which is preferably injected under high pressure.

Fluid flow from the syringe pump is directed into a cassette 3141, which may include a plurality of valves, manifolds, and/or connectors. Within the cassette 3141, the fluid flow may split along a plurality of branches 3118b to a plurality of connectors 3117b (for example, four connectors 3117b as shown in FIG. 29A) for coupling with different interventional devices. The cassette may include a valve 3116b (e.g., a ball valve) with each branch 3118b upstream of the connector 3117b. In certain embodiments, any of the valves 3116a or 3116b can be a ball valve, a stopcock valve, a rotary valve, a solenoid valve, or any other suitable valve.

Fluid flow from the second fluid source 3110b may be directed into a plurality of branches 3118c to a plurality of pumps 3134c (for example, four pumps 3134c as shown in FIG. 4), such as peristaltic pumps or rotary piston pumps. Each pump 3134 can drive the fluid (for example, saline) under pressure from the second source 3110b to a unique connector 3117c for each interventional device within the cassette 3141.

The system further includes an aspiration canister 3140 in communication with an upstream side of a filter 3144. A downstream side of the filter 3144 is in communication with a vacuum pump 3142. The aspiration cannister receives fluid from the cassette 3141 which includes a plurality of connectors 3117a each being configured to couple to a unique interventional device. A unique valve 3116a (at least two, and four in the illustrated example) may be positioned upstream of each connector 3117a. Each unique valve 3116a may be positioned along a branch 3118a.

In certain embodiments, one or more connector arrays 3146 may be arranged, each connector array 3146 configured to couple an interventional device. For example, a connector array 3146 is indicated by dashed lines in FIG. 29. As shown in FIG. 29A, the connector array 3146 can include a connector 3117a, a connector 3117b, and a connector 3117c. As shown in FIG. 29, the array 3146 may be organized with all connectors facing in the same direction on a common plane, such as a linear row.

The connector array 3146 can releasably couple to a tubing set 3143 including an aspiration tube 3154, a first fluid tube 3155, and a second fluid tube 3156. In some embodiments, the connectors 3117a, 3117b, and 3117c can be luer lock connectors. The aspiration tube 3154 can couple to the connector 3117b of the array 3146 by way of a complementary connector 3117d for aspiration from the interventional device to the aspiration container. The first fluid tube 3155 can couple to the connector 3117b of the array 3146 by way of a complementary connector 3117e to provide fluid flow from the first fluid source 3110a to the interventional device. The second fluid tube 3156 can couple to the connector 3117c of the array 3146 by way of a complementary connector 3117f to provide fluid flow from the second fluid source 3110b to the interventional device. The tubes 3154, 3155, and 3156 may be joined together over a majority of their lengths. The tubes 3154, 3155, and 3156 can each have a length of at least about three or four feet, and in certain embodiments between about 6 feet and about 8 feet.

As shown in FIG. 29A, the tubing set 3143 includes a line branch point 3100 (e.g., a two to one or a three to one wye) that can provide fluid communication between the interventional device and the tube 3154, tube 3155, and tube 3156. The line branch point 3100 may include luer lock connectors or wye connectors that interface with complementary connectors on the tubing set. In certain embodiments, a one way valve 3145 may be positioned upstream of the branch point 3108 and downstream of the cassette 3141 along the flow path of the second fluid.

In certain embodiments, the system 3100 (or other systems described herein) can direct the flow of the second fluid (for example, saline) using two different flow modes. In a low flow drip mode, a flow rate of about 1-2 drips per second or 3-6 mL/min may be provided, for example, by the pumps 3134. In some embodiments, a low flow mode rate of 1-8 mL/min may be provided. Each catheter coupled to the system may experience a different fluid resistance as described herein.

The pumps, for example pumps 3134, can be operated to provide the same flow rate in each catheter. In certain embodiments the fluid pressure within the catheter can be at least about 330 mmHg or 6.5 PSI. This pressure may be enough to overcome arterial pressure while delivering the desired drip rate. In certain embodiments, the pressure within the catheter can be greater than 330 mmHg. In certain embodiments, the delivered fluid volume can be at least about 1 liter over the length of a procedure. In some embodiments, the fluid volume can be up to 2 liters.

In a high flow flush mode, all of the fluid lines may be flushed to remove air. The flow rate can be between 100-1000 mL/min. The fluid pressure may be between 5-10 PSI. The volume delivered can be between 0.5-1 liters per procedure. Volume may depend on tubing length and diameter. In some embodiments, the high flush flow rate is at least about 20 times and in some cases between 30 to 150 times the low flow drip mode flow rate.

In certain embodiments, the first fluid (for example, contrast solution) can be provided at a flow rate of between 3-8 L/s (for example, about 4 mL/s), for example, by the pump 3152. In certain embodiments, the flow rate can be up to about 8 mL/s. In other embodiments, the flow rate can be up to about 20 mL/s. In certain embodiments, the first fluid can be provided with a pressure of about 400 PSI for a flow rate of about 4 mL/s. The amount of pressure needed may depend on flow rate and flow restriction of the fluid path. The pressure may increase proportionally with the flow rate for higher flow rates. In certain embodiments, the pressure may be up to 1200 PSI.

In certain embodiments, the high pressure pump, such as pump 3152, can provide a delivered volume of between 5-15 mL per high pressure injection. In certain embodiments, the pump can provide the 5-15 mL per high pressure injection in increments of about 1 mL per puff. In certain embodiments, the second fluid source can provide a total volume of about 200 mL per procedure. In certain embodiments, the syringe pump is sized to hold at least about 150 mL or 200 mL so as to provide uninterrupted flow throughout the procedure without the need to add additional contrast solution. In other embodiments, the second fluid source can provide a total volume of between 150-250 mL per procedure.

In certain embodiments, the flow rate may vary depending upon the anatomical location at the distal end of the catheter. For example, within the aortic arch, the flow rate may be about 20 mL/s. A total delivered volume of about 25 mL may be infused in the aortic arch. Within the common carotid artery, the flow rate may be about 20 mL/s. A total delivered volume of 12 mL may be infused in the common carotid artery. Within the subclavian artery, the flow rate may be about 6 mL/s. A total delivered volume of about 15 mL may be infused in the subclavian artery. Within the internal carotid artery, the flow rate may be about 6 mL/s. A total delivered volume of about 8 mL may be infused in the internal carotid artery. Within the external carotid artery, the flow rate may be about 3 mL/s. A total delivered volume of about 6 mL may be infused in the external carotid artery. Within the vertebral artery, the flow rate may be about 6 mL/s. A total delivered volume of 8 mL may be infused in the vertebral artery.

In certain embodiments, a motor may be provided to drive the high pressure pump, such as pump 3152, which can be controlled with a position and velocity control loop using a potentiometer as a measurement to close the loop. In certain embodiments, current control may be applied to provide approximate pressure limiting. In certain embodiments, the second fluid can be a contrast solution such as Omnipaque 300, Omnipaque 350, or Visipaque 320.

In certain embodiments, a vacuum pump, such as pump 3142, can provide a pressure of about −29.5 inHg or up to −29.5 inHg (−999 mbar). In certain embodiments, tubing used for aspiration can have an inner diameter of 0.11 inches (about 2.8 mm). In certain embodiments, the volume of the aspiration container, such as container 3140, can be at least about 0.5 L. In certain embodiments, the volume of the aspiration container can include about 0.5 L for blood and additional volume for a saline flush. In certain embodiments, the aspiration container can have a volume between 0.25-0.75 L. In certain embodiments, the vacuum pump can be configured to operate to additionally provide a low pressure/flow setting to assist a flushing process as it may be desirable that an aspiration line is full of saline at all times (except when aspirating a clot). In certain embodiments, a separate pump may be provided for the low pressure/flow setting.

Examples here include configurations, schematics, and flow diagrams of a fluidic system, including subsystems and components, that can be controlled for performing fluidic processes in a robotic catheter system including, for example, priming the saline subsystem, priming the contrast subsystem, operating a hemostasis valve, injecting contrast through one or more catheters in a set of concentric stack of catheters, aspirating a clot, and back-bleeding. Processes performed by the robotic catheter system are controlled at least in part by a controller and can be controlled based on a user input, predetermined programmed processes, parameter information, and/or based on conditions sensed by the system. FIGS. 29b-29F illustrate examples of other configurations of a fluidics system that can be used in a remotely controllable robotic catheter system to perform such processes. For example, to perform such processes, a system (e.g., including valves, pumps, and other actuatable (or movable) components) can be driven to be aligned in a position by a controller to provide contrast injection through one catheter selected from a plurality of catheters, or through more than one catheter (e.g., to provide contrast through two catheters, three catheters) selected from a plurality of catheters. In an example, a controller can configure the fluidics system to provide a contrast injection through one or more catheters in a concentric stack of two catheters. In an example, a controller can configure the fluidics system to provide a contrast injection through one or more catheters in a concentric stack of three catheters. Additional examples of performing a contrast injection using one or more catheters in a concentric stack of catheters are described in reference to FIGS. 52-63.

In some embodiments, the state or position of actuatable components (e.g., valves) can be determined by a position sensing mechanism on the component such that the controller can determine the current position of the component before, during, or after a process. In some embodiments, the positioning sensing mechanism can include a switch, encoder, etc. The sensing mechanism can be incorporated in the cassette or the mount to determine a position of a movable component (e.g., valve, pump) or on an actuator that drives the movable component. In some embodiments, a current state or position of the components can be electronically stored (e.g., in a table or file) and the controller is configured to determine the position of one or more components by accessing the stored information. In an example, before contrast is injected using a selected catheter in a system having multiple catheters, a valve coupling a selected catheter to a contrast subsystem can be determined to be aligned in an open position to provide a flow-path for contrast to the selected catheter, and one or more valves coupling non-selected catheters to the contrast subsystem can be determined to be aligned in a closed position to shutoff a flow-path of contrast to non-selected catheters. Such determinations may be made using the stored information and/or by using information from a sensing mechanism. The processes described herein are some of the processes a controller can be configured to perform by controlling fluidic equipment, either automatically or semi-automatically, based on one or more user inputs, sensed information, and/other information.

FIG. 29B is a schematic illustrating an example of a robotic catheter system that includes another embodiment of a fluidics system. The robotic catheter system illustrated in FIG. 29B can include actuatable components of a fluidics management system that are movable, as controlled by a controller, to align fluid communication channels to provide saline, contrast, and vacuum from a saline, contrast, and vacuum source (respectively) to hub assemblies 1400a-1400c. As indicated above, in some examples the hub assemblies 1400 can include a hub (first subassembly) and a mount (second subassembly). Although some of the examples described herein refer to mounts and components of mounts, this description is not limited to embodiments of hub assemblies that include a hub and a mount, rather the components and processes disclosed can also be implemented in both hub assemblies that do not include a first subassembly and a second hub assembly but instead are housed as a one-part hub assembly. Accordingly, reference to a mount(s) should be understood as also referring to either a multi-part hub assembly (e.g., two-part hub assembly having a hub and a mount) or part of a one-part hub assembly and unless specifically indicated or indicated by context. Correspondingly, reference to a hub assembly should be understood as referring to either a multi-part hub assembly (e.g., two-part hub assembly having a hub and a mount) or a one-part hub assembly.

The robotic catheter system can include certain sensors that are configured to sense a condition related to providing provide saline, contrast, and vacuum to the mounts 1400a-1400c and provide the sensed information to a controller. For example, the position of a valve, information from a pressure sensor, information from an air bubble sensor, etc. The controller can be configured to use the sensed information, user inputs, and/or stored information to control actions of the fluidics system to perform a medical procedure. In various embodiments, a pump station 2240 may include a controller to control actuators of the pump station 2240 to perform fluidic-related actions (e.g., provide saline, contrast, and vacuum to the mounts 1400a-1400c). In other embodiments, controller 2230 and/or system controller 2220 can be configured to control actuators and other components of the pump station 2240 to perform fluidic-related actions. Certain components and systems of the robotic catheter system may not be illustrated in FIG. 29B for clarity of this illustration (for example, a hub axial drive system, check valves, a catheter rotational system, controllers of the remote control system 2210, etc.).

In the embodiment illustrated in FIG. 29B, robotic catheter system includes a fluidics assembly 1100 that includes a cassette 1200 which is configured to be releasably attached to a pump station 2240. The cassette 1200 includes a housing 1209 that supports a saline subsystem 1201, a contrast subsystem 1202, and a vacuum subsystem 1203. The saline subsystem 1201, contrast subsystem 1202, and vacuum subsystem 1203 can be all or partially enclosed within the housing 1209. Portions of the saline subsystem 1201, contrast subsystem 1202, and vacuum subsystem 1203 that interface with the components on the pump station 2240 can be positioned on the surface of the housing 1209. For example, an interface for an actuator to mechanically couple to a valve in the cassette, an interface for a portion of a contrast pump or saline pump in the cassette to mechanically couple to a portion of a contrast or saline pump in the pump station 2240 (e.g., a pump driver). The cassette 1200 can be placed on the pump station 2240 in position 1200a such that components of the cassette 1200 align with corresponding components of the pump station 2240. In an example, to align valves in the cassette 1200 with valve actuators of the pump station 2240, to align a portion of a peristaltic pump in the cassette 1200 with a portion of a peristaltic pump in the pump station 2240, and/or to align contrast pump in the cassette 1200 with a contrast pump drive mechanism in the pump station 2240. In another example, an electrical interface 1236 having a plurality of electrical connections on the cassette 1200 can be aligned with a corresponding electrical interface 1112 on the pump station 2240. The saline subsystem 1201, contrast subsystem 1202, and vacuum subsystem 1203 are further described in reference to FIG. 67. The cassette 1200 also includes an electrical interface 1236 which can be coupled to pump station electrical interface 1112 using various corresponding mechanical-electrical connectors can be used. For example, using a socket and a plurality of extended connectors built into the cassette and the pump station, using an electrical pigtail on one or both of the cassette and the pump station, using corresponding pogo pins connectors, etc. In operation, after the cassette 1200 is coupled to the pump station 2240, one or more saline source 1106 can be coupled to the saline subsystem 1201, one or more contrast source 1108 can be coupled to the contrast subsystem 1202, and one or more vacuum source 1110 is coupled to the vacuum subsystem 1203.

The fluidics assembly 1100 also can include one or more mounts 1400a-c, and communication channels 1300 coupled to the cassette 1200 and to each of the mounts 1400a-c, the communication channels 1300 structured to separately communicate saline, contrast, and vacuum from the cassette 1200 to the mounts 1400. As indicated above, the terms “channels,” “tubes,” “lines,” molded apertures and structures, valves, and other structures or portions of components through which a fluid or a gas flows may be referred to herein generally as a fluid communication channel, or a “communication channel,” or a “channel.” The vacuum communication channel provides a path for aspirating material from any of the catheters 1402a-c coupled to the mounts 1400a-c, respectively, to a clot pod or vacuum canister (for example, as illustrated in FIG. 29E). In the embodiment illustrated in FIG. 29B, a guide catheter 1402a is coupled to a guide catheter mount 1400a, a procedure catheter 1402b is coupled to a procedure catheter mount 1400b, and an access catheter (or insert catheter) 1402c is coupled to an access catheter mount 1400c. In this example, the mounts 1400a-c are part of two-part hubs, the mounts 1400a-c being the first hub assemblies. The catheters 1402a-c are attached to second hub assemblies that mechanically couple to respective mounts 1400a-c and are configured to fluidically couple to mounts 1400a-c as well, for example, via connector (port) 1434a-c (FIG. 29F). The mounts 1400 are controllable to move in the same axial direction and the catheters are sized and configured to be positioned in a nested configuration, that is, the access catheter 1402c can be positioned in part of or in the entire lumen of the procedure catheter 1402b, and both the access catheter 1402c and the procedure catheter 1402b can be positioned in part of or in the entire the lumen of the guide catheter 1402a, based on the positioning of the mounts 1400.

In some embodiments, all or part of the fluidics system that is attached to the pump station is made to be disposable. In some embodiments, part of the fluidics system that is attached to the pump station is made recyclable. In either case, the system fluidics system can be advantageously designed to be as lowest cost as feasible. For example, valves in the fluidics system can be positioned in the cassette 1200 while their corresponding valve actuators can be positioned in the pump station. The contrast pump can be positioned in the cassette and the corresponding contrast pump driver can be positioned in the pump station. A clot pod can be positioned in the cassette and a vacuum pump 1267 and a vacuum regulator can be positioned external to the cassette, as illustrated in FIG. 29E. In some embodiments, a camera 1292 (FIG. 29E) is used to generate an image of a clot pod assembly 1272 (FIG. 29E) for viewing on a local or remotely located display, where the camera 1292 (capital equipment) can be positioned in the pump station 2240 or somewhere bedside (locally) and the clot pod assembly 1272 (disposable) can be positioned in the cassette. As imaging technology continues to become less expensive, in other embodiments a relatively inexpensive camera or sensor can be positioned in the cassette to generate an image of the clot pod to determine if it contains an aspirated clot.

FIG. 29C is a schematic of the example of the fluidics assembly 1100 of FIG. 29B, illustrating some components in further detail. In FIG. 29C, the fluidics assembly 1100 includes a cassette 1200 that couples to the pump station and to a saline source, a contrast source, and a vacuum source, for example as described in reference to FIG. 29B and further in reference to FIG. 29E. The fluidics assembly 1100 also includes a plurality of fluid communication channels 1300 to provide saline, contrast, and vacuum to one or more mounts 1400a-c and to the hubs (not shown) and catheters 1402a-c coupled to the mounts 1400a-c. The fluid communication channels 1300 also can include a plurality of electrical leads that connect components in the mounts (e.g., bubble sensors, pressure sensors, etc.) to the electrical interface 1236 of the cassette to provide signals to the control system 2220 when the cassette 1200 is coupled to the pump station 2240 (FIG. 29B). In systems that include a plurality of hub assemblies, different hub assemblies may be configured differently or may have different fluids communicated to them, which are provided to a catheter. For example, in a system with a plurality of hub assemblies, one hub assembly may only provide saline to a catheter, and/or one hub assembly may provide saline and contrast to a catheter, and/or one hub assembly may provide saline and vacuum to a catheter, and/or one hub assembly may provide saline, contrast, and vacuum to a catheter.

One challenge of a fluidics system that provides fluids (e.g., saline and contrast) that enter a patient via a catheter is to provide such fluids in air-free (that is, bubble-free) fluid flows. As described in detail above and further below, typically when the cassette, fluid communication channels, hubs/mounts, and catheters are provided for use in an operating room they are not filled with fluid, and thus require priming to remove air from the saline and contrast flow-paths to prevent air bubbles entering a patient during a medical procedure. Once primed, air bubble detectors in the cassette and the mounts (illustrated in FIGS. 29E and 29F) can help mitigate risks associated with air bubbles within saline and contrast flow-paths. However, because of the dangerous consequences of air entering a patient's bloodstream, where it is possible the fluid communication channels are visually inspected for air bubbles by a medical partitioner after the saline and contrast flow-paths are primed. Because of flexibility and mobility requirements for providing fluids from static saline and contrast fluid sources across a certain distance to moving mounts/catheters, at least a portion of the fluid communication channels between the cassette and the mounts can include flexible clear tubing. During the visual inspection, each centimeter of all the clear tubing supplying saline or contrast from the cassette 1300 to the mounts 1400 is carefully inspected to determine if air bubbles are present, and if so the lines can be re-primed. The length of time it takes to inspect the clear tubing for air bubbles directly relates to the total length of the contrast and saline tubing between the cassette and the mounts. In some examples (e.g., FIG. 29B), the pump station is located several feet away from the set of mounts 1400 to provide room around the patient, and the saline and contrast fluid communication channels 1300 between the cassette 1200 and mounts 1400 may be about 14 feet long. If, as illustrated in FIG. 29C, there are three mounts 1400a-c coupled to the communication channels 1300 and the communication channels include individual tubes for saline and contrast for each of three mounts 1400, this results in 84 linear feet of saline and contrast tubing to inspect. To make inspection of the tubing easier, the communication channels can be configured in a “flat” ribbon-like design with the saline and contrast communication channels next to each other such that the contrast and saline tubing can be inspected at the same time, which effectively reduces the length of tubing to be inspected to 42 feet (14′ per mount). During the inspection, as the lines are each handled from end-to-end the medical practitioner typically will change gloves for each line (e.g., three glove changes). Such an inspection can take between 7 and 10 minutes, which is a long delay before proceeding with the operation when every minute of delay of performing a thrombectomy can result in further harm to the patient.

The first tubing set 1302 includes a one saline tube (channel) 1308 in fluid communication with a first saline flow-path in the cassette 1200 and a second saline flow-path in the splitter 1310, one contrast tube (channel) 1306 in fluid communication with a first contrast flow-path in the cassette 1200 and a contrast flow-path in the splitter 1310, and one vacuum tube (channel) 1305 in fluid communication with a first vacuum flow-path in the cassette 1200 and a second vacuum flow-path in the splitter 1310. An example first saline flow-path, first contrast flow-path, and first vacuum flow-path, in the cassette, are illustrated by the saline subsystem 1201, the contrast subsystem 1202, and the vacuum subsystem 1203 in FIG. 29E. The splitter 1310 provides saline, contrast, and vacuum from the first tubing set 1302 to the second tubing set 1316, and includes channels forming a second saline flow-path, a second contrast flow-path, and a second vacuum flow-path, where the second saline flow-path, the second contrast flow-path, and the second vacuum flow-path each having a plurality of branches structured to provide outputs of saline, contrast, and vacuum to each mount 1400 from the single inputs of saline, contrast, and vacuum from the first tubing set 1302. The first tubing set 1302 can be configured in a ribbon-like design. In some embodiments, the length 1324 of the first tubing set can be between about 5 feet and about 17 feet in length based on the specific embodiment. In some examples, the ratio of the first length to the second length is greater than about 1:2 (e.g., about 1:3, 1:4, 1:5, 1:6, of 1:7) to minimize the amount of tubing that needs to be visually inspected. In a specific embodiment, the length of the first tubing set is about 12 feet in length. The tubes in the first tubing set have inner diameters to suitably provide saline, contrast and vacuum to the catheters. As a nonlimiting examples, the inner diameter of saline and contrast tubes (channels) in the first tubing set and the second tubing set can be about 0.071 inches. As a nonlimiting example, the inner diameter of vacuum tubes in the first tubing set and the second tubing set can be about 0.110 inches. The first tubing set 1302 can also include an electrical channel 1304. The electrical channel includes multiple electrical leads to communicate electrical signals between the mounts 1400 and the cassette 1200, and can be coupled to the electrical interface 1236. Sensors and electrical components in the cassette 1200 can also be coupled to the electrical interface 1236.

The second tubing set 1316 includes one or more tube groups 1318, each tube subgroup configured to provide a mount 1400 with saline, contrast, and vacuum. Each tube group 1318 can also provide each mount 1400 with one or more electrical leads. In the example in FIG. 29C, each tube group 1318a-c includes a proximal end 1320 coupled to the splitter 1310 and a distal end 1322 coupled to one of the one or more mounts 1400a-c. Each tube group 1318a-c includes a saline subchannel in communication with the second saline flow-path in the splitter 1310, a contrast subchannel in communication with the second contrast flow-path in the splitter 1310, and a vacuum subchannel in communication with the second vacuum flow-path in the splitter 1310. Each tube group 1318 can be configured in a ribbon-like design. In some embodiments, the length 1326 of the second tubing set can be between about 1 foot and about 4 feet. In a specific embodiment, the length 1326 of the second tubing set is about 2 feet. In this embodiment, the length of tubing in the first tubing set and the second tubing set needed to be inspected is significantly reduced from 42 feet to 18 feet, which correspondingly drastically reduces the inspection time to about 3-4.5 minutes, advantageously reduces set-up time such that the medical procedure (e.g., a thrombectomy) can begin much sooner, which increases the patient's likelihood of a more successful recovery. In addition, this significant reduction in the amount of tubing to inspect also minimizes glove changes by the medical practitioner inspecting the tubing set from three glove changes to one glove change. The second tubing set 1316 can also provide one or more electrical leads 1328 to another mount that does not require fluids, for example, a guide wire mount 1400d configured to axially move and rotate a guide wire 1405 coupled to the guide wire mount 1400d, the guide wire 1405 configured to be positioned partially or fully in the lumen of access catheter 1402c. In some embodiments, connectors may be used to couple the second tubing set 1306 to the cassette 1200 and/or the splitter 1310. However, use of connectors in fluidic systems can cause air bubbles to be formed and/or trapped in a portion of the connector. In this embodiment, advantageously connectors are not used, rather the second tubing set 1316 is coupled to the splitter 1310 and the mounts 1400 such that the second tubing set 1316 is not meant to be decoupled prior to or during a medical procedure (for example, permanently, semi-permanently).

FIG. 29D is a schematic of an example of the fluidics assembly 1100 illustrated in FIG. 29C illustrating some additional features. The fluidics assembly 1100 includes cassette 1200, splitter 1310, and one or more mounts 1400, first tubing set 1302 coupled to the cassette 1200 and splitter 1310, and second tubing set 1316 coupled between the splitter 1310 and the one or more mounts 1400, the first tubing set 1316 having a saline channel 1308, a contrast channel 1306, and a vacuum channel 1305 for providing saline, contrast, and vacuum to the splitter 1310. In this example, the fluidics assembly 1100 is shown having three mounts 1400a-c that receive saline, contrast, and vacuum from the cassette 1200. The first tubing set 1302 also includes an electrical channel 1304 having a plurality of electrical leads for connecting electrical components in the mounts 1400 to an electrical interface of the cassette 1200. The second tubing set 1316 includes tube groups 1318a-c providing saline, contrast, and vacuum to mounts 1400a-c. Each of the tube groups 1318ac includes a saline subchannel 1333, a contrast subchannel 1332, a vacuum subchannel 1335, and an electrical subchannel 1334.

The splitter 1310 is structured to provide fluid communication of saline from the saline channel 1308 to the multiple saline subchannels 1333 in the tube groups 1316, to provide fluid communication of contrast from the contrast channel 1306 to the multiple contrast subchannels 1336 in the tube groups 1316, to provide fluid communication of saline from the single saline channel 1308 to the multiple saline subchannels 1333 in the tube groups 1316, and to provide fluid communication of vacuum from the vacuum channel 1305 to the multiple vacuum subchannels 1335 in the tube groups 1316. Also, the splitter 1310 is structured to provide electrical connection between the electrical channel 1304 and the electrical subchannels 1334 in the tube groups 1316. As illustrated in FIG. 29D, the splitter 1310 includes a saline manifold 1333 coupled to the saline channel 1308 and the saline subchannels 1337 of each tube group 1318a-c in the second tubing set 1316, a contrast manifold 1332 coupled to the contrast channel 1308 and the contrast subchannels 1336 of each tube group 1318a-c in the second tubing set 1316, a vacuum manifold 1331 coupled to the vacuum channel 1305 and the vacuum subchannel of each tube group 1318a-c in the second tubing set 1316, and an electrical bus 1330 coupled to the electrical subchannels 1334 of each tube group 1318a-c in the second tubing set 1316. The saline manifold 1333, contrast manifold 1332, and vacuum manifold 1331 can be structured in various configurations that include tubes, splitters, manifolds, or any other structures to provide fluid communication from a single channel in the first tubing set 1302 to multiple subchannels in the second tubing set 1316. The electrical bus 1330 can be any electrical structure that provides electrical communication between the electrical subchannels of the tub groups 1318 in the second tubing set 1316 and the electrical channel 1304 in the first tubing set, including separate electrical wires (leads) or other electrical connections. For example, the electrical subchannels in the tube groups 1318 can include a plurality of electrical leads which are bundled together in the splitter 1310 and form the electrical channel 1304 in the first tubing set. As shown in FIG. 29C, the electrical bus 1330 can also electrically connect the electrical channel 1304 of the first tubing set 1302 to the electrical connection 1328 to guide wire mount 1400d. Other embodiments having more or fewer mounts and corresponding connections are also possible. In some embodiments, the splitter 1310 may be referred to as a relay cassette. In some embodiments, the splitter 1310 may be located outside of the sterile field or within the sterile field. In some embodiments, the splitter 1310, second tubing set 1316, and associated hub assemblies (e.g., mounts 1400a, 1400b, and 1440c and associated hubs) can be positioned within the sterile field.

The embodiment of communication channels 1300 illustrated in FIGS. 29C and 29D is designed to minimize the time needed for inspecting the fluid lines. In this example, the fluid communication channels 1300 include a first tubing set 1302 having a proximal end 1312 coupled to the cassette 1200 and a distal end 1314 coupled to a splitter 1310. The fluid communication channels also include a second tubing set 1316 having one or more tube groups 1318, each tube group 1318 having a proximal end 1320 coupled to the splitter 1310 and a distal end 1312 coupled to one of the mounts 1400a-c. However, use of connectors in fluidic systems can cause air bubbles to be formed and/or trapped in a portion of the connector. In some preferred embodiments, advantageously connectors (i.e., releasable connectors) are not used to couple the first tubing set 1302 to the cassette 1200 and the splitter 1310, and are not used to connect the second tubing set 1316 to the splitter 1310. Instead, the first tubing set 1302 is coupled to the cassette 1200 and the splitter 1310 in a persistent coupling (e.g., permanent connection, semi-permanent coupling using glue, clamped fittings, integrally molded portions, and/or other suitable couplings) that it is not meant to be decoupled prior to or during a medical procedure. In preferred embodiments, the first tubing set 1302 and the second tubing set 1316 comprises transparent tubing. The cassette 1200 can comprise fluid communication channels (including portions of components) configured to communicate saline, contrast, and vacuum in first saline, contrast, and vacuum flow-paths, respectively. For example, as described in reference to FIG. 29E. The splitter 1310 can comprise fluid communication channels configured to communicate saline, contrast, and vacuum in the second saline, contrast, and vacuum flow-paths, respectively. For example, as described in reference to FIG. 29D. The fluid communication channels in the cassette 1200 and the splitter 1310 can include one or more tubes, channels (e.g., molded channels), portions of components (e.g., a drip chamber, clot pod, at least a portion of peristaltic saline pump, at least a portion of a contrast pump, robotically controlled valves, etc.) and other suitable fluid communication structures. The proximal end of the first tubing set can be coupled to the cassette 1200 using a persistent coupling. In some embodiments, the proximal end of the first tubing set 1302 extends into the cassette 1200 and forms a portion of a channel in the cassette 1200. The distal end of the first tubing set can be coupled to the splitter 1310 using a persistent coupling. In some embodiments, the distal end of the first tubing set 1302 extend into the splitter 1310 and can form a portion of a channel in the splitter 1310. In some embodiments, a portion of, or all of, the fluid communication channels of the first tubing set 1302 are integrally formed with a portion of, or all of, and the fluid communication channels of the splitter 1310. In an example, tubes forming each of a saline channel 1308, a contrast channel 1306, and a vacuum channel 1305 of the first tubing set 1302 are integrally formed with tubes forming saline, contrast, and vacuum fluid communication channels of the splitter 1310. In some embodiments, a portion of, or all of, the fluid communication channels of the second tubing set 1316 are integrally formed with a portion of, or all of, and the fluid communication channels of the splitter 1310. In an example, a portion, or all of, tubes forming a saline subchannel, a contrast subchannel, and a vacuum subchannel in each of one or more tube groups 1318 are formed to be integral with structures (e.g., tubes) forming the fluid communication channels of the splitter (e.g., channel 1337, channel 1336, channel 1335, manifolds 1333, 1332, 1331). In some embodiments, all or part of the fluid communication channels of the first tubing set 1302, the splitter 1310, and the second tubing set 1316 are formed as in integral structure and are persistently connected. In some embodiments, all or part of the fluid communication channels of the first tubing set 1302, the splitter 1310, the second tubing set 1316 and at least some of the fluid communication structures in the mounts 1400 are formed as an integral structure such that they are persistently connected. In some embodiments, all of the fluid communication channels in the first tubing set 1302, the splitter 1310, the second tubing set 1316 comprise tubing. For example, tubing that is coupled to the cassette 1200, split into multiple tube groups in the splitter, and tubing that is coupled to the splitter and the mounts. In some embodiments, all of the fluid communication channels in the first tubing set 1302, the splitter 1310, the second tubing set 1316 and the mounts 1400 comprise tubing. Some embodiments can include non-persistent coupling of fluid communication channels, for example, it is determined that such connections do not heighten the likelihood trapping air bubbles. In certain embodiments, tubes that are integrally formed with one another may be referred to as “tube sections” that together form a tube.

FIG. 29E is a schematic illustrating another example of a cassette 1200 of the fluidics assembly having a housing 1209 supports portions of a saline subsystem 1201, a contrast subsystem 1202, and a vacuum subsystem 1203 enclosed in, or partially enclosed in, the housing 1209. FIG. 67 also illustrates components of an example of a saline subsystem 1201, a contrast subsystem 1202, and a vacuum subsystem 1203 coupled to the cassette 1200, according to some embodiments. Other examples of cassettes may have different components, for example, fewer or more components. The control valves in the saline subsystem 1201, contrast subsystem 1202, and vacuum subsystem 1203 are robotically controlled to be actuated based on a user input, sensor signals received by a controller, and/or executable instructions that one or more hardware processors of a controller are configured to execute to prime the saline, contrast, and vacuum subsystems 1201, 1202, 1203, to prime fluid communication channels coupled to the saline, contrast, and vacuum subsystems 1201, 1202, 1203, prime fluidic channels in hubs/mounts, and provide saline, contrast, and vacuum from the saline, contrast, and vacuum subsystems 1201, 1202, 1203 to the hubs/mounts and ultimately to catheters connected to the hubs/mounts.

One or more saline sources can be coupled to the cassette 1200 to be selectively placed in fluid communication with the saline subsystem 1201. Flow-paths 1206 communicate saline through the saline subsystem 1201. In this example, two saline sources 1106a and 1106b (e.g., saline bags) are coupled to saline input ports 1204a, 1204b for providing a flow of saline to the saline subsystem 1201. The cassette 1200 includes a saline flow-path 1206 that receives saline through input ports 1204 and provides saline to the first tubing set 1302 through saline output port 1230, and also provides saline to the contrast subsystem 1202. Control valve 1214 can be actuated to connect channel 1206c to channel 1206f which is coupled to the vacuum subsystem 1203 vacuum channel 1282a to evacuate saline and air from the saline subsystem 1201 (i.e., channel 1206f can be used as a waste line). Input ports 1204a, 1204b are in fluid communication with a saline first control valve 1220, which can be controlled to select the saline source 1106a, 1106b to receive saline from. In some embodiments the saline sources 1106a, 1106b are supported by a weight sensor 1210 that is connected to a controller (e.g., in pump station) and provides a signal associated with the weight of each saline source to the controller. The saline subsystem 1201 also includes a peristaltic pump 1213 having an inlet 1207 and an outlet 1208, and an air sensor 1205 positioned between the peristaltic pump 1213 and the first control valve 1220. In some embodiments, the air sensor 1205 is incorporated in the cassette 1200. In other embodiments, the air sensor 1205 is capital equipment (not disposable) and is not included in the cassette 1200, instead it can be included in the pump station and is positioned near channel 1206ab (e.g., near a piece of tubing 1206 ab that engages with/into the air sensor 1205. In this embodiment, the portion of the peristaltic pump 1213 in the cassette includes tubing that, when the cassette 1200 is coupled to the pump station, the tubing interfaces with a movable portion of a peristaltic pump on the pump station which is configured to move saline through the saline flow-path 1206 of the saline subsystem 1201. An air sensor 1205 is positioned to sense air in the saline flow-path (channel) 1206ab between the peristaltic pump 1213 and the first control valve 1220, and is configured to generate a signal indicative of air detected. In some embodiments, the air sensor 1205 is capital equipment and located (for example, on the pump station). In such embodiments, channel 1206ab can include a portion of tubing that engages into the air sensor. In some embodiments, the air sensor is located in the cassette 1200 and is connected to the electrical interface 1236 such that a signal generated by the air sensor 1205 can be provided to the pump station (or another controller).

The saline subsystem 1201 also includes a pressure sensor 1212, positioned downstream of the peristaltic pump 1213, and configured to generate a signal indicative of pressure in the saline flow-path. The saline subsystem further includes a saline second control valve 1214 that is configured to selectively, as controlled by a controller, provide saline to a channel 1222 which is connected to the vacuum subsystem 1203. In operation, a controller may align the second control valve 1214 to diverge the saline flow-path to the vacuum subsystem 1203 to prime the upstream portion of the saline flow-path 1206a-c, for example, when the saline sources are first connected to the saline flow-path or when the saline source is switched from the first saline source 1106a a second saline source 1106b. The saline subsystem 1201 also includes a saline third control valve 1218 that is configured to selectively, as controlled by a controller, provide saline to a channel 1256 which is connected to the contrast subsystem 1202 for priming the contrast subsystem 1202 with saline. In a normal operational configuration for providing saline to catheters, a controller aligns the first control valve 1220 to receive saline from one of the two connected saline sources 1106, aligns the second control valve 1214 to provide saline from peristaltic pump 1213 to the third control valve 1218, and the lines the third control valve 1218 to provide saline to the saline output port 1230 to flow into the first tubing set 1302.

Still referring to the example illustrated in FIG. 29E, the contrast subsystem 1202 includes a contrast flow-path 1244 that receives contrast from a contrast source 1108 through contrast inlet port 1242 along flow-path 1244ab, communicates contrast to the first tubing set 1302 via contrast outlet port 1264 along flow-path 1244bd, and communicates air and/or contrast from a contrast pump 810 to the vacuum subsystem 1203 along flow-path 1244c. In some embodiments, the contrast subsystem 1202 includes a control valve 1211 coupled to channels 1244abd that can be actuated to connect the contrast pump 810 to the contrast source 1108 (connecting channels 1244b and 1244a), or connecting the contrast pump 810 to channel 1244d and the contrast channel of the first tubing set 1302. An air sensor 1246 is positioned along the contrast flow-path between the contrast source 1108 and the contrast pump 810, and is configured to sense air in the contrast flow-path (channel) 1244a and generate a signal indicative of air being in the contrast flow-path. In some embodiments, the air sensor 1246 is capital equipment and located (for example, on the pump station). In such embodiments, channel 1244a can include a portion of tubing that engages into the air sensor 1246. In some embodiments, the air sensor 1246 is located in the cassette 1200 and is connected to the electrical interface 1236 such that a signal generated by the air sensor 1246 can be provided to the pump station (or another controller). In some embodiments, based on receiving a signal from the air sensor 1246 indicating presence of air in flow-path 1244a, the controller may pause contrast pump operation, indicate another contrast source is needed on a user display or with an audible alarm. When a new contrast source is attached to contrast inlet port 1242, in some embodiments the controller can initiate a priming sequence that includes actuating the contrast control valve 1247 to provide vacuum to the contrast pump 810 (connecting channel 1244c to port 1250) and actuating valve 1211 to connect channels 1244a and 1244b to expel air in the contrast pump 810 and channels 1244ab through channel 1244c. When changing contrast sources (bottles), channel 1244b and the contrast pump (syringe) 810 are already full of contrast, but 1244a has air in it. Instead of pulling air through the primed syringe, control valve 1247 can be actuated to block channel 1244c from being in fluid communication with port 1250 (if not already so aligned) and control valve 1211 can be actuated to connect channel 1244a to be in fluid communication with contrast pump outlet port 1254, and the contrast pump 810 can be actuated to push air in channel 1244a into the contrast source 1108.

The contrast pump 810 receives contrast into a chamber in the contrast pump 810 from the contrast source 1108 through contrast (outlet) port 1254 to fill the contrast pump 810. A contrast pump port 1250 connects the contrast pump 810 to channel 1244c, 1249 which is connected to the vacuum subsystem 1203, and may be used to remove air from the contrast pump 810. Contrast control valve 1247 is positioned between contrast pump port 1250 and the vacuum subsystem, and is selectively controlled by a controller to align the contrast control valve 1247 to open or to shut the air/contrast flow-path between the contrast pump 810 and the vacuum subsystem 1203. When filling the contrast pump 810 with contrast from contrast source 1108, in some embodiments, the contrast control valve 1247 can be opened to provide vacuum to the contrast pump 810 through port 1250 to evacuate air in the contrast pump 810 along flow-path 1244c, and help cause contrast from the contrast source to flow along flow-paths 1244a and 1244b into port 1254 to fill the pump 810. In an example, the contrast control valve 1247 is opened for a predetermined amount of time to prime the contrast pump 810. In other examples, vacuum is not used during the priming process. For example, once the contrast pump 810 is at least partially filled with contrast, the contrast pump can be actuated to expel any air in the pump through channel 1244c and/or through channel 1244b (e.g., to the contrast source). The contrast control valve 1247 is closed for operation of the contrast pump 810. FIG. 29E illustrates one example of a contrast subsystem 1202, but other configurations are also contemplated.

The vacuum subsystem 1203 of the cassette 1200 includes a vacuum port 1271 coupled to a vacuum canister 1270, which can be coupled to a vacuum filter 1269, and a vacuum source assembly (collectively “vacuum source”) 1267 which can include a vacuum pump, a vacuum regulator, and other components for providing a consistent vacuum controlled robotically. The vacuum source 1267 can be controlled by a controller to provide a desired amount of vacuum to a vacuum flow-path. As illustrated in FIG. 29E, in this example the vacuum canister 1270, and vacuum source 1267 are positioned outside of the cassette 1200. Vacuum flow-path 1282 extends from the vacuum inlet port 1271, through clot pod assembly 1272 to vacuum port 1289, which is coupled to the first tubing set 1302. A portion of the vacuum flow-path 1282 extends from the vacuum port 1271 to the clot pod assembly 1272. The contrast subsystem 1202 and the saline subsystem 1201 are connected to the vacuum flow-path 1282 through channels 1249 and 1222, respectively, connecting between the clot pod assembly 1272 and vacuum port 1271 such that fluid and air from the saline subsystem 1201 and the contrast subsystem 1202 do not go through the clot pod assembly 1272, instead flowing through vacuum port 1271 and into vacuum canister 1270. The clot pod assembly 1272 includes clot pod 1273, drip chamber 1278, and a first three-way control valve 1275 positioned in the vacuum flow-path 1282 between the drip pod 1278 and the clot pod 1273. The first control valve 1275 is controlled by a controller to align the vacuum flow-path 1282 to connect clot pod inlet line 1274 to either the drip pod outlet line 1276 or the drip pod bypass line 1277. The first control valve 1275 can also be controlled by a controller to align the vacuum flow-path 1282 to neither the drip pod outlet line 1276 nor the drip pod bypass line 1277. Drip pod inlet line 1279 connects the vacuum flow-path to second three-way control valve 1280, which is controlled by a controller to align the vacuum flow-path 1282 to vacuum port 1289 and the first tubing set 1302, or to the local atmosphere. A pressure sensor 1283 is positioned along the flow-path 1282 between control valve 1280 and vacuum port 1289. Pressure sensor 1283 also connected to the electrical interface 1236, and is configured to detect pressure in the vacuum flow-path 1282 and provide a signal indicative of the detected pressure to the electrical interface 1236.

At least a portion of the clot pod assembly 1272 can be transparent to allow a medical practitioner to view the drip chamber 1278 and the clot pod 1273. For example, to see a fluid flow into the drip chamber and to determine a level of fluid accumulated in the drip chamber, and to view and assess material captured in the clot pod. In an example, the clot pod assembly 1272 can be structured to be positioned along a surface of the cassette 1200. In some embodiments, one side of the clot pod assembly 1272 can be transparent to such that the drip chamber and the clot pod are visible to a medical practitioner when the cassette is mounted on the pump station, and the opposite side is transparent or translucent to allow light illuminate the drip chamber and/or clot pod for easier viewing. An imaging sensor (e.g., a camera) 1292 can be positioned to generate an image of the clot pod 1273 and/or the drip pod 1278, and the image can be displayed locally or remotely to provide information on the contents of the clot pod 1273 and/or the drip pod 1273. In this embodiment, the imaging sensor 1292 is not included in the cassette 1200, but instead the imaging sensor 1292 can be incorporated in the pump station or another suitable location (locally) such that it can generate an image of the clot pod 1273 and/or drip pod 1278. In some embodiments, the imaging sensor 1292 can be incorporated in the cassette, however, having the imaging sensor positioned in the disposable cassette may increase cost. In embodiments where the imaging sensor is disposed external to the cassette 1200, a portion of the cassette 1200 can be transparent to allow the imaging sensor 1292 to image the clot pod assembly 1272, or a portion thereof.

Saline subsystem 1201 can include air sensor 1205 and pressure sensor 1212, the contrast subsystem 1202 includes air sensor 1246, and the vacuum subsystem 1203 includes pressure sensor 1283. Signals from these sensors are received by a controller, which can use these signals to align control valves in the cassette 1200 to vary the saline, contrast, and vacuum flow-paths in the cassette and perform processes described herein, and other processes. A purpose of these sensors is to detect potentially hazardous situations, for example, running out of a fluid source, a kink or occlusion in a fluid line, etc. Although the saline and contrast air sensors 1205, 1246 can be located in a pump station and engage a portion of saline or contrast tubing in the cassette, or they can be located in the cassette, such air sensors can be considered and referred to as being a part of the saline subsystem 1201 or the contrast subsystem 1202.

The configuration of the cassette 1200 in FIG. 29E has a certain different features than cassettes described above, which can provide operational and cost advantages. For example, in embodiments in FIGS. 14 and 22, the vacuum canister is positioned in the cassette, where in FIG. 29E the vacuum canister 1270 (and vacuum regulator 1269 and the vacuum pump 1267) are positioned outside of the cassette in the line from the vacuum canister 1270 is coupled to the cassette at port 1271. This may allow the disposable cassette to be less expensive, and allow various configurations of vacuum source assemblies (e.g., the vacuum canister, the vacuum regulator, and the vacuum pump) to be used with the system, and any changes or improvements to the vacuum canister will not affect the design of the cassette. The configuration of the cassette 1200 in FIG. 29E includes two saline sources 1106a, 1106b in a control valve 1220 that a controller can align to select either one of the saline sources, either based on a manual input or automatically based on the sensed condition (e.g., the signal from weight sensor 1210 that a saline source is depleted below a predetermined threshold, a signal from air sensor 1205 that air is present in the saline flow-path 1206). The saline subsystem 1201 in FIG. 67 includes channel 1222 from the saline subsystem to the vacuum subsystem 1203 which allows for a simplified priming procedure compared to configurations illustrated in FIGS. 14 and 22. In some embodiments, if air is detected, the controller can be configured to automatically, or based on user input, to prime the saline subsystem by directing the saline flow-path 1206 upstream of the second control valve 1214 to vacuum such that saline and the air in the saline subsystem 1201 upstream of the second control valve 1214 is evacuated into the vacuum canister 1270. Also, the contrast subsystem 1202 has advantageously been simplified and no longer includes a contrast manifold and the three control valves can be aligned to select the catheter to receive contrast. Instead, control valves that the controller can align to provide contrast from the contrast subsystem 1202 to a catheter are located in the mounts as shown in an example, in FIG. 29F (e.g., control valve 1426). Similarly, control valves that the controller can align to provide saline from the saline subsystem 12012 a catheter located in the mounts (e.g., FIG. 29F). Having these control valves in the mounts also advantageously obviates the need for having multiple individually controllable saline lines and contrast lines extending from the cassette 1200 to the mounts 1400, such that the fluid communication channels 1300 (FIG. 29D) are also simplified and include less tubing. Also, unlike some of the previous embodiments described above, the example configuration illustrated in FIG. 29E advantageously does not include saline lines or control valves for providing saline to a femoral sheath, thus further simplifying the design of the cassette 1200 and may lower manufacturing costs. Instead, in this example, saline can be provided to a femoral sheath from a separate system.

In reference to FIG. 29B-29E, examples of a fluidics management system can comprise a cassette configured to receive saline from a saline source (e.g., two or more saline sources), receive contrast from a contrast source, and receive vacuum from a vacuum source. The cassette can include saline subsystem having a first saline flow-path; a contrast subsystem having a first contrast flow-path; and a vacuum subsystem including a first vacuum flow-path. The first saline, contrast, and vacuum flow-paths can include channels, tubes, line, and portions of other components (e.g., one or more of a valve, drip chamber, clot pod, pump, etc. in the cassette). The fluidics management system can also include a splitter having a second saline flow-path, a second contrast flow-path, and a second vacuum flow-path. Each of the second saline, contrast, and vacuum flow-paths has single proximal end and a plurality of distal ends. The single proximal end of the second saline flow-path is in fluid communication with a single saline channel in a first tubing set, the proximal end of the second contrast flow-path is in fluid communication with a single channel of the first tubing set. The second vacuum flow-path proximal end is in fluid communication with the single vacuum channel of the first tubing set. The first tubing set has a first length, as measured between the cassette and the splitter, and is coupled to the cassette and splitter. The first tubing set includes a single saline channel coupled to the first saline flow-path and the proximal end of the second saline flow-path, the single contrast channel coupled to the first contrast flow-path and the proximal end of the second contrast flow-path, and a single vacuum channel coupled to the first vacuum flow-path and the proximal end of the second vacuum flow-path. The fluidics management system can also include two or more mounts, for example, three mounts, or four mounts. The mounts can be configured in various ways to provide saline, contrast, and vacuum to catheters coupled to each respective mount. Each mount can be configured to have a third saline flow-path, a third contrast flow-path, and a third vacuum flow-path to provide saline, contrast and vacuum to a catheter via a connector configured to couple to a catheter. In some embodiments, the catheter is coupled to another assembly (e.g., a hub) which is coupled to the mount, and the lumen of the catheter is coupled to the connector to be in fluid communication with the connector. A mount can include a plurality of robotically actuated control valves controlled by a control system to selectively align the third saline flow-path, the third contrast flow-path, and the third vacuum flow-path to be in fluid communication with the connector. The fluidics management system can also include a second tubing set having a second length, the second tubing set comprising a plurality of tube groups, each tube group coupled to the splitter on a proximal end of the tube group and to one of the two or more mounts on a distal end of the tube group. Each tube group including a saline subchannel coupled to the distal end of the second saline flow-path of the splitter, a contrast subchannel coupled to the distal end of the second contrast flow-path of the splitter, and a vacuum subchannel coupled to the distal end of the second vacuum flow-path of the splitter. The first tubing set has a first length and the second tubing set has a second length, the first length being greater than the second length to minimize the amount of tubing that needs to be visually inspected for air bubbles. In some examples, first tubing set has a first length and the second tubing set has a second length, and the first length of the first tubing set is at least two times as long as the second length of the second tubing set.

Priming of the saline subsystem 1201 can include robotically actuating control valves of the saline subsystem 1201, robotically actuating a peristaltic saline pump, and robotically controlling vacuum in the vacuum subsystem to perform a priming process. In one example of priming of the saline subsystem 1201, the cassette 1200 is coupled to a pump station and the saline sources 1106 are coupled to the saline subsystem. To be able to switch from one saline source to another connected saline source seamlessly, channels connecting both saline sources to the saline subsystem 1201 must be primed. Control valve 1220 can be robotically controlled to couple a first saline source to peristaltic pump inlet 1207. The control valve 1214 can be aligned to connect the peristaltic pump outlet 1208 to channel 1222 which is connected to the vacuum subsystem 1203 to purge air from the saline subsystem. The vacuum subsystem can be robotically controlled to provide vacuum to channel 1222 to purge air during priming, or to not provide vacuum and instead channel 1222 and cannister 1270 is used to purge air without applying vacuum using channel 1222. In this configuration, the saline peristaltic pump can be run (e.g., for a predetermined time) to evacuate air in the saline flow-path from the first saline source to control valve 1214. During this priming, signals from air sensor 1205 should eventually indicate no air is detected. The control valve can be actuated to connect a second saline source to peristaltic pump inlet 1207. The control valve 1214 can continue to be aligned to connect the peristaltic pump outlet 1208 to channel 1222 which is connected to the vacuum subsystem 1203. The vacuum subsystem can again be robotically controlled to provide vacuum to channel 1222. In this configuration, the saline peristaltic pump can be run (e.g., for a predetermined time) to evacuate air in the saline flow-path from the second saline source to control valve 1214. During this priming, signals from air sensor 1205 should eventually indicate no air is detected. Control valve 1214 can be actuated to connect the outlet port 1208 to the second control valve 1218, which can be actuated to connect the outlet port 1208 to the contrast subsystem 1202 for priming the contrast subsystem, or connect outlet port 1208 to a saline communication channel to provide saline to a plurality of mounts 1400.

Priming of the contrast subsystem 1202 can include robotically actuating control valve 1247, robotically actuating contrast pump 810, and robotically controlling the vacuum subsystem 1203 to perform a priming process. In an example of a priming process of the contrast subsystem 1202, the contrast subsystem 1202 is connected to a contrast source 1108. Control valve 1247 is robotically actuated to connect outlet port 1250 to the vacuum subsystem 1203. In some embodiments of a contrast priming process, the vacuum pump 1267 is robotically actuated to provide vacuum to the contrast pump 810 and the contrast flow-path 1244, which evacuates air from the contrast flow-path (channel) 1244 and the contrast pump 810 as it fills with contrast. In other embodiments of a contrast priming process, vacuum is not provided to the contrast pump 810 via channel 1244, instead channel 1244 is used to evacuate air which evacuate air from channels 1244 and the contrast pump 810 as the pump is filled with contrast. Subsequently, control valve 1247 is closed and the contrast pump 810 can be robotically actuated to provide contrast to a prime a contrast communication channel, to prime the plurality of mounts, and to provide contrast to the plurality of mounts 1400. As part of priming the contrast communication channels connect the cassette to the mounts, control valve 1218 can robotically actuated to connect outlet port 1208 to the contrast flow-path 1244, and the saline peristaltic pump can be actuated to provide saline to the contrast subsystem such that it fills the contrast communication channels to the mounts and in the mounts. The vacuum subsystem 1203 can be robotically controlled to provide vacuum to the mounts 1400 via a vacuum communication channel. In each mount, one or more control valves can be robotically actuated to place a contrast channel in the mount, that is being provided saline from the cassette, in fluid communication with a vacuum channel to facilitate priming the contrast communication channel between the cassette and the mount, and in the mount, with saline to evacuate all air in these channels. Subsequently, control valve 1218 can be actuated to connect the outlet port 1208 with saline outlet port 1230 and the saline communication channel that is coupled to the mounts.

As indicated above for other embodiments of a fluidics system, saline and contrast channels in the fluidics assembly 1100 and in hub assemblies (e.g., mounts and/or hubs) are also primed prior to use in a medical procedure. In an example, once the saline subsystem 1201 is primed, the control valve 1218 be actuated to connect the outlet of the saline pump to the saline channels in the fluidics assembly 1100 and the saline pump 1213 can be actuated to provide saline to fill the saline 1308, the saline manifold 1333, the saline subchannels 1337 of each tube group in the second tubing set 1316, and the saline channel 1412 of each mount 1400 with saline. To facilitate the flow of saline through these saline channels, the control valves 1426 and 1428 can be actuated to connect the saline channel 1412 with the vacuum channel 1416 while controlling the vacuum subsystem 1203 to provide vacuum to the mounts 1400. Once the saline subsystem 1201 is primed, the control valve 1218 be actuated to connect the outlet of the saline pump 1213 to the contrast subsystem 1202, and the saline pump 1213 can be actuated to provide saline into the contrast subsystem 1202, to fill the contrast channel 1306, the contrast manifold 1333, the contrast subchannels 1336 of the second tubing set, and the contrast channel 1414 of each mount 1400 with saline. To facilitate the flow of saline through the contrast channels, the control valves 1426 and 1428 can be actuated to connect the contrast channel 1414 with the vacuum channel 1416 while controlling the vacuum subsystem to provide vacuum to the mount. A controller can use signals received from the saline and contrast air sensors 1418, 1420 to determine when no air is detected in the saline channel 1412 and the contrast channel 1414, indicating the saline and contrast channels between the mounts and the cassette are primed with saline. Similar to the priming processes described above, once the contrast channels from the cassette to the mounts are primed with saline, and the contrast channel 1414 is primed with saline.

FIG. 29F is a schematic illustrating an example of a fluidic system components in one or more mounts 1400 coupled to the second tubing set 1316. This example includes three mounts 1400a-c that have the same components relating to the fluidic system. Each mount 1400 has fluidic connections to the second tubing set 1316 to receive saline, contrast, and vacuum, and an electrical connection that includes one or more electrical leads, which are coupled to one or more sensors in the mounts 1400 to communicate signals from one or more sensors in the mount to the cassette, and ultimately to a controller (e.g., controller 2220) when the cassette is coupled to the pump station. The controller uses the received signals to align valves in the mounts to provide saline, contrast, and aspiration automatically or semi-automatically (e.g., based on a user input) as needed during different portions of a medical procedure. In this example, mount 1400a has a common connector 1434a for providing saline, contrast, and vacuum to the lumen of catheter 1402a, mount 1400b has a connector 1434b for providing saline, contrast, and vacuum to the lumen of catheter 1402b, and mount 1400c has a connector 1434c for providing saline, contrast, and vacuum to the lumen of catheter 1402c. The connectors 1434 can be in fluid communication with the lumens of catheters 1402 by coupling the connectors to a corresponding connector on a hub (or other assembly) that the catheters are coupled to, where the hubs can include a fluid communication channel from the connector 1434 to the lumen of the catheter coupled to the hub. Such hubs can be removably coupled to the mounts 1400. The hub can include structure that operates as a hemostasis valve, such that the hub can provide fluid to the lumen of a first catheter coupled to the hub that for discharging at the distal end of the first catheter, or can provide vacuum to the lumen of a first catheter coupled to the hub for providing aspiration at the distal end of the first catheter, while another elongated device is positioned partially or fully in the lumen of the first catheter in a concentric nested configuration. For example, when catheter 1402b is positioned in the lumen of catheter 1402a, when catheter 1402c is positioned in the lumen of catheter 1402b, or when an elongated device (e.g., a catheter, a guidewire, etc.) is positioned in the lumen of catheter 1402c. In some embodiments, catheter 1402a is a guide catheter, catheter 1402 is a procedure catheter, and catheter 1402c is an access (or insert) catheter.

Still referring to FIG. 69, a mount 1400 includes a saline port 1406, a contrast port 1408, a vacuum port 1410, and an electrical connection 1411 that are coupled to saline subchannel 1337, contrast subchannel 1336, vacuum subchannel 1335, and electrical channel 1334, respectively, of a tube group 1318 (of second tubing set 1316) coupled to the mount 1400. Preferably, the mount 1400 is coupled to the tube group 1318 without using connectors, for example, in a persistent connection that is not meant to be decoupled before or during its use in a medical procedure. An air sensor 1418 is positioned along the saline channel (flow-path) 1412 and is in electrical communication with electrical connection 1411. The air sensor 1418 is configured to detect air in the saline channel 1412 and generate a signal indicative of detecting air which is provided to the cassette 1200 and ultimately a controller, via the second tubing set 1316, splitter 1310, first tubing set 1302, and the cassette 1200. An air sensor 1420 is positioned along contrast channel (flow-path) 1414 and is in electrical communication with electrical connection 1411. The air sensor 1420 is configured to detect air in the contrast channel 1414 and generate a signal indicative of detecting air which is provided to cassette 1200 and ultimately a controller via the second tubing set 1316, splitter 1310, first tubing set 1302, and the cassette 1200.

Mount 1400 includes a first control valve 1426 that is controllable by a controller to align saline channel 1412, or contrast channel 1414, to a second control valve 1428. Check valve 1422 in the saline channel 1412 prevents any upstream fluid flow on the saline channel 1412. Along the saline channel 1412 between the first control valve 1426 and the check valve 1422 is a saline drip channel (saline restricted-flow channel) 1424 which connects the saline channel 1412 upstream of the first control valve 1426 a fluid flow-path to the second control valve 1428, bypassing the first control valve 1426. In this configuration, at least some saline that enters the mount 1400 can flow to via the saline restricted-flow channel 1424 to the second control valve 1428 regardless of the position of the first control valve 1426. The saline drip flow-path 1423 includes a saline restricted-flow channel 1424 designed to allow a desired flow-rate of saline (e.g, mL/minute) to flow to the connector 1434 and to the lumen of a catheter in fluid communication with the connector 1434 when the second control valve 1428 is aligned to provide saline or contrast to the connector 1434, regardless of the alignment of the first control valve 1426. The saline restricted-flow channel 1424 is configured to provide a lower flow-rate of saline than the saline channel 1412 provides to the saline-contrast channel 1421 through the first control valve 1426. The saline restricted-flow channel 1424 can have, for example, a smaller cross-sectional dimension then the cross-sectional dimension of the flow-path from the saline channel 1412 to the saline-contrast channel 1421 through the first control valve 1426. As an example, the saline restricted-flow channel 1424 can have narrow portion of the saline drip flow-path designed such that the saline restricted-flow channel 1424 provides about 1 mL/minute of saline to the lumen of an associated catheter. For example, in the range of about 0.85 mL/minute to about 1.35 mL/minute. In systems with multiple catheters, the catheters can be positioned in a concentric configuration, that is, such that catheter 1402c can be positioned at least partially in the lumen of catheter 1402b, and catheter 1402b can be positioned at least partially in the lumen of catheter 1402a. In the example illustrated in FIG. 69, flow restrictor FR1 is associated with catheter 1402a, flow restrictor FR2 is associated with catheter 1402b, and flow restrictor FR3 is associated with catheter 1402c. As an example, the saline restricted-flow channel 1424a can have a flow restrictor (value, size) FR1 designed such that the saline restricted-flow channel 1424 provides about 1 mL/minute of saline to the lumen of a catheter having a lumen inner diameter (ID) of about 0.088 inches (for example, a Zoom 88 catheter). As an example, the saline restricted-flow channel 1424b can have a flow restrictor FR2 designed such that the saline restricted-flow channel 1424b provides about 1 mL/minute of saline to the lumen of a catheter having an ID of about 0.071 inches (for example, a Zoom 71 catheter). As an example, the saline restricted-flow channel 1424c can have a flow restrictor FR3 designed such that the saline restricted-flow channel 1424c provides about 1 ml of saline to the lumen of a catheter having a lumen inner diameter (ID) of about 0.035 inches (for example, a Zoom 35 catheter). The flow restrictor FR1, FR2, FR3 can be all about the same size (e.g., having the same or about the same ID), or can be sized differently based on the catheter size a flow restrictor is associated with such that a saline drip flow of about 1 mL/minute is achieved to the catheter in fluid communication with the connector 1434.

The second control valve 1428 is a three-way valve that connects either vacuum to or saline/contrast to connector 1434 via a fluid primary channel 1432. The primary channel provides saline, contrast and vacuum to a catheter coupled to the mount 1400, for example, provides saline, contrast and vacuum to the lumen of the catheter through connector 1434. The primary channel also receives material (e.g., fluids, clots, etc.) from a catheter coupled to the mount 1400 when vacuum is provided to the catheter. Pressure sensor 929 (e.g., a hemodynamic pressure sensor) is positioned to detect pressure in the primary channel 1432 and is in electrical communication with electrical connection 1411. Pressure sensor 929 is configured to generate a signal, indicative of the detected pressure, which is provided to cassette 1200 and ultimately a controller via the second tubing set 1316, splitter 1310, first tubing set 1302, and the cassette 1200. The configuration of mounts 1400 includes several advantages over previous configurations described above. For example, in this configuration a mount 1400 includes an air sensor 1418 in the saline channel 1412 and includes air sensor 1420 and the contrast channel 1414, instead of a bubble filter 922 (FIG. 18) in a combined saline/contrast fluid channel 940 providing extra safety detecting air in any fluid provided to catheter 1402. If the controller receives a signal from the saline air sensor 1418 or the contrast air sensor 1420, it can provide an alarm and/or align the second control valve 1428 to evacuate fluid in saline-contrast channel 1421 and upstream of saline-contrast channel 1421 via vacuum channel (flow-path) 1416. Subsequently, the saline and/or the contrast channels can be re-primed. In some embodiments, the controller may also align the first control valve 1426 to prevent contrast and saline to flow through the first control valve 1246.

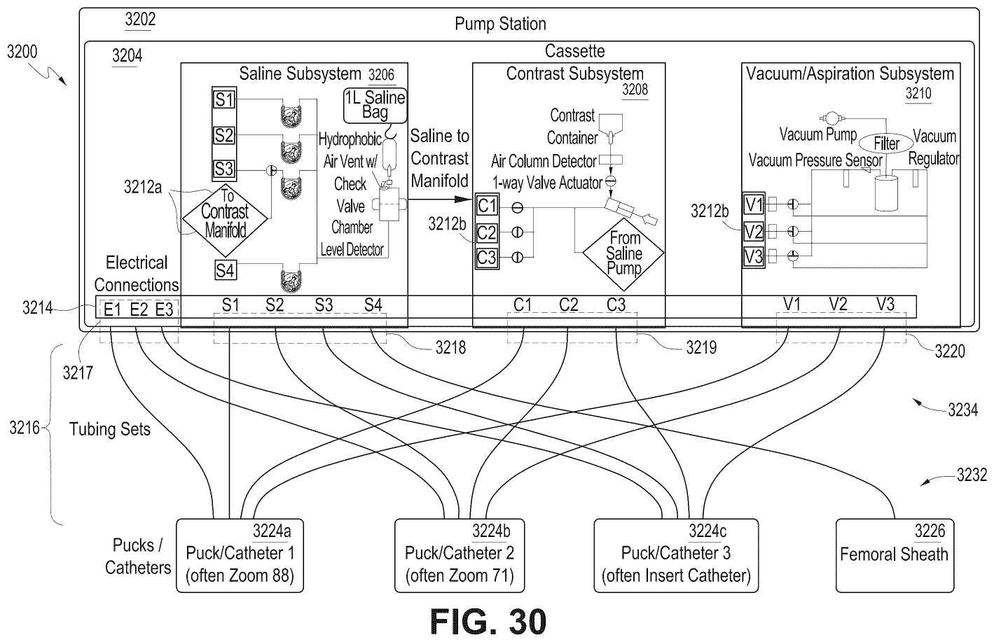

FIG. 30 illustrates certain aspects of an example of a portion of a robotic catheter system that includes an embodiment of a system 3200 for managing fluidics. As described further below, in some embodiments system 3200 can include a pump station 3202 and a cassette 3204. The cassette 3204 can include components and fluid communication channels of a saline subsystem 3206, a contrast subsystem 3208, and a vacuum subsystem 3210. For example, cassette 3204 can include connections/ports to connect to a tubing set and sources of saline, contrast, and vacuum, one or more peristaltic pumps, one or more valves, one or more sensors, one or more contrast pumps, one or more vacuum cannisters, one or more clot pods, and fluid communication channels for communicating saline, contrast, and vacuum from the saline, contrast, and vacuum sources to the hubs and catheters. The pump station 3202 can include components that interact with the subsystems in the cassette when the cassette 3204 is coupled to the pump station 3202. In an example, the pump station 3202 can include one or more valve actuators, one or more peristaltic pump drive mechanisms, one or more contrast pump drive mechanisms, configured to actuate and drive valves and pumps in the cassette that are part of the saline, contrast, and vacuum subsystems. The pump station 3202 can also include one or more sensors that are configured to sense fluid flowing in certain fluid communication channels of the cassette 3204.

In some embodiments, the cassette 3204 and the pump station 3202 include corresponding electrical contacts or connections (both referred to as “contacts”) that are connected when the cassette 3204 is coupled to the pump station 3202 to connect electrical components (e.g., sensors) in the cassette 3204 to the pump station 3202. The electrical contacts may include contacts for providing information from a component in the cassette (or the hub coupled to the cassette) to a controller in the pump station or a controller in communication with the pump station that is configured to operate the fluidics system, or another system that utilizes such information. The electrical contacts may include contacts for providing power to a component in the cassette. In some embodiments, the electrical contacts can also electrically connect the pump station 3202 to one or more hubs 3224 via the cassette 3204. Accordingly, the electrical contacts can provide power to a component in a one or more hubs via the cassette (and via electrical connections between the cassette and the one or more hubs). Certain components of the system 3200 can be configured to be disposable and certain components can be configured to be reusable. For example, the one or more hubs 3224 and catheters coupled to the hubs, a tubing set 3216, and/or cassette 3204 can be configured to be disposable. Valves related to controlling providing fluids and providing vacuum in system 3200 can be referred to collectively as a “valve assembly” for ease of reference. The valve assembly can include, but is not limited to, valves in a saline subsystem 3206, contrast subsystem 3208, and vacuum subsystem 3210 that are located in a pump station 3202, cassette 3204, and/or the one or more hubs 3224. As described in examples below, the system 3200 provides saline, contrast, and vacuum, from saline, contrast, and vacuum sources (respectively) through fluid communication channels to the one or more hubs 3224 and catheters coupled to the hubs 3224. The fluid communication channels can include channels, tubes, ports, lines connectors, and other structure to communicate fluid and provide vacuum. Unless otherwise indicated “channels,” “tubes,” and “lines” may be used synonymously herein as referring to a fluid communication channel. For example, the fluid communication channels can include channels in the cassette 3204, one or more tubes that are part of tubing set 3216, and tubes and/or channels located in a hub 3224, and the fluid communication channels can be collectively referred to as a fluid communication system.

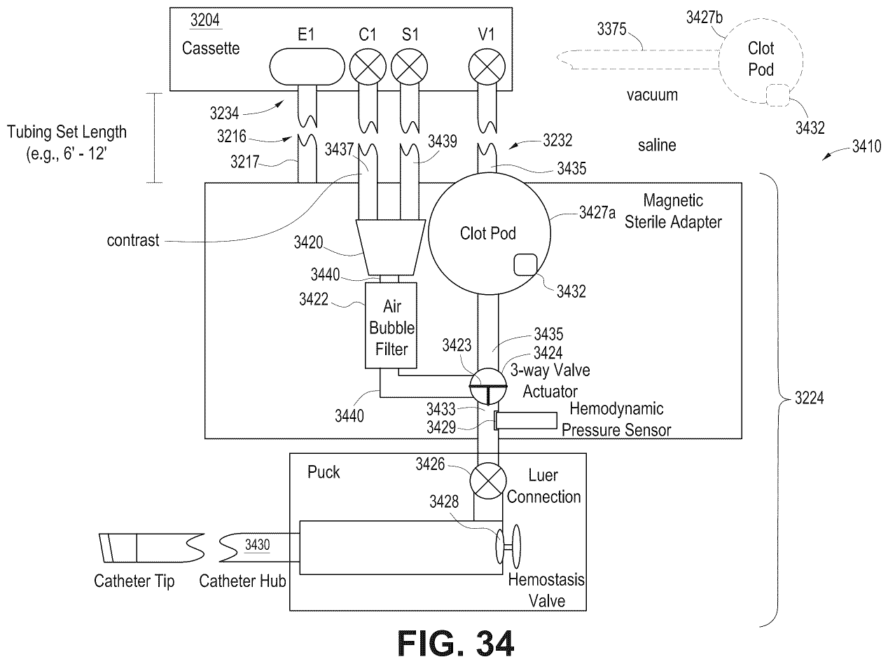

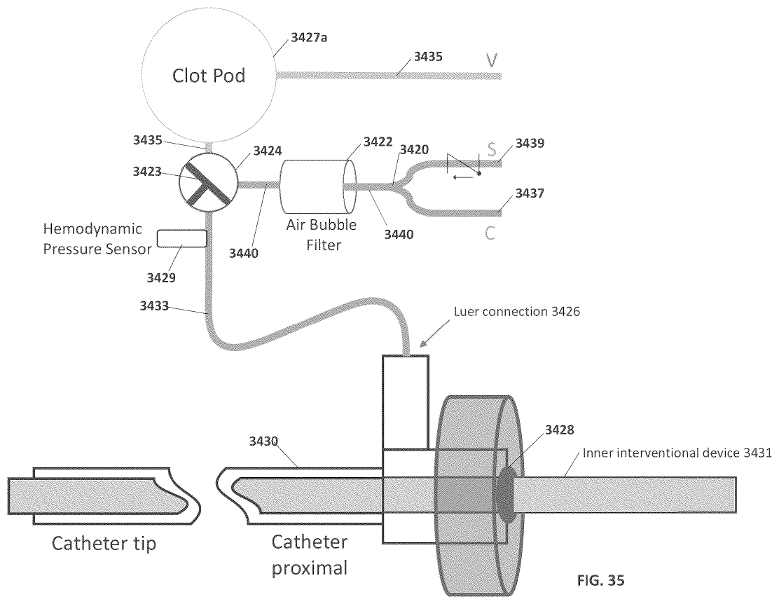

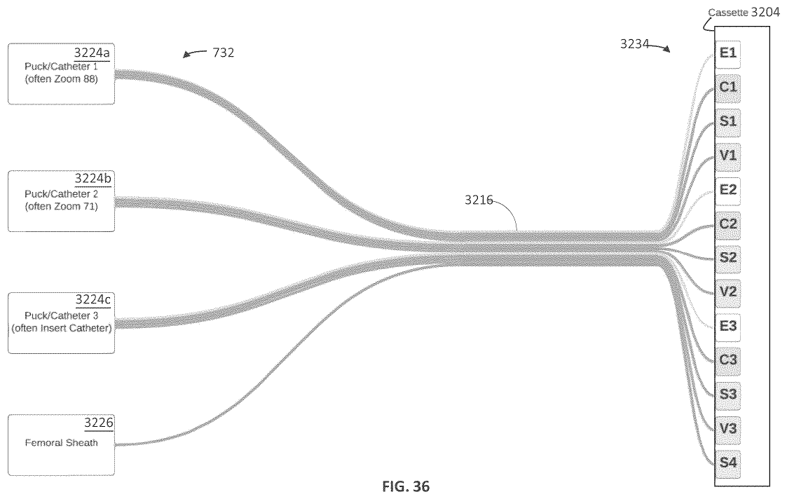

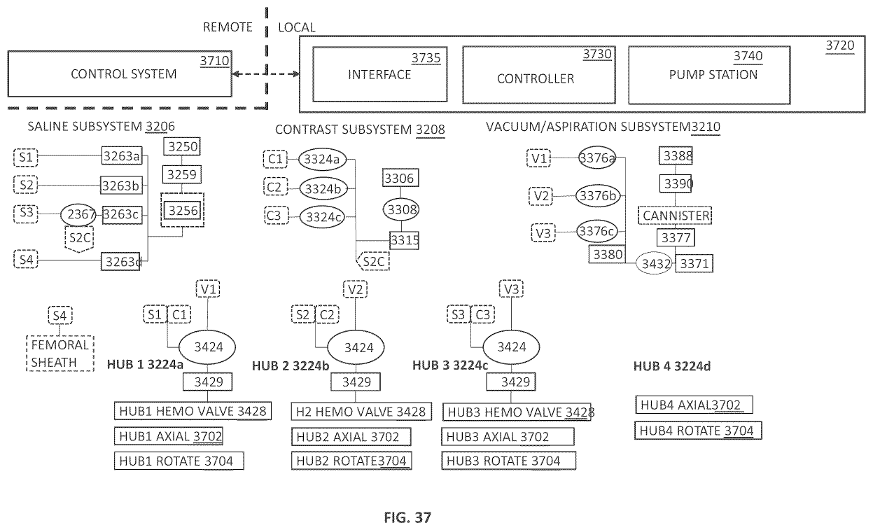

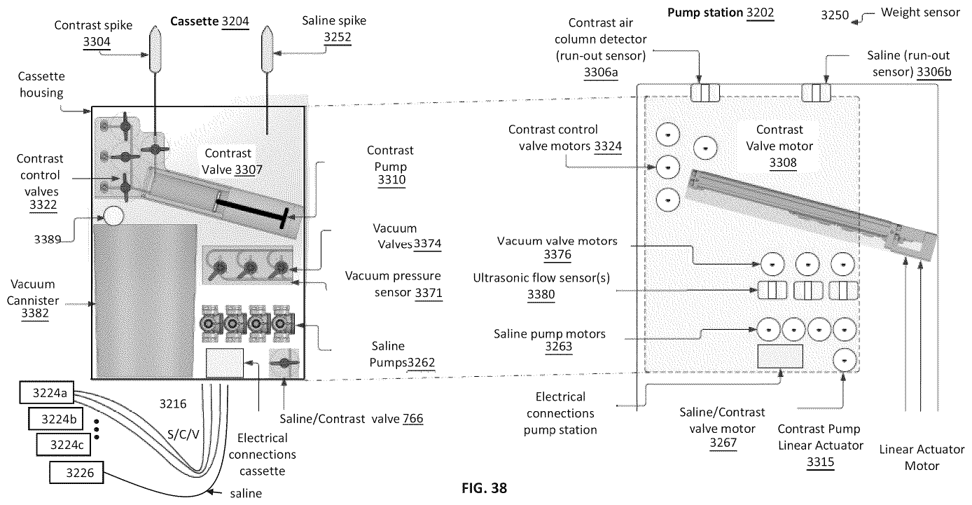

Embodiments of a saline subsystem 3206, a contrast subsystem 3208, and a vacuum subsystem 3210 that can be used to perform methods for controlling fluid administration equipment are illustrated in FIGS. 31, 32, and 33, respectively. FIGS. 34 and 35 illustrate examples of configurations portions of a fluidic system relating to a hub. FIG. 36 illustrates an example of a tubing set 3216 coupled to a cassette 3204 on one end, and one or more hubs 3224 and a femoral sheath 3226 on the other end. FIG. 37 illustrates certain components of a robotic catheter system that are part of, or related to, a fluidics system, which are actuatable by a controller or provide information to a controller, and the controller perform methods for controlling fluid administration equipment by actuating such components and based on (at least in part) such information. The fluidic subsystems and components may be controlled for performing fluidic methods on the robotic catheter system including, for example, priming the saline subsystem, priming the contrast subsystem, injecting contrast, aspirating a clot, and back-bleeding a hemostasis valve. Such processes are controlled at least in part by a controller, for example, system controller 3730. Valves, pumps and other actuatable (or movable) components can be driven to be in a certain state or position by a controller. In some embodiments, the state or position of actuatable components can be determined by sensing mechanism on the component such that the controller can determine the current position of the component before, during, or after a process. In some examples, the sensing mechanism can include a switch, encoder, and the like. In some embodiments, the current state or position of the components can be electronically stored information (e.g., in a table or file) and the controller is configured to determine the position of one or more components by accessing the stored information. In an example, before contrast is injected using a selected catheter in a system having multiple catheters, a valve coupling the selected catheter to a contrast subsystem can be determined to be aligned in an open position to provide contrast to the selected catheter, and one or more valves coupling non-selected catheters to the contrast subsystem can be determined to be aligned in a closed position to de-couple the non-selected catheters from the contrast subsystem. Such determinations may be made using the stored information or by using information from a sensing mechanism. The processes described herein are examples of some of the processes a controller can be configured to perform by controlling fluidic equipment, either automatically or semi-automatically, based on one or more user inputs, sensed information, and/other information.

As indicated above, an example of a fluidics system 3200 configured to provide saline, contrast, and vacuum to the one or more hubs 3224 and provide saline to a femoral sheath 3226 is illustrated in FIG. 30. The one or more hubs 3224 may comprise a first hub 3224a, a second hub 3224b, and a third hub 3224c, collectively hereinafter “hubs 3224a-3224c”. As shown in FIG. 30, a catheter is coupled to each of the one or more hubs, and each of the hubs 3224a-3224c is configured to provide saline, contrast, and vacuum from the fluidic system 3200 to the lumen of the catheter coupled to the hub (for example, as shown in FIGS. 34 and 35). For simplicity of disclosure, unless otherwise specified indicated by context, providing saline, contrast, or vacuum to a hub also refers to providing saline, contrast, or vacuum to the catheter coupled to the hub. The example in FIG. 30 is a multi-channel fluidics system that is configured to provide separately controllable amounts of saline, contrast, and vacuum to each of the one or more hubs 3224a-3224c and femoral sheath 3226 as needed during a medical procedure. Being able to separately control the saline, contrast, and vacuum to each hub is advantageous because each catheter has a different sized lumen due to the catheter having a different inside diameter, and the amounts of saline, contrast, and/or vacuum needed during their use may be different based on the lumen size. Each catheter can also have a different effective lumen size which may change at certain times during the procedure based on its lumen containing another catheter or guidewire nested inside its lumen. Being able to separately control the saline, contrast, and vacuum to each hub can also be advantageous because during a medical procedure different amounts of saline, contrast, or vacuum at each catheter may be desired based on what a particular catheter is being used for. In other embodiments, such a fluidics system can be configured as a one-channel fluidics system that provides controlled amounts of saline, contrast, and vacuum to one hub, or a multi-channel fluidics system configured to provide controlled amounts of saline, contrast, and vacuum to two hubs, or four or more hubs.

In various embodiments, the fluidics system 3200 illustrated in FIG. 30 can have similar or the same features as other fluidic systems described herein. In this example, fluidics system 3200 includes a pump station 3202 and a cassette 3204 which is releasably couplable to the pump station. The pump station 3202 can include components that may be capital equipment. The cassette 3204 can include components that may be designed for a one-time use and the cassette is disposable. In an example of use, the cassette 3204 is a sterilized disposable component that is coupled to the pump station 3202 prior to performing a procedure, and the cassette 3204 is removed and disposed of after the procedure is completed. The fluidic system 3200 can include numerous valves that control saline, contrast, or vacuum, located in a cassette, pump station, or hub (as illustrated in, for example, FIGS. 30-35, 37) and can be referred to collectively as a valve assembly. In various embodiments, portions of the valve assembly can be located in the pump station 3202 or in the cassette 3204. Various embodiments of the valve assembly can include additional valves, fewer valves, or different valves, than illustrated in the figures. The fluidic system 3200 can also include sensors in the saline, contrast, and vacuum subsystem, and these senses can be collectively referred to as a sensor assembly. In various embodiments, portions of the sensor assembly can be located in the pump station 3202 or in the cassette 3204. Various embodiments of the sensor assembly can include additional sensors, fewer sensors, or different sensors, than illustrated in the figures.

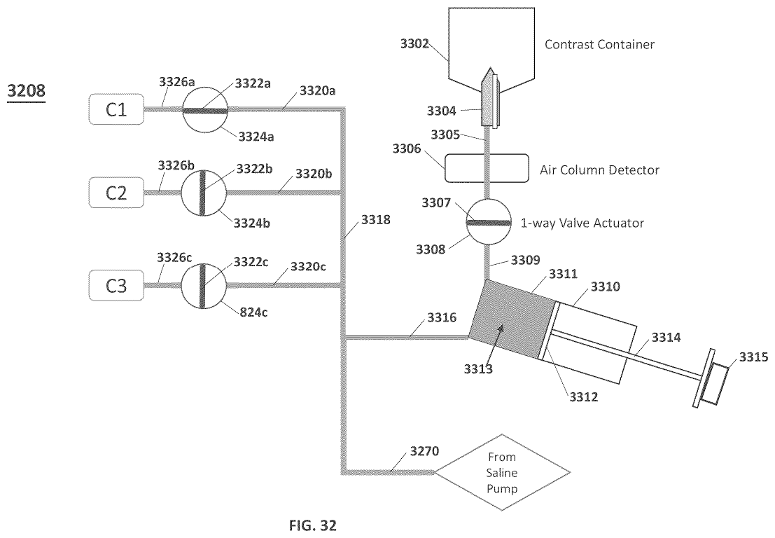

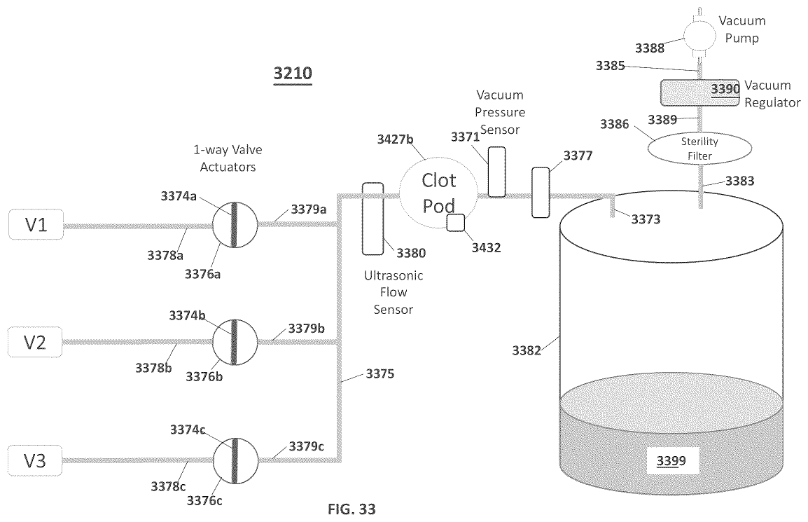

In this example, the fluidics system 3200 includes a saline subsystem 3206, a contrast subsystem 3208, and a vacuum subsystem 3210. In some embodiments, the vacuum subsystem 3210 may be a vacuum/aspiration (“V/A” or simply “vacuum”) subsystem. An example of a saline subsystem 3206 is illustrated in FIG. 31. An example of an embodiment of a contrast subsystem 3208 is illustrated in FIG. 32. An example of an embodiment of a vacuum subsystem 3210 is illustrated in FIG. 33. The embodiments illustrated in FIGS. 31, 32, and 33 can have additional components (e.g., valves, fluid channels/tubing, connectors, etc.) that are not shown for clarity of the figures (e.g., check valves). In various embodiments, certain components of the fluidics system 3200 can be part of the pump station 3202 and other components can be part of the cassette 3204. For example, a portion of the saline subsystem 3206 can be in the cassette 3204 and a portion of the saline subsystem can be in the pump station 3202. As a specific example, a portion of a peristaltic saline pump that controls saline flow can be in the cassette 3204, and a drive mechanism of the peristaltic saline pump (e.g., an actuator/drive motor, a drive member) can be in the pump station 3202. When a cassette is coupled to the pump station 3202, a drive mechanism is coupled to the portion of the peristaltic pump in the cassette such that controlling the drive mechanism controls the flow of saline through the peristaltic pump. In another example, the cassette 3204 may include a saline port to receive saline from a saline bag positioned outside of the cassette, and a weight sensor that senses the weight of the saline bag may be part of the pump station.

Also, the contrast subsystem 3208, or a portion thereof, can be in the cassette 3204. For example, a contrast syringe pump, or a portion thereof, can be located in the cassette 3204, and the cassette 3204 may include a contrast port to receive contrast from a contrast container positioned outside of the cassette 3204. Pump station 3202 may include an air column detector configured to detect air in a fluid communication channel (e.g., a tube) between the contrast container and the syringe pump.

Further, the vacuum subsystem 3210, or a portion thereof, can be included in the cassette 3204, and a portion of the vacuum subsystem can be included in the pump station. For example, a vacuum cannister and vacuum control valves can be in the cassette 3204, and a vacuum pump and actuators for the vacuum control valves can be located in the pump station 3202. As another example, the cassette 3204 may also include a vacuum port for coupling to a vacuum source of the pump station. Other configurations where certain components of the saline subsystem 3206, the contrast subsystem 3208, and the vacuum subsystem 3210 are located in the pump station 3202, and other components of the saline subsystem 3206, the contrast subsystem 3208, and the vacuum subsystem 3210 are located in the cassette 3204, are also possible.

The saline, contrast, and vacuum ports are part of a fluid communication system which includes fluid communication channels (e.g., channels, tubes, lines, etc.) to couple the catheters to the saline source, contrast source, and vacuum source. The cassette 3204 includes a portion of the fluid communication system that is couplable to the saline, contrast, and vacuum source, and valves in the cassette partially control fluid flowing through channels of the fluid communication system in the cassette. The fluid communication system also includes a tubing set 3216. The tubing set 3216 can include fluid communication channels for communicating saline, contrast, and/or vacuum to hubs 3224a-3224c and/or saline to the femoral sheath 3226. In this example, the tubing set 3216 includes saline tubes 3218, contrast tubes 3219, and vacuum tubes 3220. In preferred embodiments, the tubing set includes flexible tubes for providing saline, contrast and vacuum to the hubs. For example, tubes that may be 4′ to 10′ long, according to some embodiments. As illustrated in the embodiment of FIG. 30, the saline subsystem 3206 includes a first connector array 3212a, a second connector array 3212b, and a third connector array 3212c that represent where the saline, contrast, or vacuum is provided. Depending on the configuration of the cassette 3204, in some embodiment, the first connector array 3212a, a second connector array 3212b, and a third connector array 3212c can be arranged in a second connector array 3214 where the tubing set 3216 can be coupled to. Electrical channels 3217 can connect the first hub 3224a, the second hub 3224b, and the third hub 3224c to the cassette 3204 or to the pump station 3202, and can be used to provide control signals to the hubs or to receive information from the hubs. Although the illustrated electrical channels 3217 are physical communication channels (e.g., include wires, cables, or fiber optic), in some embodiments where the electrical channels 3217 are configured to provide control signals to the hubs 3224a-3224c or provide information from the hubs 3224a-3224c, the electrical channels 3217 can be wireless communication channels.

Each of the hubs 3224a-3224c (also referred to herein as a “hub assembly”) may include a plurality of components related to providing saline, contrast, and vacuum to a catheter, and components related to moving the hub and an interventional device attached thereto in an axial direction and rotating the catheter. The tubing set 3216 can also include electrical connections to communicate control information to the one or more hubs 3224 and/or receive information from components of the hubs 3224a-3224c (e.g., sensor information). A proximal end 3234 of the tubing set 3216 may be coupled to the cassette 3204, and a distal end 3232 of the tubing set 3216 may be coupled to the hubs 3224a-3224c and the femoral sheath 3226. In the illustrated example, each of the hubs 3224a-3224c is coupled to the cassette 3204 by an electrical channel 3217, a saline tube 3218, a contrast tube 3219, and a vacuum tube 3220. An example of fluidic components in an embodiment of a hub is illustrated in FIG. 34. In some embodiments, the hubs 3224a-3224c may include additional components. For example, a hub can include components associated with coupling an interventional device to the hub, moving the hub and the interventional device in an axial direction, and rotating the interventional device. An example of a tubing set 3216 is further illustrated in FIG. 36.

FIG. 31 illustrates certain components and connections of an example of a saline subsystem 3206, which can be the saline subsystem 3206 illustrated in FIG. 30. In this embodiment, the saline subsystem 3206 includes a saline weight sensor 3250 that is coupled to a saline bag 3251. A contrast spike 3252 is used to puncture the saline bag 3251 and allow saline to flow through a saline tube 3253 into a saline chamber 3254. An air vent 3255 on the chamber allows air to escape the saline chamber 3254 when it is being filled with saline. A chamber level detector 3256 is positioned to sense the saline level in the chamber. A line 3257 communicates saline from the saline chamber 3254 to the saline manifold 3258. Four peristaltic pumps 3262a, 3262b, 3262c, 3262d (hereinafter “peristaltic pumps 3262a-3262d”) receive saline from the saline manifold 3258 via lines 3260a, 3260b, 3260c, 3260d (hereinafter “lines 3260a-3260d”). Each peristaltic pump 3262a-3262d is driven by an actuator 3263a, 3263b, 3263c, 3263d (hereinafter “actuator 3263a-3263d”). In some embodiments including the example in FIG. 31, the peristaltic pumps are located in the cassette 3204 and the actuators 3263a-3263d are located in the pump station 3202. When the peristaltic pumps 3262a-3262d are driven by the actuators 3263a-3263d, saline flows through lines 3264a, 3264b, 3264c, 3264d (hereinafter “lines 3264a-3264d) to ports S1-S4, respectively, which can be at a connector array which is couplable to the tubing set 3216.

In the illustrated embodiment, a saline/contrast valve 3266 is included in line 3264c. The saline/contrast valve 3266 can be moved to be in a first position of a second position by a saline/contrast valve actuator 3267, which may be in the pump station 3202. The saline/contrast valve 3266 is configured such that when it is placed in a first position (shown in FIG. 31), the saline/contrast valve 3266 connects line 3264c to line 3270 to route saline from peristaltic pump 3262c to the contrast manifold 3318 (FIG. 32) for priming the contrast subsystem 3208. When the saline/contrast valve 3266 is placed in a second position, the saline/contrast valve 3266 connects line 3264c to line 3268 and to port S3.

FIG. 32 illustrates certain components and connections of an embodiment of a contrast subsystem 3208. In this embodiment, contrast subsystem 3208 includes the contrast container 3302, a connection 3304 to the contrast container (e.g., a “contrast spike”), and a line 3305 coupled to the connection 3304 and to a contrast intake valve 3307. The contrast intake valve 3307 is opened and closed by a contrast intake valve actuator 3308. A line 3309 is coupled to the contrast intake valve 3307 and also coupled to a contrast pump 3310, such that line 3309, contrast intake valve 3307, line 3305, and connection 3304 form a fluid communication channel between the contrast pump 3310 and the contrast container 3302, where the contrast intake valve 3307 is configured to open or close this fluid communication channel. An air column detector 3306 is positioned to detect air in line 3305 and generate a corresponding information (e.g., a signal) which the system controller 3800 can use as input for performing a process.

The contrast pump 3310 includes a housing 3311 that encloses a contrast chamber 3313 which receives contrast from the contrast container 3302. The contrast pump 3310 includes a wall or movable portion 3312 that can be moved to increase or decrease the size of the contrast chamber 3313. The movable portion 3312 is coupled to a movable member 3314, which can be moved by a linear contrast pump actuator 3315 which includes a motor. In some embodiments, the contrast intake valve actuator 3308, the contrast pump actuator 3315, and the air column detector 3306 (run-out sensor) can be located in the pump station 3202, and the contrast intake valve 3307 (between the connection 3304 and the contrast pump 3310), the contrast pump 3310, contrast control valves 3322a, 3322b, 3322c (hereinafter “contrast control valves 3322a-3322c”), and the lines and components that communicate contrast to ports C1, C2, C3 can be located in the cassette 3204, as shown in FIG. 30.

Contrast is provided from the contrast pump 3310 to a contrast manifold 3318 by line 3316. The contrast manifold 3318 provides a fluid communication channel to contrast ports C1, C2, and C3 via lines 3320a, 3320b, 3320c (hereinafter “lines 3320a-3320c”), contrast control valves 3322a-3322c, and lines 3326a, 3326b, 3326c (hereinafter “lines 3326a-3326c”). Contrast valve actuators 3324a, 3324b, 3324c (hereinafter “actuators 3324a-3324c”) are coupled to the contrast control valves 3322a-3322c when the cassette 3204 is coupled to the pump station 3202, and are controlled by the system controller 3800 to open and close the contrast control valves 3322a-3322c for performing preparation processes (e.g., priming) or for performing a medical procedure (e.g., injecting contrast). Line 3270 is a fluid communication channel connected to the saline subsystem 3206 connection and allows saline to flow from the saline subsystem 3206 to the contrast manifold 3318 as controlled by the saline/contrast valve 3266.

FIG. 33 illustrates an example of an embodiment of vacuum subsystem 3210 that includes a vacuum source (vacuum pump 3388), a communication channels that can be aligned to provide vacuum from the vacuum source to a catheter, and components to control providing the vacuum to the catheter. Various configuration of vacuum subsystems are possible to be able to selectively provide vacuum to portions of a fluidic system and a catheter at varying amounts of vacuum, for example, at a first level (e.g., a lower level) and at a second (e.g., a higher level). In the embodiment illustrated in FIG. 33, a vacuum pump 3388 is coupled to lines 3385, 3389, and 3383 which provide a channel to vacuum canister 3382. Specifically in this example, vacuum pump 3388 is coupled to line 3385, which is coupled to a vacuum regulator 3390 that can be controlled by a controller to provide a desired level of vacuum. Line 3389 is coupled to the vacuum regulator 3390 and also to a sterility filter 3386. Line 3383 is coupled to the sterility filter and to the vacuum canister 3382 which provides a reservoir to collect aspirated material 3399. Line 3373 is also coupled to the vacuum canister 3382 and is coupled to a vacuum manifold 3375 which is further coupled to channels that provide vacuum to multiple hubs/catheters of the robotic catheter system, for example, as illustrated in FIGS. 34 and 35. In this example, line 3373 is coupled to a vacuum regulator valve 3377 that can be controlled by a controller to provide a desired level of vacuum to the downstream vacuum subsystem (e.g., the vacuum manifold 3375, ports V1-V3, etc.). A vacuum pressure sensor 3371 can be positioned between the vacuum regulator valve 3377 and the ports V1-V3 to measure vacuum provided to hubs and catheters. In this example, the vacuum pressure sensor 3371 is positioned on line 3373 between the vacuum regulator valve 3377 and a clot pod 3427b, which is positioned on line 3373 between the vacuum manifold 3375 and the vacuum regulator valve 3377 to receive a clot aspirated by any one of a plurality of catheters connected to the vacuum subsystem 3210. In some embodiments, the clot pod 3427 includes a clot pod sensor 3432 that is in communication with a controller to provide information of the contents of the clot pod 3427 (e.g., when it contains a clot). A flow sensor 3380 (e.g., an ultrasonic flow sensor) is positioned between the clot pod 3427 and the vacuum manifold 3375 to sense the flow of material through line 3373 and provide flow information to a controller.

The vacuum manifold 3375 can be coupled to lines 3379a, 3379b, 3379c (hereinafter “lines 3279a-3379c”) and lines 3378a, 3378b, 3378c (hereinafter “lines 3378a-3378c”) which provide a vacuum channel to hubs/catheters via ports V1-V3. Vacuum control valves 3374a, 3374b, 3374c (hereinafter “vacuum control valves 3374a-3374c”) may be one-way valves and are coupled between lines 3379a-33879c and lines 3378a-3378c to control providing vacuum to ports V1-V3, and the vacuum control valves 3374a-3374c are opened and closed by vacuum control valve actuators 3276a, 876b. 876c (hereinafter “vacuum control valve actuators 3276a-876c”) of the pump station 3202, vacuum control valve actuators 3376a-3376c being controlled by a controller. To provide vacuum to one or more of the ports V1-V3, and hubs and catheters coupled to ports V1-V3, a controller opens the vacuum control valve 3374a-3374c corresponding to the desired port, actuates the vacuum pump 3388, and controls the vacuum regulator 3390 and vacuum regulator valve 3377 to produce the desired vacuum and monitors the vacuum being provided by using the vacuum pressure sensor 3371. Some embodiments can include a flow sensor 3380 that is associated with each port V1-V3 and/or a vacuum pressure sensor 3371 that is associated with each port V1-V3. However, in most procedures, vacuum is provided to one catheter at a time and in such cases multiple flow sensors and pressure sensors do not provide any operational advantage, and having a single flow sensor 3380 and a single vacuum pressure sensor 3371 positioned upstream of the vacuum manifold 3375, as shown in FIG. 33, reduces cost. In some embodiments, the vacuum pump 3388, the vacuum regulator 3390, vacuum control valve actuators 3276a-3376c, and the flow sensor 3380 are part of the pump station 3202, and the sterility filter 3386, the vacuum canister 3382, the vacuum regulator valve 3377, the vacuum pressure sensor 3371, the clot pod 3427 and vacuum control valves 3374a-3374c are located in the cassette 3204.

FIG. 34 illustrates an example of a catheter 3230 coupled to an embodiment of a tubing set 3216 in a hub portion 3410 of a fluidics management system. The distal end 3232 of the tubing set 3216 is coupled to the hub 3224 and to the catheter 3430, and the proximal end 3234 of the tubing set 3216 can be connected to a cassette 3204, which can include all or part of a saline subsystem, all or part of a contrast subsystem, and/or all or part of a vacuum subsystem of the fluidics management system. Certain components illustrated in FIG. 34 may be described as being part of the tubing set 3216, even though they may be in the hub 3224 or in a portion of a sterile adapter, because they operate to perform part of the fluid and vacuum communication functionality facilitated by the configuration of the tubing set. Means for providing fluid and electrical connections to the catheter can include one or more of the saline subsystem, the contrast subsystem, the vacuum subsystem, and/or electrical connections that are described herein.

As illustrated in FIG. 34, the catheter 3430 can be coupled to a portion of the tubing set 3216 of the fluidics management system via a luer connection 3426. In this example, the portion of the tubing set 3216 includes a contrast tube 3437 connected to port/connection point C1, a saline tube 3439 S1 connected to port/connection point, and an aspiration tube 3435 connected to port/connection point V1. One or more electrical channels 3417 are connected to port/connection point E1 which can be on the cassette 3204 or on the pump station 3202. In some embodiments, the electrical channels 3217 are part of the tubing set 3216, such that the tubing set 3216 includes tubes for communicating saline, contrast, and vacuum tubes from the cassette to the hubs, and electrical connections to the hubs, and such embodiments may be advantageous for wire/tube management. The tubing set 3216 can include a branch point in the form of a two-to-one wye connector 3420 that couples upstream of the contrast tube 3437 and the saline tube 3439 of the tubing set 3216 to a single downstream saline/contrast tube 3440. The tubing set can further include a three-way valve 3423 that can be actuated by a three-way valve actuator 3424 to selectively place the catheter 3430 in communication with the single downstream saline/contrast tube 3440 or the aspiration tube 3435. In some embodiments, the tubing set 3216 can further include a catheter coupling tube 3433 downstream of the three-way valve 3423 to couple the three-way valve 3423 with the catheter 3430. The three-way valve actuator 3424 can be actuated by a controller. In some embodiments, the three-way valve actuator 3424 comprises a drive assembly configured to move the three-way valve 3223. In some embodiments, the three-way valve actuator 3424 comprises electromechanical means for moving the three-way valve 3423, the electromechanical means controlled by a controller. In some embodiments, the three-way valve actuator 3424 comprises a motor controlled by a controller.

In some embodiments, the three-way valve 3423 can be a three-way stopcock. The three-way valve 3423 may be actuated (e.g., rotated) to selectively provide or prevent fluid communication between ports coupled to the saline/contrast tube 3440, the aspiration tube 3435, and the catheter coupling tube 3433. The three-way valve 3423 can be actuated to a first position to open a fluid communication channel between the aspiration tube 3435 and the catheter coupling tube 3433 and a second position to open a fluid communication channel between the saline/contrast tube 3440 and the catheter coupling tube 3433. In some embodiments, the three-way valve 3423 can be actuated to third position in which the aspiration tube 3435, the saline/contrast tube 3440, and the catheter coupling tube 3433 are all in fluid communication. In some embodiments, the three-way valve can be actuated to a fourth position in which the vacuum tube 3435 and the saline/contrast tube 3440 are in fluid communication. In some embodiments, the three-way valve 3423 can be actuated to a fifth position in which none of the aspiration tube 3435, the saline/contrast tube 3440, and the catheter coupling tube 3433 are in fluid communication.

While a three-way valve 3423 is shown in FIG. 34, other valve arrangements for selectively placing the catheter 3430 in communication with the saline/contrast tube 3440 or the aspiration tube 3435 may be used.

As shown in FIG. 34, an air bubble filter 3422 may be positioned between the wye connector 3420 and the three-way valve 3423. In some embodiments, a clot pod 3427 may be positioned along the aspiration tube 3435 upstream of the three-way valve 3423. A clot pod sensor 3432 (in communication to a controller) can be positioned to detect material on the clot pod 3427. In some embodiments, a clot pod 3427 may be positioned in the hub 3224 along the aspiration tube 3435 upstream of the three-way valve 3423. In some implementations, the wye connector 3420, three-way valve 3423, three-way valve actuator 3424, clot pod 3427a, and/or portions of the tubing set can be housed within a magnetic sterile adapter that may couple with the hub 3224 (which may also be referred to as a puck) and can be considered to be part of a hub assembly. In some embodiments, a clot pod 3427b can be positioned. A clot pod 3427a may be positioned along the aspiration tube 3435 closer to the vacuum canister 3282, for example, between the vacuum manifold 3275 and the vacuum canister 3282 as illustrated in the embodiment in FIG. 33.

A hemodynamic pressure sensor 3429 may be positioned between the three-way valve 3423 and the catheter 3430, for example, on the catheter coupling tube 3433. The hemodynamic pressure sensor 3429 is configured to sense a hemodynamic pressure of a patient in which the catheter 3430 is inserted, and provide information relating to the sensed pressure to a controller. FIG. 35 illustrates another embodiment of components and fluid communication channels that can be coupled to and/or positioned in a hub, and also illustrates an example of another interventional device 3431 (e.g., a catheter, a guidewire) which can be coupled to another hub and positioned to extend through the hemostasis valve 3428 and at least partially into the lumen of the catheter 3430.

FIG. 36 illustrates an example of an embodiment of a tubing set 3216 that can provide communication channels from a cassette to one or more hubs and a femoral sheath. The tubing set 3216 has a distal end 3232 and a proximal end 3234. In this example, different portions of the distal end 3232 is coupled to a first hub 3224a, a second hub 3224b, a third hub 3224c, and a femoral sheath 3226. Different portions of the proximal end 3234 are coupled to contrast, saline, vacuum, and electrical connections on the cassette. In this example, different portions of the proximal end 3234 are coupled to contrast ports (C1, C2, C3) of a contrast subsystem, saline ports (S1, S2, S3, S4) of a saline subsystem, vacuum ports (V1, V2, V3) or a vacuum subsystem to provide fluid communication channels from a contrast, saline, and vacuum source to the hubs, and provide a fluid communication channel from the saline subsystem to the femoral sheath. In this example, the tubing set 3216 also includes electrical communication channels that are connected to hubs 724a-724c and connectors E1, E2, E3 on the cassette 3204. The electrical communication channels can provide power to the hub to operate components of the hub, and/or can provide signals/information from the hub to the cassette, for example, from a hemodynamic sensor positioned in or on a hub. The cassette can include electrical connections which are coupled to corresponding electrical connections on the pump station when the cassette is attached to the pump station, such that the pump station can provide power to the hubs via the cassette and the electrical communication channels of the tubing set, and/or receive signals/information from the hubs via the electrical communication channels of the tubing set and the cassette.

FIG. 37 illustrates a schematic of an example of a robotic catheter system that includes a remotely located system (“remote system”) and a locally located system (which may be referred to a “local system,” “bedside system,” or a “near-patient system”). FIG. 37 also illustrates an example of certain components of the local system that includes certain components of a fluidic management system (“fluidics system”) including actuatable components that are actuated by a controller and sensors that provide information to a controller for the controller to control the fluidics systems and other aspects of the robotic catheter system, according to some embodiments. The remote portion includes a control system 3710 located remotely from the patient. “Remote” as used herein is a broad term and generally refers to a location other than where the patient/local system is located when the patient undergoes a medical procedure using the robotic catheter system. For example, in a different room from the patient, or in a different building, or in a different town or city or state. In some embodiments the remote portion is located hundreds or thousands of miles away from the local portion. The control system 3710 is configured to be in communication with a system controller 3800 which is part of the local portion of the system. The system controller 3800 may include multiple controllers each having one or more processors. In this example, the system controller 3800 includes an interface 3735. The interface 3735 can include multiple interfaces and may be a user interface. The interface 3735 can be configured for communicating with the remotely located control system 3710. For example, the interface 3735 can be configured to receive control signals from the control system 3710 to perform a fluidic action (e.g., inject contrast from a catheter, provide aspiration from a catheter), or to move one or more of the hubs 3224 to move (axially or rotationally) one or more catheters or a guidewire, and communicate corresponding signals to a controller (e.g., controller 3720 or pump station 3740). The interface 3735 can also be configured to receive control information from a user located locally (e.g., for a fluidic action or to control movement of the hubs and coupled to interventional devices, and to provide information (status, images, etc.) to a local user. The interface 3735 can include multiple interfaces. For example, one or more displays and displaying information relating to the robotic catheter system, the medical procedure, and/or the patient. The interface 3735 can also include one or more user input devices, for example, switches, buttons, touchscreen controls, etc.

The interface 3735 can also be configured to communicate information to the remotely located control system 3710. The communicated information can be related to a received control action, fluidic information, catheter position information, status information, images or video, audio and other communications from the robotic system or users located locally with the patient, and any other information that may be needed to control the robotic catheter system from the control system 3710. The interface 3735 can also be configured to receive inputs from users of the robotic catheter system located with the patient.

The system controller 3800 can include pump station 3740 for performing fluidic-related actions, and a controller 3720 configured for processing user inputs received locally or from the control system 3710, and processing sensor information, and controlling the pump station 3740 and other portion of the robotic control system to perform medical procedures based on the user inputs and the sensed information, including providing saline, contrast, and vacuum to hubs 3224a-3224c, and saline to the femoral sheath, as needed during a medical procedure.

The robotic catheter system illustrated in FIG. 37 can include actuatable components of a fluidics management system that are movable, as controlled by a controller, to align fluid communication channels to provide saline, contrast, and vacuum from a saline, contrast, and vacuum source (respectively) to hubs 3224a-3224c, and including certain sensors that are configured to sense a condition related to providing provide saline, contrast, and vacuum to the hubs 3224a-3224c and provide the sensed information to a controller. The controller can be configured to use the sensed information, user inputs, and/or stored information to control actions of the fluidics system to perform a medical procedure. In various embodiments, a pump station 3740 may include a controller to control actuators of the pump station 3740 to perform fluidic-related actions (e.g., provide saline, contrast, and vacuum to the hubs 3224a-3224c). In other embodiments, controller 3720 and/or system controller 3800 can be configured to control actuators and other components of the pump station 3740 to perform fluidic-related actions. Many components and systems of the robotic catheter system may not be illustrated in FIG. 37 for clarity of this illustration (for example, a hub axial drive system, check valves, a catheter rotational system, controllers of the remote control system 3710, etc.).

A cassette configured to be releasably attached to the pump station can include all or part of the saline subsystem 3206, the contrast subsystem 3208, and the vacuum subsystem 3210. A determination of what is disposable may be based on contact or near contact with patient materials (cells, blood, removed clots, etc.). In some embodiments, the cassette and its components is disposable, and the pump station 3202 and its components are non-disposable (e.g., capital equipment). In an example of a saline subsystem 3206, a pump station 3202 can include saline weight sensor 3250 and a saline drip rate sensor 3259, peristaltic pumps actuators 763a-763d, and a saline/contrast valve actuator 3267, and a cassette 3204 can include a saline level detector 3245, peristaltic pumps 762a-762d, and a saline/contrast valve 3266 (FIG. 15). In an example of a contrast subsystem 3208, a pump station 702 can include contrast intake valve actuator 3308, contrast pump actuator 3315, contrast valve actuators 3324a-824c, and air column detector 3306, and the cassette 3204 can include contrast valves 822a-822c, contrast pump 3310, and contrast intake valve 3307 (FIG. 16). In an example of a vacuum subsystem 3210, a pump station 3202 can include vacuum pump 3388, vacuum regulator 3390, vacuum control valve actuators 3276a-876c, and flow sensor 3280 (e.g., an ultrasonic flow sensor), and a cassette 3204 can include a vacuum regulator valve 3277, a vacuum pressure sensor 3271, a clot pod sensor 3432, and vacuum control valves 3274a-3274c. In various embodiments, pump station 3202 and the cassette 3204 can include additional components or fewer components. Also, in some embodiments, certain components illustrated and/or described herein as being part of a cassette can be located in a pump station, and certain components illustrated and described herein as being a part of, or supported by a cassette, can be located in a pump station.

In FIG. 37, the components shown in solid lines represent actuatable components and sensors, where the actuatable components can be controlled directly or indirectly by a controller, and the sensors provide information to a controller. Components shown in dashed lines are not sensors and not actuatable components (e.g., ports S1-S4, C1-C3, V1-V3; a saline chamber, a vacuum cannister, a femoral sheath). In this embodiment, actuatable components of saline subsystem 3206 can include peristaltic pump actuators 3263a-3263d and a saline/contrast valve actuator 3267 that can be operated to position saline/contrast valve 3266 (FIG. 31) in an first or second position to open or close a fluid communication channel between the saline subsystem 3206 and the contrast subsystem 3208; and sensors of the saline subsystem 3206 can include saline weight sensor 3250, saline level detector 3245 that senses saline level in saline chamber 3254, and saline drip rate sensor 3259. In this embodiment, actuatable components of the contrast subsystem 3208 can include contrast intake valve actuator 3308, contrast pump actuator 3315, and contrast valve actuators 3324a-3324c; sensors of the contrast subsystem can include air column detector 3306. In some embodiments, the contrast subsystem may comprise a plurality of air column detectors 3306a, 3306b. In this embodiment, actuatable components of the vacuum subsystem 3210 can include vacuum pump 3388, vacuum regulator 3390, vacuum regulator valve 3377, and vacuum control valve actuators 3376a-3376c; sensors of the vacuum subsystem can include vacuum pressure sensor 3371, clot pod sensor 3432, and flow sensor 3380 (e.g., an ultrasonic flow sensor).

The fluidics system also can include channels and components in the one or more hubs 3224. For example, as illustrated in the embodiments of FIGS. 34 and 35. Examples of actuatable components related to a hub that can be actuated by a controller for administering fluids or providing vacuum are also illustrated in FIG. 37. In this embodiment, a robotic catheter system includes three hubs 3224a-3224c that have catheters coupled thereto, and a fourth hub 3224d that has a guidewire coupled thereto. In some embodiments, the guidewire is releasably coupled to the fourth hub 3224d. Hubs 3224a-3224c include a hemostasis valve 3428 and a valve 3423 (e.g., a three-way valve) that is used to couple a catheter lumen to a fluid or vacuum. Hubs 3224a-3224d have driving mechanisms 3702 that can function to move the hubs (and an interventional device coupled thereto) forward and back along a common line (“axially”). Hubs 3224a-3224d also have driving mechanisms 3704 that can rotate an interventional device coupled to the hub, around its longitudinal axis. In various embodiments, the driving mechanisms in a hub 3224 can couple with a driving mechanism outside of the hub (e.g., magnetically, mechanically, etc.) such that the drive mechanisms interact to move the hub axially and rotate the interventional device.

FIG. 38 illustrates a representation of an embodiment of a cassette 3204 and a pump station 3202 illustrating certain components of the cassette (e.g., valves, electrical connections, etc.) and corresponding components (e.g., motors, electrical connections, etc.) of the pump station. The illustrated components in the cassette and pump station illustrated in FIG. 38 can correspond to the components described here in and illustrated in, for example, FIGS. 30-33, 36, and 37, and many of the other figures.

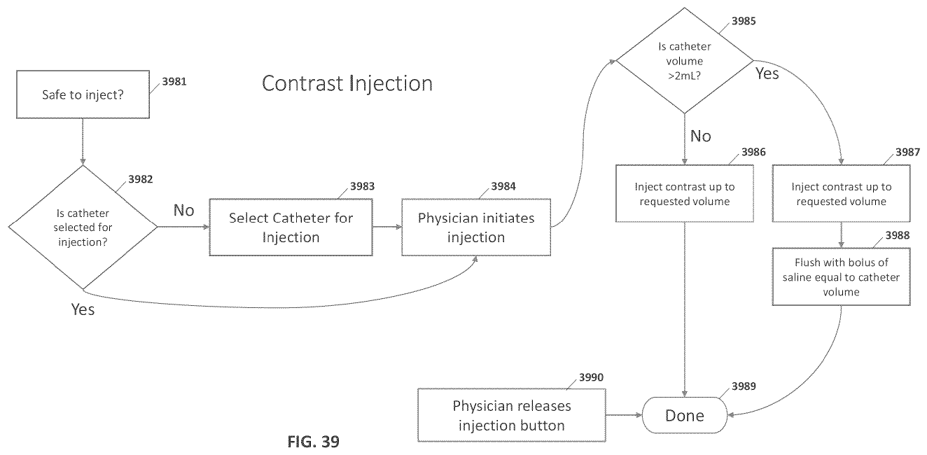

FIG. 39 illustrates an example of a process, which can include subprocesses, that can occur when the fluidic system is in the contrast injection state, according to some embodiments. This process includes actions that can be determined or performed using the robotic catheter system (or “system”) and the description of the actions relates to using the robotic catheter system to perform the actions. For example, these actions can be controlled by a controller based at partially on, for example, system information (e.g., user input from a local control interface, a user input from a remotely located control system, information received from one or more sensors, previously stored information, and/or previously stored processes that can be executed by the controller).

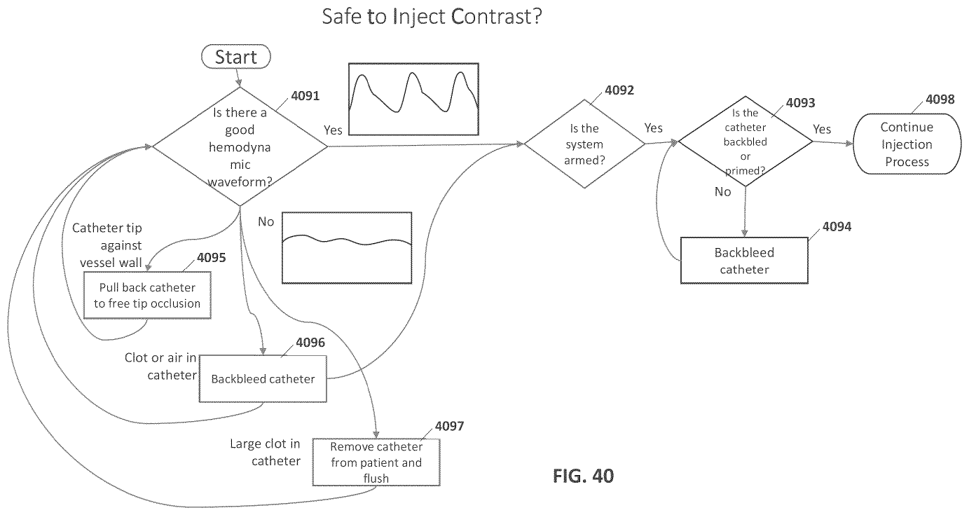

In FIG. 39, the system determines if it is safe to inject contrast at block 3981. FIG. 40 illustrates a process for determining if it's safe for the system to inject contrast. In this example, the system cannot proceed with contrast injection until it has been determined that it is safe to inject contrast. Referring to FIG. 40, the system determines if there is a good hemodynamic waveform at block 4091. The system can determine if there is a “good” hemodynamic waveform based on information received from a hemodynamic pressure sensor 3429 corresponding to the selected catheter (e.g., positioned in a hub that the selected catheter is coupled onto), for example, by comparing the received information to predetermined hemodynamic pressure information (e.g., waveforms) that indicate an expected hemodynamic waveform. Using the hemodynamic pressure sensor information, the system can determine if the tip of the selected catheter is occluded making it unsafe to inject contrast. The predetermined mode dynamic pressure information can be stored on the robotic catheter system or stored at another storage location that is accessible by the robotic catheter system. If the system determines a good hemodynamic wave does not exist, the system can initiate other actions represented in blocks 4095, 4096, 4097 to clear the occluded catheter tip. For example, the process can proceed to block 4095 and where the system moves the selected catheter to free the catheter tip in instances with the catheter tip is against a vessel wall. The process can also proceed to block 4096 where the system back-bleeds the catheter to remove a clot or error in the catheter. The process can also proceed to block 4097 where the catheter is removed from the patient flushed in order to, for example, remove a large clot in the selected catheter.

After executing any of actions represented in blocks 4095, 4096, 4097, the process can proceed back to block 4091 where the system determines if a good hemodynamic waveform exists. If after the system performs one of the actions represented in blocks 4095, 4096, 4097, and a good hemodynamic waveform still does not exist, the system can proceed to another of actions represented in blocks 4095, 4096, 4097, perform that action to proceed back to block 4091 and again determine if a good hemodynamic waveform exists. During this process, the system can provide status, to a remotely located control system 3710 and/or on an interface of the local system controller 3800, indicating an action that has taken. In some embodiments, the system receives input from a user indicating which of the actions represented in blocks 4095, 4096, 4097, or another action, to perform in order to ensure the catheter tip is clear. In some embodiments, the system determines which of the actions represented in blocks 4095, 4096, 4097, or another action, to perform, for example, based on historical data, system information, physician's preference, patient information, or other information. After the system determines a good hemodynamic waveform exists, the process can move to block 4092 with the system determine/verify if it is armed and ready to inject contrast. For example, if the contrast pump 3310 is ready to inject contrast. After the system determines it is armed, the process proceeds to block 4093 where it determines if the catheter is back-bled and/or primed. If not, the process proceeds to block 4094 with the system back-bleeds the selected catheter. After the system determines that the catheter has been back-bled or primed, the process proceeds to block 4098 where contrast injection can continue, and the process proceeds to the seventh block 3982 in FIG. 39.

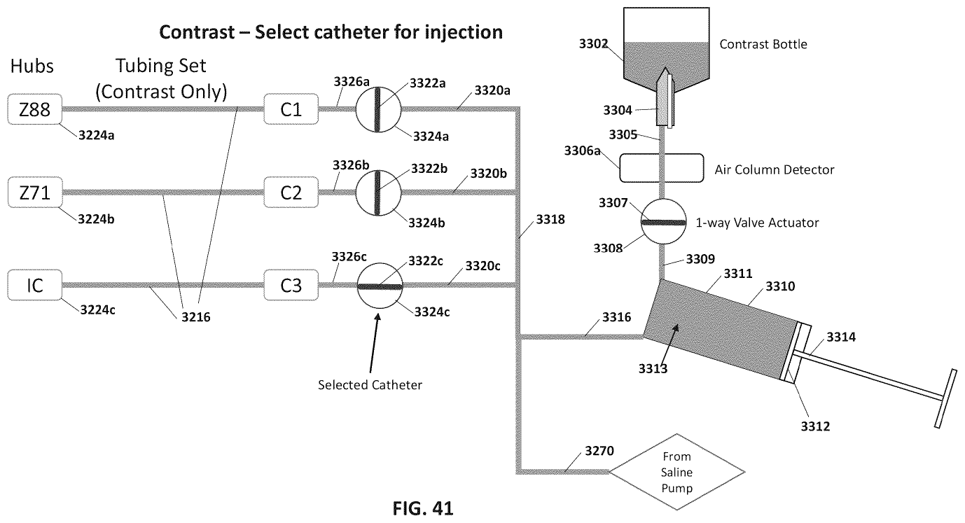

At the seventh block 3982 the system determines if a catheter has been selected for injecting contrast. If a catheter has been selected, the process proceeds to block 3984 where a user (e.g., physician) initiates contrast injection. If a catheter has not been selected, the process proceeds to block 3983 where a catheter is selected for injecting contrast, for example, based on a user input. FIG. 41 illustrates an example of the contrast subsystem 3208 configuration where an insert catheter coupled to hub 3224c has been selected for injecting contrast. When the insert catheter has been selected, the system moves the third contrast control valve 3322c to an open position connecting lines 3326c and 3320c to provide the contrast communication path from the contrast pump 3310 to the insert catheter. The system also moves the first and second contrast control valves 3322a, 3322b and the contrast intake valve 3307 to a closed position, if they are not already closed. The system can perform other actions to prepare for injecting contrast. After a catheter is selected, the process proceeds to block 984 where a user initiates contrast injection by providing an input to the system to inject contrast, for example, from the remotely located control system 3710.

The contrast injection process proceeds to block 3985 where the system can determine if the selected catheter volume is greater than a certain predetermined amount (for example, if the volume of the catheter is greater than 2 ml) based on system information. In some instances, the system determines the volume of the selected catheter based at least partially on whether the lumen of selected catheter contains another catheter, or a guidewire, which the system may determine based on information relating to the position of the hubs when contrast injection is being performed. If the volume of the selected catheter is not greater than the predetermined amount, at block 3986 the system injects contrast, for example, up to a requested volume.

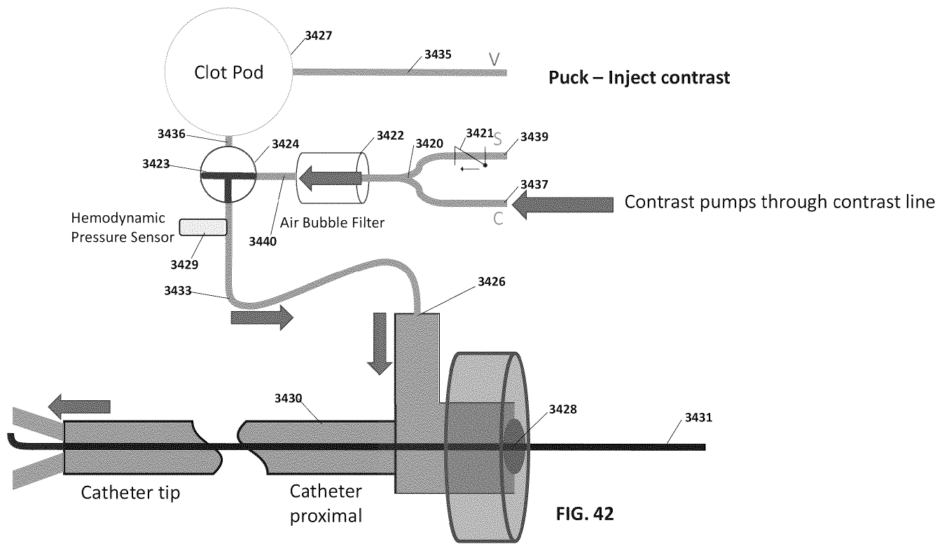

If the catheter volume is greater than the predetermined amount, at block 3987 the system injects contrast, for example, up to a requested amount. FIG. 42 illustrates an example of a configuration of the hub corresponding to the selected catheter when contrast is injected. The system actuates contrast pump 3310 to provide contrast to the hub via the contrast tube 3437. The three-way valve 3423 is positioned to connect the saline/contrast tube 3440 and the catheter coupling tube 3433 so the provided contrast flows through the catheter coupling tube 3433, through the luer connection 3426, though the catheter 3430 and exhausts at the catheter tip. In some embodiments, the system closes hemostasis valve 3428 around the interventional device 3431 a more forceful level than during saline drip to prevent contrast from leaking out of the hemostasis valve 3428.

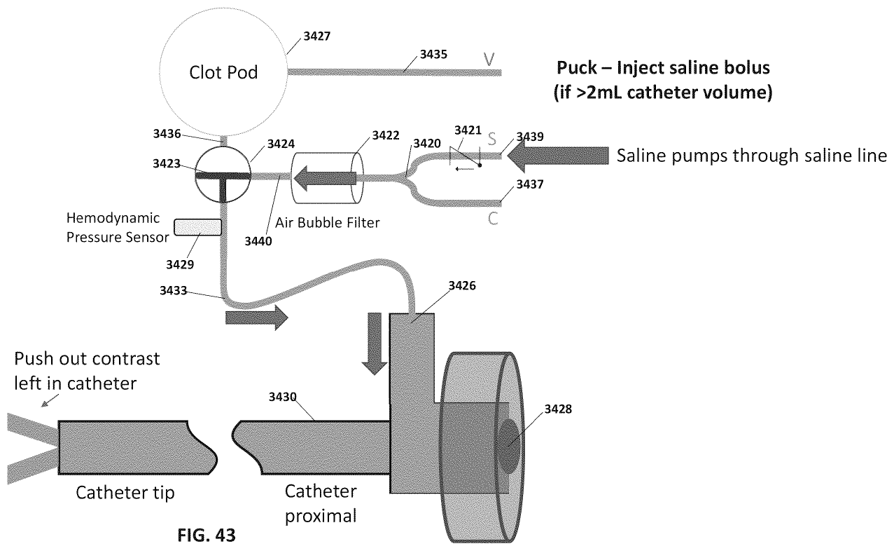

Referring again to FIG. 39, the process then proceeds to block 3988 where the system flushes the hub and the catheter with a bolus of saline equal to the catheter volume to exhaust the contrast solution of the catheter. FIG. 43 illustrates an example of a configuration of the hub of the selected catheter configured to inject a saline bolus. The peristaltic pump 3263a-3263d associated provides a certain amount of saline to the hub via the contrast tube 3437. The amount of saline provided to the hub can be based on the volume of the selected catheter (which can be part of the system information). The three-way valve 3423 is the lines to connect the saline/contrast tube 3440 to the catheter coupling tube 3433 such that the saline flows through catheter coupling tube 3433, through the luer connection 3426, through the catheter 3430 and pushes out contrast that is left in the catheter 3430 as a result of the contrast injection. Referring again to FIG. 39, after the contrast is injected to the requested volume and block 3986, in after the catheter is flushed with a bolus of saline, if needed, in block 3988, the process can indicate this particular injection process is done at block 3989. In this example, the process then proceeds to block 3990 with a physician can release the contrast injection button indicating the contrast injection is complete.

Additional embodiments are disclosed in further detail in the following examples, which are not in any way intended to limit the scope of the claims

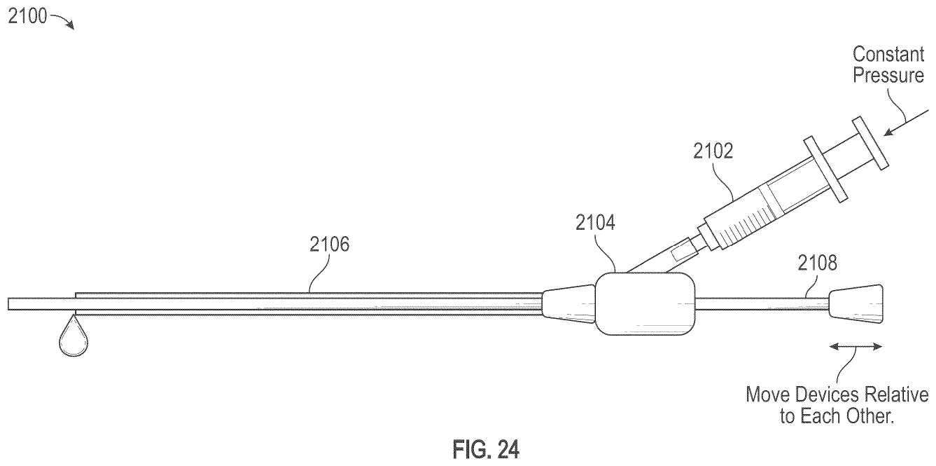

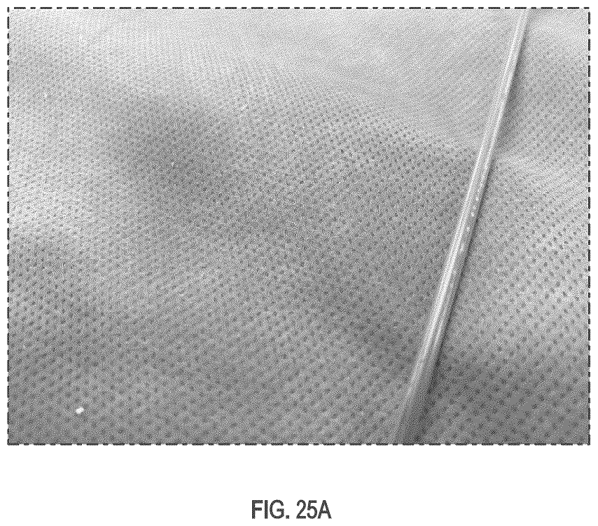

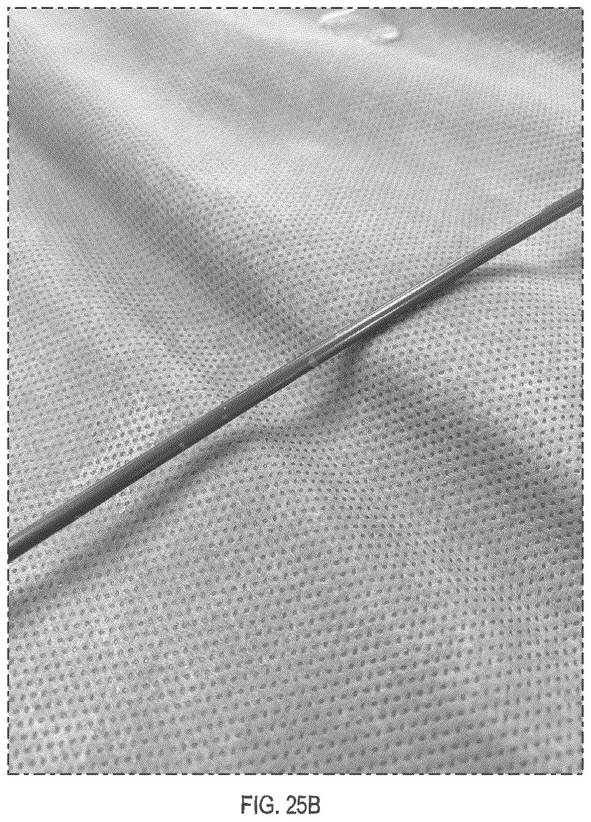

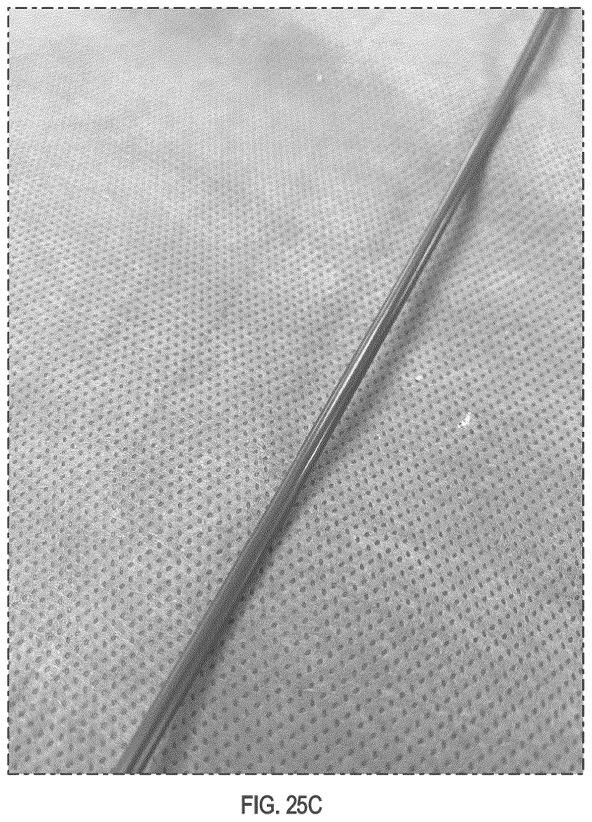

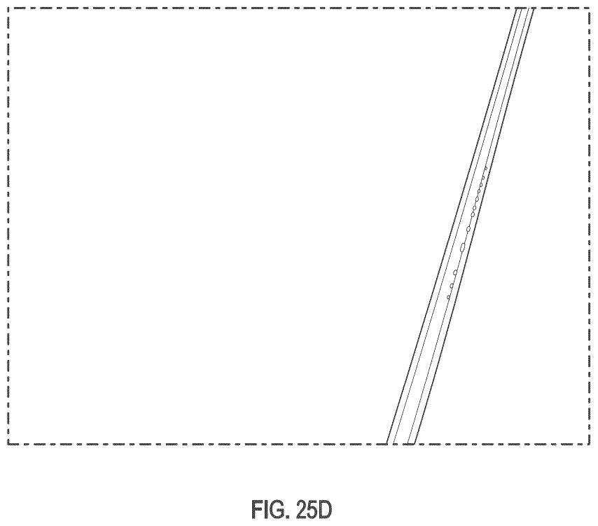

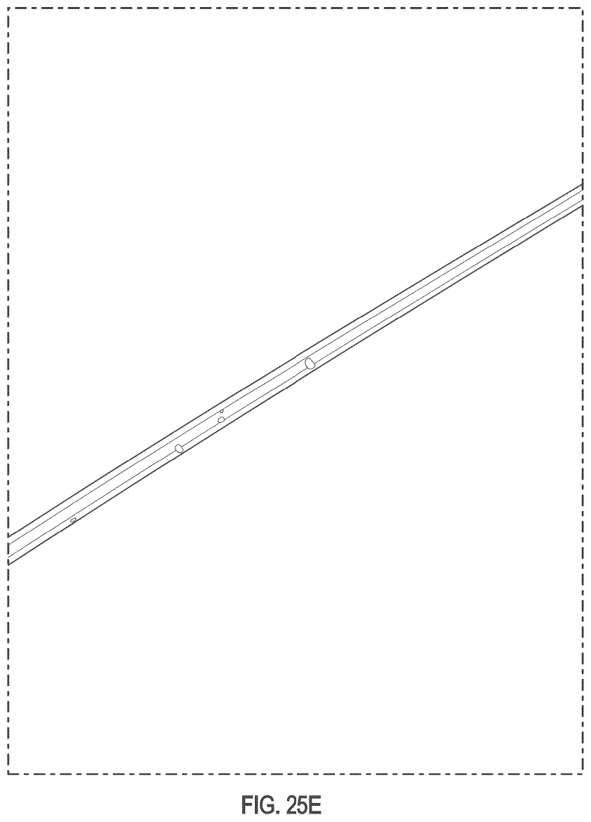

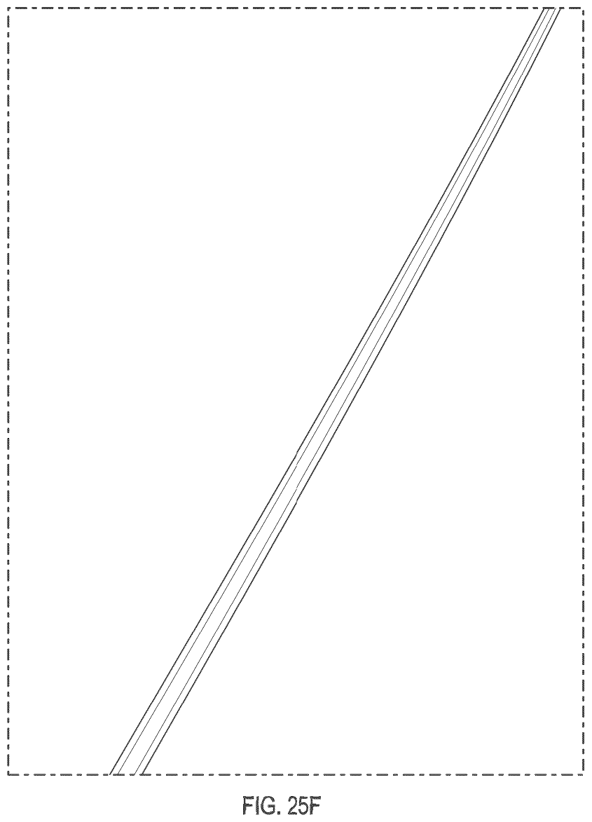

FIG. 24 is a diagram of an example of a test system that was used for detecting the removal of air bubbles between concentrically stacked catheters. The test system included an inner catheter 2108 positioned within an interior lumen of an outer catheter 2106 in a concentric stack. The outer catheter 2106 was coupled to a rotating hemostasis valve 2104. The hemostasis valve 2104 was coupled to a syringe 2102 so that fluid injected using the syringe would flow through the lumen between the inner catheter 2108 and the outer catheter 2016. In the test system, the inner catheter 2108 had a diameter of about 0.071 inches. The outer catheter 2106 had a diameter of about 0.088 inches. The outer catheter 2106 was transparent to permit visualization of bubbles within the lumen. A distal end of the outer catheter 2108 allowed for small volumes of fluid to exit the outer catheter. FIG. 25A is a photograph showing the catheter 2106 and catheter 2108 in a concentric stack, prior to injection of fluid. FIG. 25D is an illustration thereof.

### Example 1

In a first example, the syringe 2102 (FIG. 24) was used to inject water at a constant pressure of about 150 PSI through the hemostasis valve 2104 without moving the catheter 2106 or the catheter 2108. FIG. 25B is a photograph showing the catheter 2106 and catheter 2108 following the injection of water. FIG. 25E is an illustration thereof. As shown in FIG. 25B and FIG. 25D, bubbles are present within the lumen between the catheter 2106 and the catheter 2108.

### Example 2

In a second example, the syringe 2102 (FIG. 24) was used to inject water at a constant pressure of about 150 PSI through the hemostasis valve 2104. Shortly after beginning to inject water, axial reciprocal movement of the inner catheter 2108 was performed for about 10 seconds. The reciprocal movement was performed at a frequency of about 1 Hz (or less) and a stroke length of about 20 mm (or more). FIG. 25C is a photograph showing the catheter 2106 and the catheter 2108 following the axial reciprocal movement. FIG. 25F is an illustration thereof. As shown in FIG. 25C, the lumen between the catheter 2106 and the catheter 2108 was substantially free of bubbles.

### Example 3

In a third example, an outer catheter having an inner diameter of about 0.071 inches and an inner catheter having an inner diameter of about 0.035 inches were used in the test system 2100 (FIG. 24) instead of the outer catheter 2106 and the inner catheter 2108 described with respect to Examples 1 and 2. A syringe 2102 was used to inject water at a constant pressure of about 150 PSI through a hemostasis valve 2104 coupled to the outer catheter. Shortly after beginning to inject water, axial reciprocal movement of the inner catheter was performed for about 10 seconds. The reciprocal movement was performed at a frequency of about 1 Hz (or less) and a stroke length of about 20 mm (or more). Following the axial reciprocal movement, the lumen between the outer and inner catheters was found to be substantially free of bubbles by visual inspection.

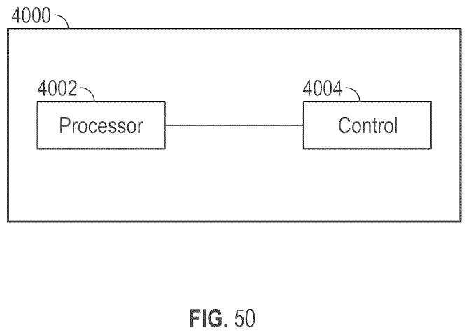

FIG. 50 illustrates a schematic view of an example of a control system 4000 that may be used to electronically control the systems and components described herein and/or perform the methods described herein. The control system 4000 may be configured to automatically adjust various motors, hub adapters, hubs, interventional devices, fluidics components (e.g., valves, pumps, etc.), and/or any other components described herein in response to commands input by an operator such as a physician. In response to command inputs by an operator, the control system 4000 may cause a responsive event to occur, or cause a series of responsive events to automatically occur.

In certain embodiments, the control system 4000 can include one or more processors 4002. The one or more processors 4002 can be configured to automatically adjust the various system components described herein in response to commands input by an operator, for example, using one or more controls 4004 of the control system 4000. A single control 4004 is shown in FIG. 50. However, any suitable number of controls may be provided to correspond to various functions of the systems described herein. For example, in certain embodiments, each interventional device may have its own unique control 4004 or set of controls 4004 that can control various functions of the interventional device (e.g., axial movement, rotational movement, supply of fluids (e.g., saline, contrast, etc.), aspiration, etc.).

In certain embodiments, one or more controls 4004 may control priming functions for one or more interventional devices. For example, one or more controls 4004 can be operated to cause the interventional devices to perform a priming procedure, as described for example, with reference to FIGS. 22A-22C. For example, one or more controls 4004 can be operated to cause axial movement of one or more interventional devices relative to one or more other interventional devices (e.g., by causing axial movement of corresponding hubs and/or hub adapters). One or more controls 4004 can be operated to cause introduction of fluid into the lumen of an interventional device to prime the interventional device.

In certain embodiments, one or more controls 4004 may be operated to cause the interventional devices to perform a priming procedure, as described for example, with reference to FIGS. 23A-23B. For example, one or more controls 4004 can be operated to cause reciprocal movement (e.g., axial and/or rotational reciprocal movement) of one or more interventional devices relative to one or more other interventional devices (e.g., by causing reciprocal movement of corresponding hubs and/or hub adapters). One or more controls 4004 can be operated to cause introduction of fluid into the lumen of an interventional device to prime the interventional device (e.g., during relative reciprocal movement).

The processor 4002 may receive signals from the one or more controls 4004 and in response, initiate corresponding actions in the components of the systems described herein. For example, the processor 4002 may be configured to generate output signals that cause responsive actions to be performed by the components of the described herein.

While the foregoing describes robotically driven interventional devices and manually driven interventional devices, the devices may be manually driven, robotically driven, or any combination of manually and robotically driven interventional devices, as will be appreciated by those of skill in the art in view of the disclosure herein.

The foregoing represents one specific implementation of a robotic control system. A wide variety of different robotic control system constructions can be made, for supporting and axially advancing and retracting two or three or four or more assemblies to robotically drive interventional devices, as will be appreciated by those of skill in the art in view of the disclosure herein.

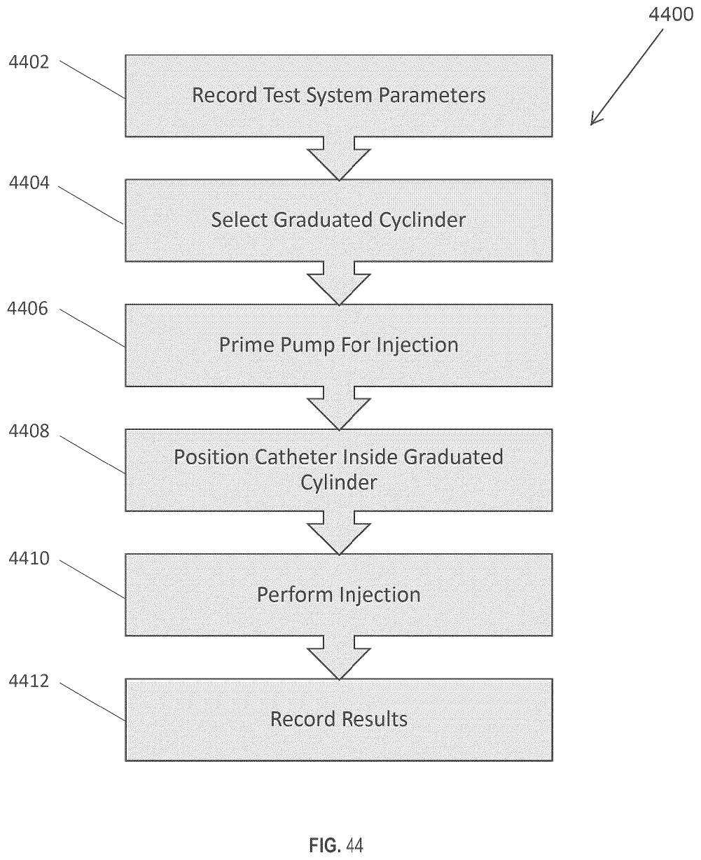

As explained above in connection to FIG. 17, in embodiments of the catheter assembly 2900 where the guidewire 2907 remains inside the annular lumen of the insert or access catheter 2902 during the injection of contrast media, factors including, but not limited to, the viscosity of the liquid being injected into the catheter assembly 2900 may affect the dimensions of the access catheter 2902 and guidewire 2907 necessary to allow for the liquid to flow at a desirable rate (e.g, 2 ml/s for 3 seconds, or 3 ml/s for 3 seconds). In order to determine viable ranges of these dimensions, trials were run on test systems using an infusion device, an access catheter, a guidewire, and an injection fluid. Different parameters of the test system were altered in each trial to determine that parameters effect on the test system's performance. For example, some trials tested the effect of the length of access catheter 2902 on the test system's performance. Other variables that were tested included the radius of the guidewire 2907, using a guidewire 2907 with a hydrophilic coating, the type of pump, the power injection setting of the pump, and the type of injection fluid run through the test system (e.g., water, Omnipaque 300 contrast, 67% Glycerin Contrast, 56% Glycerin contrast, etc.). FIG. 44 illustrates an overview of an example of a trial procedure (or process) 4400 for the contrast injection study discussed above, where contrast is injected through the system and into a graduated cylinder where it can be measured, and information relating to the process was recorded. First, at block 4402, parameters of the specific test system being used in the trial (e.g., length of the catheter, type of fluid being used, temperature of the fluid being used, and/or guidewire diameter, etc.) are recorded. At block 4404, a graduated cylinder appropriate for the volume of fluid being used (e.g., 6 ml, 30 ml, etc.) was selected. Next, at block 4406, a pump being used in the test system (e.g., Medrad pump, Acist pump, etc.) was primed for injection. At block 4408 of the process, the distal tip of the catheter was positioned in the middle of the graduated cylinder 4408, advantageously allowing the user performing the test to clearly observe when a steady stream of the fluid stopped flowing out of the tip. At block 4410, the injection sequence is initiated (e.g., by a user). The time of the injection was from the time period from when the continuous stream started until the stream turned into droplets. At block 4412, after the injection was complete, the user recorded the final volume of fluid in the graduated cylinder.

Various trials were performed using trial procedure 4400 to demonstrate the effect of various variables including catheter length, guidewire diameter, guidewire coating, programmed injection volume, the viscosity of the fluid being injected, the presence of a guidewire, and a comparison of the contrast vs glycerin mixture) on the test system. These trials are described in Examples 4-12 below.

### Example 4

In a fourth example, trial procedure 4400 was performed to compare the performance of a traditional insert catheter (e.g., a Cook-brand catheter) and the insert catheter described herein (e.g., a Telos-brand catheter). Both catheters had the same length and injection parameters.

Table I below shows a baseline comparison between the performance of the two catheters. The various settings of the power injector are listed, as well as catheter properties. The “Max Medrad pressure” column lists the maximum pressure reached during a Medrad injection. This pressure is an approximate value. The “Flow Rate Difference” column indicates how much larger the bigger value is compared to the smaller value. Finally, the “T test” column indicates the p-value calculated from performing a T test with the two samples in the trial. Trials with 3 samples show the p-value calculated from an ANOVA test. P-values of less than 0.05 indicate that the differences in samples of the trial are considered statistically significant. Underneath these p-values are values for the sample sizes, signified with “n=”.

TABLE IIA

| | | | Length | Injec- | | Max | Measured | | T Test |
|---|---|---|---|---|---|---|---|---|---|
| | Core | | from start | tion | Injection | Medrad | Flow | Flow Rate | (n = |
| | Diameter | | of tubing | Rate | Volume | Pressure | Rate | Difference | sample |
| Catheter | (in) | Fluid | (cm) | (ml/s) | (ml) | (psi) | (ml/s) | (%) | size) |
| Cook | No Guide | Water | 100 | 6 | 6 | 99.3 | 2.53 | 1 | 0.3923864 |
| VTK | Wire | | | | | | | | |
| Telos | 0.018 | Water | 100 | 6 | 6 | 95.6 | 2.50 | | n = 10 |
| NA | (coated) | | | | | | | | |

As shown by the results in Table IIA, the Telos insert catheter described herein closely mimicked the injection properties of the traditional Cook insert catheter, while keeping the guidewire in place in the Telos insert catheter during injection. Both the injection pressure and measured flow rates with the Cook catheter and the Telos catheter are very similar. There is only a 1% difference in flow rate between the two catheters, and a T-test yields a p-value of 0.39, indicating that there is not a significant difference in flow rate. This was achieved by designing the effective cross-sectional areas of the two catheters to be very similar. The Telos catheter has an effective cross-sectional area of 0.001407 square inches (exclusive of the guidewire), compared to the Cook catheter, which had an effective cross-sectional area of 0.001257 square inches (based on the lumen diameter of the catheter without a guiding element positioned in the lumen).

### Example 5

In a fifth example, trial procedure 4400 was performed to determine the effect of the length of the access catheter 2902 on the test system's performance. The trials (“Trials 1”, “Trial 2”, “Trial 3”) were conducted using access catheters 2902 with a length of 193 cm and access catheters 2902 with a length of 100 cm. The results of Trials 1, 2 and 3 are shown in Table IIB below.

TABLE IIB

| | | | | | | | | | | T test |
|---|---|---|---|---|---|---|---|---|---|---|
| | | Core | | | | | Max | Measured | Flow Rate | (n = |
| Trial | | Diameter | | Length | Injection | Injection | Medrad | Flow | Difference | sample |
| # | Pump | (in) | Contrast | (cm) | Rate | Volume | Pressure | Rate | (%) | size) |
| 1 | Medrad | N/A | Water | 193 | 6 | 6 | N/A | 2.55 | 23 | 0.0006478 |
| | | | (1 cP) | | | | | | | |
| | | N/A | Water | 100 | 6 | 6 | N/A | 3.13 | | n = 10, 13 |
| | | | 1(cP) | | | | | | | |
| 2 | Acist | 0.018 | Omni- | 193 | 4 | 7.5 | N/A | 2.17 | 20 | n = 10 |
| | | (coated) | paque | | | | | | | |
| | | | 300 | | | | | | | |
| | | 0.018 | Omni- | 100 | 4 | 7.5 | N/A | 2.59 | | |
| | | (coated) | paque | | | | | | | |
| | | | 300 | | | | | | | |
| 3 | Medrad | 0.018 | 67% | 193 | 6 | 6 | 606 | 1.48 | 34 | n = 3 |
| | | (uncoated) | Glycerin | | | | | | | |
| | | 0.018 | 67% | 100 | 6 | 6 | 471 | 1.98 | | |
| | | (uncoated) | Glycerin | | | | | | | |
| | | 0.018 | 56% | 193 | 7 | 6 | 381 | 1.34 | | |
| | | (coated) | Glycerin | | | | | | | |

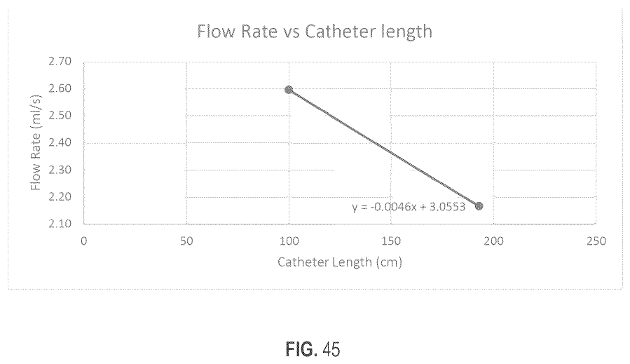

Trial 2 was chosen for analysis due to it being performed with Omnipaque 300 contrast fluid, rendering it the most representative of an actual injection. The two data points in the trial had a sample size of 10 each. A T-test of the data yielded a p-value of 1.77 E-11, indicating that there is a significant difference between the two catheter length data. FIG. 45 shows an interpolated linear relationship between the catheter length and the corresponding flow rate. This chart assumes a constant injection rate of 4 ml/s and an injection volume of 7.5 ml, as well as a 0.018″ guidewire being present in the lumen of the insert catheter. This injection rate and injection volume was empirically determined to yield the desired output flow rate of 2 ml/s with the 193 cm insert catheter and a 0.018″ guidewire. The 2 ml/s output flow rate is the physician specification for the necessary contrast injection flow rate.

The relationship between catheter length and flow rate is graphically represented in FIG. 45, which shows an inverse relationship between catheter length and flow rate. From the line of best fit of the data, an increase in 100 cm of catheter length corresponds to a 0.46 ml/s decrease in output flow rate. While this does not appear to directly match to Poiseuille's law it agrees with the inverse relationship (this apparent non-direct match may be due to errors from the procedure itself or due to assumptions made.

### Example 6

In a sixth example, trial procedure 4400 was performed to determine the effect of the guidewire diameter on the test system's performance. These trials (“Trial 4” and “Trial 5”) used an Acist pump, and a guidewire with a length of 193 cm. The results of Trials 4 and 5 are shown in Table III below.

TABLE III

| | Core | | | | | Measured | T test |
|---|---|---|---|---|---|---|---|
| | Diameter | | Injection | Injection | Max Medrad | Flow | (n = sample |
| Trial # | (in) | Contrast | Rate | Volume | Pressure | Rate | size) |
| 4 | 0.018 coated | Omnipaque | 4 | 7.5 | N/A | 2.17 | |
| | | 300 | | | | | |
| | 0.020 uncoated | Omnipaque | 4 | 7.5 | N/A | 1.90 | |
| | | 300 | | | | | |
| | 0.022 uncoated | Omnipaque | 4 | 7.5 | N/A | 1.76 | |
| | | 300 | | | | | |
| 5 | 0.018 coated | Omnipaque | 4 | 7.5 | N/A | 2.17 | n = 10 |
| | | 300 | | | | | |
| | 0.020 uncoated | Omnipaque | 4 | 7.5 | N/A | 2.16 | |
| | | 300 | | | | | |
| | 0.022 uncoated | Omnipaque | 6.5 | 7.5 | N/A | 1.98 | |
| | | 300 | | | | | |
| | 0.018 (coated) | 56% | 7 | 6 | 381 | 1.34 | |
| | | Glycerin | | | | | |

Trials 4 and 5 were conducted using guidewires with core diameters of 0.018 inch, 0.020 inch, and 0.022 inch. The different guidewire diameters were tested using the same injection parameters (e.g., injection rate, injection volume, injection fluid) and insert catheter. The difference in the flow rates was found to be significant, as the F-statistic from an ANOVA test was 0.000001.

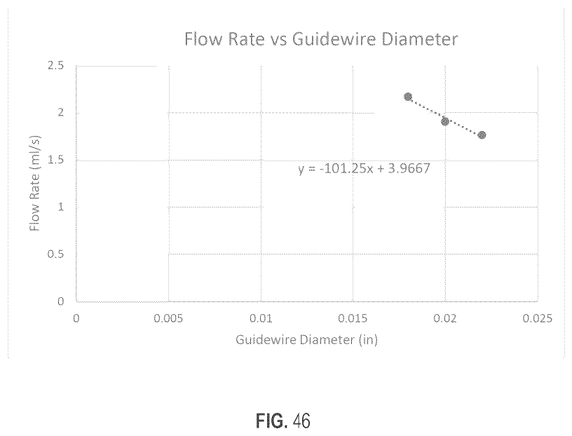

FIG. 46 graphically represents the inverse relationship between guidewire diameter and output flow rate shown by the data collected in Trials 4 and 5. An increase in diameter of 0.001 inch corresponds to a decrease in flow rate of approximately 0.1 ml/s. When using the adjusted Poiseuille Law for annular flow to determine the change in flow as a result of the change in guidewire size, it appears not to match exactly with the line of best fit shown in FIG. 46. The calculator predicts a decrease of 0.16 ml/s in flow as opposed to the 0.1 ml/s decrease seen in the data. This is most likely due to stacking of error ranging from procedural to assumptions taken to use Poiseuille's Law (such as no fluid acceleration), as well as adapting Poiseuille's law to annular flow.

An important finding during the guidewire testing occurred during testing for Trial 5. The goal of this testing was to determine the required injection rate to achieve an output flow rate of 2 ml/s for the different guidewire sizes. This was performed at an injection volume of 7.5 ml. The tests with 0.018 inch and 0.020 inch achieved 2 ml/s output flow relatively easily, but the 0.022 inch wire required very high pressures to achieve 2 ml/s.

When the test system used an Acist pump, output flow was limited due to the high required injection pressures of over 1000 PSI. This finding led to a hypothesis that a 0.024 inch guidewire would require even higher injection pressures, and therefore would not be desirable.

Therefore, Trials 4 and 5 allowed for the conclusion that guidewires (or more generally guiding elements) with an outer diameter of 0.018 inches and 0.020 inches were ideally suited, or nearly so, for the test system where the guidewire was in place in the access catheter during injection. Accordingly, guiding elements (e.g., guidewires) having an outer diameter equal to between about 0.018 inches and about 0.020 inches (e.g., about 0.019 inches) would also be ideally suitable. Also, guiding elements (e.g., guidewires) having an outer diameter smaller than about 0.018 inches can also be used. For example, guiding elements having an outer diameter of about 0.014 inches, about 0.015 inches, about 0.016 inches, about 0.017 inches, and guiding elements having an outer diameter of about between about 0.017 inches and about 0.018 inches. In some embodiments, guiding elements having an outer diameter of less than about 0.014 inches can also be used. The structure of the guiding element may be determined by stiffness and functional requirements. In some typical examples the guiding element can have an outer diameter are about 0.018 inches or about 0.020 inches, or between about 0.018 inches and 0.020 inches to satisfy functional requirements (e.g., stiffness) and fluid flow requirements while the guiding element is positioned in the access catheter when fluid is provided through the access catheter.

### Example 7

In a seventh example, trial procedure 4400 was performed to determine the effect of a guidewire with a hydrophilic coating on the test system's performance. The trial (“Trial 6”) used a Medrad pump, and a 193 cm long catheter. The same injection rate, injection volume and 56% Glycerin contrast were used. The results of Trial 6 are shown in Table IV below.

TABLE IV

| | Core | | | | | Max | Measured | Flow Rate | T test |
|---|---|---|---|---|---|---|---|---|---|
| Trial | Diameter | | Length | Injection | Injection | Medrad | Flow | Difference | (n=sample |
| # | (in) | Contrast | (cm) | Rate | Volume | Pressure | Rate | (%) | size) |
| 6 | 0.018 | 56% | 193 | 7 | 6 | 402 | 1.36 | 2% | n = 3, 9 |
| | (uncoated) | Glycerin | | | | | | | |
| | 0.018 | 56% | 193 | 7 | 6 | 381 | 1.34 | | |
| | (coated) | Glycerin | | | | | | | |

A t-test performed on the data yielded a corresponding p-value 0.6, indicating that there was not a significant difference in the flow rates of an insert catheter with a coated vs uncoated guidewire, suggesting that hydrophilic coating of the guidewire has negligible effect on flow rate.

### Example 8

In an eighth example, trial procedure 4400 was performed to determine the effect of raising injection rates on the output flow rate out of the catheter. Each trial was run using an Acist pump, an 0.018 inch-diameter coated guide wire and Omnipaque 300 contrast. The results of are shown in Table VI below.

Due to short supply of contrast, tests were not completed for the 5, 6, and 7 ml/s injection rate trials with the 7.5 volume. It was assumed that the data would be similar enough to draw conclusions with the 8 ml volume instead of 7.5 ml.

TABLE V

| Injection Rate (ml/s) | Injection Volume (ml) | Max Medrad Pressure | Measured Flow Rate |
|---|---|---|---|
| 1 | 7.5 | N/A | 0.86 |
| 2 | 7.5 | N/A | 1.47 |
| 2.5 | 7.5 | N/A | 1.66 |
| 3 | 7.5 | N/A | 2.01 |
| 4 | 7.5 | N/A | 2.17 |
| 5 | 8 | N/A | 2.26 |
| 6 | 8 | N/A | 2.49 |
| 7 | 8 | N/A | 2.49 |

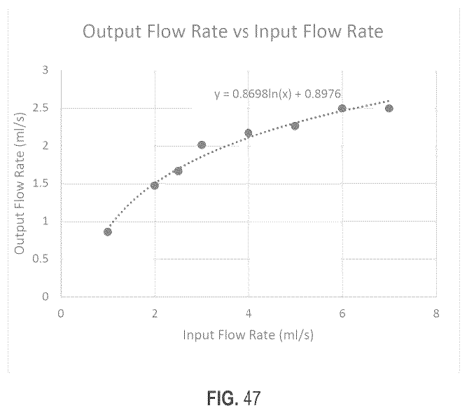

FIG. 47 graphically represents the results form Table VI, and shows a positive correlation between increasing the injection rate of the injection fluid and the output flow rate of the test system. More specifically, the relationship appears to be logarithmic in nature as seen by the plateauing of the data points on the right side of FIG. 47.

Several different factors may account for this phenomenon, one being that there is an inherent ramp up time for the power injectors, and because of the short injection times used in the experiments, the power injectors have difficulty actually achieving the higher injection rates. Another explanation is that the longer lengths of the catheters may introduce some lag into the system so that the injection rate is reached in the correct amount of time, but then the catheter continues to drain for a period of time afterwards. This is verified by the shorter recorded flow times of the 100 cm-length catheter as opposed to the 193 cm-catheter. Air bubbles present in the pressure gauge caused even more lag in preliminary experiments. Finally, the increase in input flow rate causes an increase in pressure, which in turn may cause the catheter to expand, therefore changing the control volume and decreasing output flow.

### Example 9

In a ninth example, trial procedure 4400 was performed to determine the effect of increasing injection volumes on the output flow rate of the test system. These trials (“Trial 7” and “Trial 8”) were both conducted with a Medrad pump. The results from Trials 7 and 8 can be seen in Table VI below.

TABLE VI

| | | Core | | | | | Max | | Flow Rate | T test |
|---|---|---|---|---|---|---|---|---|---|---|
| Trial | | Diameter | Contrast | Length | Injection | Injection | Medrad | Measured | Difference | (n = sample |
| # | Pump | (in) | (cP) | (cm) | Rate | Volume | Pressure | Flow Rate | (%) | size) |
| 7 | Medrad | 0.018 | 67% | 193 | 6 | 6 | N/A | 2.55 | 89% | n = 2, 3 |
| | | (uncoated) | Glycerin | | | | | | | |
| | | | (26.6 cP) | | | | | | | |
| | | 0.018 | 67% | 100 | 6 | 6 | N/A | 3.13 | | |
| | | (uncoated) | Glycerin | | | | | | | |
| | | | (26.6 cP) | | | | | | | |
| 8 | Medrad | 0.018 | 56% | 193 | 6 | 30 | 730.5 | 4.09 | 135% | n = 4, 6 |
| | | (uncoated) | Glycerin | | | | | | | |
| | | | (11.9% cP) | | | | | | | |
| | | 0.018 | 56% | 193 | 6 | 6 | 526 | 1.73 | | |
| | | (uncoated) | Glycerin | | | | | | | |
| | | | (11.9% cP) | | | | | | | |
| | | 0.018 | 56% | 193 | 7 | 6 | 381 | 1.34 | | |
| | | (coated) | Glycerin | | | | | | | |
| | | | (11.9% cP) | | | | | | | |

When testing with larger injection volumes, it was apparent that volume had a positive effect on output flow rate as seen in FIG. 46. With the higher injection volumes, it was possible to get closer to the injection rate set on the power injector. As seen on FIG. 46, a flow rate of 4 ml/s could be reached, which is far closer to the injection rate of 6 ml/s.

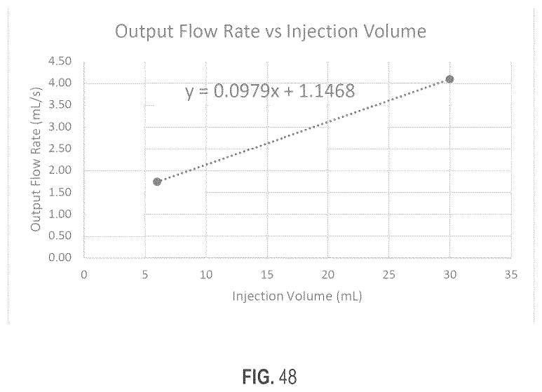

While the injection volume is an important part of an injection, it is an easily changeable parameter that can be adjusted as needed. Additionally, it was not realistic to carry out large volume tests with contrast, as it is expensive, and in short supply. While an increased volume did have a large effect on the flow rate when testing with large volumes like 30 ml, these volumes are not realistic for an actual procedure. A hypothesis for why the flow rate is so much higher for the larger volume injections is that the power injector has a ramp up time and increasing the overall injection time reduces the impact of the ramp up time on the overall flow rate. Additionally, any error in measuring the flow rate is mitigated by the longer period of steady state flow. FIG. 48 graphically represents the relationship between injection volume and output flow rate.

### Example 10

In a tenth example, trial procedure 4400 was performed to determine the effect of the viscosity of the injection fluid on the output flow rate of the test system. This trial (“Trial 9”) was conducted with a Medrad pump. The results from Trial 9 can be seen in Table VII below.

TABLE VII

| | | | | | | Max | Measured |
|---|---|---|---|---|---|---|---|
| Trial | Core Diameter | | Length | Rate | Volume | Medrad | Flow |
| # | (in) | Contrast (cP) | (cm) | Injection | Injection | Pressure | Rate |
| 9 | 0.018 (uncoated) | Water (1 cP) | 193 | 6 | 6 | 120 | 2.14 |
| | 0.018 (uncoated) | 67% Glycerin (26.6 cP) | 193 | 6 | 6 | 606 | 1.48 |
| | 0.018 (uncoated) | 56% Glycerin | 193 | 6 | 6 | 526 | 1.73 |
| | | (11.9% cP) | | | | | |
| | 0.018 (uncoated) | Water (1 cP) | 193 | 6 | 6 | 120 | 2.14 |
| | 0.018 (coated) | 56% Glycerin | 193 | 7 | 6 | 381 | 1.34 |
| | | (11.9% cP) | | | | | |

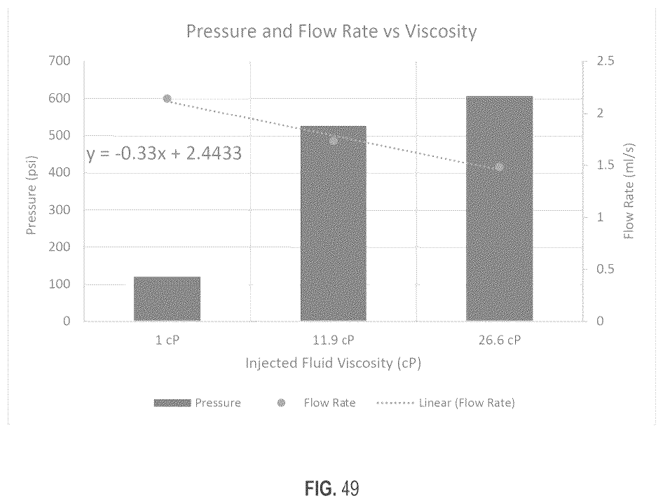

The viscosity testing was performed with varying viscosities and all the other variables held as controls. This is seen in Trial 9, where the corresponding injection pressures and flow rates are shown. This data can be visualized in FIG. 49. An important thing to note is that the 11.9 cP and 26.6 cP viscosities are from glycerin and water mixtures which do not appear to have the same properties as contrast (as seen in the glycerin and contrast comparison above). Therefore, this data should be used as only a relative comparison, and a confirmation that flow varies approximately in a linear fashion.

### Example 11

In an eleventh example, trial procedure 4400 was performed to determine the effect of the guidewire's presence within the lumen of the access catheter on the output flow rate of the test system. These trials (“Trial 10” and “Trial 11”) were conducted with a Medrad pump. The results of Trials 10 and 11 can be seen in Table VIII below.

TABLE VIII

| | Core | | | | | Max | Measured | Flow Rate | T test |
|---|---|---|---|---|---|---|---|---|---|
| Trial | Diameter | Contrast | Length | Injection | Injection | Medrad | Flow | Difference | (n = sample |
| # | (in) | (cP) | (cm) | Rate | Volume | Pressure | Rate | (%) | size) |
| 10 | N/A | Water | 193 | 6 | 6 | 92 | 2.66 | 25% | n = 6.20 |
| | | (1 cP) | | | | | | | |
| | 0.018 | Water | 193 | 6 | 6 | 120 | 2.14 | | |
| | (uncoated) | (1 cP) | | | | | | | |
| 11 | N/A | 56% | 193 | 6 | 6 | 421.5 | 1.96 | 13% | n = 10 |
| | | Glycerin | | | | | | | |
| | | (11.9% | | | | | | | |
| | | cP) | | | | | | | |
| | 0.018 | 56% | 193 | 6 | 6 | 526 | 1.73 | | |
| | (uncoated) | Glycerin | | | | | | | |
| | | (11.9% | | | | | | | |
| | | cP) | | | | | | | |

This testing was performed to act as a baseline in determining the effect that the guidewire has on the flow rate of the catheter. As expected, the flow rates were significantly different with injection over a guidewire as opposed to no guidewire. Trial 10 used water as the fluid, which yielded a p-value of 5.182E-06. Water was chosen for this test as it is well established as a Newtonian fluid, whereas the glycerin solution appears to have varied flow properties.

Trial 10 showed a 30 PSI increase in pressure when injecting over the 0.018″ guidewire, as well as a flow decrease of 0.52 ml/s. The pressure increase and flow decrease observed would be magnified using a higher viscosity fluid such as contrast.

### Example 12

In a twelfth example, trial procedure 4400 was performed to determine if a glycerin solution could be made to mimic the properties of contrast for testing purposes. This glycerin solution could then be used in lieu of contrast, due to the shortage and expense of contrast. This trial (“Trial 12”) used a Medrad pump. The results of Trial 12 can be seen in Table IX below.

TABLE IX

| | Core | | | | | | | Flow Rate | T test |
|---|---|---|---|---|---|---|---|---|---|
| Trial | Diameter | Contrast | Length | Injection | Injection | Max Medrad | Measured | Difference | (n = sample |
| # | (in) | (cP) | (cm) | Rate | Volume | Pressure | Flow Rate | (%) | size) |
| | 0.018 | 56% | 193 | 6 | 6 | 526 | 1.73 | | |
| | (uncoated) | Glycerin | | | | | | | |
| | | (11.9% cP) | | | | | | | |
| 12 | 0.018 | Omnipaque | 193 | 7 | 6 | 662.5 | 2.10 | 57% | n = 2, 9 |
| | (coated) | 300 | | | | | | | |
| | 0.018 | 56% | 193 | 7 | 6 | 381 | 1.34 | | |
| | (coated) | Glycerin | | | | | | | |
| | | (11.9% cP) | | | | | | | |

For Trial 12, power injections were performed holding all factors constant except for the fluid being injected and the corresponding pressure and output flow rate. The two fluids used were supposed to have similar viscosities at about 11.9 cP (please reference “Glycerin Viscosity Measurements” document above for how this was done). Performing the injections showed significantly different flow rates and pressures, with a p-value of 0.0235, with the glycerin having a much lower pressure and flow than the Omnipaque 300 contrast. This indicates that a glycerin mixture should not be used as a model for the contrast due to varying flow properties between the two.

### Example 13

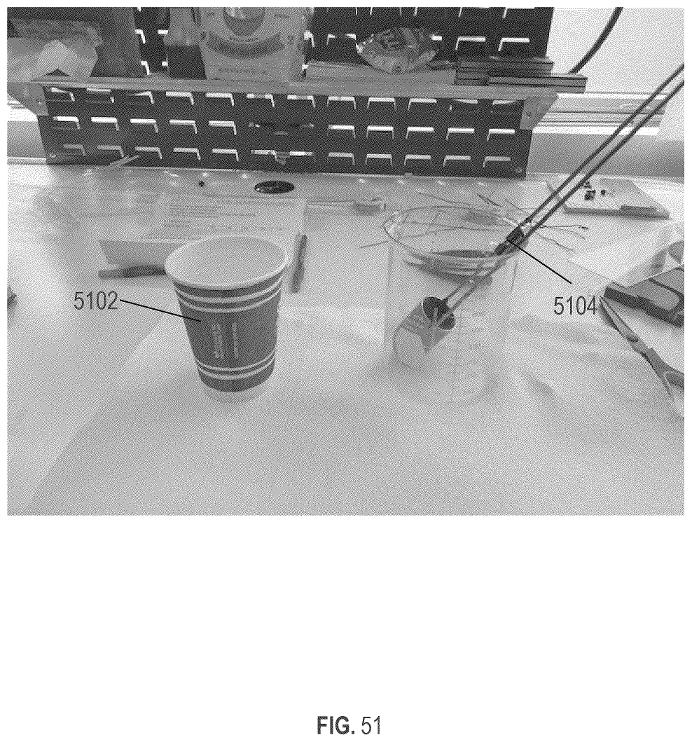

An important thing to note is that the glycerin solutions were mixed to achieve the 11.9 cP and 26.6 cP viscosities based off a calculator. FIG. 51 depicts the set-up of an example test system that was used for glycerin viscosity testing when preparing the glycerin solutions used in Trials 1-12 above. The test system included a container 5102 containing a glycerin solution and a viscosimeter 5104. For this test system, a Boekel manual viscosimeter was used, which has a range of approximately 18-56 centistokes, and functions by recording the time it takes for the liquid to flow out of a fixed volume of 44 ml. The manual viscosimeter was dipped into the coffee cup containing a glycerin solution, and the time from when the top of the cup broke the surface until the solution began to drip out of the viscosimeter, was recorded. As one would expect, the smaller the draining time the lower the viscosity.

The measured time was then converted into a centistoke viscosity value using a time-to-viscosity calculator. The centistoke value was then multiplied by the specific gravity of the glycerin solution to determine the centipoise (cP) viscosity value of the glycerin solution.

The calculated viscosity value was then checked against the expected viscosity value output by a glycerin viscosity calculator to confirm the accuracy of the method.

Using the manual viscosimeter with a solution of 64% glycerin yielded times of about 35 seconds, translating to a calculated viscosity value of about 7-8 cP. However, the expected viscosity of a 64% glycerin solution (output from the glycerin viscosity calculator) is 20 cP. Although the glycerin viscosity calculator may not be fully accurate, the fact that the percent error was over 100%, suggests that using a manual viscosimeter is not a reliable method for calculating viscosity in present application.

While the manual viscosimeter was not useful for determining exact viscosities, it was useful for relative viscosity measurements between liquids. The recorded time for the solutions to exit the fixed volume of the manual viscosimeter can be seen in Table X below:

TABLE X

| Expected Viscosity (cP) | Mixture | Time (s) |
|---|---|---|
| 11.9 | 56% Glycerin | 32.94 |
| | | 32.99 |
| | | 33.15 |
| | | 32.05 |
| 20 | 64% Glycerin | 37.04 |
| | | 36.01 |
| | | 35.91 |
| 26.6 | 67% Glycerin | 37.39 |
| | | 37.58 |
| | | 37.66 |
| 11.8 | Contrast | 31.99 |
| | | 31.26 |
| | | 31.44 |

These measurements show that the contrast and 56% glycerin mixture behave somewhat similarly, although more testing may be required to confirm whether the two solutions have the same flow properties under a power injection.

While the foregoing describes robotically driven interventional devices and manually driven interventional devices, the devices may be manually driven, robotically driven, or any combination of manually and robotically driven that are driven by a drive table, other suitable robotic drive systems or mechanisms may be used to drive the interventional devices, as will be appreciated by those of skill in the art in view of the disclosure herein.

Various systems and methods are described herein primarily in the context of a neurovascular access or procedure (e.g., neurothrombectomy). However, the catheters, systems (e.g., drive systems), and methods disclosed herein can be readily adapted for any of a wide variety of other diagnostic and therapeutic applications throughout the body, including particularly intravascular procedures such as in the peripheral vasculature (e.g., deep venous thrombosis), central vasculature (pulmonary embolism), and coronary vasculature, as well as procedures in other hollow organs or tubular structures in the body.

### Implementation Details

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the invention should not be taken to imply that the terminology is being re-defined herein to be restricted to including any specific characteristics of the features or aspects of the technology with which that terminology is associated.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Headings are included herein for reference and to aid in locating various sections. These headings are not intended to limit the scope of the concepts described with respect thereto. Such concepts may have applicability throughout the entire specification.

Many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

It will also be understood that, when a feature or element (for example, a structural feature or element) is referred to as being “connected”, “attached” or “coupled” to another feature or element, it may be directly connected, attached, or coupled to the other feature or element or intervening features or elements may be present. In contrast, when a feature or element is referred to as being “directly connected,” “directly attached” or “directly coupled” to another feature or element, there may be no intervening features or elements present. Although described or shown with respect to one embodiment, the features and elements so described or shown may apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments and implementations only and is not intended to be limiting. For example, as used herein, the singular forms “a,” “an” and “the” may be intended to include the plural forms as well, unless the context clearly indicates otherwise. It will be further understood that the terms “comprises” and/or “comprising,” when used in this specification, specify the presence of stated features, steps, operations, processes, functions, elements, and/or components, but do not preclude the presence or addition of one or more other features, steps, operations, processes, functions, elements, components, and/or groups thereof. As used herein, the term “and/or” includes any and all combinations of one or more of the associated listed items and may be abbreviated as “/.”

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

Spatially relative terms, such as “forward”, “rearward”, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features due to the inverted state. Thus, the term “under” may encompass both an orientation of over and under, depending on the point of reference or orientation. The device may be otherwise oriented (rotated 90 degrees or at other orientations) and the spatially relative descriptors used herein interpreted accordingly. Similarly, the terms “upwardly,” “downwardly,” “vertical,” “horizontal” and the like may be used herein for the purpose of explanation only unless specifically indicated otherwise.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing numeric values of magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise.

For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, may represent endpoints or starting points, and ranges for any combination of the data points. For example, if a particular data point “10” and a particular data point “15” may be disclosed, it is understood that greater than, greater than or equal to, less than, less than or equal to, and equal to 10 and 15 may be considered disclosed as well as between 10 and 15. It is also understood that each unit between two particular units may be also disclosed. For example, if 10 and 15 may be disclosed, then 11, 12, 13, and 14 may be also disclosed.

Although various illustrative embodiments have been disclosed, any of a number of changes may be made to various embodiments without departing from the teachings herein. For example, the order in which various described method steps are performed may be changed or reconfigured in different or alternative embodiments, and in other embodiments one or more method steps may be skipped altogether. Optional or desirable features of various device and system embodiments may be included in some embodiments and not in others. Therefore, the foregoing description is provided primarily for the purpose of example and should not be interpreted to limit the scope of the claims and specific embodiments or particular details or features disclosed.

What is claimed is:

1. A system for delivering fluid to a target site of a patient's vasculature, the system comprising:
a first catheter coupled to a first hub, the first catheter including a proximal end, a distal end, and a lumen;
a second catheter coupled to a second hub, the second catheter including a proximal end, a distal end, and a lumen, the second catheter configured to be positioned in the lumen of the first catheter;
a guiding element coupled to a third hub, the guiding element having a distal end and a proximal end, the guiding element configured to be positioned in the lumen of the second catheter, and
a contrast pump in fluid communication with the lumens of the first and second catheters, the system configured to actuate the contrast pump to selectively provide contrast media to the first or second catheter at an operating pressure to achieve a flow rate of at least about 1 mL/second out of the distal end of the selected catheter, the first catheter, second catheter, and guiding element configured such that
contrast media provided to the first catheter, while the second catheter or the guiding element is positioned at least partially in the lumen of the first catheter, and
contrast media provided to the second catheter, while the guiding element is positioned at least partially in the lumen of the second catheter.

2. The system of claim 1, wherein the flow rate is at least about 2 mL/second per second.

3. The system of claim 1, wherein the flow rate is at least about 3 mL/second per second.

4. The system of claim 1, wherein the first catheter is a procedure catheter, the second catheter is an insert catheter, and the guiding element is a guidewire.

5. The system of claim 1, wherein the first catheter and the second catheter are configured with dimensions such that when the second catheter is positioned in the lumen of the first catheter an effective cross-sectional area for fluid communication, between an exterior surface of the second catheter and an interior surface of the first catheter, is greater than or equal to about 0.001 square inches.

6. The system of claim 1, wherein the first catheter and the guiding element are configured such that when the guiding element is positioned in the lumen of the first catheter an effective cross-sectional area for fluid communication, between an exterior surface of the guiding element and an interior surface of the first catheter, is greater than or equal to about 0.001 square inches.

7. The system of claim 1, wherein the second catheter and the guiding element are configured such that when the guiding element is positioned in the lumen of the second catheter an effective cross-sectional area for fluid communication, between an exterior surface of the guiding element and an interior surface of the second catheter, is greater than or equal to about 0.001 square inches.

8. The system of claim 1, wherein the second catheter is an insert catheter with an inner diameter of between about 0.035″ and about 0.055″.

9. The system of claim 1, wherein the operating pressure is based at least in part on viscosity of the contrast media and on an effective cross-sectional area of the lumen of the catheter receiving the contrast media.

10. The system of claim 9, wherein the operating pressure is less than or equal to about 400 PSI when the effective cross-sectional area is greater than or equal to about 0.001 square inches.

11. A method for delivering fluid to a target site of a patient's vasculature, the method comprising:
selecting, from a concentric stack of a first catheter, a second catheter, and a guiding element, one of the first catheter or the second catheter to inject contrast media,
the first catheter having a proximal end, a distal end, and a lumen defined by an interior surface,
the second catheter having a proximal end, a distal end, an exterior surface, and a lumen defined by an interior surface, the second catheter configured to be positioned in the lumen of the first catheter, and
a guiding element having a distal end, a proximal end, and an exterior surface, the guiding element configured to be positioned in the lumen of the second catheter;
providing contrast media from a contrast pump at an operating pressure to
the proximal end of the first catheter to achieve a flow rate of at least about 1 mL/second out of the distal end of the first catheter while the second catheter or the guiding element is positioned at least partially in the lumen of the first catheter, if the first catheter is the selected catheter, and
the proximal end of the second catheter to achieve a flow rate of at least about 1 mL/second out of the distal end of the second catheter while the guiding element is positioned at least partially in the lumen of the second catheter, if the selected catheter is the second catheter.

12. The method of claim 11, wherein the effective cross-sectional area of the selected catheter is greater than or equal to about 0.001 square inches.

13. A system for delivering a fluid to a target site of a patient's vasculature, the system comprising:
a fluidics system having a contrast pump and a plurality of contrast fluid channels to provide contrast to a plurality of catheters;
a concentric stack of elongated devices having two or more catheters;
a controller configured to selectively cause any one or more of the two or more catheters in the concentric stack to be in fluid communication with the contrast fluid channels, and actuate the contrast pump to provide contrast to a selected catheter of any one or more of the two or more catheters while a lumen of the selected catheter contains a catheter or a guide wire of the concentric stack.

14. The system of claim 21, wherein the lumen of the selected catheter, and the catheter or guide wire in the lumen of the selected catheter, are sized so the lumen of the selected catheter has an effective cross-sectional area of about 0.001 square inches or higher.

15. The system of claim 22, wherein the contrast pump is configured to provide contrast at a pressure above about 300 PSI.

16. A method for delivering a fluid to a target site of a patient's vasculature using a robotic catheter system, the method comprising:
moving a distal end of a concentric stack of elongated devices towards the target site, the concentric stack of elongated devices including two or more catheters;
selectively causing, by a controller, any one or more of the two or more catheters in the concentric stack to be in fluid communication with contrast fluid channels of a fluidics system; and
actuating a high pressure contrast pump of the fluidics system to selectively provide contrast to a lumen of any one or more of the two or more catheters while the lumen contains a catheter or a guide wire of the concentric stack of elongated devices such that contrast flows out of a distal end of the lumen at 1 mL/second or greater.

17. The method of claim 16, wherein the flow rate is at least about 2 mL/second per second.

18. The method of claim 16, wherein the lumen that contains a catheter or a guidewire of the concentric stack of elongated devices has an effective cross-sectional area for fluid communication of greater than or equal to about 0.001 square inches.

19. The method of claim 16, wherein actuating a high pressure contrast pump includes operating the contrast pump to provide contrast at a pressure of greater than 250 PSI.

20. The method of claim 16, wherein actuating a high pressure contrast pump includes operating the contrast pump to provide contrast at a pressure of less than or equal to about 400 PSI.
